SEQUENCE LISTING (10 sequences)

SEQ ID NO: 1 - DNA, 1761 bp, Homo sapiens
  source 1..1761: mol_type = other DNA
gacgctcacaagtccgaagtcgctcacagattcaaggacttgggtgaagaaaacttcaaggctttggtcttgatcgctttcgctcaatacttgcaacaatgtccattcgaagatcacgtcaagttggtcaacgaagttaccgaattcgctaagacttgtgttgctgacgaatccgcggaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggctagctccgcaaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccctcgagaagtgttgtgctgctgctgacccacacgaatgttacgctaaggttttcgatgaattcaagccattggtcgaagaaccacaaaacttgatcaagcaaaactgtgaattgttcgaacaattgggtgaatacaagttccaaaacgctttgttggttagatacactaagaaggtcccacaagtctccaccccaactttggttgaagtctctagaaacttgggtaaggtcggttctaagtgttgtaagcacccagaagctaagagaatgccatgtgctgaagattacttgtccgtcgttttgaaccaattgtgtgttttgcacgaaaagaccccagtctctgatagagtcaccaagtgttgtactgaatctttggttaacagaagaccatgtttctctgctttggaagtcgacgaaacttacgttccaaaggaattcaacgctgaaactttcaccttccacgctgatatctgtaccttgtccgaaaaggaaagacaaattaagaagcaaactgctttggttgaattggtcaagcacaagccaaaggctactaaggaacaattgaaggctgtcatggatgatttcgctgctttcgttgaaaagtgttgtaaggctgatgataaggaaacttgtttcgctgaagaaggtaagaagttggtcgctgcttcccaagctgccttaggtttgtaataa

SEQ ID NO: 2 - protein, 585 aa, Homo sapiens [sequence omitted]

SEQ ID NO: 3 - protein, 608 aa, Mus musculus
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 4 - protein, 608 aa, Rattus norvegicus
MKWVTFLLLLFISGSAFSRGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAEIEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 5 - protein, 608 aa, Macaca mulatta
MKWVTFISLLFLFSSAYSRGVFRRDTHKSEVAHRFKDLGEEHFKGLVLVAFSQYLQQCPFEEHVKLVNEVTEFAKTCVADESAENCDKSLHTLFGDKLCTVATLRETYGEMADCCAKQEPERNECFLQHKDDNPNLPPLVRPEVDVMCTAFHDNEATFLKKYLYEVARRHPYFYAPELLFFAARYKAAFAECCQAADKAACLLPKLDELRDEGKASSAKQRLKCASLQKFGDRAFKAWAVARLSQKFPKAEFAEVSKLVTDLTKVHTECCHGDLLECADDRADLAKYMCENQDSISSKLKECCDKPLLEKSHCLAEVENDEMPADLPSLAADYVESKDVCKNYAEAKDVFLGMFLYEYARRHPDYSVMLLLRLAKAYEATLEKCCAAADPHECYAKVFDEFQPLVEEPQNLVKQNCELFEQLGEYKFQNALLVRYTKKVPQVSTPTLVEVSRNLGKVGAKCCKLPEAKRMPCAEDYLSVVLNRLCVLHEKTPVSEKVTKCCTESLVNRRPCFSALELDEAYVPKAFNAETFTFHADMCTLSEKEKQVKKQTALVELVKHKPKATKEQLKGVMDNFAAFVEKCCKADDKEACFAEEGPKFVAASQAALA

SEQ ID NO: 6 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLASSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 7 - protein, 607 aa, Sus scrofa
MKWVTFISLLFLFSSAYSRGVFRRDTYKSEIAHRFKDLGEQYFKGLVLIAFSQHLQQCPYEEHVKLVREVTEFAKTCVADESAENCDKSIHTLFGDKLCAIPSLREHYGDLADCCEKEEPERNECFLQHKNDNPDIPKLKPDPVALCADFQEDEQKFWGKYLYEIARRHPYFYAPELLYYAIIYKDVFSECCQAADKAACLLPKIEHLREKVLTSAAKQRLKCASIQKFGERAFKAWSLARLSQRFPKADFTEISKIVTDLAKVHKECCHGDLLECADDRADLAKYICENQDTISTKLKECCDKPLLEKSHCIAEAKRDELPADLNPLEHDFVEDKEVCKNYKEAKHVFLGTFLYEYSRRHPDYSVSLLLRIAKIYEATLEDCCAKEDPPACYATVFDKFQPLVDEPKNLIKQNCELFEKLGEYGFQNALIVRYTKKVPQVSTPTLVEVARKLGLVGSRCCKRPEEERLSCAEDYLSLVLNRLCVLHEKTPVSEKVTKCCTESLVNRRPCFSALTPDETYKPKEFVEGTFTFHADLCTLPEDEKQIKKQTALVELLKHKPHATEEQLRTVLGNFAAFVQKCCAAPDHEACFAVEGPKFVIEIRGILA

SEQ ID NO: 8 - protein, 607 aa, Equus caballus
MKWVTFVSLLFLFSSAYSRGVLRRDTHKSEIAHRFNDLGEKHFKGLVLVAFSQYLQQCPFEDHVKLVNEVTEFAKKCAADESAENCDKSLHTLFGDKLCTVATLRATYGELADCCEKQEPERNECFLTHKDDHPNLPKLKPEPDAQCAAFQEDPDKFLGKYLYEVARRHPYFYGPELLFHAEEYKADFTECCPADDKLACLIPKLDALKERILLSSAKERLKCSSFQNFGERAVKAWSVARLSQKFPKADFAEVSKIVTDLTKVHKECCHGDLLECADDRADLAKYICEHQDSISGKLKACCDKPLLQKSHCIAEVKEDDLPSDLPALAADFAEDKEICKHYKDAKDVFLGTFLYEYSRRHPDYSVSLLLRIAKTYEATLEKCCAEADPPACYRTVFDQFTPLVEEPKSLVKKNCDLFEEVGEYDFQNALIVRYTKKAPQVSTPTLVEIGRTLGKVGSRCCKLPESERLPCSENHLALALNRLCVLHEKTPVSEKITKCCTDSLAERRPCFSALELDEGYVPKEFKAETFTFHADICTLPEDEKQIKKQSALAELVKHKPKATKEQLKTVLGNFSAFVAKCCGREDKEACFAEEGPKLVASSQLALA

SEQ ID NO: 9 - protein, 608 aa, Oryctolagus cuniculus
MKWVTFISLLFLFSSAYSRGVFRREAHKSEIAHRFNDVGEEHFIGLVLITFSQYLQKCPYEEHAKLVKEVTDLAKACVADESAANCDKSLHDIFGDKICALPSLRDTYGDVADCCEKKEPERNECFLHHKDDKPDLPPFARPEADVLCKAFHDDEKAFFGHYLYEVARRHPYFYAPELLYYAQKYKAILTECCEAADKGACLTPKLDALEGKSLISAAQERLRCASIQKFGDRAYKAWALVRLSQRFPKADFTDISKIVTDLTKVHKECCHGDLLECADDRADLAKYMCEHQETISSHLKECCDKPILEKAHCIYGLHNDETPAGLPAVAEEFVEDKDVCKNYEEAKDLFLGKFLYEYSRRHPDYSVVLLLRLGKAYEATLKKCCATDDPHACYAKVLDEFQPLVDEPKNLVKQNCELYEQLGDYNFQNALLVRYTKKVPQVSTPTLVEISRSLGKVGSKCCKHPEAERLPCVEDYLSVVLNRLCVLHEKTPVSEKVTKCCSESLVDRRPCFSALGPDETYVPKEFNAETFTFHADICTLPETERKIKKQTALVELVKHKPHATNDQLKTVVGEFTALLDKCCSAEDKEACFAVEGPKLVESSKATLG

SEQ ID NO: 10 - protein, 609 aa, Homo sapiens [sequence omitted]

(19) United States
(12) Patent Application Publication
(10) Pub. No.: US 2026/0240766 A1
(43) Pub. Date: Aug. 20, 2026

(54) FORMULATIONS AND USES THEREOF

(71) Applicant: Sartorius Albumedix Limited, Nottingham (GB)

(72) Inventors: Philip Harvey Morton, Nottingham (GB); Neil Dodsworth, Nottingham (GB); Eleonora Cerasoli, Nottingham (GB); Jason Cameron, Nottingham (GB)

(21) Appl. No.: 19/472,337

(22) PCT Filed: Apr. 5, 2024
(86) PCT No.: PCT/EP2024/059382

§ 371 Date: Oct. 3, 2025

(30) Foreign Application Priority Data
Apr. 5, 2023 (EP) 23166867.4

(51) Int. Cl. A61K 9/06; A61K 39/00; A61K 39/12; A61K 47/02; A61K 47/10; A61K 47/14; A61K 47/18; A61K 47/22; A61K 47/26; A61K 47/42; A61K 48/00; C12N 7/00

(52) U.S. Cl. CPC A61K 9/06; A61K 39/12; A61K 47/02; A61K 47/10; A61K 47/14; A61K 47/183; A61K 47/22; A61K 47/26; A61K 47/42; A61K 48/0041; C12N 7/00; A61K 2039/5252; A61K 2039/5254; A61K 2039/545; C12N 2750/14151

(57) ABSTRACT

The invention provides formulations of albumin with desirably high levels of free thiol and desirably low levels of albumin polymer. The invention further provides uses of a formulation comprising: (i) albumin, wherein the albumin is in a substantially unaggregated form; and (ii) an anion having multiple negative charges housed on a flexible backbone, to stabilise a virus. The invention also provides a formulation and a kit of parts comprising (i) albumin, wherein the albumin is in a substantially unaggregated form; and (ii) an anion having multiple negative charges housed on a flexible backbone and its use to stabilise a virus.

This application contains a Sequence Listing in computer readable form. The computer readable form is incorporated herein by reference.

This specification is in two parts, named herein as Part A and Part B.

### PART A

### PART A—REFERENCE TO THE SEQUENCE LISTING

This application contains a Sequence Listing in computer readable form. The computer readable form is incorporated herein by reference.

### PART A—FIELD OF THE INVENTION

The present invention relates to formulations of albumin with desirable high levels of free thiol and desirable low levels of albumin polymer and uses thereof.

### PART A—BACKGROUND OF THE INVENTION

Human serum albumin (HSA, SEQ ID NO: 2) contains 1 free thiol at the cysteine residue located at position 34 (Cys34). This thiol conveys much of the anti-oxidant properties of albumin. The thiol provided by Cys34 is also available for binding and conjugation to other molecules.

These other molecules, in vivo, include cysteine and glutathione, but could also include molecules such as drugs (chemicals or peptides with therapeutic applications). However, molecules may have relatively short stability either in formulation or when administered to patients. It is desirable to increase the efficacy and stability of molecules such as therapeutics and consequently to reduce the dose and frequency of dosing required by patients. Pharmaceutical companies have been investigating technologies to improve the stability and to prolong the efficacy of drugs. Technologies include different formulations, packages and storage conditions of a drug or modifying the drug molecule itself by linking it to another molecule e.g. PEGylation. However, not all of these approaches are viable e.g. due to cost, safety, reproducibility and immunogenicity concerns.

Linking a drug to a larger molecule such as albumin, e.g. via conjugation to the thiol provided by Cys34, has been shown to improve the stability of the drug (e.g. Holmes et al. (2000) Bioconjug. Chem. 11, 439-444). An advantage of using wild-type albumin is that it is a nature identical molecule with a proven record of safety and consistency. The theoretical free thiol level, due to the unpaired cysteine at position 34, is 1 free thiol per albumin molecule. However, during prolonged storage, the free thiol on wild-type albumin may be substantially decreased by oxidation and polymer levels may increase due to aggregation.

Maintaining a high level of free thiol is desirable to maximize the efficiency of conjugation of a drug to albumin. Maintaining a low level of polymer is desirable to minimize the likelihood of adverse immunogenic reactions in subjects receiving the albumin or a product containing the albumin. Polymer levels have been addressed by formulating albumin with, for example, fatty acids or amino acids (Anraku et al. (2004) Biochimica et Biophysica Acta. 1702, 9-17). However, when investigating the effect of fatty acid on albumin, the present inventors have observed that increasing levels of fatty acid results in a desirable decrease in polymer level, however it is accompanied by an undesirable decrease in the level of free thiol.

To date, an albumin formulation having both a high level of free thiol and a low level of polymer following storage is not available. Such a formulation would be desirable, for example, for use in conjugating albumin to conjugation partners such as drugs.

### PART A—SUMMARY OF THE INVENTION

The invention provides a liquid formulation of albumin or fragment or fusion thereof comprising at least 0.75 moles free thiol per mole albumin and at most 1% (w/w) albumin polymer after storage or incubation at at least 40° C. for at least 1 month.

The invention also provides a method of preparing a formulation of an albumin or fragment or fusion thereof having a high free thiol level and low polymer level, the method comprising formulating the albumin or fragment or fusion thereof in a buffer comprising from 2.5 to 7.5 mM fatty acid, at least 175 mM cation and a pH from 5.5 to 6.5.

The invention further provides use of a buffer as described herein to maintain a high free thiol level and low polymer level in an albumin formulation, for example following storage or incubation of the albumin formulation.

The invention also provides a method of making a conjugate comprising conjugating albumin or fragment or fusion thereof with a conjugation partner

The invention further provides a method of conjugating albumin or fragment or fusion thereof to a partner at at least 75% efficiency relative to albumin, the method comprising contacting a partner with a formulation of albumin or fragment or fusion thereof according to the first, second, third, or fourth aspect of the invention.

The invention also provides use of formulation of albumin or fragment or fusion thereof as described herein for high efficiency conjugation to a conjugation partner.

The invention further provides a conjugate comprising albumin and a conjugation partner.

The invention also provides for use of a formulation of albumin of fragment or fusion thereof as described herein, or produced by a method as described herein, to increase the half-life of a molecule.

The invention further provides a conjugate as described herein for treatment of disease, treatment of illness and/or for diagnosis.

The invention also provides a conjugate as described herein for the manufacture of a medicament for the treatment of disease, treatment of illness and/or for diagnosis.

### PART A—BRIEF DESCRIPTION OF THE FIGURES

FIG. 1 shows (A) the design space (no shading) for the sodium (Na, mM) and octanoate (Oct, mM) concentration for an albumin formulation having a free thiol level of at least 0.75 moles free thiol per mole albumin and a polymer level of at most 1% (w/w) at a set pH of 6.0 following incubation at 40° C. for 1 month, with the solid shaded area showing the design space having a free thiol level lower than 0.75 moles free thiol per mole albumin, the diagonal shaded area showing the design space having a polymer level greater than 1% (w/w) and the intersection of the vertical and horizontal lines showing a formulation having 250 mM Na and 5 mM octanoate; (B) is a 3-dimensional representation of the relationship between octanoate, sodium and thiol levels at a set pH of 6.0 following incubation at 40° C. for 1 month and (C) is a 3-dimensional representation of the relationship between octanoate, sodium and polymer levels at a set pH of 6.0 following incubation at 40° C. for 1 month.

FIG. 2 shows (A) the design space (no shading) for the pH and octanoate (Oct, mM) concentration for an albumin formulation having a free thiol level of at least 0.75 moles free thiol per mole albumin and a polymer level of at most 1% (w/w) at a set sodium (Na) concentration of 250 mM following incubation at 40° C. for 1 month, with the solid shaded area showing the design space having a free thiol level lower than 0.75 moles free thiol per mole albumin, the diagonal shaded area showing the design space having a polymer level greater than 1% (w/w) and the intersection of the vertical and horizontal lines showing a formulation having pH 6.0 and 5 mM octanoate; (B) is a 3-dimensional representation of the relationship between octanoate, pH and thiol levels at a set sodium concentration of 250 mM following incubation at 40° C. for 1 month and (C) is a 3-dimensional representation of the relationship between octanoate, pH and polymer levels at a set sodium concentration of 250 mM following incubation at 40° C. for 1 month.

FIG. 3 shows (A) the design space (no shading) for the sodium (Na, mM) concentration and pH for an albumin formulation having a free thiol level of at least 0.75 moles free thiol per mole albumin and a polymer level of at most 1% (w/w) at a set octanoate concentration of 5 mM following incubation at 40° C. for 1 month, with the solid shaded area showing the design space having a free thiol level lower than 0.75 moles free thiol per mole albumin, the diagonal shaded area showing the design space having a polymer level greater than 1% (w/w) and the intersection of the vertical and horizontal lines showing a formulation having 250 mM Na and pH 6.0; (B) is a 3-dimensional representation of the relationship between pH, sodium and thiol levels at a set octanoate concentration of 5 mM following incubation at 40° C. for 1 month and (C) is a 3-dimensional representation of the relationship between pH, sodium and polymer levels at a set octanoate concentration of 5 mM following incubation at 40° C. for 1 month.

### PART A—DEFINITIONS

The term “albumin” means a protein having the same and/or very similar tertiary structure as human serum albumin (HSA) or one or more (e.g. several) HSA domains and has similar properties of HSA or the relevant domains. Similar tertiary structures are for example the structures of the albumins from the species mentioned herein. Some of the major properties of albumin are i) its ability to regulate plasma volume or osmotic pressure, ii) a long plasma half-life of around 19 days±5 days, iii) ligand-binding, e.g. binding of endogenous molecules such as acidic, lipophilic compounds including bilirubin, fatty acids, hemin and thyroxine (see also Table 1 of Kragh-Hansen et al, 2002, Biol. Pharm. Bull. 25, 695, hereby incorporated by reference), iv) binding of small organic compounds with acidic or electronegative features e.g. drugs such as warfarin, diazepam, ibuprofen and paclitaxel (see also Table 1 of Kragh-Hansen et al, 2002, Biol. Pharm. Bull. 25, 695, hereby incorporated by reference). Not all of these properties need to be fulfilled to in order to characterize a protein or fragment as an albumin. If a fragment, for example, does not comprise a domain responsible for binding of certain ligands or organic compounds the variant of such a fragment will not be expected to have these properties either. The term albumin includes variants, and/or derivatives such as fusions and/or fragments of an albumin or of an albumin variant.

The term “albumin fusion” means a genetic fusion of albumin (or a variant or fragment thereof) and a non-albumin protein or peptide. The non-albumin protein or peptide may be a therapeutic, prophylactic, or diagnostic protein or peptide. Examples of albumin fusions are provided in EP624195, WO 2001/079271, WO 2003/059934, WO 2003/060071, WO 2011051489, WO 2011/124718 and EP11164955 (each incorporated herein by reference in their entirety).

The term “conjugate” means an albumin (or a variant or fragment or fusion thereof) to which a non-albumin moiety is chemically conjugated. The non-albumin moiety may be a therapeutic, prophylactic, or diagnostic protein. Examples of albumin conjugations are provided in WO 2011/124718 and EP11164955 (incorporated herein by reference in their entirety).

The term “fragment” means a polypeptide having one or more (several) amino acids deleted from the amino and/or carboxyl terminus of a mature polypeptide and/or from an internal region of albumin. Fragments may consist of one uninterrupted sequence derived from albumin or may comprise two or more sequences derived from different parts of the albumin.

The fragments according to the invention have a size of more than approximately 20 amino acid residues, preferably more than 30 amino acid residues, more preferred more than 40 amino acid residues, more preferred more than 50 amino acid residues, more preferred more than 75 amino acid residues, more preferred more than 100 amino acid residues, more preferred more than 200 amino acid residues, more preferred more than 300 amino acid residues, even more preferred more than 400 amino acid residues and most preferred more than 500 amino acid residues. In a preferred embodiment a fragment corresponds to one or more of the albumin domains. Preferred albumin domains of the invention are domains having at least 70, 75, 80, 85, 90, 95, 96, 97, 98, 99, 99.5% or 100% identity to HSA domain I consisting of amino acid residues 1 to 194±1 to 15 amino acids of SEQ ID NO: 2; at least 70, 75, 80, 85, 90, 95, 96, 97, 98, 99, 99.5% or 100% identity to HSA domain II consisting of amino acid residues 192 to 387±1 to 15 amino acids of SEQ ID NO: 2 and at least 70, 75, 80, 85, 90, 95, 96, 97, 98, 99, 99.5% or 100% identity to HSA domain III consisting of amino acid residues 381 to 585±1 to 15 amino acids of SEQ ID NO: 2 or a combination of one or more (several) of these domains, e.g. domain I and II, domain II and Ill or domain I and III fused together. No generally accepted convention for the exact borders of the albumin domains exists and the overlap in the above mentioned ranges and the allowance of a varying length of plus or minus 1, 2, 3, 4, 5, 6, 7, 8, 9, 10, 11, 12, 13, 14 or 15 from amino acids, preferably from 1 to 15 amino acids, more preferably from 1 to 10 amino acids, most preferably from 1 to 5 amino acids, at the N-terminal and/or C-terminal of the domains, allowing for a total variance in length of up to 30 amino acids, preferably up to 20 amino acids, more preferably up to 10 amino acids for each domain reflects this fact and that there may be some diverging opinions on the amino acid residues in the border between the domains belongs to one or the other domain. For the same reason it may be possible to find references to the amino acid residues of albumin domains that diverge from the numbers above, however, the skilled person will appreciate how to identify the albumin domains based on the teaching in the literature and the teaching above. Corresponding domains of non-human albumins can be identified by alignment with HSA using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 3.0.0 or later. The optional parameters used are gap open penalty of 10, gap extension penalty of 0.5, and the EBLOSUM62 (EMBOSS version of BLOSUM62) substitution matrix. Alternative alignment tools can also be used, for example MUSCLE as described herein. The domains may also be defined according to Dockal or Kjeldsen: Dockal et al (The Journal of Biological Chemistry, 1999, Vol. 274(41): 29303-29310) defines the domains of HSA as: Domain I: amino acids 1 to 197, Domain II: amino acids 189 to 385 of SEQ ID NO:2, Domain III: amino acids 381 to 585 of SEQ ID NO:2. Kjeldsen et al (Protein Expression and Purification, 1998, Vol 13:163-169) defines the domains as: Domain I: amino acids 1 to 192, Domain II: amino acids 193 to 382, Domain III: amino acids 383 to 585.

Therefore, in this invention, the following domain definitions are preferred. The amino acid numbers correspond to those of SEQ ID NO: 2 (HSA). However, using these numbers, the skilled person can identify corresponding domains in other albumin sequences. Domain I may or may not start at amino acid 1 and may or may not end at any of amino acids 192, 193, 194, 195, 196 or 197, preferably any of amino acids 192, 194 or 197. Domain II may or may not start at amino acid 189, 190, 191, 192 or 193, preferably any of amino acids 189, 192 or 193, and may or may not end at amino acid 382, 383, 384, 385, 386 or 387, preferably any of amino acids 382, 385 or 387. Domain III may or may not start at amino acid 381, 382 or 383, preferably amino acid 381 or 383, and may or may not end at amino acid 585. Domains in non-human albumins may have the same or different amino acid lengths and/or residue numbers as HSA. For example, a multiple alignment or pair-wise alignment may be prepared using HSA and one or more (several) other albumins, fragments, derivatives, variants and/or fusions in order to identify domains corresponding to domains 1, 2 and/or 3 of HSA.

Each domain is itself made up of two homologous subdomains namely 1-105, 120-194, 195-291, 316-387, 388-491 and 512-585, with flexible inter-subdomain linker regions comprising residues Lys106 to Glu119, Glu292 to Val315 and Glu492 to Ala511.

The term “free thiol” means a thiol group which is available for reaction with another group. For example, a free thiol is not already connected to another thiol group via a disulphide bond or is not oxidized.

The term “parent” or “parent albumin” means an albumin to which an alteration is made to produce the albumin variants of the present invention. The parent may be a naturally occurring (wild-type) polypeptide or an allele thereof. In a preferred embodiment the parent albumin is a wild-type albumin, more preferably a wild-type albumin from Homo sapiens (e.g. UNIPROT: P02768.2, SEQ ID NO: 10). Preferably the wild-type albumin from Homo sapiens is the mature form of the albumin such as SEQ ID NO: 2. The mature form of the albumin sequence may be encoded by the nucleotide sequence SEQ ID NO: 1. Alternative wild-type albumins can be selected from primate serum albumin, such as Orangutan serum albumin (UNIPROT: Q5NVH5.2), chimpanzee serum albumin (NCBI: XP_517233), and macaque serum albumin (UNIPROT: Q28522.1), or from rodent serum albumin such as rabbit serum albumin (UNIPROT: P49065.2), guinea pig serum albumin (UNIPROT: Q6WDN9), hamster serum albumin (UNIPROT: A6YF56), mouse serum albumin (UNIPROT: P07724.3) and rat serum albumin (UNIPROT: P02770.2), or from serum albumin from hoofed animals such as bovine serum albumin (UNIPROT: P02769.4), horse serum albumin (UNIPROT: P35747.1), donkey serum albumin (UNIPROT: Q5XLE4.1), goat serum albumin (GENBANK: ACF1039.1), sheep serum albumin (UNIPROT: P14639.1), and pig serum albumin (UNIPROT: P08835.2), or from dog serum albumin (NCBI NP_001003026), or from chicken serum albumin (UNIPROT: P19121.2) or from any of the other albumin species indicated in the table above. Preferably the parent albumin is a mature albumin. In another embodiment the parent albumin is at least 70%, more preferably 75%, more preferably at least 80%, more preferably at least 85%, even more preferably at least 90%, most preferably at least 95%, at least 96%, at least 97%, at least 98%, at least 99% at least 99.5% or at least 99.8% identical to SEQ ID NO: 2, and maintains at least one of the major properties of albumin or a similar tertiary structure as albumin, such as HSA. It is preferred if the parent albumin is a mature albumin, for example one without the leader sequence.

The term “partner” in the context of “fusion partner” means a non-albumin moiety that can be genetically fused to the albumin or fragment or fusion thereof, and in the context of “conjugation partner” means a non-albumin moiety that can be chemically conjugated to the albumin or fragment or fusion thereof. A conjugation partner may or may not be bioactive. A conjugation partner may or may not be a therapeutic compound. A conjugation partner may or may not be a radiopharmaceutical. A conjugation partner may or may not be an imaging agent.

The term “polymer” means a high molecular weight form of, or aggregate of albumin, which elutes in the void volume of a gel permeation HPLC column which separates molecules in the molecular weight range 10000 to 500000 Da, e.g. a TSK G3000SWxL. Polymer does not include dimer or trimer.

The term “thiol” means a carbon-bonded sulfhydryl (—C—SH or R-SH, where R may be selected from the group consisting of an alkane, alkene or other carbon-containing group).

The term “variant” means a polypeptide derived from a parent albumin comprising an alteration, i.e., a substitution, insertion, and/or deletion of an or more than one amino acids, at one or more (e.g. several) positions. A substitution means a replacement of an amino acid occupying a position with a different amino acid; a deletion means removal of an amino acid occupying a position; and an insertion means adding 1-3 amino acids adjacent to an amino acid occupying a position. The altered polypeptide (variant) can be obtained through human intervention by modification of the polynucleotide sequence encoding the parent albumin. The variant albumin is preferably at least 70%, preferably at least 75%, more preferably at least 80%, more preferably at least 85%, even more preferably at least 90%, most preferably at least 95%, at least 96%, at least 97%, at least 98%, at least 99%, or 100% identical to SEQ ID NO: 2 and maintains at least one (e.g. several) of the major properties of the parent albumin or a similar tertiary structure as HSA. For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows: (Identical Residues×100)/(Length of Alignment−Total Number of Gaps in Alignment).

The variant may possess altered binding affinity to the neonatal Fc receptor (FcRn) and/or an altered rate of transcytosis across endothelia, epithelia and/or mesothelia mono cell-layer when compared to the parent albumin. The variant albumin sequence is preferably one which is not found in nature. The variant may be as described in WO 2010/092135, WO 2011/051489, WO 2011/124718, WO 2012/059486, WO 2012/150319, WO 2013/135896, WO2014/072481, WO 2014/125082 or WO 2015/036579.

The term “wild-type” (WT) albumin means an albumin having the same amino acid sequence as an albumin naturally found in an animal or in a human being. Human serum albumin (HSA, SEQ ID NO:1 for cDNA or SEQ ID NO: 2 for amino acid sequence) is an example of a wild-type albumin and is from Homo sapiens. The “wild-type” (WT) human albumin (HSA) sequence is given by GenBank Accession number AAA98797.1 (Minghetti, P. P. et al “Molecular structure of the human albumin gene is revealed by nucleotide sequence within q11-22 of chromosome 4”, J. Biol. Chem. 261 (15), 6747-6757 (1986)).

### PART A—DETAILED DESCRIPTION OF THE INVENTION

A first aspect of the invention provides a liquid formulation of albumin or fragment or fusion thereof, the formulation comprising at least 0.75 moles free thiol per mole albumin and/or at most 1% (w/w) albumin polymer. The liquid formulation may comprise at least 0.80, 0.85, 0.90, 0.95, 0.96, 0.97, 0.98 or 0.99 moles free thiol per mole albumin or fragment or fusion thereof. The liquid formulation may comprise 1 mole free thiol per mole albumin or fragment or fusion thereof.

The albumin may be sourced from serum or a recombinant source. Advantageously, the liquid formulation may comprise a recombinant albumin. That is, the albumin may be sourced from a recombinant organism such as a recombinant microorganism, recombinant plant or recombinant animal. Since some users prefer animal-free ingredients, it is more preferred that the albumin is sourced from a non-animal recombinant source, such as a recombinant microorganism or recombinant plant. Preferred organisms include prokaryotes and, more preferably, eukaryotes such as animals, plants, fungi or yeasts, for example, but not limited to, the following species in which albumins have been successfully expressed as recombinant proteins, for example see the following citations which are incorporated herein by reference:
fungi (including but not limited to Aspergillus (WO06066595), Kluyveromyces (Fleer 1991, Bio/technology 9, 968-975), Pichia (Kobayashi 1998 Therapeutic Apheresis 2, 257-262) and Saccharomyces (Sleep 1990, Bio/technology 8, 42-46))
animals (Barash 1993, Transgenic Research 2, 266-276)
plants (including but not limited to potato and tobacco (Sijmons 1990, Bio/technology 8, 217 and Farran 2002, Transgenic Research 11, 337-346) and rice e.g. Oryza sativa)
mammalian cells such as CHO and HEK
prokaryotes (Pandjaitab 2000, J. Allergy Clin. Immunol. 105, 279-285)) e.g. E. coli (EP73,646).

Preferably the albumin is recombinant sourced from a fungal host, preferably from a genus selected from Saccharomyces (e.g. Saccharomyces cerevisiae) or Pichia (e.g. Pichia pastoris), more preferably a Saccharomyces, most preferably Saccharomyces cerevisiae. The albumin expressed by the host may comprise a leader sequence, for example the albumin may comprise SEQ ID NO: 10 or variant or fragment or fusion thereof as describe herein. The first nineteen residues of SEQ ID NO: 10 correspond to a signal sequence, the following five residues correspond to a propeptide and the following 585 residues correspond to the mature peptide according to SEQ ID NO: 2.

The albumin or fragment thereof may comprise one or more (e.g. several) free thiol groups (“thiols”) per molecule of albumin or fragment thereof, for example at least 1, 2, 3, 4, 5, 6, 7, 8, 9 or 10 free thiol groups. For an albumin, or fragment or fusion thereof, comprising one free thiol group per molecule of albumin or fragment or fusion thereof, such as human serum albumin which contains a free thiol provided by Cys34, the theoretical free thiol content is 1 mole free thiol per mole albumin or fragment or fusion thereof. For an albumin, or fragment or fusion thereof, comprising two free thiol groups per molecule of albumin or fragment thereof, such as a variant of human serum albumin which contains a free thiol provided by Cys34 and an additional free thiol provided by another Cys residue, the theoretical free thiol content is 2 moles free thiol per mole albumin or fragment thereof. Preferably, the liquid formulation comprises at least 75% of the theoretical free thiol level, more preferably at least 80%, 85%, 90%, 95%, 96%, 97%, 98% or 99% of the theoretical free thiol level. Consequently, for an albumin or fragment or fusion thereof containing 2 free thiols per molecule of albumin or fragment thereof, a free thiol level of at least 75% of the theoretical free thiol level is 0.75*2, i.e. an average (mean) of at least 1.5 moles of free thiol per mole of albumin or fragment or fusion thereof. The liquid formulation may comprise 100% of the theoretical free thiol level.

Preferably the desirable free thiol level, per mole albumin or fragment or fusion thereof, is observed after storage or incubation at at least 25° C. for at least 1 month, more preferably for at least 2, 3, 4, 5, 6, 9 or 12 months. More preferably, the free thiol level, per mole albumin or fragment thereof, is observed after storage or incubation at at least 40° C. for at least 1 month, more preferably for at least 2, 3, 4, 5 or 6 months. Preferably the free thiol level, per mole albumin or fragment thereof, is observed after storage or incubation at a temperature of from 2 to 8° C., such as from 2, 3, 4, 5, 6, or 7 to 3, 4, 5, 6, 7, or 8° C., for example at about 4° C., for a duration of at least 1 month, more preferably for at least 2, 3, 4, 5 or 6 months or for at least 1, 2, 3, 4, 5, or 6 years.

The free thiol level may be measured by a free thiol assay such as a colourimetric free thiol assay, for example a DTNB assay or a DTDP assay. A DTDP assay is preferred.

A suitable DTDP assay comprises the following method, the volumes of components may be adjusted, the ratio of the components should remain as set out below:
a) mixing albumin (50 μL (microliters) 50 mg·mL⁻¹ albumin) and buffer (950 μL (microliters), e.g. 0.1M sodium phosphate, 1 mM EDTA, pH 7.4) to make an albumin ‘sample’, using 0.65 mM glutathione and 950 μL (microliters) buffer as a control (e.g. see Example 1),
b) measuring the absorbance of the sample, the control and a buffer ‘blank’ at a wavelength of 324 nm,
c) adding 50 μL (microliters) 4 mM 4,4′-Dithiodipyridine (DTDP) to the sample, the control and the buffer ‘blank’,
d) incubating for 10 minutes at room temperature (15 to 25° C., 20° C. is preferred),
e) measuring the increase in absorbance at a wavelength of 324 nm of the albumin sample, the control and the buffer ‘blank’, and
f) determining the free thiol level of the sample.

A suitable DTNB assay comprises the following method, the volumes of components may be adjusted, the ratio of the components should remain as set out below:
a) mixing albumin (80 μL (microliters) 50 mg·mL⁻¹ albumin) and buffer (920 μL (microliters), e.g. 0.1M TRIS-HCl, 0.01M EDTA, pH 8.0) to make an albumin ‘sample’, using 80 μL (microliters) 0.65 mM glutathione and 920 μL (microliters) buffer as a control (e.g. see Example 1)
b) measuring the absorbance of the sample, the control and a buffer ‘blank’ at a wavelength of 412 nm,
c) adding 50 μL (microliters) 0.01M 5,5′-Dithiobis-(2-Nitrobenzoate) (DTNB) to the sample, the control and the buffer ‘blank’,
d) incubating for 10 minutes at room temperature (15 to 25° C., 20° C. is preferred), and
e) measuring the increase in absorbance at a wavelength of 412 nm of the sample, the control and the buffer ‘blank’, and
f) determining the free thiol level of the sample.

Preferably at least 70%, more preferably, at least 72, 74, 76, 78, 80, 82, 84, 85, 86, 88, 90, 92, 94, 85, 96, 97, 98, 99% of the free thiol level observed at TO (time point zero e.g. measured within 24 or 48 hours of formulation) is maintained after storage or incubation at at least 25° C. for at least 1 month, more preferably for at least 2, 3, 4, 5 or 6 months or for at least 1, 2, 3, 4, 5, or 6 years. Preferably at least 70%, more preferably, at least 72, 74, 76, 78, 80, 82, 84, 85, 86, 88, 90, 92, 94, 85, 96, 97, 98, 99% of the free thiol level observed at TO (e.g. measured within 24 or 48 hours of formulation) is maintained after storage or incubation at at least 40° C. for at least 1 month more preferably for at least 2, 3, 4, 5 or 6 months. Preferably at least 70%, more preferably, at least 72, 74, 76, 78, 80, 82, 84, 85, 86, 88, 90, 92, 94, 85, 96, 97, 98, 99% of the free thiol level observed at TO (e.g. measured within 24 or 48 hours of formulation) is maintained after storage or incubation at a temperature of from 2 to 8° C., such as from 2, 3, 4, 5, 6, or 7 to 3, 4, 5, 6, 7, or 8° C., for example at about 4° C. for at least 1 month, more preferably for at least 2, 3, 4, 5 or 6 months or for at least 1, 2, 3, 4, 5, or 6 years.

Preferably, the formulation contains at most 1% polymer, more preferable at most 0.9, 0.8, 0.7, 0.6, 0.5, 0.4, 0.3, 0.2, 0.1 or 0% polymer. Preferably the at most 1%, 0.9, 0.8, 0.7, 0.6, 0.5, 0.4, 0.3, 0.2, 0.1 or 0% polymer level is observed at TO (e.g. measured within 24 or 48 hours of formulation). Preferably the at most 1%, 0.9, 0.8, 0.7, 0.6, 0.5, 0.4, 0.3, 0.2, 0.1 or 0% polymer level is observed after storage or incubation at at least 25° C. for at least 1 month, more preferably at at least 40° C. for at least 1 month. Preferably the polymer level following incubation at 25° C. or 40° C. for 2, 3, 4, 5 or 6 months is at most 5, 4, 3, or preferably at most 2-fold that observed after incubation at the same temperature for 1 month. For example, the polymer level following incubation at 40° C. for 3 months is at most 4-fold that observed after incubation at the same temperature for 1 month. Preferably at most 1% polymer, more preferable at most 0.9, 0.8, 0.7, 0.6, 0.5, 0.4, 0.3, 0.2, 0.1 or 0% polymer observed at TO (e.g. measured within 24 or 48 hours of formulation) is maintained after storage or incubation at a temperature of from 2 to 8° C., such as from 2, 3, 4, 5, 6, or 7 to 3, 4, 5, 6, 7, or 8° C., for example at about 4° C., for a duration of at least 1 month, more preferably for at least 2, 3, 4, 5 or 6 months or for at least 1, 2, 3, 4, 5, or 6 years. By 1% polymer, we mean that 1% of the albumin is present in polymer form. Polymer levels may be measured by chromatography e.g. gel permeation high-performance liquid chromatography (GP.HPLC), for example using albumin at 5 mg·mL⁻¹.

A preferred liquid formulation according to the first aspect of the invention comprises from 2.5 to 7.5 mM fatty acid, such as a fatty acid selected from C₆ fatty acids or larger, such as C₆, C₈, or C₁₀ fatty acid, preferably C₇ fatty acids or larger, most preferred C₈ fatty acid (octanoate). The fatty acid concentration may be from about 2.5, 2.75, 3, 3.25, 3.5, 3.75, 4, 4.25, 4.5, 4.75, 5, 5.25, 5.5, 5.75, 6, 6.25, 6.5, 6.75, 7 or 7.25 to about 2.75, 3, 3.25, 3.5, 3.75, 4, 4.25, 4.5, 4.75, 5, 5.25, 5.5, 5.75, 6, 6.25, 6.5, 6.75, 7, 7.25 or 7.5 mM. A fatty acid concentration of from about 3 mM to about 7 mM, such as from about 4 mM to about 6 mM is preferred, particularly about 5 mM.

A preferred liquid formulation according to the first aspect of the invention comprises a cation concentration of at least 175 mM, such as a cation concentration from about 175, 200, 225, 250, 275, or 300 mM to about 200, 225, 250, 275, 300, 350, 400, 450, 500, 600, 700, 800, 900 or 1000 mM. A preferred cation concentration is from about 200 to about 300 mM, particularly from about 225, 230, 235, 240, 245, 250, 255, 260, 265, or 270 to about 230, 235, 240, 245, 250, 255, 260, 265, 270 or 275 mM, particularly about 250 mM.

The cations of the composition may be provided by any cation and may be provided by one or more (e.g. several) classes or species as described below. For example, the cations may be either mono or bivalent, monoatomic or polyatomic and may be provided by one or more (e.g. several) of an alkali metal (such as sodium, potassium), an alkaline earth metal (such as calcium, magnesium) or ammonium. It is preferred that the cations are provided by sodium and/or potassium and/or magnesium, most preferably sodium or magnesium.

Cations may be provided by a salt of an inorganic acid (e.g. a group 1 or 2 metal or ammonium salt such as sodium chloride), a salt of a divalent acid (e.g. a group 1 or group 2 metal or ammonium sulphate or phosphate such as sodium sulphate) or a salt of an organic acid (e.g. a group 1 or group 2 metal or ammonium salt of acetate or citrate such as sodium acetate). For the liquid formulation of albumin of the invention, sodium chloride is a preferred source of cation.

Cations and anions used to stabilise the albumin may be provided by (i) salts and/or (ii) pH buffers such as described herein. Therefore, there may be more than one (e.g. several) species of cation or anion, such as 2 or 3 species. There may be more than one (e.g. several) source of a single cation, for example Na which may be provided by both a pH buffer (such as sodium phosphate) and a salt (such as NaCl).

Anions useful to the invention include inorganic anions such as phosphate, and halides such as chloride, and organic anions such as acetate and citrate. Anions may be either mono or bivalent, monoatomic or polyatomic. Preferred anions include sulphate, acetate phosphate and chloride, particularly chloride, sulphate and acetate.

Therefore, the composition may comprise one or more (e.g. several) of an alkali metal phosphate or chloride (such as sodium phosphate, potassium phosphate, sodium chloride or potassium chloride), an alkaline earth metal phosphate (such as calcium phosphate, magnesium phosphate, calcium chloride, magnesium chloride) or ammonium phosphate or ammonium chloride.

A preferred liquid formulation according to the first aspect of the invention has a pH of from about 5.5, 5.6, 5.7, 5.8, 5.9, 6.0, 6.1, 6.2, 6.3, or 6.4 to about 5.6, 5.7, 5.8, 5.9, 6.0, 6.1, 6.2, 6.3, 6.4 or 6.5. A preferred pH is from about 5.7 to about 6.2, more preferred about 6.0. Particularly preferred liquid formulations have a formulation which lies within a design space defined by both of the following equations:
(a) Free thiol=1.1786−0.05167*pH−0.0001544*cation−0.01133*FA+(pH−6)*((cation−198.3)*0.0002659)+(pH−6)*((FA−7.125)*0.003160)+(cation−198.3)*((FA−7.125)*0.000001935)+(pH−6)*((pH−6)*−0.0500)+(cation−198.3)*((cation−198.3)*0.000002583)+(FA−7.125)*((FA−7.125)*0.0001413)+(pH−6)*((cation−198.3)*((FA−7.125)*−0.000009879)), and
(b) Polymer=9.1677−0.8417*pH−0.01044*cation−0.2690*FA+(pH−6)*((cation−198.3)*−0.004796)+(pH−6)*((FA−7.125)*0.06523)+(cation−198.3)*((FA−7.125)*0.001494)+(pH−6)*((pH−6)*2.25)+(cation−198.3)*((cation−198.3)*0.00006768)+(FA−7.125)*((FA−7.125)*0.02490)+(pH−6)*((cation−198.3)*((FA−7.125)*0.001237));
where
   ‘free thiol’ means the level of free thiol with 1.0 meaning 1 mole thiol per mole albumin
   ‘cation’ means the cation concentration in mM (preferably sodium)
   ‘FA’ means the fatty acid concentration in mM (preferably octanoate)

The albumin, or fragment thereof, may or may not be genetically fused to a partner. An advantage of an albumin fusion according to the present invention is that a first partner can be genetically fused to the albumin or fragment thereof and a second partner may be chemically conjugated to the albumin or fragment or fusion thereof. The first and second partner may be different moieties or copies of the same moiety. Examples of partners are disclosed below the ninth aspect of the invention.

The free thiol, or thiols, of the albumin or fragment or fusion thereof may be provided by one or more (e.g. several) cysteine residue(s) at one or more (e.g. several) positions in albumin or fragment or fusion thereof. For example, a free thiol may be provided by a cysteine at a position corresponding to one or more (e.g. several) of positions selected from 34, 1, 2, 4, 38, 40, 48, 52, 55, 58, 60, 75, 76, 79, 80, 82, 83, 83, 86, 91, 104, 113, 115, 116, 121, 122, 124, 125, 129, 168, 169, 177, 229, 236, 266, 269, 270, 273, 283, 298, 300, 301, 303, 304, 308, 313, 314, 316, 318, 320, 321, 324, 325, 355, 360, 361, 364, 365, 368, 369, 371, 375, 379, 386, 390, 396, 397, 435, 439, 443, 471, 478, 479, 490, 496, 498, 501, 503, 504, 505, 506, 508, 512, 538, 541, 542, 546, 549, 550, 558, 560, 562, 564, 565, 566, 567, 573, 574, 577, 578, 580, 581, 582, 584, and 585 of SEQ ID NO: 2. Preferably, free thiol is provided by a cysteine at a position corresponding to position 34 of SEQ ID NO: 2.

Preferably, the albumin or fragment or fusion thereof has at least 70% sequence identity to a mammalian albumin, preferably from an albumin selected from the group consisting of human (SEQ ID NO: 2), mouse (SEQ ID NO: 3), rat (SEQ ID NO: 4), macaque (SEQ ID NO: 5), bovine (SEQ ID NO: 6), pig (SEQ ID NO: 7), horse (SEQ ID NO: 8), or rabbit (SEQ ID NO: 9) albumin. Preferably, the albumin or fragment thereof has at least 70% sequence identity to HSA (SEQ ID NO: 2), more preferably from 70, 75, 80, 85, 90, 91, 92, 93, 94, 95, 96, 97, 98, 99, 99.2, 99.4, 99.6, or 99.8 to 75, 80, 85, 90, 91, 92, 93, 94, 95, 96, 97, 98, 99, 99.2, 99.4, 99.6, 99.8 or 100% identity to HSA (SEQ ID NO: 2). For example, a preferred albumin or fragment or fusion thereof has, at most, 12, 11, 10, 9, 8, 7, 6, 5, 4, 3, 2 or 1 amino acid mutations relative to wt HSA (SEQ ID NO: 2) or fragment or fusion thereof.

A fusion of an albumin or fragment thereof comprises the albumin or fragment, thereof as described herein, genetically fused to a fusion partner. The fusion partner may in principle be any polypeptide but generally it is preferred that the fusion partner is a polypeptide having bioactive, therapeutic, prophylactic (including vaccine), radiopharmaceutical, diagnostic, imaging or other beneficial properties. Such properties may be referred to as ‘pharmaceutically beneficial properties’. Fusion polypeptides comprising albumin or fragments thereof are known in the art. It has been found that such fusion polypeptides comprising albumin or a fragment thereof and a fusion partner polypeptide have a longer plasma half-life compared to the unfused fusion partner polypeptide alone. The fusion may be one or more (e.g. several) of an N-terminal fusion (i.e. fusion partner genetically fused to the N-terminus of the albumin or fragment thereof), a C-terminal fusion (i.e. fusion partner genetically fused to the C-terminus of the albumin or fragment thereof), or an insertion within the sequence of the albumin or fragment thereof, such as insertion into a loop.

Preferably, the albumin fragment or fusion thereof comprises at least 175 contiguous amino acids from an albumin having at least 70% sequence identity to HSA (SEQ ID NO: 2), such as from 175, 200, 225, 250, 275, 300, 325, 350, 375, 400, 425, 450, 475, 500, 525, 550 or 575 to 200, 225, 250, 275, 300, 325, 350, 375, 400, 425, 450, 475, 500, 525, 550, 575 or 580 amino acids. A fragment may comprise, consist of or substantially correspond to one or more (e.g. several) domains of albumin such as HSA (SEQ ID NO: 2), or variant thereof, such as amino acids corresponding to Domain I (residues 1 to 194±1, 2, 3, 4, 5, 6, 7, 8, 9, 10, 11, 12, 13, 14 or 15 amino acids), Domain II (residues 192 to 387±1, 2, 3, 4, 5, 6, 7, 8, 9, 10, 11, 12, 13, 14 or 15 amino acids) or Domain III (residues 381 to 585±1, 2, 3, 4, 5, 6, 7, 8, 9, 10, 11, 12, 13, 14 or 15 amino acids).

The liquid formulation may comprise albumin or fragment or fusion thereof at from about 5 to about 30% (w/v), for example from about 5, 7.5, 10, 12.5, 15, 17.5, 20, 22.5, 25 or 27.5% to about 7.5, 10, 12.5, 15, 17.5, 20, 22.5, 25, 27.5 or 30% (w/v), most preferable about 10% or about 20%.

It is preferred that the liquid formulation comprises at least 50% albumin or fragment or fusion thereof, more preferably at least 60%, 70%, 75%, 80%, 85%, 90%, 95%, 96%, 97%, 98%, 99%, 99.5, 99.6, 99.7, 99.8, or 99.9% albumin or fragment or fusion thereof. Most preferably, the liquid formulation is substantially free, or completely free, of protein that is not an albumin, fragment or fusion thereof.

It is preferred that the liquid formulation is aqueous.

The liquid formulation of albumin or fragment or fusion thereof may or may not comprise an inorganic buffer. The inorganic buffer may or may not be selected from sodium phosphate, potassium phosphate, or an organic buffer such as sodium acetate or sodium citrate. The buffer may stabilise pH. Preferably the buffer is a pharmaceutically acceptable buffer. The liquid formulation of albumin or fragment or fusion thereof may or may not comprise a detergent such as polysorbate 80. For example, the liquid formulation may or may not comprise from 0, 0.001, 0.01, 0.1, 0.5, 1, 2, 3, 4, 5, 10, or 15 mg·L⁻¹ detergent to 0.001, 0.01, 0.1, 0.5, 1, 2, 3, 4, 5, 10, 15, or 20 mg·L⁻¹ detergent such as polysorbate 80. The liquid formulation may be substantially free, or completely free, of detergent such as polysorbate 80.

The liquid formulation of albumin or fragment or fusion thereof may or may not comprise amino acids (i.e. ‘free’ amino acids which are not in the albumin fragment, or fusion thereof), such as tryptophan e.g. N-acetyl tryptophan. For example, the liquid albumin formulation may or may not comprise from 0, 0.001, 0.005, 0.01, 0.1, 0.5, 1, 2, 3, or 4 mM free amino acids to 0.001, 0.005, 0.01, 0.1, 0.5, 1, 2, 3, 4, or 5 mM free amino acids such as tryptophan. The liquid albumin formulation may be substantially free, or completely free, of free amino acids such as tryptophan.

The liquid formulation of albumin or fragment or fusion thereof may or may or may not be substantially free, or completely free, of components other than the albumin, cation source and fatty acid.

The liquid formulation may be provided in a container such as a rigid or flexible vial, bottle or bag e.g. a BPC, bioprocess container. Suitable container volumes include from about 50 mL to about 10 000 mL, e.g. 50 mL, 1000 mL, 5000 mL and 10 000 mL. It is preferred that the container comprises one or more (e.g. several) inlets or outlets to allow filling of the container and/or dispensing from the container. The albumin composition may be sterilized, e.g. prior to or after being filled in the container. The liquid formulation may or may not be provided in unit dosage form.

A surprising advantage of the formulation of the first aspect of the invention is that it provides a high free thiol level while also providing a low polymer level, these features are desirable when conjugating a partner to albumin because the high free thiol level allows for good conjugation efficiency and the low polymer level minimizes the likelihood of adverse immunogenic response if delivered to a subject such as a patient. Therefore, the invention provides a liquid formulation for maximising free thiol level and minimizing polymer level of albumin.

A second aspect of the invention provides a method of preparing a formulation of an albumin or fragment or fusion thereof having a high free thiol level and low polymer level, the method comprising formulating the albumin or fragment or fusion thereof in a buffer comprising from 2.5 to 7.5 mM fatty acid, at least 175 mM cation and a pH from 5.5 to 6.5, and optionally filling the albumin or fragment or fusion thereof into a container. The formulation may maintain the high free thiol level and low polymer level following storage or incubation as described herein.

A third aspect of the invention provides use of a buffer as described herein to maintain a high free thiol level and low polymer level in an albumin formulation, for example following storage or incubation of the albumin formulation.

The options and preferences for the first aspect of the invention apply to the second and third aspects of the invention.

A fourth aspect of the invention provides a method of making a conjugate comprising conjugating albumin or fragment or fusion thereof with a conjugation partner, in which the albumin or fragment or fusion thereof is a formulation according to the first aspect of the invention or made by the method of the second aspect of the invention.

Optionally, the conjugation is via a linker.

Conjugation may be carried out by methods known in the art (for example those provided by Pierce, Thermo Fisher Scientific, Rockford, IL, USA in the 2012 edition of the “Thermo Scientific Crosslinking Technical Handbook” (document identifier: 1602163 10/12) which is available online at https://tools.lifetechnologies.com/content/sfs/brochures/1602163-Crosslinking-Reagents-Handbook.pdf). These include, but are not limited to incorporating or engineering a thiol reactive group into or onto the conjugation partner, for example by incorporating or engineering another free thiol present on the conjugation partner; or by incorporating or engineering a pyridyl disulphide group on the conjugation partner; or by incorporating or engineering an haloacetyl group on the bioactive compound or by incorporating or engineering a maleimide group on the conjugation partner, or by incorporating or engineering a thiosulfonate group on the conjugation partner, or by incorporating or engineering vinylsulfone group on the conjugation partner. For example, but not limited to, N-ethylmaleimide (NEM, Pierce), 2-amino-2′-aminoethanethiolsulfonate (Pierce), N-beta-maleimidopropionic acid (BMPA Pierce), methyl methane thiosulfonate (MMTS, Pierce), fluorescein-5-maleimide (Pierce), 5-iodoacetamido-fluorescein (5-IAF, Pierce) or N-[6-7-amino-4-methylcoumarin-3-acetamido) hexyl]-3′-[2′-pyridyldithio] propionamide (AMCA-HPDP, Pierce).

If the conjugation partner contains at least one (e.g. several) thiol group, then the conjugation partner may be cross-linked to the albumin mutein of the invention by methods known to the art such as, but not limited to, oxidation or by the use of cross-linking reagents such as, but not limited to, 1,4-Bis-maleimidibutane (BMB, Pierce); 1,4-Bis-maleimidyl-2,3-dihydroxybutane (BMDB, Pierce); Bis-maleimidohexane (BMH, Pierce), Bis-maleimidoethane (BMOE, Pierce); 1,8-Bis-Maleimidotriethyleneglycol (BM[PEO]3 Pierce); 1,11-Bis-Maleimidotetraethyleneglycol (BM[PEO]4 Pierce); 1,4-Di-[3′-(2′-pyridyldithio)-propionamido]butane (DPDPB, Pierce); dithio-bis-maleimidoethane (DTME Pierce); 1,6-Hexane-bis-vinylsulfone (HBVS, Pierce) and Tris-[2-maleimimidoethyl]amine (TMEA, Pierce).

If the conjugation partner does not contain a thiol reactive group then it may be modified to incorporate one or more (e.g. several) such groups by either chemical modification or genetic engineering by methods know to the art (Chapman, A. P. (2002) Adv. Drug Deliv. Rev., 54 531-545: Humphreys, D. P. et al. Protein Engineering, Design & Selection vol. 20 no. 5 pp. 227-234, 2007, incorporated herein by reference). While these two references describe methodologies to cross-link PEG to an engineered free thiol within an antibody or antibody fragment, the techniques may be used to cross-link a conjugation partner to an engineered free thiol within the albumin mutein of the invention. Alternatively the Drug Affinity Complex (DAC™) technology developed by ConjuChem Inc. (Montreal, Quebec, Canada, H2X 3Y8) may be used, e.g. as described in WO 2000/69902 (incorporated herein by reference). There are three parts of each DAC™ construct: 1) the drug component (the portion responsible for biologic activity); 2) a linker attached to the drug component, and 3) a reactive chemistry group at the opposite end of the linker, usually a soft electrophile selective for thiols; a maleimide is the most useful embodiment. Other applicable conjugation methods are described in WO 2007/071068 (incorporated herein by reference).

If the conjugation partner does not contain a thiol reactive group but does contain one or more (e.g. several) amino groups then it may be modified to incorporate one or more (e.g. several) thiol reactive groups by chemical modification by methods known to the art such as the use of cross-linking reagents such as, but not limited to, N-5-azido-2-nitrobenzoyloxysuccinimide (AMAS, Pierce), N-[beta-maleimidopropyloxy] succinimide ester (BMPS, Pierce), N-eta-maleimidocaproic acid (EMCA, Pierce), N-[eta-maleimidocaproyloxy]succinimide ester (EMCS, Pierce), N-[eta-maleimidocaproyloxy]sulfosuccinimide ester (sulfo-EMCS, Pierce), N-[gamma-maleimidobutyryloxy]succinimide ester (GMBS, Pierce), N-[gamma- maleimidobutyryloxy]sulfosuccinimide ester (sulfo-GMBS, Pierce), N-kappa- maleimidoundecanioic acid (KMUA, Pierce), N-[kappa- maleimidoundecanoyloxy]sulfosuccinimide ester (sulfo-KMUS, Pierce), m-maleimidobenzoyl-N-hydroxysuccinimide (MBS, Pierce), m-maleimidobenzoyl-N-hydroxysulfosuccinimide ester (sulfo-MBS, Pierce), N-succinimidyl S-acetylthio-acetate (SATA, Pierce), N-succinimidyl S-acetylthiopropionate (SATP, Pierce), succinimidyl 3-[bromoacetamido]propionate (SBAP, Pierce), N-succinimidyl iodoacetate (SIA, Pierce), N-succinimidyl [4-iodoacetyl]aminobenzoate (SIAB, Pierce), sulfosuccinimidyl [4-iodoacetyl]aminobenzoate (sulfo-SIAB, Pierce), succinimidyl [4-[N-maleimidomethyl]cyclohexane-1-carboxylate (SMCC, Pierce), sulfosuccinimidyl [4-[N-maleimidomethyl]cyclohexane-1-carboxylate (sulfo-SMCC, Pierce), succinimidyl-[4-[N-maleimidomethyl]cyclohexane-1-carboxy-[6-amidocaproate (LC-SMCC, Pierce), 4-succinimidyloxycarbonyl-methyl-alpha [2-pyridyldithio]toluene (SMPT, Pierce), sulfosuccinimidyl6-[alpha-methyl-alpha-(2-pyridyldithio) toluamido]hexanoate (sulfo-LC-SMPT, Pierce), succinimidyl 4-[p-maleimidophenyl]-butyrate (SMPB, Pierce), sulfosuccinimidyl 4-[p-maleimidophenyl]-butyrate (sulfo-SMPB, Pierce), succinimidyl-6-[(beta-maleimidopropionamido) hexanoate] (SMPH, Pierce), N-succinimidyl 3-[2-pyridyldithio]propionate (SPDP, Pierce), succinimidyl [3-(2-pyridyldithio) propionamido]hexanoate (LC-SPDP, Pierce), sulfosuccinimidyl [3′-(2-pyridyldithio) propionamido]hexanoate (sulfo-LC-SPDP, Pierce) and N-succinimidyl-[4-vinylsulfonyl]benzoate (SVSB Pierce). It may be advantageous to block certain amine residues as described by Kavimandan et al., (2006) Bioconjugate Chem. 17, 1376-1384 (incorporated herein by reference).

If the conjugation partner does not contain a thiol reactive group but does contain one or more (e.g. several) carbonyl (oxidised carbohydrate) groups then it can be modified to incorporate one or more (e.g. several) thiol reactive groups by chemical modification by methods known to the art such as the use of cross-linking reagents such as, but not limited to, (N-β-maleimidopropionic acid hydrazide (BMPH, Pierce) N-[eta-maleimidocaproic acid]hydrazide (EMCH, Pierce), 4-[N-maleimidomethyl]cyclohexane-1carboxylhydrazide·HCl·1/2 dioxane (MMCCH, Pierce), 3-maleimidophenyl boronic acid (MPBH, Pierce), N-[kappa-maleimidoundecanoic acid]hydrazide (KMUH, Pierce) and 3-[2-pyridyldithio]propionyl hydrazide (PDPH, Pierce).

If the conjugation partner does not contain a thiol reactive group but does contain one or more (e.g. several) hydroxyl groups then it may be modified to incorporate one or more (e.g. several) thiol reactive groups by chemical modification by methods known to the art such as the use of cross-linking reagents such as, but not limited to, N-[p-maleimidophenyl]isocyanate (PMPI, Pierce).

Optionally, the method comprises providing the conjugate in a container or filling the conjugate into a container e.g. as described above. Optionally the method comprises providing the conjugate in a unit dosage form.

A fifth aspect of the invention provides a method of conjugating albumin or fragment or fusion thereof to a partner at at least 75% efficiency relative to albumin, preferably at at least 80, 85, 90, 95, 96, 97, 98, 99% efficiency or 100% efficiency, the method comprising contacting a formulation of albumin or fragment or fusion thereof according to the first, second, third, or fourth aspect of the invention. Optionally the method of conjugation includes a linker.

A sixth aspect of the invention provides use of an albumin formulation according to according to the first aspect of the invention or made by the method of the second aspect of the invention for high efficiency conjugation to a conjugation partner. ‘High efficiency’ includes conjugation of at least 75, 80, 85, 90, 95, 96, 97, 98, 99% efficiency or 100% efficiency relative to the albumin component. For example, 75% efficiency means that at completion, 75% of the free thiols, e.g. in the albumin component, have been conjugated to partner moieties. The high level of free thiol in the formulation of albumin, fragment or fusion thereof enables this high efficiency conjugation.

A seventh aspect of the invention provides a conjugate comprising an albumin according to the first aspect of the invention, or prepared according to the second aspect of the invention, and a conjugation partner. The seventh aspect of the invention also provides a conjugate obtained or obtainable by the method of the fourth or fifth aspect of the invention.

A conjugation partner may be a bioactive compound such as a peptide, polypeptide, protein or small molecule pharmaceutical, radiopharmaceutical or imaging agent.

The conjugate may comprise 2 or more (several, for example 2, 3, 4, 5, 6, 7, 8, 9 or 10) conjugation partners which may each be different to each other and/or may be multiple copies of the same compound.

Preferably, each conjugation partner is linked to the albumin through a conjugation-competent cysteine residue of the albumin however conjugation partners may be linked by other means for example by a genetic fusion or covalent bonds to non-cysteine amino acids such as lysine, as described or the fourth aspect of the invention.

An eighth aspect of the invention provides for use of an albumin formulation as described herein, or produced by a method as described herein, to increase the half-life of a molecule such as a bioactive agent, therapeutic agent, radiopharmaceutical, an imaging agent, a diagnostic agent, a contrast agent or a therapeutic compound. Half-life may be increased by at least 10, 20, 30, 40, 50, 60, 70, 80, 90, 100, 150, 200% compared with the molecule which is not conjugated to albumin.

A ninth aspect of the invention provides a conjugate as described herein for treatment of disease, treatment of illness and/or for diagnosis.

A tenth aspect of the invention provides a conjugate as described herein for the manufacture of a medicament for the treatment of disease, treatment of illness and/or for diagnosis.

### Conjugation Partner

The term ‘conjugation partner’ includes bioactive agents, imaging agents, diagnostic agents, contrast agents, radiopharmaceuticals and therapeutic compounds such as chemotherapeutic drugs and radiopharmaceuticals.

### Imaging Agents, Diagnostic Compounds, Contrast Agents and Therapeutic Compounds

The use of diagnostic agents, imaging agents and biological “contrast” agents are well known to the art. A diagnostic agent is any pharmaceutical product used as part of a diagnostic test (i.e. together with the equipment and procedures that are needed to assess the test result). The diagnostic agent may be used in vivo, ex vivo or in vitro.

The ability of albumin to accumulate in damaged muscle fibres of dystrophic muscle has been well described. For example, a Gadolinium-DTPA-albumin conjugate may be used as a combined diagnostic and therapeutic tool to visualize and monitor, for example, dystrophic muscle by magnetic resonance imaging (MRI) and for the delivery of putative therapeutics bound to albumin for effective targeting to dystrophic muscle (Amthor et al. (2004) Neuromuscular Disorders 14912:791-796). Malignant tumours often show an increased uptake and metabolism of albumin. The use of gadolinium-albumin conjugate has also been described for improved imaging of malignant tumours and to determine by MRI tumours sensitive to a therapy with drug-conjugated albumin (Kiessling et al. (2002) Investigative Radiology 37(4): 93-198).

Current imaging agents often degrade quickly whilst longer-lasting agents are often toxic. The use of albumin conjugates may be especially useful to increase the half-life of imaging agents and would therefore permit imaging over an extended period of time. WO 2005/082423 (incorporated herein by reference) describes the use of serum albumin conjugated to fluorescent substances for imaging.

Albumin may be conjugated to two or more (e.g. several) molecules selected from bioactive agents, therapeutic agents, radiopharmaceuticals, imaging agents, diagnostic agents, therapeutic compounds and contrast agents.

Tumours (and muscle degeneration) show enhanced uptake of albumin (EPR: Enhanced Permeation and Retention). Albumin conjugates may be used for enhanced imaging, and also to assess whether tumours (or other tissues and organs) would be suitable for albumin conjugated drugs.

### Bioactive Compounds

The bioactive compound may be a therapeutic or diagnostic compound. The therapeutic compound may be a chemotherapy drug for use in cancer chemotherapy. It may be cytostatic or cytotoxic; it may be a tumor-inhibiting agent.

The bioactive compound may already contain a free thiol group, e.g. a polypeptide containing a cysteine residue with a free thiol group. Alternatively, the bioactive compound may be modified so as to contain a free thiol group. Thus, the amino acid sequence of a polypeptide may be altered so as to include a cysteine residue with a free thiol group, or the bioactive compound may be chemically derivatized to include a free thiol group.

The bioactive compound may be a polypeptide (protein), particularly a recombinant protein pharmaceutical. It may be a chemotherapy or radiotherapy drug used to treat cancers and other related diseases.

Albumin can be conjugated via the free thiol group, or groups if the albumin mutein of the invention contains more than one free thiol, to at least one (e.g. several) bioactive compound by methods know to the art. The bioactive compound includes but is not limited to, peptides, polypeptides or proteins (either natural, recombinant, or synthetic) (Debinski, (2002) Cancer Investigation 20, 801-809, O'Keefe and Draper et al., (1985) JBC 260, 932-937, Xia et al., (2000) J. Pharmacology Experimental Therapeutics 295, 594-600, Kavimandan et al., (2006) Bioconjugate Chem. 17, 1376-1384, Humphries, et al., (1994) J. Tissue Culture Methods 16, 239-242, Wenning et al., (1998) Biotech. Bioeng. 57, 484-496, Yazdi and Murphy, (1994) Cancer Research 54, 6387-6394, Weaver and Laske (2003) J. Neuro-Oncology 65, 3-13, Widera et al., (2003) Pharmaceutical Research 20, 1231-1238, Daniels, T. R. et al. (2006) Clinical Immunology 121, 159-176 and the references included therein); therapeutic and diagnostic drugs or compounds (Mishra et al., (2006) J. Drug Targeting 14, 45-53, Lim and Shen, (2004) Pharmaceutical Research 21, 1985-1992, Fritzer et al., (1996) Biochemical Pharmacology 51, 489-493, Lubgan and Jozwiak (2002) Cell. Mol. Biol. Lett. 7, 98, Daniels, T. R. et al. (2006) op. ci.t and the references included therein); high molecular weight complexes including but not limited to liposomes, viruses and nanoparticles (Mishra et al., (2006) J. Drug Targeting 14, 45-53, Daniels, T. R. et al. (2006) op. cit, and the references included therein); nucleic acids and radionuclides, including DNA, RNA (including siRNA) and their analogs (Lee et al., (2005) Arch. Pharm. Res. 28, 722-729, Huang et al., (2007) FASEB J. 21, 1117-1125, Daniels, T. R. et al. (2006) op. cit, and the references included therein) and devices (Humphries, et al., (1994) J. Tissue Culture Methods 16, 239-242 and the references included therein). Additionally the entity can itself be modified by methods known to the art.

### Therapeutic Compounds

Examples of therapeutic compounds include: 4-1BB ligand, 5-helix, A human C-C chemokine, A human L105 chemokine, A human L105 chemokine designated huL105_3., A monokine induced by gamma-interferon (MIG), A partial CXCR4B protein, A platelet basic protein (PBP), α1-antitrypsin, ACRP-30 Homologue; Complement Component C1q C, Adenoid-expressed chemokine (ADEC), aFGF; FGF-1, AGF, AGF Protein, albumin, an etoposide, angiostatin, Anthrax vaccine, Antibodies specific for collapsin, antistasin, Anti-TGF beta family antibodies, antithrombin III, APM-1; ACRP-30; Famoxin, apo-lipoprotein species, Arylsulfatase B, b57 Protein, BCMA, Beta-thromboglobulin protein (beta-TG), bFGF; FGF2, Blood coagulation factors, BMP Processing Enzyme Furin, BMP-10, BMP-12, BMP-15, BMP-17, BMP-18, BMP-2B, BMP-4, BMP-5, BMP-6, BMP-9, Bone Morphogenic Protein-2, calcitonin, Calpain-10a, Calpain-10b, Calpain-10c, Cancer Vaccine, Carboxypeptidase, C-C chemokine, MCP2, CCR5 variant, CCR7, CCR7, CD11a Mab, CD137; 4-1BB Receptor Protein, CD20 Mab, CD27, CD27L, CD30, CD30 ligand, CD33 immunotoxin, CD40, CD40L, CD52 Mab, Cerebus Protein, Chemokine Eotaxin., Chemokine hIL-8, Chemokine hMCP1, Chemokine hMCP1a, Chemokine hMCP1b, Chemokine hMCP2, Chemokine hMCP3, Chemokine hSDF1b, Chemokine MCP-4, chemokine TECK and TECK variant, Chemokine-like protein IL-8M1 Full-Length and Mature, Chemokine-like protein IL-8M10 Full-Length and Mature, Chemokine-like protein IL-8M3, Chemokine-like protein IL-8M8 Full-Length and Mature, Chemokine-like protein IL-8M9 Full-Length and Mature, Chemokine-like protein PF4-414 Full-Length and Mature, Chemokine-like protein PF4-426 Full-Length and Mature, Chemokine-like protein PF4-M2 Full-Length and Mature, Cholera vaccine, Chondromodulin-like protein, c-kit ligand; SCF; Mast cell growth factor; MGF; Fibrosarcoma-derived stem cell factor, CNTF and fragment thereof (such as CNTFAx15 (Axokine™)), coagulation factors in both pre and active forms, collagens, Complement C5 Mab, Connective tissue activating protein-III, CTAA16.88 Mab, CTAP-III, CTLA4-Ig, CTLA-8, CXC3, CXC3, CXCR3; CXC chemokine receptor 3, cyanovirin-N, Darbepoetin, designated exodus, designated huL105_7, DIL-40, Dnase, EDAR, EGF Receptor Mab, ENA-78, Endostatin, Eotaxin, Epithelial neutrophil activating protein-78, EPO receptor; EPOR, erythropoietin (EPO) and EPO mimics, Eutropin, Exodus protein, Factor IX, Factor VII, Factor VIII, Factor X and Factor XIII, FAS Ligand Inhibitory Protein (DcR3), FasL, FasL, FasL, FGF, FGF-12; Fibroblast growth factor homologous factor-1, FGF-15, FGF-16, FGF-18, FGF-3; INT-2, FGF-4; gelonin, HST-1; HBGF-4, FGF-5, FGF-6; Heparin binding secreted transforming factor-2, FGF-8, FGF-9; Glia activating factor, fibrinogen, flt-1, flt-3 ligand, Follicle stimulating hormone Alpha subunit, Follicle stimulating hormone Beta subunit, Follitropin, Fractalkine, myofibrillar protein Troponin I, FSH, Galactosidase, Galectin-4, G-CSF, GDF-1, Gene therapy, Glioma-derived growth factor, glucagon, glucagon-like peptides, Glucocerebrosidase, glucose oxidase, Glucosidase, Glycodelin-A; Progesterone-associated endometrial protein, GM-CSF, gonadotropin, Granulocyte chemotactic protein-2 (GCP-2), Granulocyte-macrophage colony stimulating factor, growth hormone, Growth related oncogene-alpha (GRO-alpha), Growth related oncogene-beta (GRO-beta), Growth related oncogene-gamma (GRO-gamma), hAPO-4; TROY, hCG, Hepatitus B surface Antigen, Hepatitus B Vaccine, HER2 Receptor Mab, hirudin, HIV gp120, HIV gp41, HIV Inhibitor Peptide, HIV Inhibitor Peptide, HIV Inhibitor Peptide, HIV protease inhibiting peptides, HIV-1 protease inhibitors, HPV vaccine, Human 6CKine protein, Human Act-2 protein, Human adipogenesis inhibitory factor, human B cell stimulating factor-2 receptor, Human beta-chemokine H1305 (MCP-2), Human C-C chemokine DGWCC, Human CC chemokine ELC protein, Human CC type chemokine interleukin C, Human CCC3 protein, Human CCF18 chemokine, Human CC-type chemokine protein designated SLC (secondary lymphoid chemokine), Human chemokine beta-8 short forms, Human chemokine C10, Human chemokine CC-2, Human chemokine CC-3, Human chemokine CCR-2, Human chemokine Ckbeta-7, Human chemokine ENA-78, Human chemokine eotaxin, Human chemokine GRO alpha, Human chemokine GROalpha, Human chemokine GRObeta, Human chemokine HCC-1, Human chemokine HCC-1, Human chemokine I-309, Human chemokine IP-10, Human chemokine L105_3, Human chemokine L105_7, Human chemokine MIG, Human chemokine MIG-beta protein, Human chemokine MIP-1alpha, Human chemokine MIP1beta, Human chemokine MIP-3alpha, Human chemokine MIP-3beta, Human chemokine PF4, Human chemokine protein 331D5, Human chemokine protein 61164, Human chemokine receptor CXCR3, Human chemokine SDF1alpha, Human chemokine SDF1beta, Human chemokine ZSIG-35, Human Chr19Kine protein, Human CKbeta-9, Human CKbeta-9, Human CX3C 111 amino acid chemokine, Human DNAX interleukin-40, Human DVic-1 C-C chemokine, Human EDIRF I protein sequence, Human EDIRF II protein sequence, Human eosinocyte CC type chemokine eotaxin, Human eosinophil-expressed chemokine (EEC), Human fast twitch skeletal muscle troponin C, Human fast twitch skeletal muscle troponin I, Human fast twitch skeletal muscle Troponin subunit C, Human fast twitch skeletal muscle Troponin subunit I Protein, Human fast twitch skeletal muscle Troponin subunit T, Human fast twitch skeletal muscle troponin T, Human foetal spleen expressed chemokine, FSEC, Human GM-CSF receptor, Human gro-alpha chemokine, Human gro-beta chemokine, Human gro-gamma chemokine, Human IL-16 protein, Human IL-1RD10 protein sequence, Human IL-1RD9, Human IL-5 receptor alpha chain, Human IL-6 receptor, Human IL-8 receptor protein hIL8RA, Human IL-8 receptor protein hIL8RB, Human IL-9 receptor protein, Human IL-9 receptor protein variant #3, Human IL-9 receptor protein variant fragment, Human IL-9 receptor protein variant fragment #3, Human interleukin 1 delta, Human Interleukin 10, Human Interleukin 10, Human interleukin 18, Human interleukin 18 derivatives, Human interleukin-1 beta precursor, Human interleukin-1 beta precursor, Human interleukin-1 receptor accessory protein, Human interleukin-1 receptor antagonist beta, Human interleukin-1 type-3 receptor, Human Interleukin-10 (precursor), Human Interleukin-10 (precursor), Human interleukin-11 receptor, Human interleukin-12 40 kD subunit, Human interleukin-12 beta-1 receptor, Human interleukin-12 beta-2 receptor, Human Interleukin-12 p35 protein, Human Interleukin-12 p40 protein, Human interleukin-12 receptor, Human interleukin-13 alpha receptor, Human interleukin-13 beta receptor, Human interleukin-15, Human interleukin-15 receptor from clone P1, Human interleukin-17 receptor, Human interleukin-18 protein (IL-18), Human interleukin-3, human interleukin-3 receptor, Human interleukin-3 variant, Human interleukin-4 receptor, Human interleukin-5, Human interleukin-6, Human interleukin-7, Human interleukin-7, Human interleukin-8 (IL-8), Human intracellular IL-1 receptor antagonist, Human IP-10 and HIV-1 gp120 hypervariable region fusion protein, Human IP-10 and human Muc-1 core epitope (VNT) fusion protein, human liver and activation regulated chemokine (LARC), Human Lkn-1 Full-Length and Mature protein, Human mammary associated chemokine (MACK) protein Full-Length and Mature, Human mature chemokine Ckbeta-7, Human mature gro-alpha, Human mature gro-gamma polypeptide used to treat sepsis, Human MCP-3 and human Muc-1 core epitope (VNT) fusion protein, Human MI10 protein, Human MI1A protein, Human monocyte chemoattractant factor hMCP-1, Human monocyte chemoattractant factor hMCP-3, Human monocyte chemotactic proprotein (MCPP) sequence, Human neurotactin chemokine like domain, Human non-ELR CXC chemokine H174, Human non-ELR CXC chemokine IP10, Human non-ELR CXC chemokine Mig, Human PAI-1 mutants, Human protein with IL-16 activity, Human protein with IL-16 activity, Human secondary lymphoid chemokine (SLC), Human SISD protein, Human STCP-1, Human stromal cell-derived chemokine, SDF-1, Human T cell mixed lymphocyte reaction expressed chemokine (TMEC), Human thymus and activation regulated cytokine (TARC), Human thymus expressed, Human TNF-alpha, Human TNF-alpha, Human TNF-beta (LT-alpha), Human type CC chemokine eotaxin 3 protein sequence, Human type II interleukin-1 receptor, Human wild-type interleukin-4 (hIL-4) protein, Human ZCHEMO-8 protein, Humanized Anti-VEGF Antibodies, and fragments thereof, Humanized Anti-VEGF Antibodies, and fragments thereof, Hyaluronidase, ICE 10 kD subunit, ICE 20 kD subunit, ICE 22 kD subunit, Iduronate-2-sulfatase, Iduronidase, IL-1 alpha, IL-1 beta, IL-1 inhibitor (IL-1i), IL-1 mature, IL-10 receptor, IL-11, IL-11, IL-12 p40 subunit, IL-13, IL-14, IL-15, IL-15 receptor, IL-17, IL-17 receptor, IL-17 receptor, IL-17 receptor, IL-19, IL-1i fragments, IL1-receptor antagonist, IL-21 (TIF), IL-3 containing fusion protein, IL-3 mutant proteins, IL-3 variants, IL-3 variants, IL-4, IL-4 mutein, IL-4 mutein Y124G, IL-4 mutein Y124X, IL-4 muteins, IL-5 receptor, IL-6, II-6 receptor, IL-7 receptor clone, IL-8 receptor, IL-9 mature protein variant (Met117 version), immunoglobulins or immunoglobulin-based molecules or fragment of either (e.ga Small Modular ImmunoPharmaceutical™ (“SMIP”) or dAb, Fab′ fragments, F(ab′) 2, scAb, scFv or scFv fragment), including but not limited to plasminogen, Influenza Vaccine, Inhibin alpha, Inhibin beta, insulin, insulin-like growth factor, Integrin Mab, inter-alpha trypsin inhibitor, inter-alpha trypsin inhibitor, Interferon gamma-inducible protein (IP-10), interferons (such as interferon alpha species and sub-species, interferon beta species and sub-species, interferon gamma species and sub-species), Interleukin 6, Interleukin 8 (IL-8) receptor, Interleukin 8 receptor B, Interleukin-1alpha, Interleukin-2 receptor associated protein p43, interleukin-3, interleukin-4 muteins, Interleukin-8 (IL-8) protein, interleukin-9, Interleukin-9 (IL-9) mature protein (Thr117 version), interleukins (such as IL10, IL11 and IL2), Japanese encephalitis vaccine, Kalikrein Inhibitor, Keratinocyte growth factor, Kunitz domain protein (such as aprotinin, amyloid precursor protein and those described in WO 03/066824, with or without albumin fusions), LACI, lactoferrin, Latent TGF-beta binding protein II, leptin, Liver expressed chemokine-1 (LVEC-1), Liver expressed chemokine-2 (LVEC-2), LT-alpha, LT-beta, Luteinization Hormone, Lyme Vaccine, Lymphotactin, Macrophage derived chemokine analogue MDC (n+1), Macrophage derived chemokine analogue MDC-eyfy, Macrophage derived chemokine analogue MDC-yl, Macrophage derived chemokine, MDC, Macrophage-derived chemokine (MDC), Maspin; Protease Inhibitor 5, MCP-1 receptor, MCP-1a, MCP-1b, MCP-3, MCP-4 receptor, M-CSF, Melanoma inhibiting protein, Membrane-bound proteins, Met117 human interleukin 9, MIP-3 alpha, MIP-3 beta, MIP-Gamma, MIRAP, Modified Rantes, monoclonal antibody, MP52, Mutant Interleukin 6 S176R, myofibrillar contractile protein Troponin I, Natriuretic Peptide, Nerve Growth Factor-beta, Nerve Growth Factor-beta2, Neuropilin-1, Neuropilin-2, Neurotactin, Neurotrophin-3, Neurotrophin-4, Neurotrophin-4a, Neurotrophin-4b, Neurotrophin-4c, Neurotrophin-4d, Neutrophil activating peptide-2 (NAP-2), NOGO-66 Receptor, NOGO-A, NOGO-B, NOGO-C, Novel beta-chemokine designated PTEC, N-terminal modified chemokine GroHEK/hSDF-1alpha, N-terminal modified chemokine GroHEK/hSDF-1beta, N-terminal modified chemokine met-hSDF-1 alpha, N-terminal modified chemokine met-hSDF-1 beta, OPGL, Osteogenic Protein-1; OP-1; BMP-7, Osteogenic Protein-2, OX40; ACT-4, OX40L, Oxytocin (Neurophysin I), parathyroid hormone, Patched, Patched-2, PDGF-D, Pertussis toxoid, Pituitary expressed chemokine (PGEC), Placental Growth Factor, Placental Growth Factor-2, Plasminogen Activator Inhibitor-1; PAI-1, Plasminogen Activator Inhibitor-2; PAI-2, Plasminogen Activator Inhibitor-2; PAI-2, Platelet derived growth factor, Platelet derived growth factor Bv-sis, Platelet derived growth factor precursor A, Platelet derived growth factor precursor B, Platelet Mab, platelet-derived endothelial cell growth factor (PD-ECGF), Platelet-Derived Growth Factor A chain, Platelet-Derived Growth Factor B chain, polypeptide used to treat sepsis, Preproapolipoprotein “milano” variant, Preproapolipoprotein “paris” variant, pre-thrombin, Primate CC chemokine “ILINCK”, Primate CXC chemokine “IBICK”, proinsulin, Prolactin, Prolactin2, prosaptide, Protease inhibitor peptides, Protein C, Protein S, pro-thrombin, prourokinase, RANTES, RANTES 8-68, RANTES 9-68, RANTES peptide, RANTES receptor, Recombinant interleukin-16, Resistin, restrictocin, Retroviral protease inhibitors, ricin, Rotavirus Vaccine, RSV Mab, saporin, sarcin, Secreted and Transmembrane polypeptides, Secreted and Transmembrane polypeptides, serum cholinesterase, serum protein (such as a blood clotting factor), Soluble BMP Receptor Kinase Protein-3, Soluble VEGF Receptor, Stem Cell Inhibitory Factor, Straphylococcus Vaccine, Stromal Derived Factor-1 alpha, Stromal Derived Factor-1 beta, Substance P (tachykinin), T1249 peptide, T20 peptide, T4 Endonuclease, TACI, Tarc, TGF-beta 1, TGF-beta 2, Thr117 human interleukin 9, thrombin, thrombopoietin, Thrombopoietin derivative1, Thrombopoietin derivative2, Thrombopoietin derivative3, Thrombopoietin derivative4, Thrombopoietin derivative5, Thrombopoietin derivative6, Thrombopoietin derivative7, Thymus expressed chemokine (TECK), Thyroid stimulating Hormone, tick anticoagulant peptide, Tim-1 protein, TNF-alpha precursor, TNF-R, TNF-RII; TNF p75 Receptor; Death Receptor, tPA, transferrin, transforming growth factor beta, Troponin peptides, Truncated monocyte chemotactic protein 2 (6-76), Truncated RANTES protein (3-68), tumour necrosis factor, Urate Oxidase, urokinase, Vasopressin (Neurophysin II), VEGF R-3; flt-4, VEGF Receptor; KDR; flk-1, VEGF-110, VEGF-121, VEGF-138, VEGF-145, VEGF-162, VEGF-165, VEGF-182, VEGF-189, VEGF-206, VEGF-D, VEGF-E; VEGF-X, von Willebrand's factor, Wild type monocyte chemotactic protein 2, Wild type monocyte chemotactic protein 2, ZTGF-beta 9.

### Chemotherapy Drugs

Examples of chemotherapy drugs include: 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Asparaginase, ATRA, Avastin®, Azacitidine, BCG, BCNU, Bevacizumab, Bexarotene, BEXXAR®, Bicalutamide, BICNU, Blenoxane®, Bleomycin, Bortezomib, Busulfan, Busulfex®, C225, Calcium Leucovorin, Campath®, Camptosar®, Camptothecin-11, Capecitabine, Carac™, Carboplatin, Carmustine, Carmustine Wafer, Casodex®, CC-5013, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin diftitox, DepoCyt™, Dexamethasone, Dexamethasone acetate, Dexamethasone Sodium Phosphate, Dexasone, Dexrazoxane, DHAD, DIC, Diodex, Docetaxel, Doxil®, Doxorubicin, Doxorubicin liposomal, Droxia™, DTIC, DTIC-Dome®, Duralone®, Efudex®, Eligard™, Ellence™, Eloxatin™, Elspar®, Emcyt®, Epirubicin, Epoetin alfa, Erbitux™, Erlotinib, Erwinia L-asparaginase, Estramustine, Ethyol, Etopophos®, Etoposide, Etoposide Phosphate, Eulexin®, Evista®, Exemestane, Fareston®, Faslodex®, Femara®, Filgrastim, Floxuridine, Fludara®, Fludarabine, Fluoroplex®, Fluorouracil, Fluoxymesterone, Flutamide, Folinic Acid, FUDR®, Fulvestrant, G-CSF, Gefitinib, Gemcitabine, Gemtuzumab ozogamicin, Gemzar®, Gleevec™, Gliadel® Wafer, GM-CSF, Goserelin, Granulocyte-Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Kidrolase®, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar® Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, Raloxifene, Revlimid®, Rheumatrex®, Rituxan®, Rituximab, Roferon-A® (Interferon Alfa-2a), Rubex®, Rubidomycin hydrochloride, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Tositumomab, Trastuzumab, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®.

### Radiopharmaceuticals

Examples of radiopharmaceuticals include: Carbon-11, Carbon-14, Chromium-51, Cobalt-57, Cobalt-58, Erbium-169, Fluorine-18, Gallium-67, Gold-198, Indium-111, Indium-113m, lodine-123, lodine-125, lodine-131, Iron-59, Krypton-81m, Nitrogen-13, Oxygen-15, Phosphorous-32, Rhenium-186, Rubidium-82, Samarium-153, Selenium-75, Strontium-89, Technetium-99m, Thallium-201, Tritium, Xenon-127, Xenon-133, Yttrium-90.

### Imaging Agents

Examples of imaging agents include: Gadolinium, magnetite, manganese, technetium, |125, 1131, P32, TI201, lopamidol, PET-FDG.

The conjugation partner as mentioned herein may be a peptide, organic chemical or small molecule pharmaceutical. The conjugation partner may be a bioactive, therapeutic, prophylactic (including vaccine), diagnostic, imaging, or radiopharmaceutical moiety or moiety having other beneficial properties.

Preferably the biological activity of the conjugation partner is maintained at at least 50, 60, 70, 80, 90, or 95% when conjugation to the albumin of the invention compared with the biological activity of the conjugation partner in a non-conjugated state. Preferably the biological activity of the conjugation partner is not reduced by conjugation to an albumin of the invention.

### PART A—PREFERRED EMBODIMENTS

1. A liquid formulation of albumin or fragment thereof comprising at least 0.75 moles free thiol per mole albumin or fragment thereof and at most 1% (w/w) albumin polymer after storage or incubation at at least 40° C. for at least 1, 2, 3, 4, 5 or 6 months.
2. A liquid formulation of albumin or fragment thereof comprising at least 0.75 moles free thiol per mole albumin or fragment thereof and at most 1% (w/w) albumin polymer after storage or incubation at at least 25° C. for at least 1, 2, 3, 4, 5, 6, 9 or 12 months.
3. A liquid formulation of albumin or fragment thereof comprising at least 0.75 moles free thiol per mole albumin and at most 1% (w/w) albumin polymer or fragment thereof after storage or incubation at a temperature of from 2 to 8° C., such as from 2, 3, 4, 5, 6, or 7 to 3, 4, 5, 6, 7, or 8° C., for example at about 4° C., for a duration of at least 1, 2, 3, 4, 5 or 6 months or at least 1, 2, 3, 4, 5 or 6 years.
4. The liquid formulation according to any preceding embodiment comprising at least 0.80, 0.85, 0.90, 0.95, 0.96, 0.97, 0.98 or 0.99 moles free thiol per mole albumin or fragment thereof.
5. The liquid formulation according to embodiment 4 comprising at least 1 mole free thiol per mole albumin or fragment thereof.
6. The liquid formulation according to any preceding embodiment comprising at least 75% of the theoretical free thiol level, more preferably at least 80%, 85%, 90%, 95%, 96%, 97%, 98% or 99% of the theoretical free thiol level.
7. The liquid formulation according to embodiment 6 comprising 100% of the theoretical free thiol level.
8. The liquid formulation according to any preceding embodiment comprising from about 2.5 to about 7.5 mM fatty acid.
9. The liquid formulation according to embodiment 8 comprising from about 2.5, 3, 3.5, 4, 4.5, 5, 5.5, 6, 6.5, 7, 7.5 to about 3, 3.5, 4, 4.5, 5, 5.5, 6, 6.5, 7 or 7.5 mM fatty acid.
10. The liquid formulation according to embodiment 9 comprising from about 4 to about 6 mM fatty acid.
11. The liquid formulation according to embodiment 10 comprising about 5 mM fatty acid.
12. The liquid formulation according to any preceding embodiment having a cation concentration of at least about 175 mM.
13. The liquid formulation according to embodiment 12 having a cation concentration of from, about 200, 225, 250, 275, or 300 mM to about 225, 250, 275, 300, 350, 400, 450, 500, 600, 700, 800, 900 or 1000 mM.
14. The liquid formulation according to embodiment 13 having a cation concentration of from, about 200 to about 300 mM.
15. The liquid formulation according to embodiment 14 having a cation concentration of from, about 225 to about 275 mM.
16. The liquid formulation according to any preceding embodiment having a pH of from about 5.5 to about 6.5.
17. The liquid formulation according to embodiment 16 having a pH from about 5.5, 5.6, 5.7, 5.8, 5.9, 6.0, 6.1, 6.2, 6.3, 6.4 to about 5.6, 5.7, 5.8, 5.9, 6.0, 6.1, 6.2, 6.3, 6.4, or 6.5.
18. The liquid formulation according to embodiment 17 having a pH from about 5.7 to about 6.2.
19. The liquid formulation according to embodiment 18 having a pH about 6.
20. A liquid formulation comprising albumin, fatty acid and cation, in which the formulation lies within a design space defined by both of the following equations:

$$Free thiol = 1.1786 - 0.05167 * pH - 0.0001544 * cation - 0.01133 * FA + ( pH - 6 ) * ( ( cation - 198.3 ) * 0.0002659 ) + ( pH - 6 ) * ( ( FA - 7.125 ) * 0.00316 ) + ( cation - 198.3 ) * ( ( FA - 7.125 ) * 0.000001935 ) + ( pH - 6 ) * ( ( pH - 6 ) * - 0.05 ) + ( cation - 198.3 ) * ( ( cation - 198.3 ) * 0.000002583 ) + ( FA - 7.125 ) * ( ( FA - 7.125 ) * 0.0001413 ) + ( pH - 6 ) * ( ( cation - 198.3 ) * ( ( FA - 7.125 ) * - 0.000009879 ) ) , and \qquad ( a )$$

$$Polymer = 9.1677 - 0.8417 * pH - 0.01044 * cation - 0.269 * FA + ( pH - 6 ) * ( ( cation - 198.3 ) * - 0.004796 ) + ( pH - 6 ) * ( ( FA - 7.125 ) * 0.06523 ) + ( cation - 198.3 ) * ( ( FA - 7.125 ) * 0.001494 ) + ( pH - 6 ) * ( ( pH - 6 ) * 2.25 ) + ( cation - 198.3 ) * ( ( cation - 198.3 ) * 0.00006768 ) + ( FA - 7.125 ) * ( ( FA - 7.125 ) * 0.0249 ) + ( pH - 6 ) * ( ( cation - 198.3 ) * ( ( FA - 7.125 ) * 0.001237 ) ) ; \qquad ( b )$$

where
   ‘free thiol’ means the level of free thiol
   ‘cation’ means the cation concentration in mM
   ‘FA’ means the fatty acid concentration in mM.
21. The formulation according to embodiment 20 comprising at least 0.75 moles free thiol per mole albumin or fragment thereof and at most 1% (w/w) albumin polymer, for example after storage or incubation at at least 40° C. for at least 1, 2, 3, 4, 5 or 6 months.
22. The liquid formulation according to any preceding embodiment, in which the formulation lies within a design space defined by both of the following equations:

$$Free thiol = 1.1786 - 0.05167 * pH - 0.0001544 * cation - 0.01133 * FA + ( pH - 6 ) * ( ( cation - 198.3 ) * 0.0002659 ) + ( pH - 6 ) * ( ( FA - 7.125 ) * 0.00316 ) + ( cation - 198.3 ) * ( ( FA - 7.125 ) * 0.000001935 ) + ( pH - 6 ) * ( ( pH - 6 ) * - 0.05 ) + ( cation - 198.3 ) * ( ( cation - 198.3 ) * 0.000002583 ) + ( FA - 7.125 ) * ( ( FA - 7.125 ) * 0.0001413 ) + ( pH - 6 ) * ( ( cation - 198.3 ) * ( ( FA - 7.125 ) * - 0.000009879 ) ) , and \qquad ( a )$$

$$Polymer = 9.1677 - 0.8417 * pH - 0.01044 * cation - 0.269 * FA + ( pH - 6 ) * ( ( cation - 198.3 ) * - 0.004796 ) + ( pH - 6 ) * ( ( FA - 7.125 ) * 0.06523 ) + ( cation - 198.3 ) * ( ( FA - 7.125 ) * 0.001494 ) + ( pH - 6 ) * ( ( pH - 6 ) * 2.25 ) + ( cation - 198.3 ) * ( ( cation - 198.3 ) * 0.00006768 ) + ( FA - 7.125 ) * ( ( FA - 7.125 ) * 0.0249 ) + ( pH - 6 ) * ( ( cation - 198.3 ) * ( ( FA - 7.125 ) * 0.001237 ) ) ; \qquad ( b )$$

where
   ‘free thiol’ means the level of free thiol
   ‘cation’ means the cation concentration in mM
   ‘FA’ means the fatty acid concentration in mM.
23. The liquid formulation according to any of embodiments 8 to 22 in which the fatty acid is selected from C₆ fatty acids or larger, such as C₆, C₈ or C₁₀ fatty acid, preferably C₇ fatty acids or larger, most preferred C₈ fatty acid (octanoate).
24. The liquid formulation according to any of embodiments 12 to 23 in which the cation is selected from sodium, potassium, calcium, magnesium and ammonium, preferably sodium.
25. The liquid formulation according to any preceding embodiment comprising from about 225 to about 275 mM cation, preferably sodium, and from 4 to 6 mM fatty acid, preferably octanoate, and having a pH from about 5.7 to about 6.2.
26. The liquid formulation according to embodiment 25 wherein the formulation comprises about 250 mM cation, preferably sodium, about 5 mM fatty acid, preferably octanoate, and about pH 6.0.
27. The liquid formulation according to any preceding embodiment in which the albumin or fragment thereof is fused to a partner.
28. The liquid formulation according to any preceding embodiment in which the albumin, fragment or fusion thereof comprises at least 1, 2, 3, 4, 5, 6, 7, 8, 9 or 10 free thiols.
29. The liquid formulation according to any preceding embodiment in which the free thiol or thiols are provided by a cysteine residue or residues at one or more (e.g. several) positions in albumin or fragment or fusion thereof.
30. The liquid formulation according to embodiment 29 in which the free thiol is provided by a cysteine at a position corresponding to one or more (e.g. several) of positions selected from 34, 1, 2, 4, 38, 40, 48, 52, 55, 58, 60, 75, 76, 79, 80, 82, 83, 83, 86, 91, 104, 113, 115, 116, 121, 122, 124, 125, 129, 168, 169, 177, 229, 236, 266, 269, 270, 273, 283, 298, 300, 301, 303, 304, 308, 313, 314, 316, 318, 320, 321, 324, 325, 355, 360, 361, 364, 365, 368, 369, 371, 375, 379, 386, 390, 396, 397, 435, 439, 443, 471, 478, 479, 490, 496, 498, 501, 503, 504, 505, 506, 508, 512, 538, 541, 542, 546, 549, 550, 558, 560, 562, 564, 565, 566, 567, 573, 574, 577, 578, 580, 581, 582, 584, and 585 of SEQ ID NO: 2.
31. The liquid formulation according to embodiment 30 in which the free thiol is provided by a cysteine at a position corresponding to position 34 of SEQ ID NO: 2.
32. The liquid formulation according to any preceding embodiment in which the albumin or fragment thereof is selected from a mammalian albumin or fragment thereof, preferably from the group consisting of HSA (SEQ ID NO: 2), mouse (SEQ ID NO: 3), rat (SEQ ID NO: 4), macaque (SEQ ID NO: 5), bovine (SEQ ID NO: 6), pig (SEQ ID NO: 7), horse (SEQ ID NO: 8), or rabbit (SEQ ID NO: 9).
33. The liquid formulation according to any preceding embodiment in which the albumin or fragment or fusion thereof has at least 70% sequence identity to SEQ ID NO: 2.
34. The liquid formulation according to embodiment 33 in which the albumin or fragment or fusion thereof has at least 75, 80, 85, 90, 91, 92, 93, 94, 95, 96, 97, 98 or 99% sequence identity to SEQ ID NO: 2.
35. The liquid formulation according to embodiment 33 or 34 in which the albumin or fragment or fusion thereof has from 1, 2, 3, 4, 5, 6, 7, 8 or 9 to 2, 3, 4, 5, 6, 7, 8, 9 or 10 amino acid differences compared with SEQ ID NO: 2.
36. The liquid formulation according to embodiment 35 in which the albumin or fragment or fusion thereof comprises SEQ ID NO: 2.
37. The liquid formulation according to any preceding embodiment in which the albumin fragment comprises at least 175 amino acids.
38. The liquid formulation according to any preceding embodiment in which the albumin fragment comprises at least 200, 225, 250, 275, 300, 325, 350, 375, 400, 425, 450, 475, 500, 525, 500 or 575 amino acids.
39. The liquid formulation according to any preceding embodiment in which the albumin fragment comprises at least one (e.g. several) of Domain I, Domain II or Domain III of HSA (SEQ ID NO: 2).
40. The liquid formulation according to any preceding embodiment in which the albumin, fragment or fusion thereof is recombinant.
41. The liquid formulation according to any preceding embodiment in which the albumin, fragment or fusion thereof is produced in a eukaryotic host or prokaryotic host.
42. The liquid formulation according to embodiment 41 in which the eukaryotic host is selected from fungus, plant and mammal.
43. The liquid formulation according to embodiment 42 in which the fungus is selected from Aspergillus, Kluyveromyces, Pichia (e.g. Pichia pastoris) and Saccharomyces (e.g. Saccharomyces cerevisiae).
44. The liquid formulation according to embodiment 43 in which the Saccharomyces is Saccharomyces cerevisiae.
45. The liquid formulation according to embodiment 42 in which the plant is selected from potato, tobacco and rice.
46. The liquid formulation according to embodiment 45 in which the rice is Oryza sativa.
47. The liquid formulation according to embodiment 41 in which the mammal is a mammalian cell.
48. The liquid formulation according to embodiment 47 in which the mammalian cell is CHO or HEK.
49. The liquid formulation according to any of embodiments 1 to 48 in which the albumin is from human or animal serum.
50. The liquid formulation according to any preceding embodiment in which the albumin or fragment or fusion thereof is present at from 5 to 30% (w/v), preferably from 10 to 20% (w/V), most preferably about 20%,
51. The liquid formulation according to any preceding embodiment in which the protein content of the formulation is at least 50% albumin or fragment or fusion thereof, more preferably at least 60%, 70%, 75%, 80%, 85%, 90%, 95%, 96%, 97%, 98%, 99%, 99.5% albumin or fragment or fusion thereof.
52. The liquid formulation according to any preceding embodiment which is substantially free, or completely free, of protein that is not an albumin, fragment or fusion thereof.
53. The liquid formulation according to any preceding embodiment in which the formulation is aqueous.
54. The liquid formulation according to any preceding embodiment presented in a container.
55. A method of preparing a formulation of an albumin or fragment or fusion thereof having a high free thiol level and low polymer level, comprising formulating the albumin or fragment or fusion thereof in a buffer comprising from 2.5 to 7.5 mM fatty acid at least 175 mM cation and a pH from 5.5 to 6.5 and optionally filling the albumin or fragment or fusion thereof into a container.
56. Use of a buffer comprising from 2.5 to 7.5 mM fatty acid at least 175 mM cation and a pH from 5.5 to 6.5 to maintain a high free thiol level and low polymer level in an albumin formulation.
57. The method or use according to embodiment 55 or 56 in which the fatty acid is selected from Ce fatty acids or larger, such as C₆, C₈ or C₁₀ fatty acid, preferably C₇ fatty acids or larger, most preferred C₈ fatty acid (octanoate).
58. The method or use according to any of embodiments 55 to 57 in which the cation is selected from sodium, potassium, calcium, magnesium and ammonium, preferably sodium.
59. A method of making a conjugate comprising conjugating albumin or fragment or fusion thereof having a formulation according to any of embodiments 1 to 54 with a conjugation partner optionally via a linker, and optionally filling the conjugate into a container and optionally providing the conjugate in a unit dosage form.
60. A method of conjugating albumin or fragment or fusion thereof to a partner at at least 75% efficiency relative to albumin, the method comprising contacting a formulation of albumin or fragment or fusion thereof according to any of embodiments 1 to 54 with the partner and, optionally, with a linker.
61. Use of an albumin formulation according to any of embodiments 1 to 54 for high efficiency conjugation to a conjugation partner.
62. The use according to embodiment 61 in which the high efficiency conjugation is at least 75% efficient, more preferably at least 80, 85, 90, 95 efficient.
63. A method or use according to any of embodiments 55 to 62 in which the conjugation partner is a peptide, organic chemical or small molecule pharmaceutical.
64. Use of an albumin formulation according to any of embodiments 1 to 54, to increase the half-life of a molecule such as a bioactive agent, an imaging agent, a diagnostic agent, a contrast agent or a therapeutic compound.
65. Use according to embodiment 63 in which the half-life is increased by at least 10, 20, 30, 40, 50, 60, 70, 80, 90, 100, 150, 200% compared with the molecule not conjugated to albumin.
66. A conjugate comprising an albumin according to any of embodiments 1 to 54, or produced by a method according to any of embodiments 55 to 64, for treatment of disease, treatment of illness and/or for diagnosis.
67. A conjugate comprising an albumin according to any of embodiments 1 to 53, or produced by a method according to any of embodiments 55 to 63, for the manufacture of a medicament for the treatment of disease, treatment of illness and/or for diagnosis.

The present invention is further described by the following examples that should not be construed as limiting the scope of the invention.

### PART A—EXAMPLES

### Example 1: Free Thiol Assays

The level of free thiol in albumin samples was determined by a DTNB assay or by a DTDP assay.

### DTNB Assay

Ellman's Reagent, 5,5′-Dithiobis-(2-Nitrobenzoate) (DTNB) was used to detect free thiol groups such as cys-SH (Cys 34 in the case of HSA, SEQ ID NO: 2)). The reaction between DTNB and a sulfhydryl group releases the 5 thio-2-nitrobenzoate ion TNB²⁻ which has an absorption maximum at 412 nm. By measuring the increase in absorbance at 412 nm, the free thiol content of the albumin was calculated.

Buffer 1 (0.1M TRIS-HCl, 0.01M EDTA, pH 8.0): 4.44 g Tris Hydrochloride (tris(hydroxymethyl)aminomethane hydrochloride, (Sigma)), 2.64 g Tris base (tris(hydroxymethyl)aminomethane, (Sigma)), 1.86 g EDTA (ethylenediaminetetraacetic Acid, di-sodium salt (Sigma)), made up to a final volume of 500 mL with laboratory grade water.

Buffer 2 (0.05M Sodium Phosphate pH 7.0): 2.73 g Na₂HPO₄·2H₂O (di-sodium hydrogen orthophosphate dihydrate, Fisher Scientific), 1.52 g NaH₂PO₄·2H₂O (sodium dihydrogen orthophosphate dihydrate, Fisher Scientific), made up to 500 mL with laboratory grade water.

DTNB Reagent (0.01M DTNB in phosphate buffer): 40 mg DTNB (5, 5′-Dithiobis(2-Nitrobenzoic Acid), (Sigma)) in 10 mL of Buffer 2.

Glutathione Standard (6.5 mM Stock Glutathione Solution): 2.0 g glutathione (L-glutathione, reduced, Sigma), made up to 1000 mL with laboratory grade water.

0.65 mM Glutathione Test Control: 1 mL 6.5 mM Glutathione Standard, 9 mL of laboratory grade water.

### Procedure

1. The albumin samples were diluted to 50 mg·mL⁻¹ with laboratory grade water in a 1.5 ml microfuge tube to generate an ‘albumin test sample’.
2. 1 mL reduced volume plastic cuvettes, each in triplicate, were used for each for the following solutions; the blank, each albumin test sample and the glutathione test control.
3. 1.0 mL of Buffer 1 was pipetted into the “blank” cuvettes.
4. 80 μL (microliters) of diluted albumin test sample was pipetted into the test sample cuvettes.
5. 80 μL (microliters) of the 0.65 mM glutathione test control was pipetted into the glutathione test control cuvettes.
6. 0.92 mL Buffer 1 was added to each test sample and glutathione test control cuvette and mixed gently.
7. The spectrophotometer was set to zero ‘on air’, i.e. without any cuvette in place.
8. The initial absorbances of the blanks, test samples and glutathione test controls were measured at a wavelength of 412 nm (A1).
9. Step 8 was repeated for all subsequent samples.
10. After completing the initial absorbance readings of all the cuvettes, 50 μL (microliters) of DTNB Reagent was added to all cuvettes (i.e. blanks, test sample and glutathione test control) and mixed gently. The samples were incubated for 10 minutes at room temperature (approximately 15 to 25° C.).
11. The final absorbance (A2) of each cuvette was then measured.
12. The free thiol levels of the albumin test samples and glutathione test controls were calculated using the equations below.

### Calculation of Molar Ratio of Free Thiol to Albumin (SH/Albumin)

1. The increase in A₄₁₂ (A₃) for the blanks and samples was calculated as follows:

$${A}_{3} = {A}_{2} - {A}_{1}$$

where A₁ is the initial absorbance i.e. pre-DTNB
where A₂ is the final absorbance i.e. post-DTNB
2. The true absorbance change for each sample was calculated by subtracting the mean of the three blank values, A₃, from each sample.

$$Δ {A}_{412} = {A}_{3} sample - Mean {A}_{3} blank$$

3. The measured free thiol (SH, nmol) was calculated for each sample and test control replicate:

$$nmol Free SH = \frac{Δ {A}_{412} \times 1.05 \times 1000}{{E}_{412}}$$

$$where {E}_{412} = 13.6 {mM}^{- 1} \cdot {cm}^{- 1} for {TNB}^{2 -} at pH 8 \text{.0}$$

4. The quantity of albumin assayed (nmol) was calculated:

$$nmol albumin = \frac{\begin{matrix}Kjeldahl Protein Concentration in diluted \\ sample ( mg \cdot {mL}^{- 1} ) \times 80 μL \times {10}^{3}\end{matrix}}{66 , 438 ( Molecular Weight of human albumin )}$$

for example, 50 mg·mL⁻¹ albumin is 60.2 nmols per cuvette
5. The quantity of glutathione in the test control solution (nmol) was calculated:

$$\frac{\begin{matrix}Weight of glutathione ( g ) in 1000 mL \times 80 ( volume \\ of test control in the cuvette in μL ) \times 100\end{matrix}}{307.32 ( Molecular weight of glutathione )}$$

For example, 2 g glutathione in 1000 mL, i.e. 6.5 mM glutathione, is 52.1 nmols per cuvette
6. The molar ratio of free thiol to albumin (SH/Albumin) in each sample replicate was calculated:

$$Molar Ratio = \frac{nmol Free SH produced}{nmol albumin used in assay}$$

7. The molar ratio of free thiol to glutathione in each test control replicate was calculated:

$$Molar Ratio = \frac{nmol Free SH produced}{nmol glutathione used in assay}$$

8. The mean of each of the triplicate molar ratios was calculated for each test sample and test control.
9. A mean molar ratio for the test control in the range 0.95 to 1.04 indicated that the assay was valid.

### DTDP Assay

4,4′-Dithiodipyridine (DTDP) was also used to detect free thiol groups in albumin samples. The reaction between DTDP and a sulfhydryl group releases the 4-thiopyridone ion (4-TP) which has an absorption maximum at 324 nm. By measuring the increase in absorbance at 324 nm, the free thiol content of albumin was calculated.

Buffer (0.1M Sodium Phosphate, 1 mM EDTA, PH 7.4): 14.56 g disodium hydrogen orthophosphate, dihydrate, (Fisher), 3.04 g sodium dihydrogen orthophosphate, dihydrate (Fisher), 0.37 g EDTA (ethylenediaminetetraacetic acid), di-sodium salt, dihydrate (Sigma), made up to 1000 mL with laboratory grade water.

DTDP Reagent (4 mM DTDP in dilute hydrochloric acid): 0.88 g of DTDP (Aldrithiol™-4, Sigma), dissolved in 50 ml of laboratory grade water initially plus 1000 μL concentrated hydrochloric acid and finally made up to a volume of 1000 mL in laboratory grade water. 1 mL aliquots were stored at −20° C. for single use.

Glutathione Standard (6.5 mM Stock Glutathione Solution) was the same as that described for the DTNB assay (above). 0.65 mM Glutathione Test Control was the same as that described for the DTNB assay (above).

### Procedure

The principle, and general steps, of the DTDP assay is the same as the DTNB assay, i.e. a reagent which reacts specifically with the free thiol groups is added to albumin and the change in absorption is proportional to the amount of free thiol.
1. The albumin samples were diluted to 50 mg·mL⁻¹ with laboratory grade water in a 1.5 ml microfuge tube to generate an ‘albumin test sample’.
2 Blank cuvettes containing 1 mL Buffer and test control cuvettes containing glutathione test control were included as for the DTNB assay (above).
3. For the DTDP assay, 50 μL (microliters) of diluted albumin test sample (or of the 0.65 mM glutathione test control) were pipetted into the test sample cuvettes.
4. 0.95 mL of Buffer was added to each test sample and glutathione test control cuvette.
5. The initial absorbances of the blanks, test samples and glutathione test controls were measured at 324 nm (A₁).
6. After completing the initial absorbance readings of all the cuvettes, 50 μL (microliters) of the DTDP Reagent was added to all cuvettes (i.e. blanks, test sample and glutathione test controls). The samples were incubated for 10 minutes at room temperature (approximately 15 to 25° C.).
7. The final absorbance (A₂) of each cuvette was then measured.
8. The free thiol levels of the albumin test samples and glutathione test controls were calculated using the equations below.

### Calculation of Molar Ratio of Free Thiol to Albumin (SH/Albumin)

1. The increase in A₃₂₄ (A₃) for the blanks and samples was calculated as follows:

$$( {A}_{3} = {A}_{2} - {A}_{1} )$$

where A₁ is the initial absorbance i.e. pre-DTDP
where A₂ is the final absorbance i.e. post-DTDP
2. The true absorbance change (A A₃₂₄) was calculated by subtracting the mean of the three blank values, A₃, from each sample.

$${A}_{324} = {A}_{3} sample - Mean {A}_{3} blank$$

3. The measured Free thiol (nmol) was calculated for each sample and test control replicate:

$$nmol Free SH = \frac{Δ {A}_{324} \times 1.05 \times 1000}{{E}_{324}}$$

$$where {E}_{324} = 21.4 {mM}^{- 1} . {cm}^{- 1} for 4 - TP at pH 7.4$$

4. The quantity of albumin assayed (nmol) was calculated:

$$nmol albumin = \frac{Kjeldahl Protein Concentration in diluted sample ( mg . {mL}^{- 1} ) \times {50}_{μL} \times {10}^{3}}{66 , 438 ( Molecular Weight of human albumin )}$$

For example, 50 mg·mL⁻¹ albumin should be 37.6 nmols per cuvette
5. The quantity of glutathione in the test control solution (nmol) was calculated:

$$\frac{( Weight of glutathione ( g ) in 1000 mL \times 50 ( volume of test control in the cuvette in μ L ) \times 100 )}{307.32 ( Molecular weight of glutathione )}$$

2 g glutathione in 1000 ml (i.e. 6.5 mM stock) is 32.5 nmols per cuvette
6. The molar ratio of free thiol to albumin (SH/Albumin) in each sample replicate was calculated:

$$Molar Ratio = \frac{nmol Free SH produced}{nmol albumin used in assay}$$

7. The molar ratio of free thiol to glutathione in each test control replicate was calculated:

$$Molar Ratio = \frac{nmol Free SH produced}{nmol glutathione used in assay}$$

8. The mean of each of the triplicate molar ratios was calculated for each test sample and test control.
9. A mean molar ratio for the test control in the range 0.95 to 1.04 indicated that the assay was valid.

### Example 2: Determination of Polymer Level, by Gel Permeation High-Performance Liquid Chromatography (GP.HPLC)

GP.HPLC separates proteins according to size. This method was used to separate monomer from polymer which may be present in samples. The polymer level was determined by measuring its peak area relative to the total peak areas present in the sample.

GP.HPLC analysis was carried out using Shimadzu HPLC equipment (LC2010 system) which included two LC 10AD pumps, an SPD-M10Avp UV/Vis detector set to 280 nm and a CTO 10ACvp column oven set to 30° C.

Separation was performed on a TSK G3000 SWxL Analytical Column, 7.8 mm id×30 cm length and TSK SWXL Guard Column, 6.0 mm id×4 cm length.

10× Stock GP.HPLC Buffer (0.25M sodium phosphate, 1.0M sodium sulphate, 0.5% (w/v) sodium azide, pH 6.5): 272.5 g (±1.36 g) of di-sodium hydrogen orthophosphate dodecahydrate, 75.0 g (±0.375 g) sodium dihydrogen orthophosphate dehydrate, 710.0 g (±3.55 g) anhydrous sodium sulphate, 125 mL 20% (w/v) sodium azide, made up to 5 L with laboratory grade water and filtered through a 0.22 μm filter prior to use. The pH of the solution was measured and acceptable if between 6.3 and 6.8.

Working GP.HPLC Buffer (25 mM sodium phosphate, 0.1M sodium sulphate, 0.05% sodium azide, pH 7.0): 10× Stock GP.HPLC Buffer diluted 10 fold with laboratory grade water and filtered through a 0.22 μm membrane. The pH was measured, and where necessary, adjusted to between 6.8 and 7.2 using 1M NaOH or 1M HCl.
20% (w/v) Sodium Azide: 20 g in 100 mL laboratory grade water.

Autoinjector Buffer: 1 mL 30% BRIJ 35 solution (i.e. BRIJ 35 (Polyoxyethylene (23) lauryl ether; C₁₂E₂₃) solution diluted with water), 1 mL 20% sodium azide, made up to 1 L with laboratory grade water.

Albumin Reference Standard (10 mg·mL⁻¹ recombinant albumin (SEQ ID NO: 2, in 0.9% NaCl solution).

### Procedure

The Shimadzu HPLC equipment was started with a flow rate of 1 mL·min⁻¹ and equilibrated for 30 minutes. The system was calibrated by injecting 25 μL of recombinant albumin reference standard three times. The samples were diluted with laboratory grade water so that the sample concentration was less than 10 mg·mL⁻¹ (typically approximately 5 mg·mL⁻¹) and 25 μL was injected into the HPLC equipment. The samples were quantitated for total protein concentration by peak height, and the percentage of the monomer and polymers was calculated using peak areas.

### Example 3: Preparation of Albumin Formulations for Examples 4 and 5

Stock recombinant human albumin (100 g/L, 25 mM phosphate, 250 mM NaCl, pH 6.5, SEQ ID NO: 2) was reformulated into formulations, using 4 different combinations of NaCl and octanoate each at 3 different pH's at 200 mg/ml, as shown in Table 1.

TABLE 1
Target Formulations

| | | NaCl | Octanoate |
|---|---|---|---|
| Sample | pH | (mM) | (mM) |
| 1 | 6.0 | 145 | 0 |
| 2 | 6.0 | 250 | 0 |
| 3 | 6.0 | 145 | 16 |
| 4 | 6.0 | 145 | 32 |
| 5 | 6.5 | 145 | 0 |
| 6 | 6.5 | 250 | 0 |
| 7 | 6.5 | 145 | 16 |
| 8 | 6.5 | 145 | 32 |
| 9 | 7.0 | 145 | 0 |
| 10 | 7.0 | 250 | 0 |
| 11 | 7.0 | 145 | 16 |
| 12 | 7.0 | 145 | 32 |

In summary, twenty-eight 50 ml vials of 10% (w/v) stock albumin samples were pooled, resulting in approximately 1.4 L of material. This was concentrated by ultra-filtration, using a Centramate Omega 10000 molecular weight cut off membrane (Pall) until 750 ml permeate, and 680 mL retentate, was produced. The resulting material was then diafiltered against 7 times the original volume of 50 mM NaCl buffer. The diafiltered retentate, which had reached a volume of approximately 750 mL, was then further concentrated until a final volume of 500-550 mL was achieved. The apparatus was then rinsed out with a final 100 ml of 50 mM NaCl to maximize recovery of the albumin. The final volume of albumin solution was approximately 650 mL.

The anticipated concentration of the albumin solution was greater than or equal to 250 mg·mL⁻¹, and this was quantitated by GP.HPLC as 245 mg·mL⁻¹.

This albumin material was then divided into three 200 mL aliquots for pH adjustment to pH6.0, 6.5 and 7.0. The pH of the starting material was measured as pH6.45 and the pH adjustment was performed by addition of approximately 1-2 mL 1M NaOH or 1M HCl. The resultant albumin samples were sterile filtered and stored at 5° C.

After confirming the correct pH, a sample of each formulation was analyzed for pre-formulation concentrations, specifically for Na by Flame Assisted Emission Spectroscopy and by Specific Gravity and then for octanoate by Gas Chromatography. The data confirmed Na concentrations of 63.2 mM, 64.6 mM and 74.1 mM respectively, and octanoate concentration of 0.5555 mM, 0.5630 mM and 0.5660 mM for all samples.

The required amounts of NaCl, octanoate and water to add back to the samples in order to produce each of the 12 target formulations (Table 1) were calculated. Each of the 3 samples was split into 4 aliquots of approximately 45 mL. Stock solutions of 5M NaCl, 2M octanoate and laboratory grade water were added to the aliquots in a laminar flow hood until the correct amounts to achieve the target values had been achieved.

The protein contents of the samples were quantitated by GP.HPLC at approximately 200-220 mg·mL⁻¹ and finally readjusted using the same approach as above with 5M NaCl, 2M octanoate and laboratory grade water. The final concentrations were all approximately 190 mg·mL⁻¹ protein and these were the final formulations used in the stability study.

The final material was filled into baked 2 mL Type II glass vials (Adelphi) in a laminar flow cabinet, sealed and transferred to controlled incubators at 25° C. and 40° C.

T0 vials were retained for analyses according to Examples 3, 4 and 5.

### Example 4: Stability of Albumin Samples at 25° C., pH 6.0 to 7.0

Samples prepared according to Example 3 (Table 1) were analyzed for stability at T0, T1m (1 month), T2m (2 months), and T3m (3 months) at 25° C. to compare changes in free thiol levels, using the DTNB assay described in Example 1, and polymer levels, according to Example 2, throughout this time course. Stability tests included a colourimetric free thiol assay, a GP.HPLC assay for polymer levels (using the assay as described in Example 2, above. The data are shown in Tables 2 and 3.

TABLE 2
Free thiol level of albumin formulations incubated at 25° C.

| | Sample details | | | Free thiol level (SH/ | | | |
|---|---|---|---|---|---|---|---|
| | | NaCl | Oct | HSA) at time point | | | |
| | PH | (mM) | (mM) | T0 | T1m | T2m | T3m |
| 1 | 6.0 | 145 | 0 | 0.96 | 0.90 | 0.89 | 0.89 |
| 2 | 6.0 | 250 | 0 | 0.96 | 0.91 | 0.86 | 0.90 |
| 3 | 6.0 | 145 | 16 | 0.97 | 0.86 | 0.78 | 0.78 |
| 4 | 6.0 | 145 | 32 | 0.95 | 0.83 | 0.74 | 0.74 |
| 5 | 6.5 | 145 | 0 | 0.97 | 0.92 | 0.85 | 0.86 |
| 6 | 6.5 | 250 | 0 | 0.96 | 0.91 | 0.85 | 0.86 |
| 7 | 6.5 | 145 | 16 | 0.97 | 0.87 | 0.78 | 0.77 |
| 8 | 6.5 | 145 | 32 | 0.93 | 0.81 | 0.73 | 0.71 |
| 9 | 7.0 | 145 | 0 | 0.95 | 0.86 | 0.80 | 0.80 |
| 10 | 7.0 | 250 | 0 | 0.93 | 0.86 | 0.81 | 0.79 |
| 11 | 7.0 | 145 | 16 | 0.94 | 0.82 | 0.75 | 0.74 |
| 12 | 7.0 | 145 | 32 | 0.95 | 0.79 | 0.70 | 0.65 |

TABLE 3
Polymer level of albumin formulations incubated at 25° C.

| | Sample details | | | Polymer (%) | | | |
|---|---|---|---|---|---|---|---|
| | | NaCl | Oct | at time point | | | |
| | pH | (mM) | (mM) | T0 | T1m | T2m | T3m |
| 1 | 6.0 | 145 | 0 | 0 | 0 | 0 | 0 |
| 2 | 6.0 | 250 | 0 | 0 | 0 | 0 | 0 |
| 3 | 6.0 | 145 | 16 | 0 | 0 | 0 | 0 |
| 4 | 6.0 | 145 | 32 | 0 | 0 | 0 | 0 |
| 5 | 6.5 | 145 | 0 | 0 | 0 | 0 | 0 |
| 6 | 6.5 | 250 | 0 | 0 | 0 | 0 | 0 |
| 7 | 6.5 | 145 | 16 | 0 | 0 | 0 | 0 |
| 8 | 6.5 | 145 | 32 | 0 | 0 | 0 | 0 |
| 9 | 7.0 | 145 | 0 | 0 | 0 | 0 | 0 |
| 10 | 7.0 | 250 | 0 | 0 | 0 | 0 | 0 |
| 11 | 7.0 | 145 | 16 | 0 | 0 | 0 | 0 |
| 12 | 7.0 | 145 | 32 | 0 | 0 | 0 | 0 |

The data show that, following incubation at 25° C., free thiol levels are increased by decreasing the amount of octanoate present in the formulation. Polymerization of albumin was not observed at 25° C. between 0 and 3 months incubation.

### Example 5: Stability of Albumin Samples at 40° C., pH 6.0 to 7.0

Samples prepared according to Example 3 (Table 1) were analyzed for stability at T0, T1m, T2m, and T3m at 40° C. to compare changes in free thiol levels and polymer levels throughout this time course. The data are shown in Tables 4 and 5. The free thiol and polymer assays were the same as those used in Example 4. 10

TABLE 4
Free thiol level of albumin formulations incubated at 40° C.

| | Sample details | | | Free thiol level (SH/ | | | |
|---|---|---|---|---|---|---|---|
| | | NaCl | Oct | HSA) at time point | | | |
| | PH | (mM) | (mM) | T0 | T1m | T2m | T3m |
| 1 | 6.0 | 145 | 0 | 0.96 | 0.87 | 0.85 | 0.77 |
| 2 | 6.0 | 250 | 0 | 0.96 | 0.88 | 0.84 | 0.78 |
| 3 | 6.0 | 145 | 16 | 0.97 | 0.81 | 0.69 | 0.63 |
| 4 | 6.0 | 145 | 32 | 0.95 | 0.76 | 0.65 | 0.56 |
| 5 | 6.5 | 145 | 0 | 0.97 | 0.82 | 0.79 | 0.73 |
| 6 | 6.5 | 250 | 0 | 0.96 | 0.86 | 0.81 | 0.74 |
| 7 | 6.5 | 145 | 16 | 0.97 | 0.78 | 0.67 | 0.59 |
| 8 | 6.5 | 145 | 32 | 0.93 | 0.75 | 0.61 | 0.51 |
| 9 | 7.0 | 145 | 0 | 0.95 | 0.78 | 0.74 | 0.65 |
| 10 | 7.0 | 250 | 0 | 0.93 | 0.81 | 0.71 | 0.64 |
| 11 | 7.0 | 145 | 16 | 0.94 | 0.77 | 0.65 | 0.56 |
| 12 | 7.0 | 145 | 32 | 0.95 | 0.73 | 0.57 | 0.45 |

TABLE 5
Polymer level of albumin formulations incubated at 40° C.

| | Sample details | | | Polymer (%) | | | |
|---|---|---|---|---|---|---|---|
| | | NaCl | | at time point | | | |
| | pH | (mM) | Oct | T0 | T1m | T2m | T3m |
| 1 | 6.0 | 145 | 0 | 0 | 3.2 | 1.7 | 5.2 |
| 2 | 6.0 | 250 | 0 | 0 | 1 | 1.2 | 2.7 |
| 3 | 6.0 | 145 | 16 | 0 | 0 | 0.1 | 0.2 |
| 4 | 6.0 | 145 | 32 | 0 | 0 | 0 | 0 |
| 5 | 6.5 | 145 | 0 | 0 | 4.1 | 3.2 | 6 |
| 6 | 6.5 | 250 | 0 | 0 | 1.1 | 1.5 | 2.9 |
| 7 | 6.5 | 145 | 16 | 0 | 0.1 | 0.1 | 0.2 |
| 8 | 6.5 | 145 | 32 | 0 | 0 | 0 | 0 |
| 9 | 7.0 | 145 | 0 | 0 | 6.7 | 4.5 | 9.4 |
| 10 | 7.0 | 250 | 0 | 0 | 2.9 | 3.3 | 5.1 |
| 11 | 7.0 | 145 | 16 | 0 | 0.1 | 0.2 | 0.3 |
| 12 | 7.0 | 145 | 32 | 0 | 0 | 0 | 0 |

The data show that, following incubation at 40° C., free thiol levels are increased by decreasing the amount of octanoate present in the formulation. The data show that addition of octanoate minimizes polymerization of albumin. The data show that decreasing the pH increases free thiol levels and reduces polymerization of albumin.

### Example 6: Preparation of Albumin Formulations for Example 7 and 8

Samples prepared in the same manner as Example 3 were analyzed in a further study for stability at T0, T1m (1 month), T2m (2 months), and T3m (3 months) at 40° C. to compare changes in free thiol levels, using the DTDP assay described in Example 1, and polymer levels, using the assay of Example 2, throughout this time course. The data are shown in Tables 6 and 7.

TABLE 6
Free thiol level of albumin formulations incubated at 40° C.

| | Sample details | | | Free thiol level (SH/ | | | |
|---|---|---|---|---|---|---|---|
| | | NaCl | Oct | HSA) at time point | | | |
| | pH | (mM) | (mM) | T0 | T1m | T2m | T3m |
| 1 | 5.5 | 145 | 0.5 | 0.96 | 0.89 | 0.81 | 0.8 |
| 2 | 5.5 | 145 | 4 | 0.96 | 0.88 | 0.78 | 0.76 |
| 3 | 5.5 | 145 | 8 | 0.92 | 0.74 | 0.69 | 0.66 |
| 4 | 5.5 | 145 | 16 | 0.9 | 0.7 | 0.63 | 0.58 |
| 5 | 5.5 | 200 | 0.5 | 0.94 | 0.84 | 0.81 | 0.81 |
| 6 | 5.5 | 200 | 4 | 0.97 | 0.82 | 0.78 | 0.76 |
| 7 | 5.5 | 200 | 8 | 0.95 | 0.73 | 0.7 | 0.65 |
| 8 | 5.5 | 200 | 16 | 0.94 | 0.69 | 0.63 | 0.58 |
| 9 | 5.5 | 250 | 0.5 | 0.96 | 0.85 | 0.84 | 0.8 |
| 10 | 5.5 | 250 | 4 | 0.94 | 0.82 | 0.78 | 0.76 |
| 11 | 5.5 | 250 | 8 | 0.94 | 0.74 | 0.7 | 0.64 |
| 12 | 5.5 | 250 | 16 | 0.91 | 0.66 | 0.62 | 0.57 |
| 13 | 6.0 | 145 | 0.5 | 0.95 | 0.87 | 0.81 | 0.75 |
| 14 | 6.0 | 145 | 4 | 0.98 | 0.8 | 0.75 | 0.7 |
| 15 | 6.0 | 145 | 8 | 0.94 | 0.74 | 0.69 | 0.63 |
| 16 | 6.0 | 145 | 16 | 0.87 | 0.68 | 0.62 | 0.55 |
| 17 | 6.0 | 200 | 0.5 | 0.96 | 0.83 | 0.8 | 0.76 |
| 18 | 6.0 | 200 | 4 | 0.96 | 0.81 | 0.77 | 0.73 |
| 19 | 6.0 | 200 | 8 | 0.95 | 0.75 | 0.71 | 0.64 |
| 20 | 6.0 | 200 | 16 | 0.92 | 0.66 | 0.59 | 0.54 |
| 21 | 6.0 | 250 | 0.5 | 0.96 | 0.84 | 0.79 | 0.77 |
| 22 | 6.0 | 250 | 4 | 0.91 | 0.81 | 0.76 | 0.72 |
| 23 | 6.0 | 250 | 8 | 0.94 | 0.75 | 0.69 | 0.64 |
| 24 | 6.0 | 250 | 16 | 0.92 | 0.66 | 0.59 | 0.52 |
| 25 | 6.5 | 145 | 0.5 | 0.94 | 0.79 | 0.72 | 0.68 |
| 26 | 6.5 | 145 | 4 | 0.96 | 0.76 | 0.71 | 0.66 |
| 27 | 6.5 | 145 | 8 | 0.95 | 0.73 | 0.67 | 0.6 |
| 28 | 6.5 | 145 | 16 | 0.95 | 0.65 | 0.58 | 0.5 |
| 29 | 6.5 | 200 | 0.5 | 0.96 | 0.78 | 0.73 | 0.68 |
| 30 | 6.5 | 200 | 4 | 0.96 | 0.76 | 0.7 | 0.65 |
| 31 | 6.5 | 200 | 8 | 0.9 | 0.72 | 0.66 | 0.58 |
| 32 | 6.5 | 200 | 16 | 0.89 | 0.65 | 0.56 | 0.48 |
| 33 | 6.5 | 250 | 0.5 | 0.95 | 0.78 | 0.74 | 0.69 |
| 34 | 6.5 | 250 | 4 | 0.91 | 0.76 | 0.71 | 0.66 |
| 35 | 6.5 | 250 | 8 | 0.94 | 0.72 | 0.65 | 0.57 |
| 36 | 6.5 | 250 | 16 | 0.93 | 0.64 | 0.56 | 0.48 |

TABLE 7
Polymer level of albumin formulations incubated at 40° C.

| | Sample details | | | Polymer (%) | | | |
|---|---|---|---|---|---|---|---|
| | | NaCl | Oct | at time point | | | |
| | pH | (mM) | (mM) | T0 | T1m | T2m | T3m |
| 1 | 5.5 | 145 | 0.5 | 0 | 5.1 | NR | 9.5 |
| 2 | 5.5 | 145 | 4 | 0 | 3.6 | 6.8 | 5.6 |
| 3 | 5.5 | 145 | 8 | 0 | 1.2 | 2.5 | 2.6 |
| 4 | 5.5 | 145 | 16 | 0 | 0.5 | 1.1 | 1.4 |
| 5 | 5.5 | 200 | 0.5 | 0 | 4.5 | 8 | 9 |
| 6 | 5.5 | 200 | 4 | 0 | 2.5 | 5 | 5.2 |
| 7 | 5.5 | 200 | 8 | 0 | 0.5 | 2.1 | 2 |
| 8 | 5.5 | 200 | 16 | 0 | 0.3 | 0.6 | 0.9 |
| 9 | 5.5 | 250 | 0.5 | 0 | 3.6 | 6.5 | 8.1 |
| 10 | 5.5 | 250 | 4 | 0 | 1.9 | 3.9 | 4.9 |
| 11 | 5.5 | 250 | 8 | 0 | 0.8 | 1.6 | 2.4 |
| 12 | 5.5 | 250 | 16 | 0 | 0.3 | 0.6 | 0.9 |
| 13 | 6.0 | 145 | 0.5 | 0 | 4.3 | 7.3 | 9.1 |
| 14 | 6.0 | 145 | 4 | 0 | 1.5 | 3.1 | 4.3 |
| 15 | 6.0 | 145 | 8 | 0 | 0.5 | 1 | 1.7 |
| 16 | 6.0 | 145 | 16 | 0 | 0.1 | 0.3 | 0.5 |
| 17 | 6.0 | 200 | 0.5 | 0 | 2.5 | 4.7 | 6.1 |
| 18 | 6.0 | 200 | 4 | 0 | 0.9 | 2.1 | 3.2 |
| 19 | 6.0 | 200 | 8 | 0 | 0.4 | 0.8 | 1.3 |
| 20 | 6.0 | 200 | 16 | 0 | 0.1 | 0.2 | 0.3 |
| 21 | 6.0 | 250 | 0.5 | 0 | 1.8 | 3.6 | 4.3 |
| 22 | 6.0 | 250 | 4 | 0 | 0.6 | 1.6 | 2.2 |
| 23 | 6.0 | 250 | 8 | 0 | 0.2 | 0.5 | 0.9 |
| 24 | 6.0 | 250 | 16 | 0 | 0.1 | 0.1 | 0.2 |
| 25 | 6.5 | 145 | 0.5 | 0 | 5.5 | 8.2 | 9.1 |
| 26 | 6.5 | 145 | 4 | 0 | 2.1 | 4 | 4.8 |
| 27 | 6.5 | 145 | 8 | 0 | 0.5 | 1.3 | 1.8 |
| 28 | 6.5 | 145 | 16 | 0 | 0.1 | 0.2 | 0.4 |
| 29 | 6.5 | 200 | 0.5 | 0 | 3 | 5.2 | 6.1 |
| 30 | 6.5 | 200 | 4 | 0 | 0.8 | 1.8 | 2.6 |
| 31 | 6.5 | 200 | 8 | 0 | 0.2 | 0.7 | 1 |
| 32 | 6.5 | 200 | 16 | 0 | 0.1 | 0.2 | 0.2 |
| 33 | 6.5 | 250 | 0.5 | C | 1.7 | 3.7 | 4.6 |
| 34 | 6.5 | 250 | 4 | 0 | 0.4 | 1.3 | 2 |
| 35 | 6.5 | 250 | 8 | 0 | 0.2 | 0.4 | 0.7 |
| 36 | 6.5 | 250 | 16 | 0 | 0.1 | 0.1 | 0.2 |

NR: No Result: sample precipitation meant that the polymer content could not be accurately determined.

### Example 7: Prediction of Optimum Formulation

Using the T1m and T3m data from Tables 6 and 7, a Design of Experiments (“DoE”) approach was used to predict the optimum albumin formulation to maximise free thiol stability while minimising polymer formation.

The software used was JMP version 11.1.1 (32 bit) from SAS Institute Inc. The data design was through a full factorial DoE with pH set at 5.5, 6.0 and 6.5; sodium (“Na”) set at 145, 200 and 250 mM; and octanoate (“Oct”) set at 0.5, 4, 8 and 16 mM. Result data was then added and statistical analysis performed using a quadratic model using the following script:

```
Fit Model(
  Effects(
    :pH,
    :Na,
    :Oct,
    :pH * :Na,
    :pH * :Oct,
    :Na * :Oct,
    :pH * :pH,
    :Na * :Na,
    :Oct * :Oct,
    :pH * :Na * :Oct
    ),
    Y( :Thiol data ),
    Y( :Polymer data ),
    PERSONALITY( Standard Least Squares )
)
```

where Thiol data and Polymer data relate to the appropriate temperature and time point being tested.

No weighting or data transformations were used. To select the optimum formulation, an equal weighting was given to maximising the free thiol and minimising the polymer. This script was used for both time points (T1m and T3m).

The fitting of the data for both time points for both thiol and polymer was very good with a significant model fit (predicted P<0.0001) for both. The results showed similar trends with pH and octanoate predominantly affecting thiol levels and all parameters having an effect on the polymer levels. Both time points also gave similar optimum formulations of pH 5.9 to 6.0, 250 mM sodium and 4 to 6 mM octanoate. Consequently, a preferred albumin formulation is about 20% albumin, about pH 6.0, about 250 mM cation (e.g. sodium) and about 5 mM fatty acid (e.g. octanoate).

As both time point results (T1m, T3m,) gave similar results and trends, the T1 month data for 40° C. can be used to rapidly predict the stability of a proposed formulation. Consequently, a model formula was derived from the T1 month data as follows:

$$Thiol level = 1.1786 - 0.05167 * : pH - 0.0001544 * : Na - 0.01133 * : Oct + ( : pH - 6 ) * ( ( : Na - 198.3 ) * 0.0002659 ) + ( : pH - 6 ) * ( ( : Oct - 7.125 ) * 0.00316 ) + ( : Na - 198.3 ) * ( ( : Oct - 7.125 ) * 0.000001935 ) + ( : pH - 6 ) * ( ( : pH - 6 ) * - 0.05 ) + ( : Na - 198.3 ) * ( ( : Na - 198.3 ) * 0.000002583 ) + ( : Oct - 7.125 ) * ( ( : Oct - 7.125 ) * 0.0001413 ) + ( : pH - 6 ) * ( ( : Na - 198.3 ) * ( ( : Oct - 7.125 ) * - 0.000009879 ) )$$

$$Polymer Level = 9.1677 - 0.8417 * : pH - 0.01044 * : Na - 0.269 * : Oct + ( : pH - 6 ) * ( ( : Na - 198.3 ) * - 0.004796 ) + ( : pH - 6 ) * ( ( : Oct - 7.125 ) * 0.06523 ) + ( : Na - 198.3 ) * ( ( : Oct - 7.125 ) * 0.001494 ) + ( : pH - 6 ) * ( ( : pH - 6 ) * 2.25 ) + ( : Na - 198.3 ) * ( ( : Na - 198.3 ) * 0.00006768 ) + ( : Oct - 7.125 ) * ( ( : Oct - 7.125 ) * 0.0249 ) + ( : pH - 6 ) * ( ( : Na - 198.3 ) * ( ( : Oct - 7.125 ) * 0.001237 ) )$$

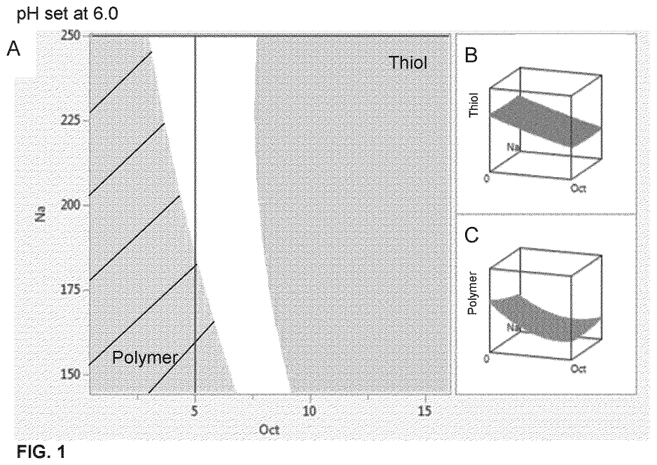

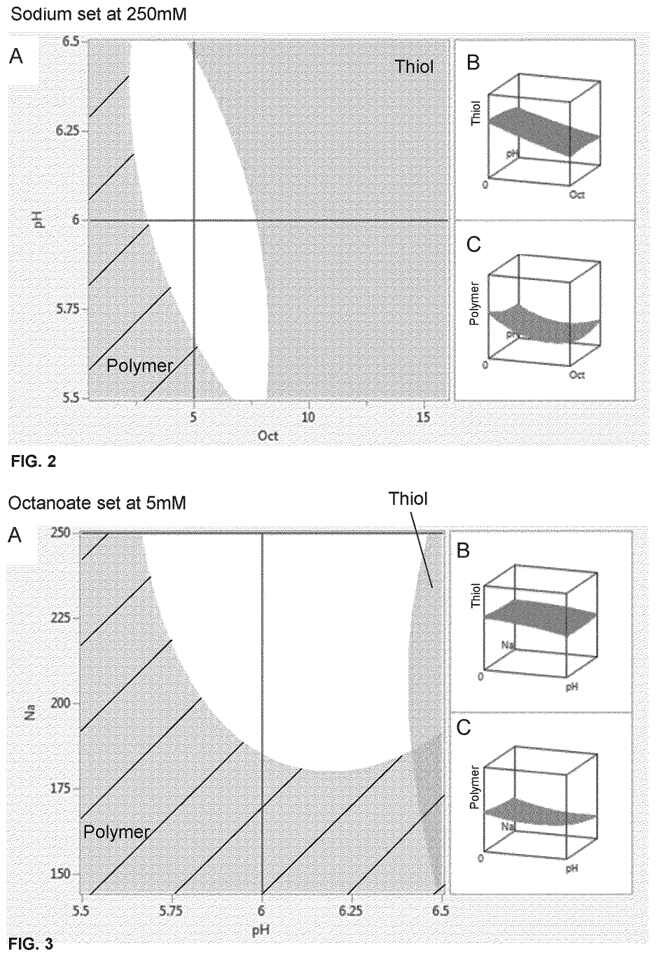

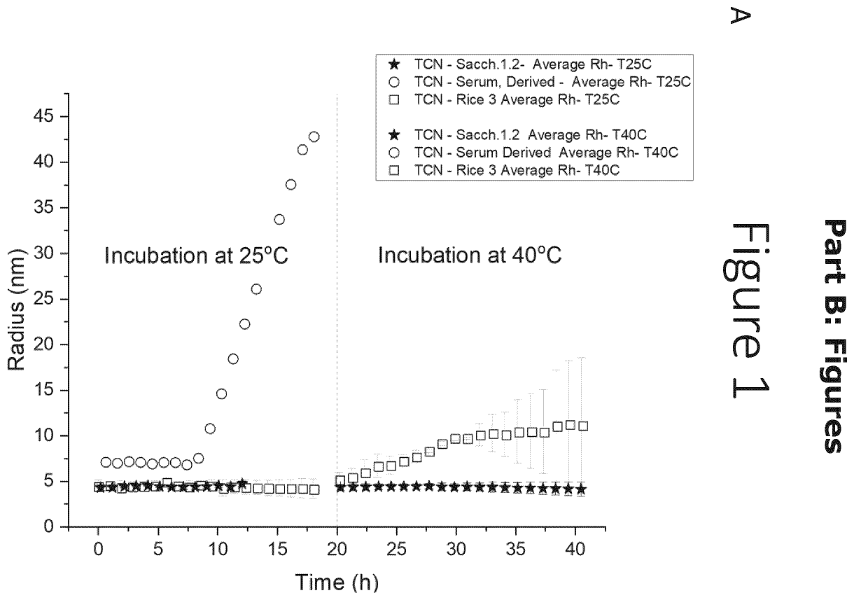

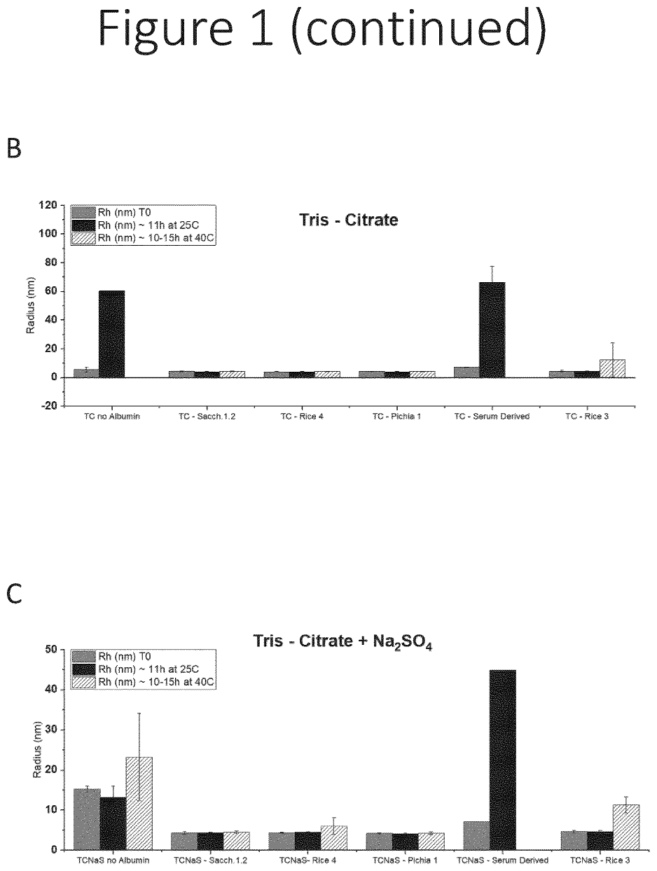

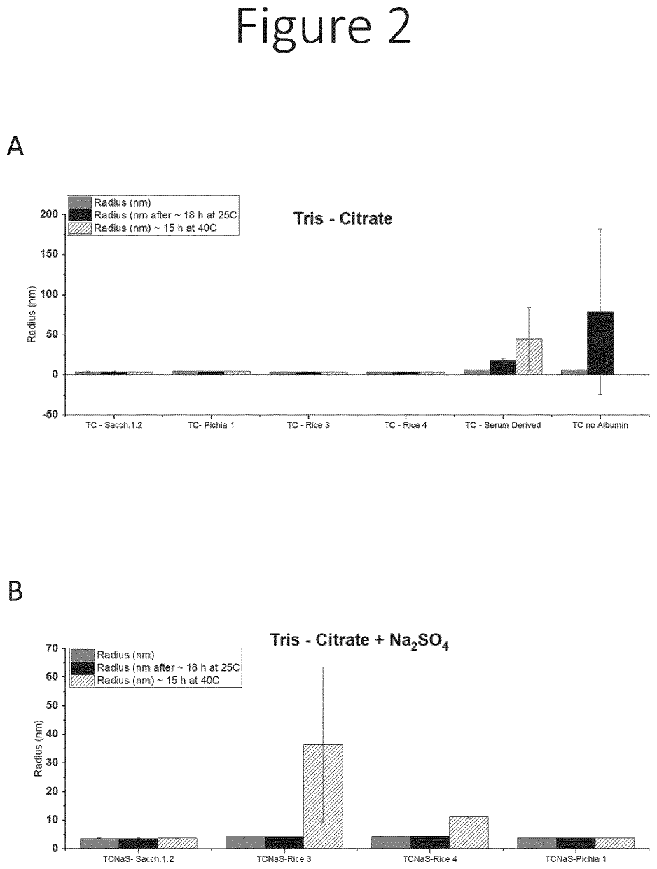

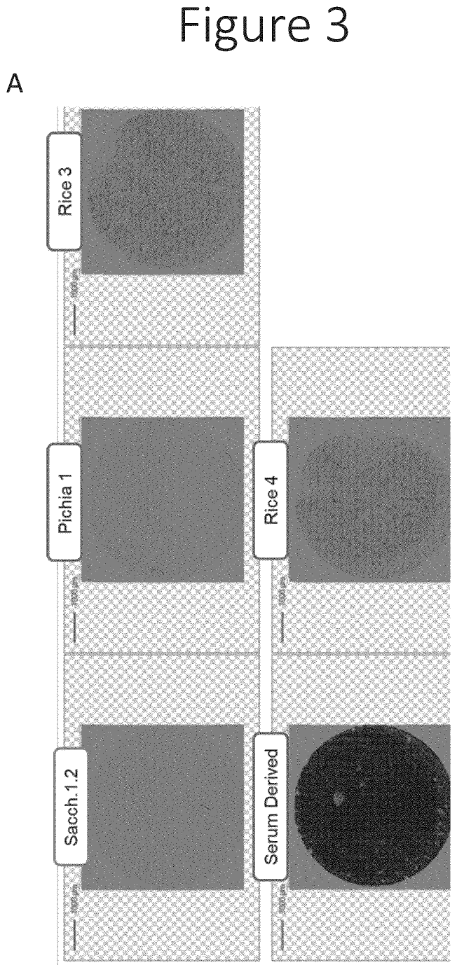

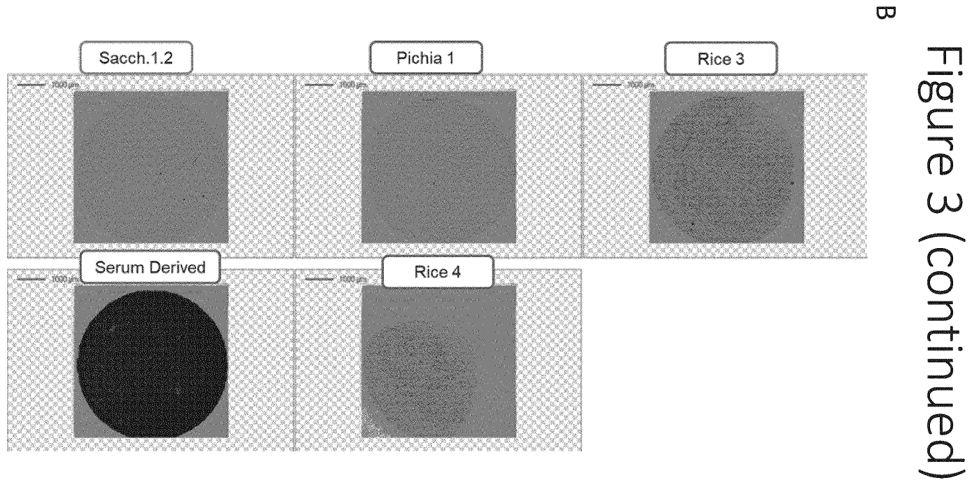

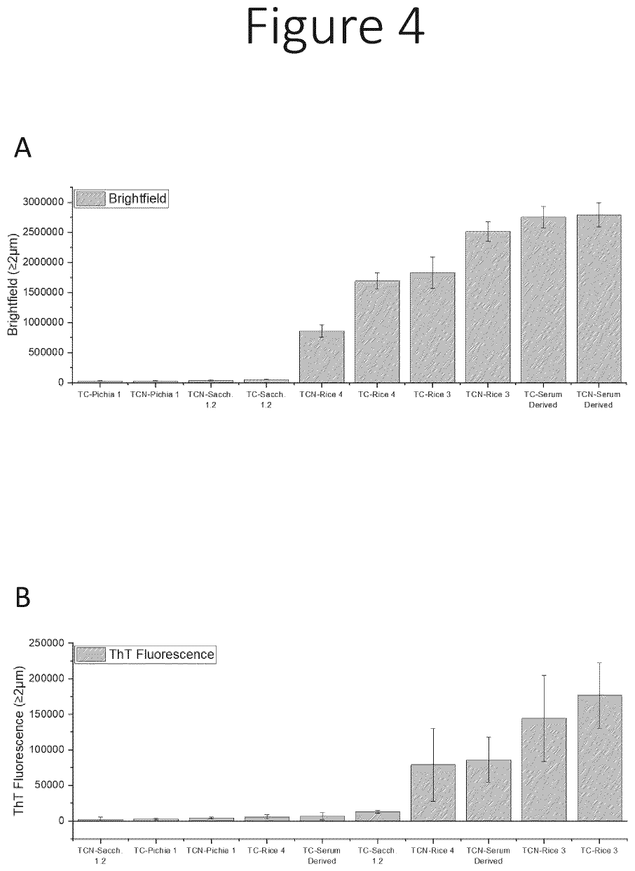

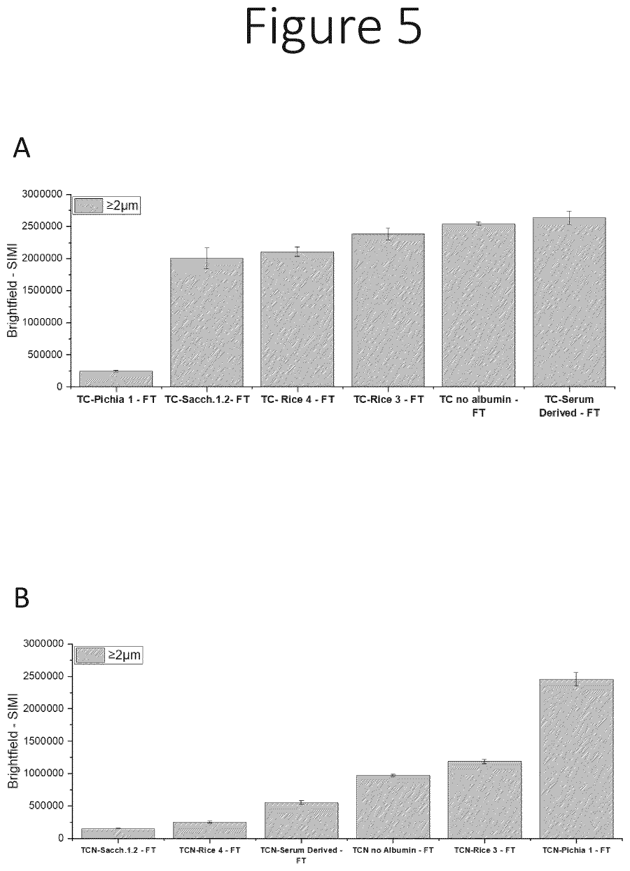

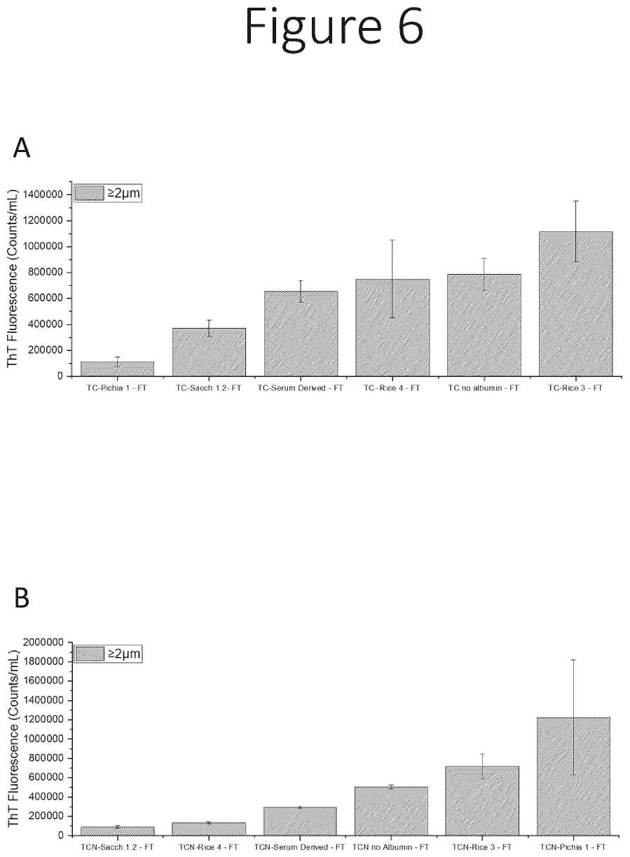

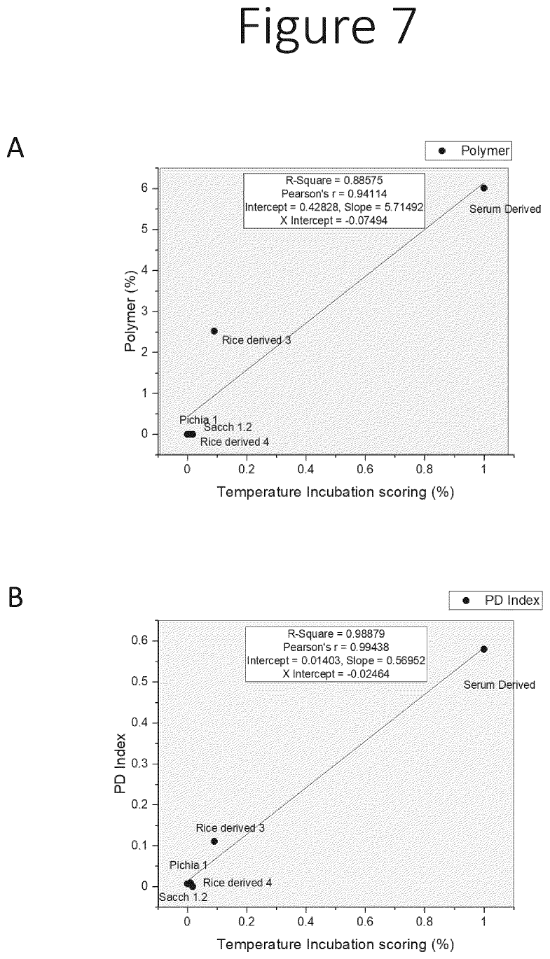

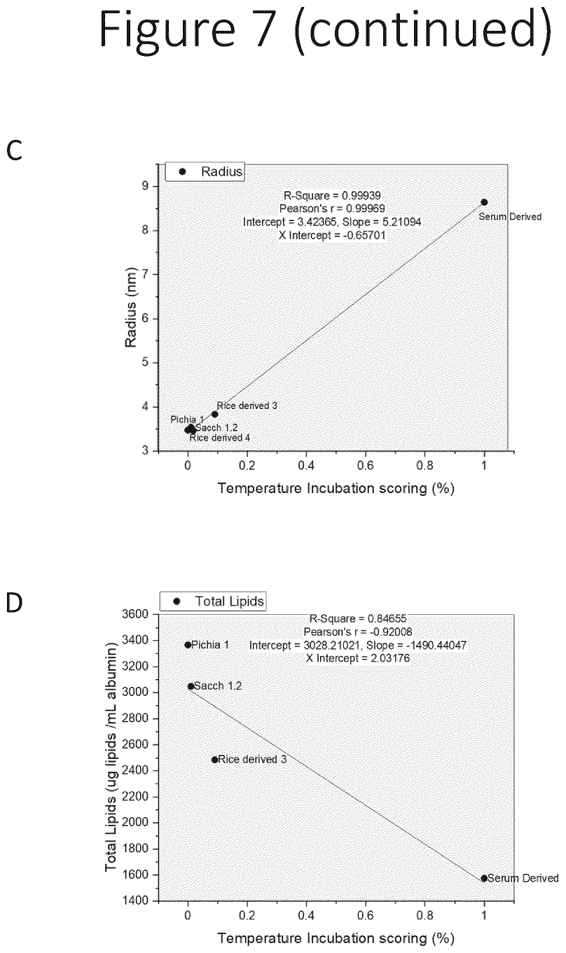

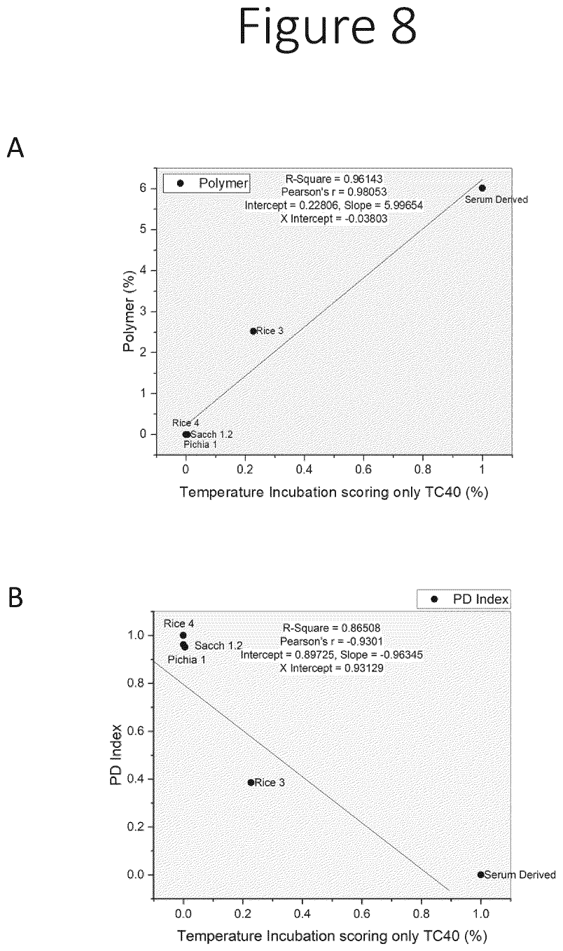

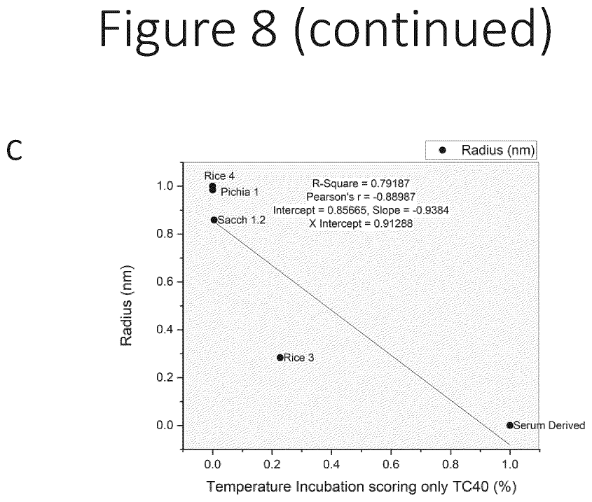

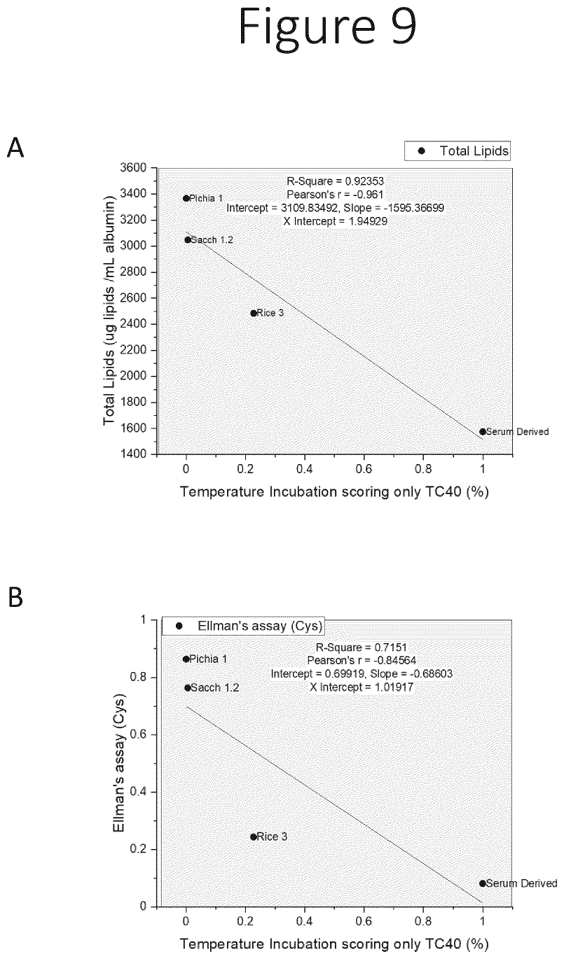

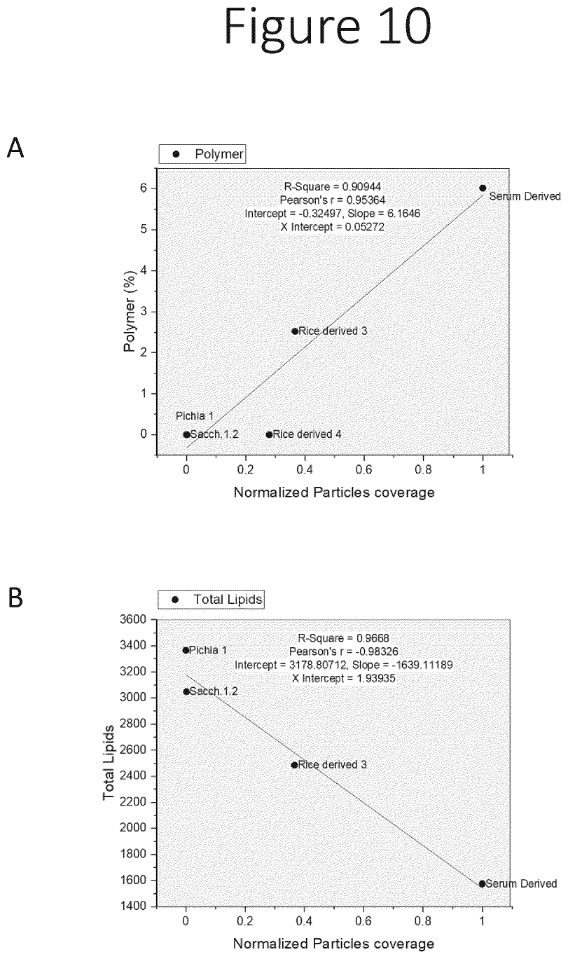

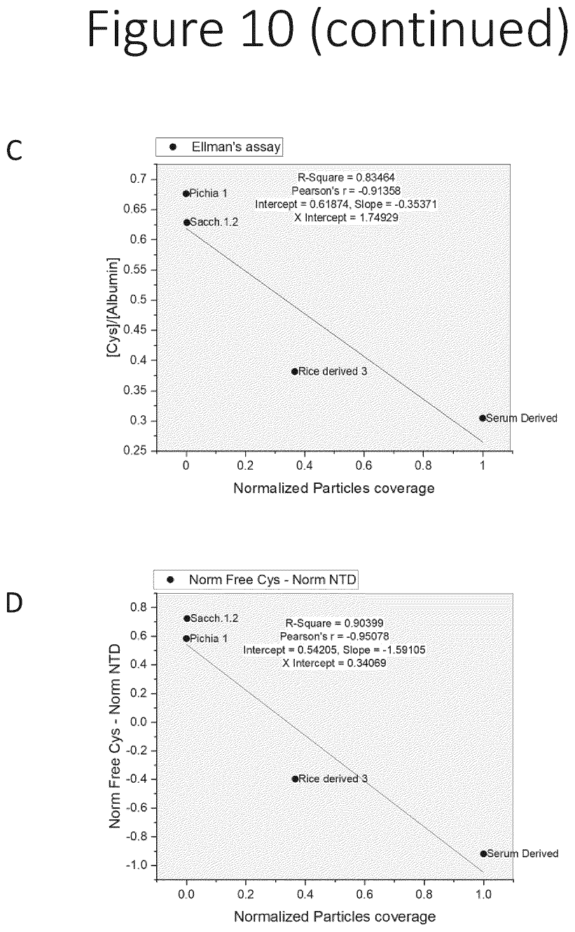

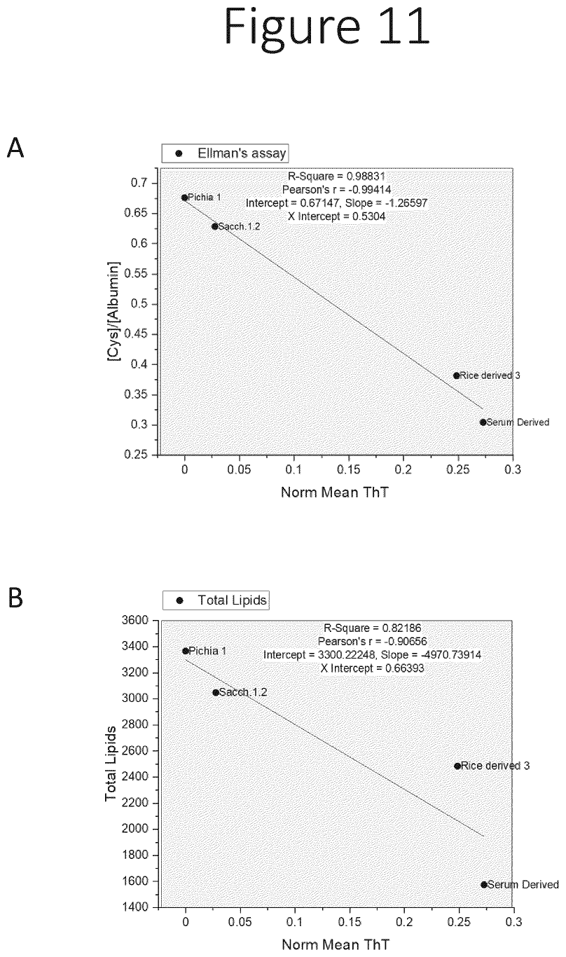

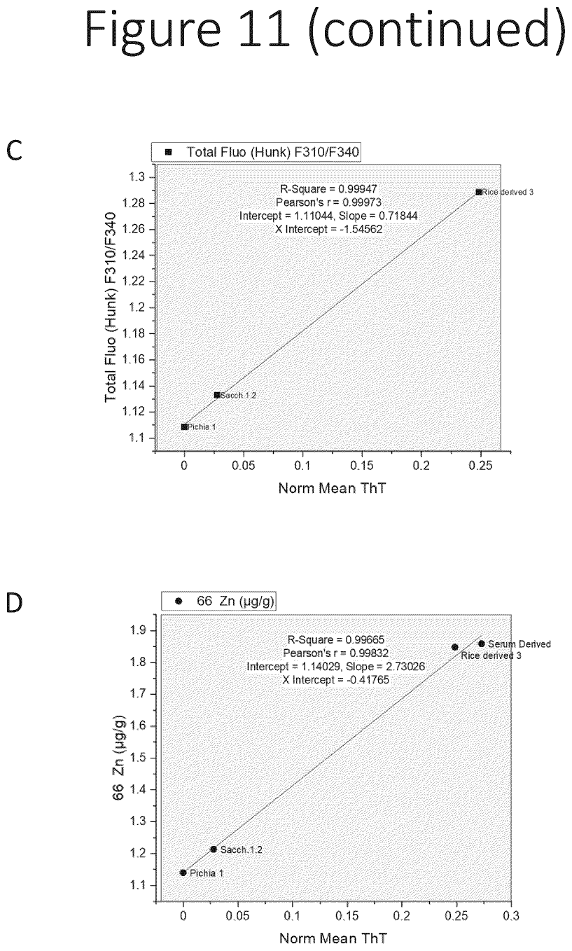

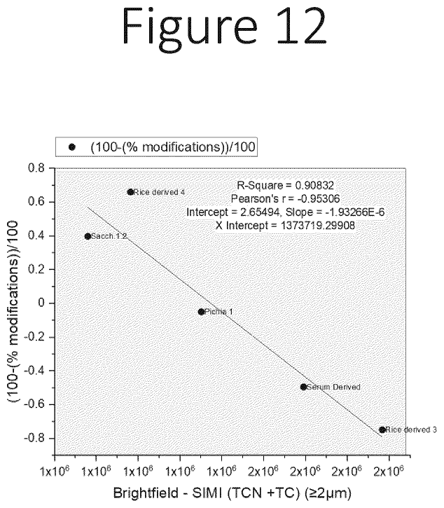

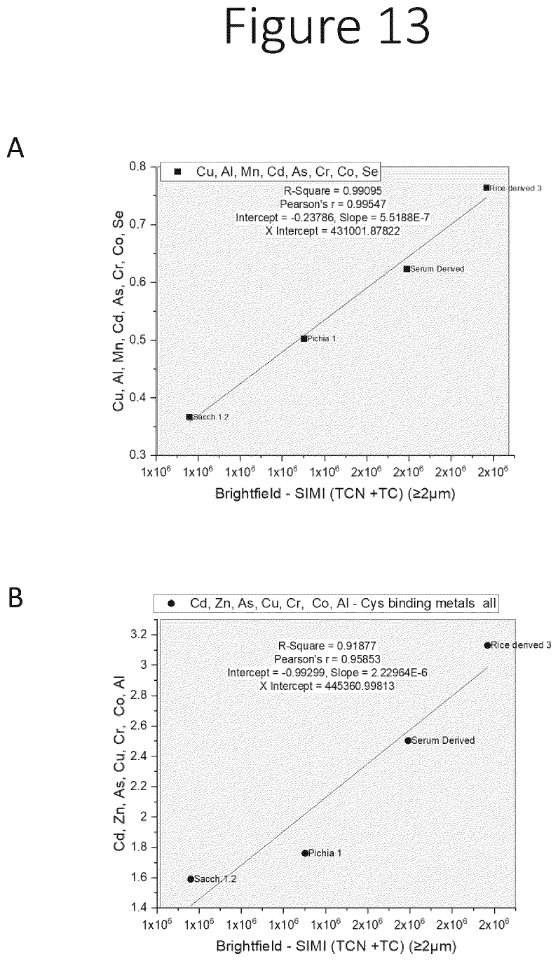

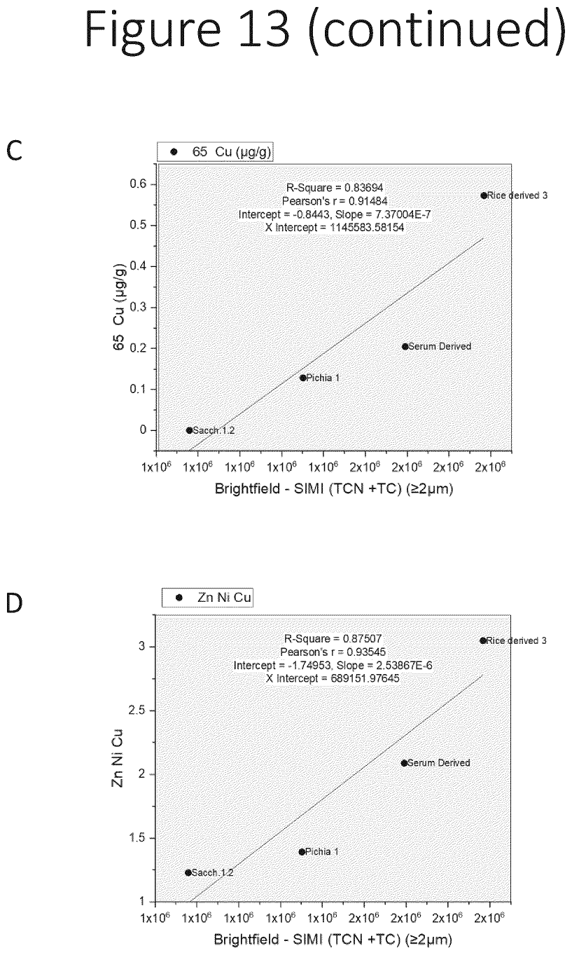

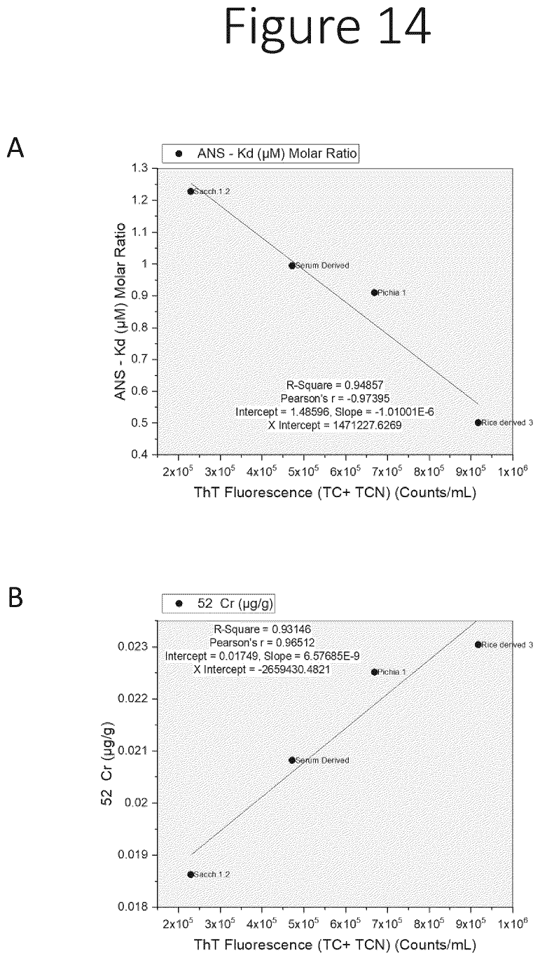

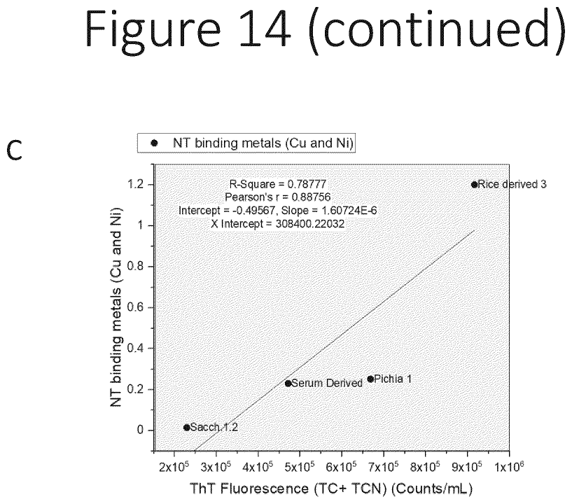

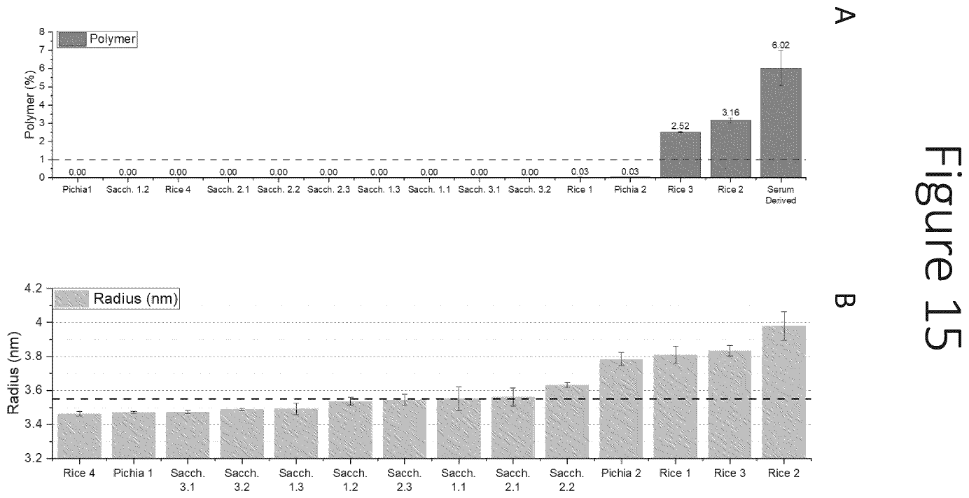

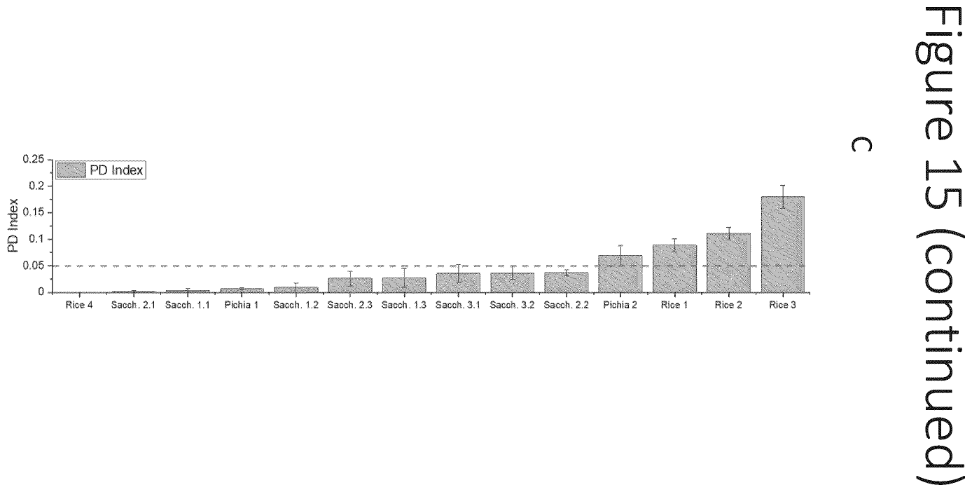

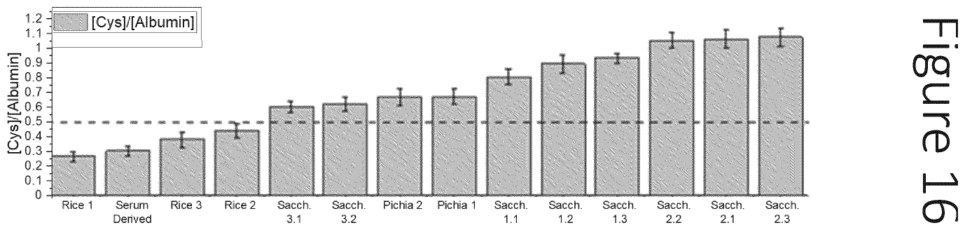

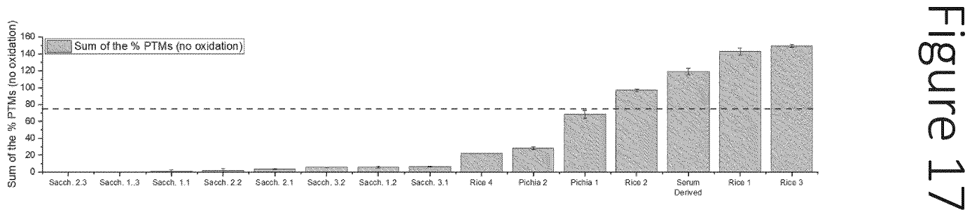

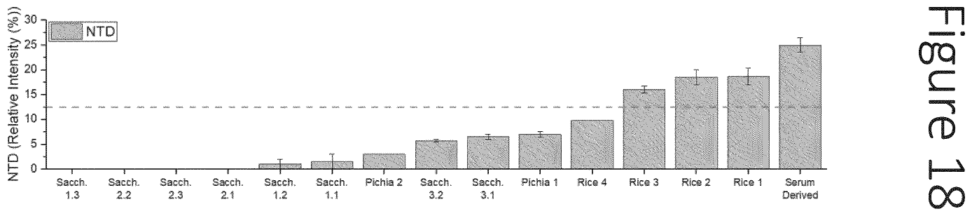

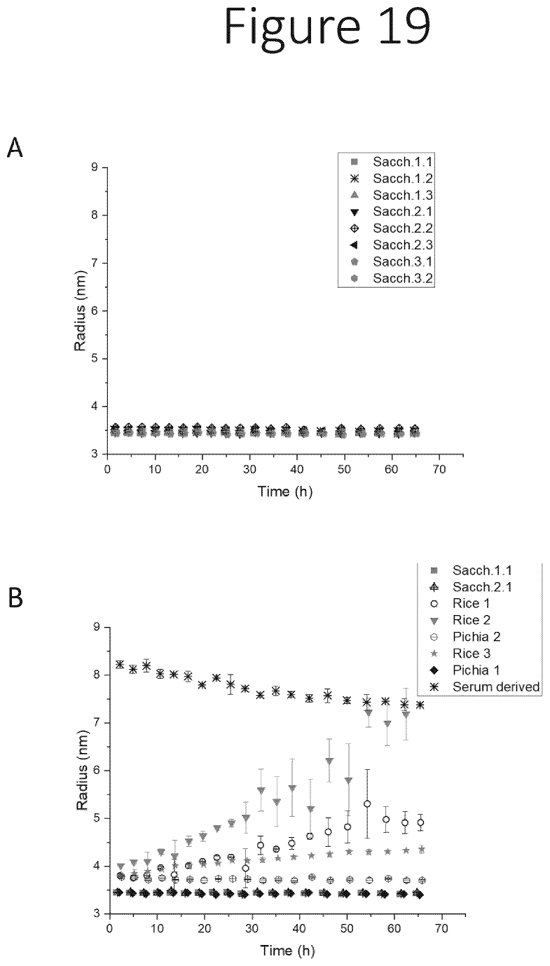

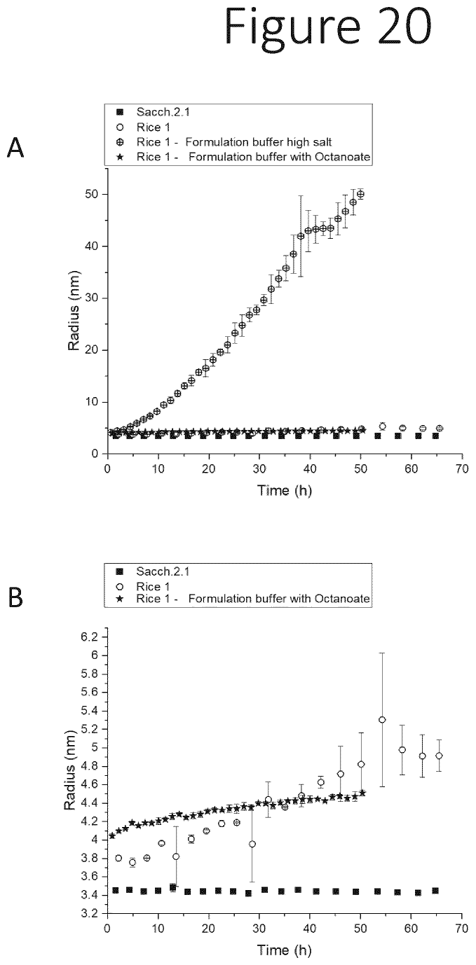

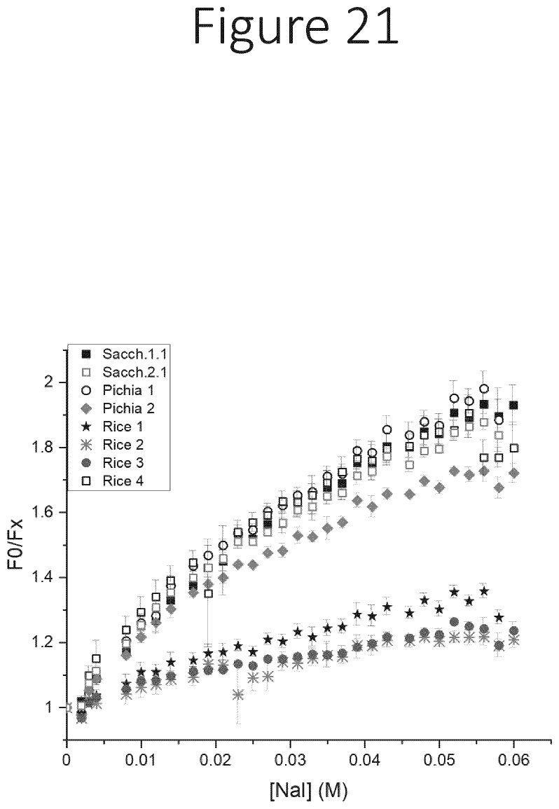

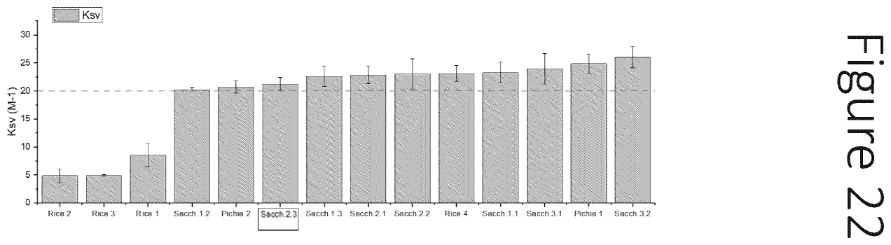

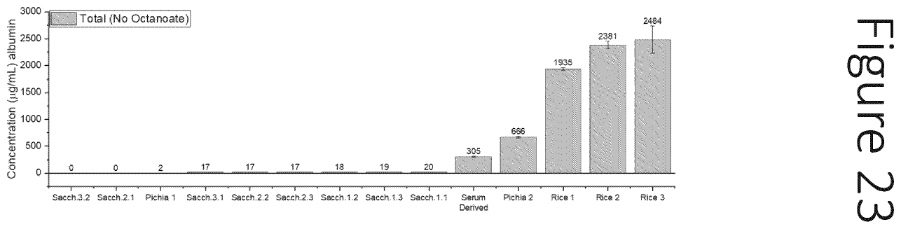

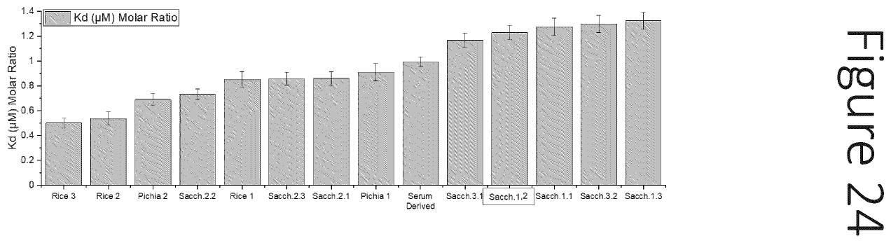

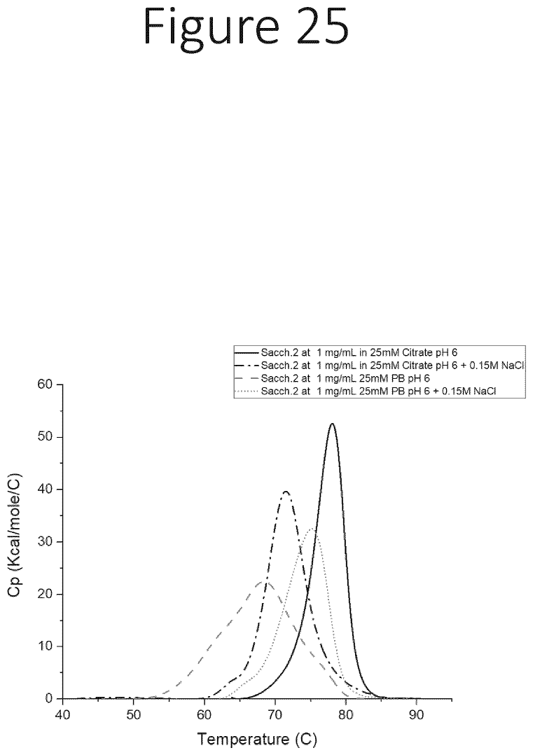

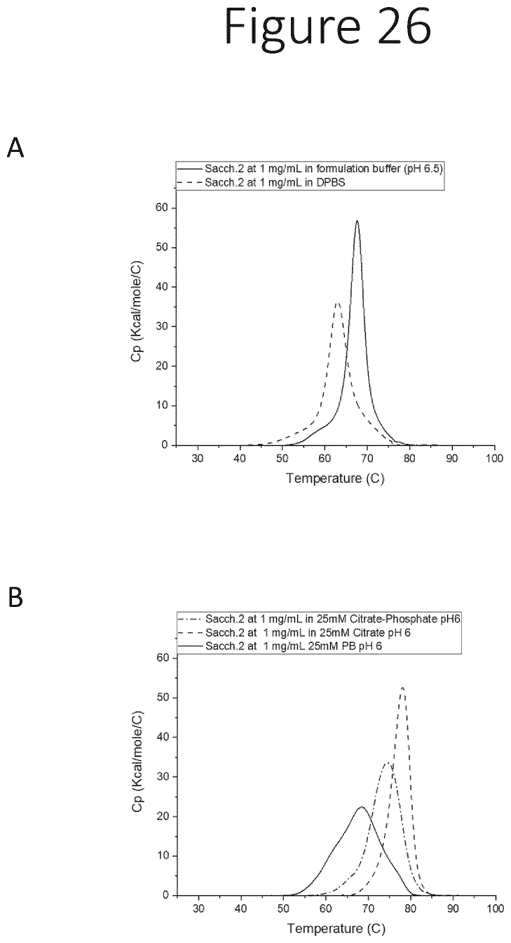

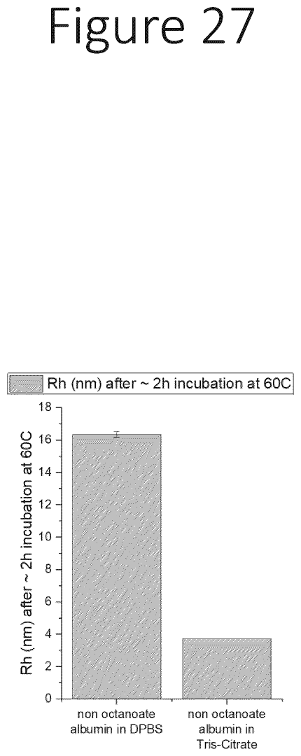

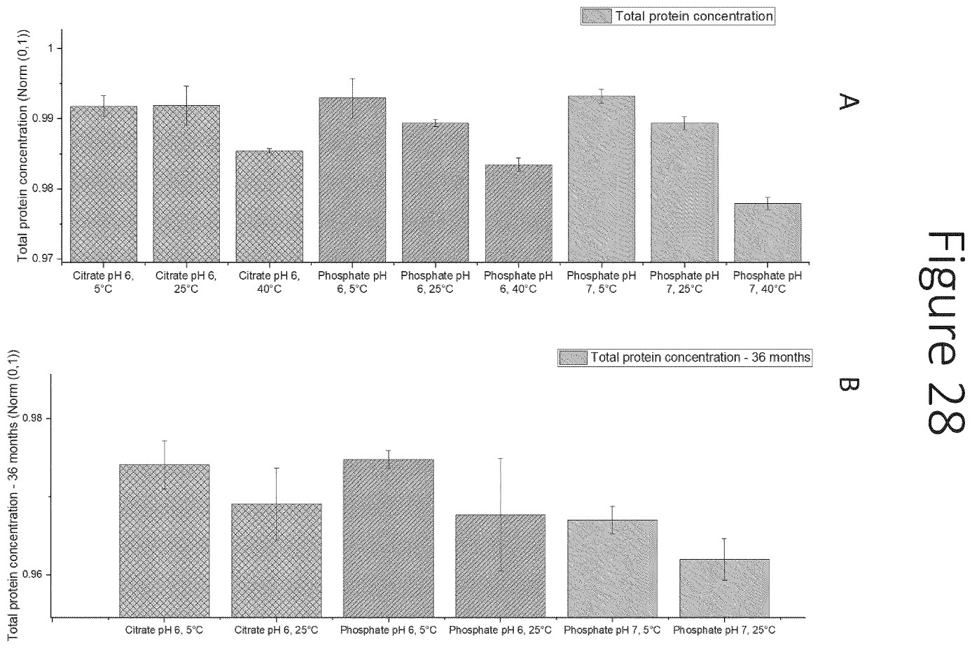

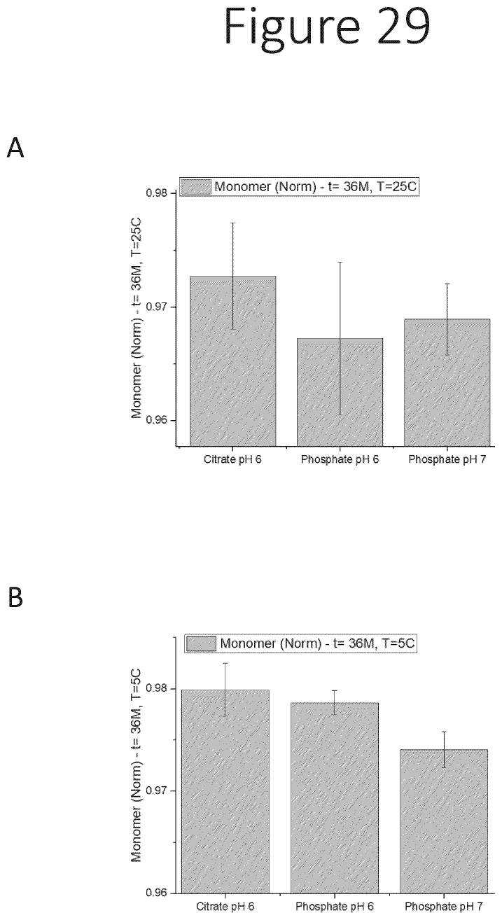

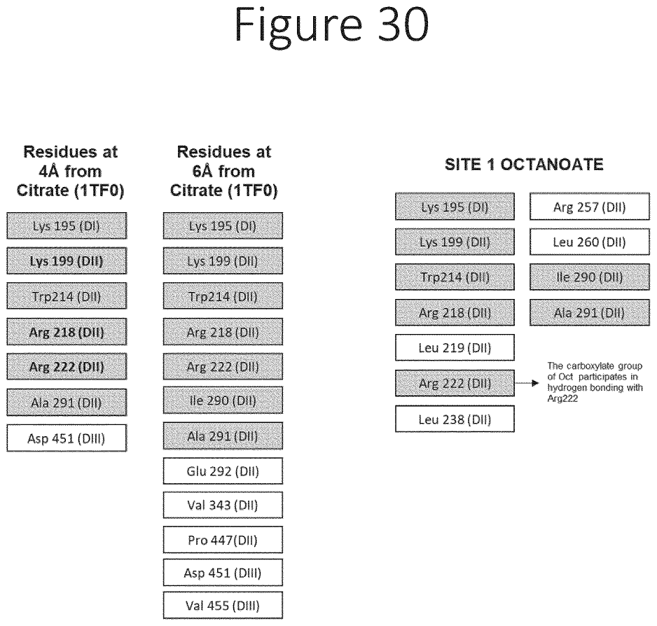

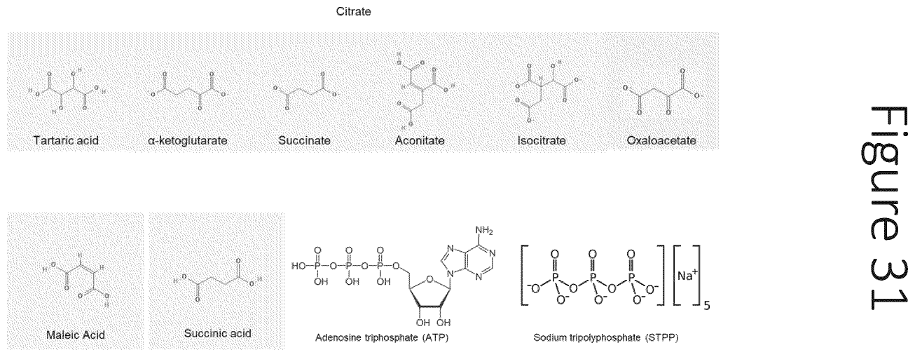

Based on these equations formulations were defined that gave acceptable thiol and polymer levels after storage or incubation for 1 month at 40° C. For example, by setting limits for the thiol level (e.g. at least 0.75 mol per mol) and polymer e.g. less than or equal to 1%), the required limits for pH, cation (e.g. sodium) and fatty acid (e.g. octanoate) were identified. These limits cannot be easily represented in a single graph and so are represented in FIGS. 1, 2 and 3 as 2-dimensional graphs with the third parameter set at the level of the preferred formulation, i.e. pH 6.0 (FIG. 1), 250 mM sodium (FIG. 2) and 5 mM octanoate (FIG. 3).

In each case (see graph A of each of FIGS. 1, 2 and 3) the solid shaded area marks where the thiol level is outside the proposed limit, the diagonal shaded area is where the polymer is outside the proposed limit and the unshaded white area is where the polymer and thiol are within the proposed limits. The intersection of the black lines is the preferred formulation, i.e. about 20% albumin, about pH 6.0, about 250 mM cation e.g. sodium and about 5 mM fatty acid e.g. octanoate.

Statistical analysis showed that this is a very good mathematical model describing the relationship between (a) pH, cation and fatty acid and their effect on (b) thiol level and polymer level. Based on this analysis and the stability studies of Examples 4, 5 and 6, the preferred formulation is about 20% albumin, about pH 6.0, about 250 mM sodium and about 5 mM octanoate.

### List of Sequences Described Herein

>SEQ ID NO: 1
gacgctcacaagtccgaagtcgctcacagattcaaggacttgggtgaag
aaaacttcaaggctttggtcttgatcgctttcgctcaatacttgcaaca
atgtccattcgaagatcacgtcaagttggtcaacgaagttaccgaattc
gctaagacttgtgttgctgacgaatccgcggaaaactgtgacaagtcct
tgcacaccttgttcggtgataagttgtgtactgttgctaccttgagaga
aacctacggtgaaatggctgactgttgtgctaagcaagaaccagaaaga
aacgaatgtttcttgcaacacaaggacgacaacccaaacttgccaagat
tggttagaccagaagttgacgtcatgtgtactgctttccacgacaacga
agaaaccttcttgaagaagtacttgtacgaaattgctagaagacaccca
tacttctacgctccagaattgttgttcttcgctaagagatacaaggctg
ctttcaccgaatgttgtcaagctgctgataaggctgcttgtttgttgcc
aaagttggatgaattgagagacgaaggtaaggctagctccgcaaagcaa
agattgaagtgtgcttccttgcaaaagttcggtgaaagagctttcaagg
cttgggctgtcgctagattgtctcaaagattcccaaaggctgaattcgc
tgaagtttctaagttggttactgacttgactaaggttcacactgaatgt
tgtcacggtgacttgttggaatgtgctgatgacagagctgacttggcta
agtacatctgtgaaaaccaagactctatctcttccaagttgaaggaatg
ttgtgaaaagccattgttggaaaagtctcactgtattgctgaagttgaa
aacgatgaaatgccagctgacttgccatctttggctgctgacttcgttg
aatctaaggacgtttgtaagaactacgctgaagctaaggacgtcttctt
gggtatgttcttgtacgaatacgctagaagacacccagactactccgtt
gtcttgttgttgagattggctaagacctacgaaactaccctcgagaagt
gttgtgctgctgctgacccacacgaatgttacgctaaggttttcgatga
attcaagccattggtcgaagaaccacaaaacttgatcaagcaaaactgt
gaattgttcgaacaattgggtgaatacaagttccaaaacgctttgttgg
ttagatacactaagaaggtcccacaagtctccaccccaactttggttga
agtctctagaaacttgggtaaggtcggttctaagtgttgtaagcaccca
gaagctaagagaatgccatgtgctgaagattacttgtccgtcgttttga
accaattgtgtgttttgcacgaaaagaccccagtctctgatagagtcac
caagtgttgtactgaatctttggttaacagaagaccatgtttctctgct
ttggaagtcgacgaaacttacgttccaaaggaattcaacgctgaaactt
tcaccttccacgctgatatctgtaccttgtccgaaaaggaaagacaaat
taagaagcaaactgctttggttgaattggtcaagcacaagccaaaggct
actaaggaacaattgaaggctgtcatggatgatttcgctgctttcgttg
aaaagtgttgtaaggctgatgataaggaaacttgtttcgctgaagaagg
taagaagttggtcgctgcttcccaagctgccttaggtttgtaataa
>SEQ ID NO: 2
DAHKSEVAHRFKDLGEENFKALVLIAFAQYLQQCPFEDHVKLVNEVTEF
AKTCVADESAENCDKSLHTLFGDKLCTVATLRETYGEMADCCAKQEPER
NECFLQHKDDNPNLPRLVRPEVDVMCTAFHDNEETFLKKYLYEIARRHP
YFYAPELLFFAKRYKAAFTECCQAADKAACLLPKLDELRDEGKASSAKQ
RLKCASLQKFGERAFKAWAVARLSQRFPKAEFAEVSKLVTDLTKVHTEC
CHGDLLECADDRADLAKYICENQDSISSKLKECCEKPLLEKSHCIAEVE
NDEMPADLPSLAADFVESKDVCKNYAEAKDVFLGMFLYEYARRHPDYSV
VLLLRLAKTYETTLEKCCAAADPHECYAKVFDEFKPLVEEPQNLIKQNC
ELFEQLGEYKFQNALLVRYTKKVPQVSTPTLVEVSRNLGKVGSKCCKHP
EAKRMPCAEDYLSVVLNQLCVLHEKTPVSDRVTKCCTESLVNRRPCFSA
LEVDETYVPKEFNAETFTFHADICTLSEKERQIKKQTALVELVKHKPKA
TKEQLKAVMDDFAAFVEKCCKADDKETCFAEEGKKLVAASQAALGL
>SEQ ID NO: 3
MKWVTFLLLLFVSGSAFSRGVFRREAHKSEIAHRYNDLGEQHFKGLVLI
AFSQYLQKCSYDEHAKLVQEVTDFAKTCVADESAANCDKSLHTLFGDKL
CAIPNLRENYGELADCCTKQEPERNECFLQHKDDNPSLPPFERPEAEAM
CTSFKENPTTFMGHYLHEVARRHPYFYAPELLYYAEQYNEILTQCCAEA
DKESCLTPKLDGVKEKALVSSVRQRMKCSSMQKFGERAFKAWAVARLSQ
TFPNADFAEITKLATDLTKVNKECCHGDLLECADDRAELAKYMCENQAT
ISSKLQTCCDKPLLKKAHCLSEVEHDTMPADLPAIAADFVEDQEVCKNY
AEAKDVFLGTFLYEYSRRHPDYSVSLLLRLAKKYEATLEKCCAEANPPA
CYGTVLAEFQPLVEEPKNLVKTNCDLYEKLGEYGFQNAILVRYTQKAPQ
VSTPTLVEAARNLGRVGTKCCTLPEDQRLPCVEDYLSAILNRVCLLHEK
TPVSEHVTKCCSGSLVERRPCFSALTVDETYVPKEFKAETFTFHSDICT
LPEKEKQIKKQTALAELVKHKPKATAEQLKTVMDDFAQFLDTCCKAADK
DTCFSTEGPNLVTRCKDALA
>SEQ ID NO: 4
MKWVTFLLLLFISGSAFSRGVFRREAHKSEIAHRFKDLGEQHFKGLVLI
AFSQYLQKCPYEEHIKLVQEVTDFAKTCVADENAENCDKSIHTLFGDKL
CAIPKLRDNYGELADCCAKQEPERNECFLQHKDDNPNLPPFQRPEAEAM
CTSFQENPTSFLGHYLHEVARRHPYFYAPELLYYAEKYNEVLTQCCTES
DKAACLTPKLDAVKEKALVAAVRQRMKCSSMQRFGERAFKAWAVARMSQ
RFPNAEFAEITKLATDVTKINKECCHGDLLECADDRAELAKYMCENQAT
ISSKLQACCDKPVLQKSQCLAEIEHDNIPADLPSIAADFVEDKEVCKNY
AEAKDVFLGTFLYEYSRRHPDYSVSLLLRLAKKYEATLEKCCAEGDPPA
CYGTVLAEFQPLVEEPKNLVKTNCELYEKLGEYGFQNAVLVRYTQKAPQ
VSTPTLVEAARNLGRVGTKCCTLPEAQRLPCVEDYLSAILNRLCVLHEK
TPVSEKVTKCCSGSLVERRPCFSALTVDETYVPKEFKAETFTFHSDICT
LPDKEKQIKKQTALAELVKHKPKATEDQLKTVMGDFAQFVDKCCKAADK
DNCFATEGPNLVARSKEALA
>SEQ ID NO: 5
MKWVTFISLLFLFSSAYSRGVFRRDTHKSEVAHRFKDLGEEHFKGLVLV
AFSQYLQQCPFEEHVKLVNEVTEFAKTCVADESAENCDKSLHTLFGDKL
CTVATLRETYGEMADCCAKQEPERNECFLQHKDDNPNLPPLVRPEVDVM
CTAFHDNEATFLKKYLYEVARRHPYFYAPELLFFAARYKAAFAECCQAA
DKAACLLPKLDELRDEGKASSAKQRLKCASLQKFGDRAFKAWAVARLSQ
KFPKAEFAEVSKLVTDLTKVHTECCHGDLLECADDRADLAKYMCENQDS
ISSKLKECCDKPLLEKSHCLAEVENDEMPADLPSLAADYVESKDVCKNY
AEAKDVFLGMFLYEYARRHPDYSVMLLLRLAKAYEATLEKCCAAADPHE
CYAKVFDEFQPLVEEPQNLVKQNCELFEQLGEYKFQNALLVRYTKKVPQ
VSTPTLVEVSRNLGKVGAKCCKLPEAKRMPCAEDYLSVVLNRLCVLHEK
TPVSEKVTKCCTESLVNRRPCFSALELDEAYVPKAFNAETFTFHADMCT
LSEKEKQVKKQTALVELVKHKPKATKEQLKGVMDNFAAFVEKCCKADDK
EACFAEEGPKFVAASQAALA
>SEQ ID NO: 6
MKWVTFISLLLLFSSAYSRGVFRRDTHKSEIAHRFKDLGEEHFKGLVLI
AFSQYLQQCPFDEHVKLVNELTEFAKTCVADESHAGCEKSLHTLFGDEL
CKVASLRETYGDMADCCEKQEPERNECFLSHKDDSPDLPKLKPDPNTLC
DEFKADEKKFWGKYLYEIARRHPYFYAPELLYYANKYNGVFQECCQAED
KGACLLPKIETMREKVLASSARQRLRCASIQKFGERALKAWSVARLSQK
FPKAEFVEVTKLVTDLTKVHKECCHGDLLECADDRADLAKYICDNQDTI
SSKLKECCDKPLLEKSHCIAEVEKDAIPENLPPLTADFAEDKDVCKNYQ
EAKDAFLGSFLYEYSRRHPEYAVSVLLRLAKEYEATLEECCAKDDPHAC
YSTVFDKLKHLVDEPQNLIKQNCDQFEKLGEYGFQNALIVRYTRKVPQV
STPTLVEVSRSLGKVGTRCCTKPESERMPCTEDYLSLILNRLCVLHEKT
PVSEKVTKCCTESLVNRRPCFSALTPDETYVPKAFDEKLFTFHADICTL
PDTEKQIKKQTALVELLKHKPKATEEQLKTVMENFVAFVDKCCAADDKE
ACFAVEGPKLVVSTQTALA
>SEQ ID NO: 7
MKWVTFISLLFLFSSAYSRGVFRRDTYKSEIAHRFKDLGEQYFKGLVLI
AFSQHLQQCPYEEHVKLVREVTEFAKTCVADESAENCDKSIHTLFGDKL
CAIPSLREHYGDLADCCEKEEPERNECFLQHKNDNPDIPKLKPDPVALC
ADFQEDEQKFWGKYLYEIARRHPYFYAPELLYYAIIYKDVFSECCQAAD
KAACLLPKIEHLREKVLTSAAKQRLKCASIQKFGERAFKAWSLARLSQR
FPKADFTEISKIVTDLAKVHKECCHGDLLECADDRADLAKYICENQDTI
STKLKECCDKPLLEKSHCIAEAKRDELPADLNPLEHDFVEDKEVCKNYK
EAKHVFLGTFLYEYSRRHPDYSVSLLLRIAKIYEATLEDCCAKEDPPAC
YATVFDKFQPLVDEPKNLIKQNCELFEKLGEYGFQNALIVRYTKKVPQV
STPTLVEVARKLGLVGSRCCKRPEEERLSCAEDYLSLVLNRLCVLHEKT
PVSEKVTKCCTESLVNRRPCFSALTPDETYKPKEFVEGTFTFHADLCTL
PEDEKQIKKQTALVELLKHKPHATEEQLRTVLGNFAAFVQKCCAAPDHE
ACFAVEGPKFVIEIRGILA
>SEQ ID NO: 8
MKWVTFVSLLFLFSSAYSRGVLRRDTHKSEIAHRFNDLGEKHFKGLVLV
AFSQYLQQCPFEDHVKLVNEVTEFAKKCAADESAENCDKSLHTLFGDKL
CTVATLRATYGELADCCEKQEPERNECFLTHKDDHPNLPKLKPEPDAQC
AAFQEDPDKFLGKYLYEVARRHPYFYGPELLFHAEEYKADFTECCPADD
KLACLIPKLDALKERILLSSAKERLKCSSFQNFGERAVKAWSVARLSQK
FPKADFAEVSKIVTDLTKVHKECCHGDLLECADDRADLAKYICEHQDSI
SGKLKACCDKPLLQKSHCIAEVKEDDLPSDLPALAADFAEDKEICKHYK
DAKDVFLGTFLYEYSRRHPDYSVSLLLRIAKTYEATLEKCCAEADPPAC
YRTVFDQFTPLVEEPKSLVKKNCDLFEEVGEYDFQNALIVRYTKKAPQV
STPTLVEIGRTLGKVGSRCCKLPESERLPCSENHLALALNRLCVLHEKT
PVSEKITKCCTDSLAERRPCFSALELDEGYVPKEFKAETFTFHADICTL
PEDEKQIKKQSALAELVKHKPKATKEQLKTVLGNFSAFVAKCCGREDKE
ACFAEEGPKLVASSQLALA
>SEQ ID NO: 9
MKWVTFISLLFLFSSAYSRGVFRREAHKSEIAHRENDVGEEHFIGLVLI
TFSQYLQKCPYEEHAKLVKEVTDLAKACVADESAANCDKSLHDIFGDKI
CALPSLRDTYGDVADCCEKKEPERNECFLHHKDDKPDLPPFARPEADVL
CKAFHDDEKAFFGHYLYEVARRHPYFYAPELLYYAQKYKAILTECCEAA
DKGACLTPKLDALEGKSLISAAQERLRCASIQKFGDRAYKAWALVRLSQ
RFPKADFTDISKIVTDLTKVHKECCHGDLLECADDRADLAKYMCEHQET
ISSHLKECCDKPILEKAHCIYGLHNDETPAGLPAVAEEFVEDKDVCKNY
EEAKDLFLGKFLYEYSRRHPDYSVVLLLRLGKAYEATLKKCCATDDPHA
CYAKVLDEFQPLVDEPKNLVKQNCELYEQLGDYNFQNALLVRYTKKVPQ
VSTPTLVEISRSLGKVGSKCCKHPEAERLPCVEDYLSVVLNRLCVLHEK
TPVSEKVTKCCSESLVDRRPCFSALGPDETYVPKEFNAETFTFHADICT
LPETERKIKKQTALVELVKHKPHATNDQLKTVVGEFTALLDKCCSAEDK
EACFAVEGPKLVESSKATLG
>SEQ ID NO: 10
MKWVTFISLLFLFSSAYSRGVFRRDAHKSEVAHRFKDLGEENFKALVLI
AFAQYLQQCPFEDHVKLVNEVTEFAKTCVADESAENCDKSLHTLFGDKL
CTVATLRETYGEMADCCAKQEPERNECFLQHKDDNPNLPRLVRPEVDVM
CTAFHDNEETFLKKYLYEIARRHPYFYAPELLFFAKRYKAAFTECCQAA
DKAACLLPKLDELRDEGKASSAKQRLKCASLQKFGERAFKAWAVARLSQ
RFPKAEFAEVSKLVTDLTKVHTECCHGDLLECADDRADLAKYICENQDS
ISSKLKECCEKPLLEKSHCIAEVENDEMPADLPSLAADFVESKDVCKNY
AEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQ
VSTPTLVEVSRNLGKVGSKCCKHPEAKRMPCAEDYLSVVLNQLCVLHEK
TPVSDRVTKCCTESLVNRRPCFSALEVDETYVPKEFNAETFTFHADICT
LSEKERQIKKQTALVELVKHKPKATKEQLKAVMDDFAAFVEKCCKADDK
ETCFAEEGKKLVAASQAALGL

### PART B

### PART B—REFERENCE TO THE SEQUENCE LISTING

This application contains a Sequence Listing in computer readable form. The computer readable form is incorporated herein by reference.

### PART B—FIELD OF THE INVENTION

The present invention relates to formulations of albumin in a substantially unaggregated form and uses thereof.

### PART B—BACKGROUND OF THE INVENTION

Virus production is a key application of cell culture systems for vaccine manufacturing, gene therapy and cell therapy.

Gene and cell therapy provide new solutions for the treatment of disease and disorders caused by genetic or epigenetic defects. Major breakthroughs in these types of therapy have meant further research into optimising formulations and delivery methods. These therapies involve multiple steps including production, purification, storage, distribution and administration of viruses.

Existing research shows that the low stability of viruses poses a major challenge when carrying out these steps. This is because viruses are typically unstable and lose their biological activity or inactivate completely when left for extended periods of time at storage temperatures higher than −80° C. Therefore, viruses must be stored and transported in a frozen form and must be used soon after thawing (Harrington et al, 2017; WO 2016100364).

A low stability can affect the quality and efficacy of the administered formulation. This is characterised by a significant reduction in the titre of the virus over time. Therefore, it is desirable to increase the efficacy and stability of molecules such as therapeutics for example, in order to reduce the dose and frequency of dosing required by patients.

Cost is a significant factor when considering improvement to vaccination programs in developing countries. One way to reduce vaccine costs is to generate more stable vaccines because vaccines that do not require a cold chain, for example, should be cheaper to deliver and store. Furthermore, a loss in activity of the vaccine under suboptimal storage conditions is thought to contribute significantly to less effective vaccination programs (Brandau et al, 2003). The World Health Organisation (WHO) requires a minimum titre of vaccine formulations after one week storage at 37° C. However, many vaccines can lose over 50% of their potency when stored for only 1 hour at room temperature (Plotkin & Orenstein, 2004).

Existing technologies for increasing the stability of viruses include keeping the formulations at very low temperatures (−90° C. to −70° C.); however, loss of viability and/or infectivity of the virus occurs following multiple rounds of freeze-thaw cycles or after very short periods of time unfrozen. This can lead to wasting non-negligible amounts of expensive drug or vaccine formulations.

Another approach includes the use of natural virus mutants obtained by selection or mutagenesis (Domingo et al, 2006); however, this is not a one-size fits all approach and mutations that work at stabilising some viruses do not work in stabilising other viruses.

Yet another approach includes drying the virus (e.g. by freeze-drying/lyophilisation, spray-drying, foam-drying) (Lovalenti et al, 2016). However, drying requires specialised equipment for sample preparation (e.g. freeze dryer, vacuum pumps etc.) and exposes the virus to extreme temperatures and/or pressure conditions (Ohtake et al, 2011).

Still a further approach includes the addition of stabilising pharmaceutical excipients to the formulation (Brandau et al, 2003). Excipients are substances which exclude the active pharmaceutical ingredient (API) (e.g. a virus), that are intentionally included in the drug delivery system. A variety of excipients can be used, such as carbohydrates (sucrose, lactose, maltose, and trehalose), sugar alcohols (such as sorbitol and mannitol) (Bovarnick et al, 1950; Bedu-Addo et al, 2004), chitosan, glutamate (Kissmann et al, 2008).

The present inventors have observed that an albumin formulation comprising an albumin in substantially unaggregated form and an anion having multiple negative charges housed on a flexible backbone has beneficial properties for stabilising viruses, including preserving the integrity of the viral structure and viral infectivity during storage at cold and ambient temperatures, as well as over multiple freeze-thaw cycles. Such a formulation would help to reduce the constraints during manufacture, transportation, storage and use of the virus, by providing flexibility without unacceptable loss of stability, structure and/or infectivity.

### PART B—SUMMARY OF THE INVENTION

The invention provides the use of a formulation comprising: (i) albumin, wherein the albumin is in a substantially unaggregated form; and (ii) an anion having multiple negative charges housed on a flexible backbone, to stabilise a virus.

The invention also provides a formulation comprising: (i) albumin, wherein the albumin is in a substantially unaggregated form; and (ii) an anion having multiple negative charges housed on a flexible backbone.

The invention also provides a pharmaceutical composition comprising a formulation as described herein, and a pharmaceutically excipient, carrier or diluent thereof.

The invention also provides a formulation as described herein for use in medicine.

The invention also provides a formulation as described herein for use in gene therapy.

The invention further provides a formulation as described herein for use in cell therapy.

The invention further provides a formulation as described herein for use in vaccination and/or immunisation.

The invention further provides a formulation as described herein for use in immunotherapy, optionally wherein the immunotherapy is oncolytic virotherapy.

The invention yet further provides a method of stabilising a virus comprising combining the virus with a formulation or with a pharmaceutical composition as described herein.

The invention still further provides a method of selecting a suitable albumin for use in stabilising a virus, the method comprising determining whether the albumin is in a substantially unaggregated form.

Part B—Detailed Description of the Invention

A first aspect of the invention provides a use of a formulation comprising (i) albumin, wherein the albumin is in a substantially unaggregated form; and (ii) an anion having multiple negative charges housed on a flexible backbone, to stabilise a virus.

The term ‘albumin’ as described herein includes the meaning of a protein having the same and/or very similar tertiary structure as human serum albumin (HSA; in particular SEQ ID NO: 2) or HSA domains and has similar properties of HSA or the relevant domains. Similar tertiary structures are, for example, the structures of the albumins from species other than human, e.g. non-human primate albumin, (such as chimpanzee albumin (e.g. predicted sequence GenBank XP_517233.2), gorilla albumin or macaque albumin (e.g. GenBank NP_001182578)), rodent albumin (such as hamster albumin (e.g. GenBank A6YF56), guinea pig albumin (e.g. UniProt Q6WDN9-1), mouse albumin (e.g. GenBank AAH49971 or UniProt P07724-1 Version 3) and rat albumin (e.g. GenBank AAH85359 or UniProt P02770-1 Version 2)), bovine albumin (such as cow albumin (e.g. UniProt P02769-1)), equine albumin (such as horse albumin (e.g. UniProt P35747-1) or donkey albumin (e.g. UniProt Q5XLE4-1)), rabbit albumin (e.g. UniProt P49065-1 Version 2), goat albumin (e.g. GenBank ACF10391), sheep albumin (e.g. UniProt P14639-1), dog albumin (e.g. NCBI NP_001003026), chicken albumin (e.g. UniProt P19121-1 Version 2) and pig albumin (e.g. UniProt P08835-1 Version 2), or any one of SEQ ID NOs: 4 to 19 of WO 2013/006675, which are incorporated herein by reference. All of these albumins are included in the scope of the present invention. Mature forms of albumin (e.g. forms where all post-translational modifications and/or processing steps have been completed) are particularly preferred, and the skilled person is able to identify mature forms using publicly available information such as protein databanks and/or by using signal peptide recognition software such as SignalP (e.g. SignalP (Nielsen et al, 1997, Protein Engineering 10(1):1-6)). SignalP Version 4.0 is preferred (Petersen et al, 2011, Nat Methods 8(10):785-786). An albumin preparation for use in the methods and compositions of the present invention may comprise one or more (several) albumins. For the avoidance of doubt, SEQ ID numbers provided in Part B of the present application correspond to the SEQ ID numbers provided in Part A.

Some of the major properties of albumin are i) its ability to regulate plasma volume, ii) a long plasma half-life of around 19 days+5 days, iii) ligand binding, e.g. binding of endogenous molecules such as acidic, lipophilic compounds including bilirubin fatty acids, hemin and thyroxine (see also Table 1 of Kragh-Hansen et al, 2002, Biol Pharm Bull 25(6):695-704, hereby incorporated by reference), iv) binding of small organic compounds with acidic or electronegative features, e.g. drugs such as warfarin, diazepam, ibuprofen and paclitaxel (see also 1 of Kragh-Hansen et al, 2002, Biol Pharm Bull 25(6):695-704, hereby incorporated by reference). Not all of these properties need to be fulfilled in order to characterise a protein or fragment as an albumin. If a fragment, for example, does not comprise a domain responsible for binding certain ligands or organic compounds, the variant of such a fragment is not expected to have these properties either.

The term ‘albumin’ includes fragments, variants, and/or derivatives such as fusions and/or conjugations of an albumin or of an albumin variant. The term “fusion” includes the meaning a genetic fusion of albumin (or a variant or fragment thereof) and a non-albumin protein or peptide. The non-albumin protein or peptide may be a therapeutic, prophylactic, or diagnostic protein or peptide. Examples of albumin fusions are provided in EP624195, WO 2001/079271, WO 2003/059934, WO 2003/060071, WO 2011051489, WO 2011/124718 and EP11164955 (each incorporated herein by reference in their entirety).

By ‘variant’ we include the meaning of a polypeptide derived from a parent albumin comprising an alteration, i.e. a substitution, insertion and/or deletion, at one or more (several) positions. It is preferred if the parent albumin is a mature albumin, for example one without the leader sequence. A substitution includes the meaning of a replacement of an amino acid occupying a position with a different amino acid; a deletion includes the meaning of the removal of an amino acid occupying a position; and an insertion includes the meaning of adding amino acids (e.g. 1-3 amino acids) adjacent to an amino acid occupying a position. For example, the altered polypeptide (variant) can be obtained through human intervention by modification of the polynucleotide sequence encoding the parent albumin. The mature form of the albumin sequence may be encoded by the nucleotide sequence SEQ ID NO: 1. The variant albumin is preferably at least 70%, preferably at least 75%, more preferably at least 80%, more preferably at least 85%, even more preferably at least 90%, most preferably at least 91%, at least 92%, at least 93%, at least 94%, at least 95%, at least 96%, at least 97%, at least 98%, at least 99%, or 100% identical to SEQ ID NO: 2:

SEQ ID NO: 2 - wild-type human albumin:
DAHKSEVAHRFKDLGEENFKALVLIAFAQYLQQCPFEDHVKLVNEVTEF
AKTCVADESAENCDKSLHTLFGDKLCTVATLRETYGEMADCCAKQEPER
NECFLQHKDDNPNLPRLVRPEVDVMCTAFHDNEETFLKKYLYEIARRHP
YFYAPELLFFAKRYKAAFTECCQAADKAACLLPKLDELRDEGKASSAKQ
RLKCASLQKFGERAFKAWAVARLSQRFPKAEFAEVSKLVTDLTKVHTEC
CHGDLLECADDRADLAKYICENQDSISSKLKECCEKPLLEKSHCIAEVE
NDEMPADLPSLAADFVESKDVCKNYAEAKDVFLGMFLYEYARRHPDYSV
VLLLRLAKTYETTLEKCCAAADPHECYAKVFDEFKPLVEEPQNLIKQNC
ELFEQLGEYKFQNALLVRYTKKVPQVSTPTLVEVSRNLGKVGSKCCKHP
EAKRMPCAEDYLSVVLNQLCVLHEKTPVSDRVTKCCTESLVNRRPCFSA
LEVDETYVPKEFNAETFTFHADICTLSEKERQIKKQTALVELVKHKPKA
TKEQLKAVMDDFAAFVEKCCKADDKETCFAEEGKKLVAASQAALGL

Preferably, the albumin has at least 70% sequence identity to a mammalian albumin, preferably from an albumin selected from the group consisting of human (SEQ ID NO: 2), mouse (GenBank AAH49971 or UniProt P07724-1 Version 3), rat (GenBank AAH85359 or UniProt P02770-1 Version 2), macaque (GenBank NP_001182578), bovine (such as cow albumin (e.g. UniProt P02769-1), pig (UniProt P08835-1 Version 2), horse (UniProt P35747-1), rabbit (UniProt P49065-1 Version 2), dog (Canis lupus familiaris, Accession number NP_001003026, where residues 1 to 18 are the signal peptide, 19 to 24 are the propeptide and residues 25 to 608 are the mature sequence) or guinea pig (Cavia porcellus, UniProt Q6WDN9-1 where residues 1 to 18 are the signal peptide and 25 to 608 are the mature sequence) albumin. Preferably, the albumin has at least 70% sequence identity to a mammalian albumin selected from any of HSA (mature sequence provided in SEQ ID NO: 2, immature sequence provided in SEQ ID NO: 10 in which residues 1 to 18 of the immature sequence are the signal peptide, 19 to 24 are the propeptide and residues 25 to 609 are the mature sequence), mouse serum albumin (SEQ ID NO: 3 where residues 1 to 18 are the signal peptide, 19 to 24 are the propeptide and residues 25 to 608 are the mature sequence), rat serum albumin (SEQ ID NO: 4 where residues 1 to 18 are the signal peptide, 19 to 24 are the propeptide and residues 25 to 608 are the mature sequence), macaque serum albumin (SEQ ID NO: 5 where residues 1 to 18 are the signal peptide, 19 to 24 are the propeptide and residues 25 to 608 are the mature sequence), bovine serum albumin (SEQ ID NO: 6 where residues 1 to 18 are the signal peptide, 19 to 24 are the propeptide and residues 25 to 607 are the mature sequence), pig serum albumin (SEQ ID NO: 7 where residues 1 to 18 are the signal peptide, 19 to 24 are the propeptide and residues 25 to 607 are the mature sequence), horse serum albumin (SEQ ID NO: 8 where residues 1 to 18 are the signal peptide, 19 to 24 are the propeptide and residues 25 to 607 are the mature sequence), or rabbit serum albumin (SEQ ID NO: 9 where residues 1 to 18 are the signal peptide, 19 to 24 are the propeptide and residues 25 to 608 are the mature sequence). It is preferred if the albumin variant is a variant of a mature form of albumin. For example, when the parent albumin is mouse serum albumin, preferably, the variant has at least 70% sequence identity to residues 25 to 608 of SEQ ID NO: 3, and so on for the other mammalian sequences disclosed herein. Preferably, the albumin has at least 70% sequence identity to HSA (SEQ ID NO: 2), more preferably from 70, 75, 80, 85, 90, 91, 92, 93, 94, 95, 96, 97, 98, 99, 99.2, 99.4, 99.6, or 99.8 to 75, 80, 85, 90, 91, 92, 93, 94, 95, 96, 97, 98, 99, 99.2, 99.4, 99.6, 99.8 or 100% identity to HSA (SEQ ID NO: 2). For example, a preferred albumin has, at most, 12, 11, 10, 9, 8, 7, 6, 5, 4, 3, 2 or 1 amino acid mutations relative to wild-type HSA (SEQ ID NO: 2). For the avoidance of doubt, SEQ ID numbers provided in Part B of the present application correspond to the SEQ ID numbers provided in Part A.

Preferably, the albumin comprises at least 175 contiguous amino acids from an albumin having at least 70% sequence identity to HSA (SEQ ID NO: 2), such as from 175, 200, 225, 250, 275, 300, 325, 350, 375, 400, 425, 450, 475, 500, 525, 550 or 575 to 200, 225, 250, 275, 300, 325, 350, 375, 400, 425, 450, 475, 500, 525, 550, 575 or 580 amino acids. A fragment may comprise, consist of or substantially correspond to one or more (e.g. several) domains of albumin such as HSA (SEQ ID NO: 2), or variant thereof, such as amino acids corresponding to Domain I (residues 1 to 194±1, 2, 3, 4, 5, 6, 7, 8, 9, 10, 11, 12, 13, 14 or 15 amino acids), Domain II (residues 192 to 387±1, 2, 3, 4, 5, 6, 7, 8, 9, 10, 11, 12, 13, 14 or 15 amino acids) or Domain III (residues 381 to 585±1, 2, 3, 4, 5, 6, 7, 8, 9, 10, 11, 12, 13, 14 or 15 amino acids).

Typically, the variant albumin maintains at least one of the major properties of the parent albumin or a similar tertiary structure as HSA. For the purposes of the present invention, the sequence identity between two amino acid sequences may be determined using the Needleman-Wunsch algorithm (Needleman & Wunsch, 1970, J Mol Biol 48(3):443-453) as implemented in the Needle program of the EMBOSS package (EMBOSS: The European Molecular Biology Open Software Suite, Rice et al, 2000, Trends Genet 16(6):276-277), preferably version 5.0.0 or later. The typical parameters used are gap open penalty of 10, gap extension penalty of 0.5, and the EBLOSUM62 (EMBOSS version of BLOSUM62) substitution matrix. The output of Needle labelled “longest identity” (obtained using the—nobrief option) may be used as the percent identity and may be calculated as follows: (Identical Residues×100)/(Length of Alignment−Total Number of Gaps in Alignment).

Examples of albumin variants include those described in WO 2011/051489, WO 2011/124718, WO 2012/059486, WO 2012/150319, WO 2014/072481, WO 2013/135896, WO 2015/036579, WO 2010/092135, WO 2013/075066, WO 2014/179657, WO 2009/126920, WO 2010/059315, WO 2011/103076, WO 2012/112188, WO 2015/063611, and WO 2017/029407 (the contents of which are incorporated herein by reference in their entirety, and in particular references to albumin variants).

The term ‘parent’ or ‘parent albumin’ as described herein includes the meaning of a human or other animal (e.g. mammalian) albumin. Preferably, the other mammalian albumin is an albumin from a clinically relevant animal such as a mouse, rat, rabbit, dog or guinea pig. The parent may be a naturally occurring (wild-type) polypeptide or an allele thereof or a variant as described above.

The term ‘wild-type’ (WT) albumin as described herein includes the meaning of an albumin having the same amino acid sequence as the predominant allelic variant of albumins naturally found in an animal or in a human being. SEQ ID NO: 2 is an example of a wild-type albumin, being wild-type albumin from Homo sapiens.

By the term ‘albumin in a substantially unaggregated form’ we include the meaning that the majority of the albumin molecules are present in the formulation in an unaggregated form, and so are not, for example, aggregated with other albumin molecules and/or non-albumin molecules. Proteins such as albumin can form covalent or non-covalent aggregates, and it will be appreciated that this can lead to the formation of high molecular weight aggregates. Examples of covalent aggregation may or may not include aggregates formed by cross linking of cysteine or lysine. Examples of non-covalent aggregation may or may not include aggregates formed by electromagnetic interactions such as electrostatic interactions (e.g. ionic, hydrogen, halogen bonding), van der Waals forces, TT-effects and hydrophobic interactions. In the formulation of the present invention, it is understood that the majority of the albumin molecules are not present in such high molecular weight aggregates, but rather are present in an unaggregated form. For example, at least 80% of the albumin molecules in the formulation may be present in an unaggregated form, such as at least 85%, 90%, 95%, or 99% of the albumin molecules, or all of the albumin molecules. By the term ‘unaggregated’ we include the meaning of monomers, dimers and trimers of albumin, especially monomers. By the term ‘aggregated’ we include the meaning of tetramers, pentamers, hexamers and higher order polymers of albumin. Examples of higher order polymers may or may not include fibrils such as amyloid fibrils or protein fibrils. Preferably, the albumin of the present invention is substantially unaggregated (e.g. exists in the form of monomers and/or dimers and/or trimers), and/or preferably any aggregation of albumin referred to herein is non-covalent aggregation.

Various methods may be used to assess whether the albumin is present in a substantially unaggregated form as are well known in the art and described below. Any suitable method may be used.

For example, the percentage of albumin in a polymeric form may be used to indicate whether the albumin is present in a substantially unaggregated form. Hence, in a preferred embodiment, the albumin that is substantially present in an unaggregated form is one wherein less than 0.025% (w/w) of albumin in the formulation is in a polymeric form. By the term ‘polymer’ we include the meaning of a high molecular weight form of, or aggregate, of albumin which elutes in the void volume of a gel permeation HPLC column which separates molecules in the molecular weight range 10000 to 500000 Da, e.g. a TSK G3000SWXL. For the avoidance of doubt, the term ‘polymer’ does not include dimer or trimer; rather, we include the meaning of tetramers and higher order aggregates/multimers, for example tetramers, pentamers, hexamers. Preferably, less than 0.025% (w/w) albumin, and more preferably less than 0.024%, 0.02%, 0.015%, 0.010%, 0.005%, 0.004%, 0.003%, 0.002%, 0.001%, or 0% (w/w) albumin in the formulation is present in a polymeric form. Preferably, the less than 0.025%, 0.02%, 0.015%, 0.010%, 0.005%, 0.004%, 0.003%, 0.002%, 0.001% or 0% polymer level is observed at TO (e.g. time point zero measured within 24 or 48 hours of formulation and/or filling into a container such as a vial, bottle or bag). However, it will be appreciated that the albumin is preferably in a substantially unaggregated form during/throughout its shelf life at the recommended storage temperature, and so, for example, it is preferred that less than 0.025% (w/w) of albumin in the formulation is in a polymeric form throughout its shelf life. Typical shelf lives range from 6 months up to 5 years, and so it is preferred if less than 0.025% (w/w) of albumin is in a polymeric form for at least 1 month, 2 months, 3 months, 4 months, 5 months, more preferably at least 6 months, 7 months, 8 months, 9 months, 10 months, 11 months, 12 months, 1 year, 2 years, 3 years, 4 years or 5 years after TO. Recommended storage temperatures of liquid formulations are typically in the range of 2-8° C., such as about 2, 3, 4, 5 to about 6, 7, 8° C. Recommended storage temperatures of lyophilised formulations are typically either 2-8° C. (e.g. from about 2° C., 3° C., 4° C., 5° C. to about 6° C., 7° C., 8° C.) or −24 to −16° C. (e.g. from about −24° C., −23° C., −22° C., −21° C., −20° C. to about −19° C., −18° C., −17° C., −16° C.).

Albumin polymer levels may be measured by any suitable technique in the art such as chromatography e.g. High-Performance Liquid Size Exclusion Chromatography (SEC-HPLC), for example using albumin provided at, or diluted to, a concentration within the dynamic range of the assay, such as less than 100 mg/mL, and preferably 20 to 40 mg/mL. In a preferred embodiment, the albumin that is substantially present in an unaggregated form is one wherein less than 0.025% (w/w) of albumin in the formulation is in a polymeric form as measured by

High-Performance Liquid Size Exclusion Chromatography (SEC-HPLC) using an injection sample of albumin provided at or diluted to less than 100 mg/mL, and preferably 20 to 40 mg/mL. Further explanations of how to assess albumin aggregation by assessing polymer levels using chromatography are provided in the Examples, and in particular the method in Example 3.

In another example, dynamic light scattering (DLS) may be used to indicate whether the albumin in the formulation is present in a substantially unaggregated form.

Hence, in another embodiment, the albumin that is substantially present in an unaggregated form is one wherein the hydrodynamic radius (R_{H}) of albumin as assessed by DLS does not substantially change when the albumin is incubated at 40° C. for a set period of time. Preferably, the hydrodynamic radius of the albumin does not change more than 4.5% when incubated at 40° C. for a duration of at least 3 days, such as no more than 4, 3.5, 3, 2.5, 2, 1.5, 1, 0.5, 0.25 or 0%. Preferably, the no more than 4.5, 4, 3.5, 3, 2.5, 2, 1.5, 1, 0.5, 0.25 or 0% change is observed at 3 days. It will be understood that the hydrodynamic radius of albumin (e.g. at a concentration of around 5 mg/mL) is around 3.5 nm, and so the albumin that is present in a substantially unaggregated form may be one where the hydrodynamic radius of the albumin is maintained at around 3.5±0.2 nanometres (nm), preferably +0.15, 0.125, 0.1, 0.075, 0.05, 0.025 or 0 nm, when incubated at 40° C. for a duration of at least 3 days. Measuring the hydrodynamic radii of albumin by Dynamic Light Scattering (DLS) is a standard technique in the art that may be carried out, for example using albumin at 5 mg/mL. Further explanations of how to use DLS to assess the hydrodynamic radius are provided in the Examples, including DLS cumulants analysis. It will be appreciated that the measurement of hydrodynamic radii of albumin by DLS may be carried out in the presence or absence of octanoate. In the presence of octanoate, aggregation of albumin is expected to take longer.

In yet another embodiment, the albumin that is substantially present in an unaggregated form is one wherein the polydispersity index (PD index) of albumin as assessed by DLS when the albumin is in solution (e.g. in PBS) in the absence of a virus, is less than or equal to 0.05. Preferably, the PD index is less than 0.05, and more preferably less than 0.04, 0.03, 0.02, 0.01 or 0. Preferably, the less than 0.05, 0.04, 0.03, 0.02, 0.01 or 0 is observed at TO (e.g. time point zero measured within 24 or 48 hours of formulation) and/or during/throughout its shelf life at the recommended storage temperature. Preferred durations of shelf life and preferred storage temperatures include those mentioned above. Preferably, the PD is determined for albumin provided at or diluted to about 5 mg/mL. The PD index is a number calculated from a simple two-parameter fit to the correlation data (the cumulant analysis). The PD index is dimensionless and scaled such that values smaller than 0.05 are rarely seen other than with highly monodisperse standards. Values greater than 0.7 indicate that the sample has a very broad size distribution. The calculations for these parameters are defined in the ISO standard document 13321:1996 E and ISO 22412:2008 (https://www.malvernpanalytical.com/en/learn/knowledge-center/whitepapers/WP111214DLSTermsDefined). Measuring the PD index of albumin by Dynamic Light Scattering (DLS) is a standard technique in the art that may be carried out, for example using albumin at 5 mg/mL. Further explanations of how to use DLS to assess the PD index are provided in the Examples including DLS cumulants analysis.

As described further in the Examples, the inventors have found that an albumin that is substantially in an unaggregated form, which meets the following parameters:
there is less than 0.025% (w/w) of albumin in the formulation is in a polymeric form
the polydispersity index of albumin is equal or less than 0.05; and
the hydrodynamic radius of albumin does not change more than 4.5% when incubated at 40° C. for a duration of 3 days;
is especially good at stabilising viruses. The inventors have therefore designated such an albumin as a “good albumin” herein. It is particularly preferred therefore that the albumin in the formulation is one that meets one or more (e.g. two or more, or all three of) the following parameters:
there is less than 0.025% (w/w) of albumin in the formulation is in a polymeric form;
the polydispersity index of albumin is equal or less than 0.05; and
the hydrodynamic radius of albumin does not change more than 4.5% when incubated at 40° C. for a duration of 3 days.

Further desirable characteristics of the albumin that have also been identified by the inventors are described below.

In one embodiment, the total amount of lipids (excluding octanoate) in the formulation is equal or less than 50 μg (microgram) lipids/mL of 100 mg/mL of albumin, such as less than or equal to 40 μg lipids/mL, 30 μg lipids/mL, 20 μg lipids/mL or 10 μg lipids/mL. The total amount of lipids (excluding octanoate) may be measured by any suitable technique in the art, including gas chromatography-mass spectrometry (GC-MS). Different lipids are characterised by their peaks and can be identified using standard calibration curves as known in the art. Throughout this document, the term “lipids” includes “fatty acids”.

In another embodiment, the albumin is one where the N-terminal degradation of the albumin is less than 12.5%, such as less than 12%, less than 11%, less than 10%, less than 9%, less than 8%, less than 7%, less than 6%, less than 5% or less than 4%, or from 0, 1, 2, 3, 4, 5, 6, 7, 8, 9, 10, 11, or 12%, to 1, 2, 3, 4, 5, 6, 7, 8, 9, 10, 11, 12, or 12.5%. An albumin having N-terminal degradation of less than 8% is particularly preferred. The phrase ‘the N-terminal degradation of the albumin is less than 12.5%’ includes the meaning of less than 12.5% of the albumin molecules showing some level of N-terminal degradation.

Preferably, the N-terminal degradation of albumin is less than 12.5, 12, 11.5, 11, 10.5, 10, 9.5, 9, 8.5, 8, 7.5, 7, 6.5, 6, 5.5, 5, 4.5, 4, 3.5, 3, 2.5, 2, 1.75, 1.5, 1.25, 1, 0.75, 0.50, 0.25 or 0%, for example from 0, 0.25, 0.5, 0.75, 1, 1.1, 1.2, 1.3, 1.4, 1.5, 1.6, 1.7, 1.8, 1.9, 1.95, 1.96, 1.97, 1.98, 1.99, 2, 2.5, 3, 3.5, 4, 4.5, 5, 5.5, 6, 6.5, 7, or 7.5%, to 0.25, 0.5, 0.75, 1, 1.1, 1.2, 1.3, 1.4, 1.5, 1.6, 1.7, 1.8, 1.9, 1.95, 1.96, 1.97, 1.98, 1.99, 2, 2.5, 3, 3.5, 4, 4.5, 5, 5.5, 6, 6.5, 7, 7.5, 8, 8.5, 9, 9.5, 10, 10.5, 11, 11.5, 12, or 12.5% is observed at TO (e.g. time point zero measured within 24 or 48 hours of formulation) and/or during shelf life. Preferred durations of shelf life and preferred storage temperatures include those mentioned above. The percentage of N-terminal degradation of albumin may be assessed using any suitable technique in the art, including mass spectrometry such as electrospray ionisation mass spectrometry (ESI-MS). For example, when ESI-MS is used, the peak with the highest abundance (attributable to non-N-terminally degraded albumin) may be normalised and set to 100% and all peaks attributable to N-terminal degraded albumins should be less than 12.5% of the “100% peak”. A preferred ESI-MS protocol for assessing the intact mass of albumin is provided in Example 5.

Albumin may or may not be modified, such as cysteinylated. By “cysteinylated” we include addition of a cysteine residue by disulphide bonding to an existing cysteine residue such as Cys34 of wild-type HSA (SEQ ID NO: 2), or a corresponding Cys in albumins from other species or in albumin variants. Such cysteinylation may generate a “branch” off the albumin sequence. Calculation of the relative intensity of N-terminal degradation may preferably include both the amount of intact unmodified (e.g. non-cysteinylated) albumin and intact modified (e.g. cysteinylated) albumin, and the amount unmodified (e.g. non-cysteinylated) albumin demonstrating N-terminal degradation and the amount modified (e.g. cysteinylated) albumin demonstrating N-terminal degradation. Alternatively, the relative intensity of N-terminal degradation may be calculated based on the amount of intact unmodified (e.g. non-cysteinylated) albumin and the amount of unmodified (e.g. non-cysteinylated) albumin demonstrating N-terminal degradation. Preferably, calculation of the relative intensity of N-terminal degradation does not include the amount of intact modified (e.g. cysteinylated) albumin and the amount of modified (e.g. cysteinylated) albumin demonstrating N-terminal degradation.

In yet another embodiment, the albumin is one where the sum % of all post-translational modifications however excluding all oxidised species are less than 75%. By the “sum % of all post-translational modifications however excluding all oxidised species being less than 75%” we include the meaning that less than 75% of albumin molecules are post-translationally modified by means other than oxidation. Preferably, the sum % of all post-translational modifications other than oxidation of albumin is less than 75%, such as less than 70, 65, 60, 55, 50, 45, 40, 35, 30, 25, 20, 15, 14, 13, 12, 11, 10, 9.9, such as less than 9.8, 9.75, 9.5, 9.25, 9, 8, 7, 6, 5, 4, 3, 2, 1 or is about 0%. Preferably, the sum % of all post-translational modifications other than oxidation of less than 75, 70, 65, 60, 55, 50, 45, 40, 35, 30, 25, 20, 15, 14, 13, 12, 11, 10, 10, 9.9, 9.75, 9.5, 9.25, 9, 8, 7, 6, 5, 4, 3, 2, 1 or about 0% is observed at TO (e.g. time point zero measured within 24 or 48 hours of formulation) and/or during shelf life. Especially preferred albumins have a sum % of all post-translation modifications other than oxidation of less than 30%, more preferred less than 25%, even more preferred less than 10%. Preferred durations of shelf life and preferred storage temperatures include those mentioned above. The percentage of post-translational modifications may be assessed using any suitable technique in the art, including mass spectrometry such as electrospray ionisation mass spectrometry (ESI-MS). Preferably, the ESI-MS is performed using a Bruker MicroTOF II with injection via a Waters™ Acquity UPLC wherein the sensitivity is more than 25 μmol (pico moles) and the resolution is about 3500. It will be appreciated therefore that the albumin may be one where the sum % of all post-translational modifications however excluding oxidised species are less than 75% as determined using such an instrument and parameters.

For example, when ESI-MS is used, the peak with the highest abundance (for instance the peak attributable to non-post-translationally modified albumin) is normalised and set to 100%. All the peaks attributable to post-translationally modified albumin (e.g. glycation and cysteinylation) are summed to create a total which should be less than 75% of the “100% peak”. The measurement is an intensity height measurement and “75%” corresponds to “75% of the intensity of the highest peak”.

In yet another embodiment, the albumin is one where the amount of copper (Cu) in the formulation is less than 0.15 μg/g (microgram/gram) of albumin. Preferably, the amount of copper in the formulation is less than 0.14 μg/g of albumin, such as less than 0.13, 0.12, 0.11, 0.10, 0.09, 0.08, 0.07, 0.06, 0.05, 0.04, 0.03, 0.02, 0.01 or is about 0 g/g of albumin.

Preferably, the amount of copper in the formulation of less than 0.15, 0.14, 0.13, 0.12, 0.11, 0.10, 0.09, 0.08, 0.07, 0.06, 0.05, 0.04, 0.03, 0.02, 0.01 or is about 0 μg/g of albumin is observed at T0 (e.g. time point zero measured within 24 or 48 hours of formulation) and/or during shelf life. Preferred durations of shelf life and preferred storage temperatures include those mentioned above. The amount of copper can be assessed by any suitable technique in the art, including mass spectrometry such as inductively coupled plasma mass spectrometry (ICP-MS).

The inventors have found that albumins having a certain level of free thiol groups are particularly good at stabilising viruses, and so in one embodiment, the formulation contains at least 0.5 moles free thiol per mole albumin. Thus, the formulation may comprise at least 0.55, 0.60, 0.65, 0.70, 0.75, 0.80, 0.85, 0.90, 0.95, 0.96, 0.97, 0.98 or 0.99 moles free thiol per mole albumin. The formulation may comprise at least 1 mole, or 1 mole free thiol per mole albumin. The term ‘free thiol’ includes the meaning of a thiol group which is available for reaction with another group. For example, a free thiol is not already connected to another thiol group via a disulfide bond or is not oxidised. Free thiols may be provided by cysteine amino acids, such as Cys34 of HSA (SEQ ID NO: 2), or equivalent Cys in albumin variants or in albumins from other species.

Thus, it will be appreciated that in one embodiment, the albumin may comprise one or more (e.g. several) free thiol groups (“thiols”) per molecule of albumin, for example at least 1, 2, 3, 4, 5, 6, 7, 8, 9 or 10 free thiol groups. For an albumin comprising one free thiol group per molecule of albumin such as human serum albumin which contains a free thiol provided by Cys34, the theoretical free thiol content is 1 mole free thiol per mole albumin or fragment or fusion thereof.

For an albumin comprising two free thiol groups per molecule of albumin or fragment thereof, such as a variant of human serum albumin which contains a free thiol provided by Cys34 and an additional free thiol provided by another Cys residue, the theoretical free thiol content is 2 moles free thiol per mole albumin. And so on. Albumins containing 2 or more cysteine residues that provide free thiol include mouse albumin and variants of albumin.

Preferably the desirable free thiol level per mole albumin is observed after storage or incubation at at least 25° C. for at least 1 month, more preferably for at least 2, 3, 4, 5, 6, 9 or 12 months. More preferably, the free thiol level, per mole albumin is observed after storage or incubation at at least 40° C. for at least 1 month, more preferably for at least 2, 3, 4, 5 or 6 months. Preferably the free thiol level, per mole albumin is observed after storage or incubation at a temperature of from 2 to 8° C., such as from 2, 3, 4, 5, 6 or 7 to 3, 4, 5, 6, 7 or 8° C., for example at about 4° C., for a duration of at least 1 month, more preferably for at least 2, 3, 4, 5 or 6 months or for at least 1, 2, 3, 4, 5, or 6 years.

The free thiol level may be measured by any suitable free thiol assay known in the art, such as a colourimetric free thiol assay, for example a DTNB assay or a DTDP assay. A DTNB assay is preferred.

A suitable DTNB assay that is particularly preferred comprises the following method, the volumes of components may be adjusted, the ratio of the components should remain as set out below:
a) preparing a stock solution of DTNB at 4 mg/mL in assay buffer (0.1 M phosphate buffer, pH 8.0) on the day of the measurement and protecting from light. For the standard curve, a 1.5 mM stock solution of L-Cysteine (Sigma #30089; MW=121.165 g/mol) in water on the same day of the assay.
b) from this stock, preparing Cysteine standards solutions by dilution in assay buffer at the following concentrations: 1.5, 1.25, 1.0, 0.75, 0.5, 0.25, 0 mM.
c) preparing assay solutions mixing 500 μL (microlitres) of assay buffer with 50 μL of standard or sample,
d) adding 10 μL (microliters) 0.01M 5,5′-Dithiobis-(2-Nitrobenzoate) (DTNB) to the sample, the control and the buffer ‘blank’,
e) incubating for 10 minutes in the dark at room temperature (15 to 25° C., 20° C. is preferred), and
f) measuring the increase in absorbance at a wavelength of 412 nm of the sample, the control and the buffer ‘blank’, and
g) determining the free thiol level of the sample.

In one embodiment, the albumin in the formulation is one that meets one or more of (preferably all three of the following parameters):
there is less than 0.025% (w/w) of albumin in the formulation is in a polymeric form;
the polydispersity index of albumin is equal or less than 0.05; and
the hydrodynamic radius of albumin does not change more than 4.5% when incubated at 40° C. for a duration of 3 days, and meets one or more of the following parameters:
i) the total amount of lipids (excluding octanoate) in the formulation is equal or less than 50 μg (microgram) lipids/mL albumin;
ii) the albumin is one where the N-terminal degradation of the albumin is less than 12.5%
iii) the albumin is one where the sum % of all post-translational modification is less than 75%;
iv) the albumin is one where the amount of copper (Cu) in the formulation is less than 0.15 μg/g of albumin (microgram/gram); and
v) the formulation contains at least 0.5 moles free thiol per mole albumin.

The albumin may be sourced from serum or a recombinant source. Advantageously, the formulation may comprise a recombinant albumin. That is, the albumin may be sourced from a recombinant organism such as a recombinant microorganism, recombinant plant or recombinant animal. Since some users prefer animal-free ingredients, it is more preferred that the albumin is sourced from a non-animal recombinant source, such as a recombinant microorganism or recombinant plant. Preferred organisms include prokaryotes and, more preferably, eukaryotes such as animals, plants, fungi or yeasts, for example, but not limited to, the following species in which albumins have been successfully expressed as recombinant proteins, for example see the following citations which are incorporated herein by reference:
fungi (including but not limited to Aspergillus (WO06066595), Kluyveromyces (Fleer 1991, Bio/technology 9, 968-975), Pichia (Kobayashi 1998 Therapeutic Apheresis 2, 257-262) and Saccharomyces (Sleep 1990, Bio/technology 8, 42-46))
animals (Barash 1993, Transgenic Research 2, 266-276)
plants (including but not limited to potato and tobacco (Sijmons 1990, Bio/technology 8, 217 and Farran 2002, Transgenic Research 11, 337-346) and rice e.g. Oryza sativa)
mammalian cells such as CHO and HEK
prokaryotes (Pandjaitab 2000, J. Allergy Clin. Immunol. 105, 279-285)) e.g. E. coli (EP73,646).

Preferably the albumin is recombinant sourced from a fungal host (e.g. yeast host), preferably from a genus selected from Saccharomyces (e.g. Saccharomyces cerevisiae) or Pichia (e.g. Pichia pastoris) or Kluyveromyces (e.g. Kluyveromyces lactis, Kluyveromyces marxianus), Hansenula polymorpha, Schizosaccharomyces pombe, Yarrowia lipolytica and Arxula adeninivorans, Candida (e.g. Candida utilis), Zygosaccharomyces bailii more preferably a Saccharomyces, most preferably Saccharomyces cerevisiae (https://doi.org/10.1016/j.biotechadv.2011.09.011 i.e. Celik & Calik, 2012; https://doi.org/10.1007/978-1-4939-9024-5_1 i.e. Gunduz Ergun et al, 2019).

In an embodiment, the formulation comprises from 0.4 to 300 mg/mL, such as 0.5 to 300 mg/ml of albumin. For example, the formulation may comprise from about 25, 50, 75, 100, 125, 150, 175, 200, 225, 250, or 275 mg/mL to about 75, 100, 125, 150, 175, 200, 225, 250, 275 or 300 mg/ml albumin. In a particular example, the formulation may comprise from about 50 to 300 mg/mL, such as from about 100 to 200 mg/mL albumin.

Preferably, the formulation in the use of the first aspect of the invention comprises from 0.4 to 50 mg/mL, such as 0.5 to 50 mg/ml of albumin. For example, the formulation may comprise from about 0.4, 0.5, 1, 2.5, 5, 10, 15, 20, 25, 30, 35 or 40 to about 25, 30, 35, 40, 45, or 50 mg/ml of albumin. An albumin concentration of from about 1 to 20 mg/ml is preferred, and albumin concentrations of about 1 mg/mL, about 2.5 mg/mL, about 5 mg/ml are particularly preferred.

It will be appreciated that the albumin in the formulation to be used for stabilising a virus according to the first aspect of the invention, may itself be provided by an already prepared formulation (herein referred to as an “albumin composition”). By the albumin in the formulation being “provided by an albumin composition” we include the meaning that the albumin in the formulation is provided by reformulating an already prepared albumin composition, for example by (i) combining the albumin composition with an anion having multiple negative charges housed on a flexible backbone, and optionally the one or more other components such as a buffer, as described herein in relation to the first aspect of the invention, and optionally (ii) diluting the albumin composition to obtain the desired concentration of albumin. Preferred examples of suitable albumin compositions include any of those selected from the group consisting of: (a) a composition comprising 130 to 160 mM, preferably 145 mM, of sodium, 29 to 35 mM, preferably 32 mM, of octanoate, 10 to 20 mg/L, preferably 10 to 15 mg/L, of Polysorbate 80, 190 to 210 mg/mL, preferably 200 mg/mL, of albumin (w/v), wherein the pH is 6.7 to 7.3, preferably 7 and wherein the percentage of albumin (w/w) in a polymeric form is 0 to 1%, more preferably less than 0.025%; (b) a composition comprising 120 to 160 mM, preferably 145 mM, of sodium, 4 to 12 mM, preferably 8 mM, of octanoate, 0 to 50 mg/L, preferably 25 to 45 mg/L, of Polysorbate 80, 95 to 105 mg/mL, preferably 100 mg/mL, of albumin (w/v), wherein the pH is 6.4 to 7.4, preferably 7, and wherein the percentage of albumin (w/w) in a polymeric form is 0 to 1%, more preferably less than 0.025%; (c) a composition comprising 120 to 160 mM, preferably 145 mM, of sodium, 8 to 24 mM, preferably 16 mM, of octanoate, 0 to 100 mg/L, preferably 50 to 70 mg/L, of Polysorbate 80, 190 to 210 mg/ml, preferably 200 mg/mL, of albumin (w/v), wherein the pH is 6.4 to 7.4, preferably 7, and wherein the percentage of albumin (w/w) in a polymeric form is 0 to 1%, more preferably less than 0.025%; (d) a composition comprising 200 to 300 mM, preferably 230 to 260 mM, of sodium, 0 to 3 mM, preferably 0 to 1 mM of octanoate, 95 to 105 mg/mL, preferably 100 mg/mL, of albumin (w/v), wherein the pH is 6 to 7, preferably 6.5, and wherein the percentage of albumin (w/w) in a polymeric form is 0 to 1%, more preferably less than 0.025%. It will be appreciated that the albumin in these albumin compositions are considered “good” albumins.

Particularly preferred examples of albumins (e.g. recombinant human albumin) in a substantially unaggregated form include those manufactured in yeast, particularly in Saccharomyces cerevisiae, such as the following known commercial presentations of recombinant yeast-derived albumin: Recombumin® Prime (formerly Recombumin®), Recombumin® Elite (formerly AlbiX®), Recombumin® Alpha (formerly Albucult®) (all sourced from Albumedix Limited), or any preparation that is similar thereto. It will be appreciated that all these albumins will be considered “good” albumins.

It will further be appreciated that the formulation that is used to stabilise the virus in accordance with the first aspect of the invention may be conveniently prepared from a stock solution of albumin that contains a higher concentration of albumin than the concentration of albumin in the formulation ready for use to stabilise the virus. In one example, an albumin stock solution may comprise from 50 to 300 mg/mL albumin, such as from 100 to 200 mg/ml (e.g. about 100 or about 200 mg/mL albumin) and may be diluted so that the concentration of albumin in the formulation ready for use to stabilise a virus is from 0.4 to 50 mg/mL, such as 0.5 to 50 mg/mL of albumin (e.g. from about 1 to 20 mg/mL such as 1 mg/mL, 2.5 mg/ml or 5 mg/mL). It will be understood that any of the already prepared albumin compositions (a)-(d) or any of the commercial presentations of albumin described above may constitute such a stock solution.

It will be appreciated that the formulation described herein may or may not comprise octanoate and/or that the formulation described herein may or may not comprise phosphate.

In a preferred embodiment, the formulation that is used to stabilise a virus is a liquid formulation.

In an embodiment, the formulation that is used to stabilise a virus is substantially free of a sugar (such as sucrose, lactose, maltose, or trehalose) and/or a sugar alcohol (such as sorbitol or mannitol). The inventors found that, generally, mannitol on its own did not stabilise the virus, and that mannitol-containing samples resulted in oligomerisation and aggregate formation. However, it is believed that mannitol may be included when used in combination with other excipients and/or salts.

By the term ‘anion having multiple negative charges housed on a flexible backbone’ we include the meaning of any flexible backbone that houses two or more negative charges, such as three or more, four or more, or five or more negative charges. Thus, the anion may be multivalent such as divalent, trivalent or tetravalent. In one embodiment, the multiple negative charges housed on a flexible backbone are non-consecutive. In another embodiment, the multiple negative charges housed on a flexible backbone are consecutive. By ‘flexible backbone’ we include the meaning of a backbone of single covalent bonds that connects the atoms carrying the negatively charged groups, such that there is rotational freedom between the negative charges. In yet another embodiment, the multiple negative charges housed on a flexible backbone are located at least at both ends of the backbone.

Examples of suitable anions having multiple (such as two, three, four, five) negative charges housed on a flexible backbone include multivalent organic anions, such as organic carboxylic acids. For instance, the anion may be a divalent or a trivalent carboxylic acid. The anion may be one which has a carboxylic acid group at both ends of the flexible backbone. The anion may be one that has 2, 3 or 4 carboxylic acid groups, and which does not have one or more amino groups attached to the backbone. The anion may be one that has at least two carboxylic acid groups (e.g. at least 2, 3, 4 or 5 carboxylic acid groups) and which does not contain one or more amide groups. In one embodiment, the anion is an intermediate of the citric acid cycle, such as any of citrate, isocitrate, α-ketoglutarate, succinyl CoA, succinate, fumarate, malate, or oxaloacetate. In a further embodiment, the anion is any of tartaric acid, maleic acid, succinic acid, aconitate, adenosine triphosphate or sodium tripolyphosphate. Inorganic anions are also included, such as EDTA. In a preferred embodiment, the anion is an intermediate of the tricarboxylic acid cycle, preferably citrate or oxaloacetate, more preferably citrate.

In one embodiment, the anion having multiple negative charges housed on a flexible backbone is not one of, or several of, EDTA (ethylenediaminetetraacetic acid), glutamate, glutamic acid, a glutamic acid alkali metal salt, a glycosamino glycan, a branch of a negatively charged polysaccharide or a polysaccharide. In one embodiment, the anion having multiple negative charges housed on a flexible backbone is not any of EDTA, glutamate, glutamic acid, a glutamic acid alkali metal salt, a glycosamino glycan, a branch of a negatively charged polysaccharide or a polysaccharide.

In one embodiment, the anion having multiple negative charges housed on a flexible backbone is not one of, or both of, glutamic acid and ATP.

Preferably, the anion having multiple negative charges housed on a flexible backbone thermally stabilises the albumin.

By ‘the anion thermally stabilises the albumin’, we include the meaning that the anion increases the thermal stability of the albumin more than the thermal stability of albumin in the absence of the anion.

Thermal stability can be assessed by measuring stability at a given temperature as a function of time. It can readily be determined, for example, by measuring binding to a binding partner or by using spectroscopic methods such as fluorescence, Circular Dichroism (CD), light scattering or Differential Scanning calorimetry (DSC) at a particular temperature, for example using albumin at 5 mg/mL. For instance, the ability of albumin to retain binding to a binding partner following incubation for a set period of time at a particular temperature may be used to determine the thermostability of the albumin. Alternatively, the tertiary structure of the albumin following incubation for a set period of time at a particular temperature may be assessed, for example by spectroscopic methods. It will be appreciated that the tertiary structure may be assessed directly (e.g. by spectroscopic methods) or indirectly (e.g. by assessing any one or more activities of albumin that are dependent on its structure). Since conformational stability can be related to stabilisation against thermal stress, it will be understood that the anion that increases the thermal stability of the albumin may also increase the conformational stability of the albumin.

In one embodiment, the anion having multiple negative charges housed on a flexible backbone is one that does not cause a change in the hydrodynamic radius of the albumin in the formulation of more than 5% when incubated at 40° C. for a duration of at least 20 hours. In another embodiment, the anion having multiple negative charges housed on a flexible backbone is one that maintains the hydrodynamic radius of the albumin at around 3.5±0.2 nanometres (nm) when incubated at 40° C. for a duration of at least 20 hours.

Without wishing to be bound by theory, the inventors believe that the anion thermally stabilises the albumin by binding to it and ‘stapling’ the helices. By ‘stapling’, we include the meaning that the helices are constrained by virtue of binding to the albumin. In other words, the anion might be thermally stabilising the albumin by binding to it and ‘co-ordinating’ the helices. By ‘co-ordinating’, we also include the meaning that the helices are constrained by virtue of intra-molecular bonding to the albumin which may be driven by direct interaction or indirect charge/solvent effects. By locking the albumin conformation in a more stable one, the anion may preserve the stabilising potential even in conditions where possible structural modifications could impair it (e.g. stressing the formulated product). Binding can be assessed directly (e.g. by Isothermal Titration calorimetry (ITC)) or indirectly (e.g. by the increase in thermal stability) by any suitable technique known in the art.

In a further embodiment, the anion having multiple negative charges housed on a flexible backbone may increase the conformational stability and/or the chemical stability of the albumin.

Preferably, the anion having multiple negative charges housed on a flexible backbone leads to a higher protein recovery in the formulation following storage for 1 to 36 months (e.g. 2 to 36 months) at 25° C. Preferably, the anion having multiple negative charges housed on a flexible backbone leads to a higher monomer recovery in the formulation following 36 months at 5° C. or 25° C. By ‘higher protein or monomer recovery’ we include the meaning that the recovery of protein or monomer is higher in the presence of the anion compared to the recovery of protein or monomer in the absence of the anion. Protein and monomer recovery may be measured using any suitable technique in the art such as mass spectrophotometry. For example, the amount of total protein or the amount of monomeric protein can be measured at time point zero using any suitable technique in the art such as SEC-HPLC. This allows determination of the starting protein or monomer concentration (mg/mL) before any aggregation that takes places during storage. By then measuring the amount of protein or monomer at subsequent time intervals during storage, and normalising these values to the amount of total protein or monomeric protein at time point zero, it is possible to calculate the protein recovery rate or monomer recovery rate in a way that can be compared between formulations. The starting amount of protein or monomer can be set to 1, and the recovery rate expressed on a scale of 0 to 1. For example, if the total protein concentration at time point zero is 9.8 mg/mL, a recovery of total protein of 0.985 is 0.985×9.8 mg/mL=9.653 mg/mL, and so on. When measured using SEC-HPLC, any decrease in the amount of total protein indicates lost material on the pre-column and indicates the presence of aggregates which are filtered out in the pre-column, and any decrease in the amount of monomeric protein correlates to the appearance of other peaks (i.e. dimers, trimers and non-filtered out polymers).

In one embodiment, the anion having multiple negative charges housed on a flexible backbone is one that enables at least 0.985 normalised total protein recovery at 25° C. after at least 1 month, for example after at least 2, 3, 4, 5 or 6 months, and/or one that enables 0.99 normalised total protein recovery at 5° C. after at least 1 month for example after at least 2, 3, 4, 5 or 6 months.

In another embodiment, the anion having multiple negative charges housed on a flexible backbone is one that enables at least 0.97 normalised monomer amount at 25° C. after at least 36 months and/or at least 0.98 normalised monomer amount at 5° C. after at least 36 months.

It will be appreciated that albumin has a tendency to deamidate, to oxidise, to N-terminally degrade and/or to fragment over time.

In one embodiment, the anion having multiple negative charges housed on a flexible backbone protects against deamidation of the albumin. Conveniently, deamidation is measured by analysing ammonia concentration using an enzymatic assay (e.g. Ammonia Assay Kit AA0100 (sigmaaldrich.com)). The decrease in absorbance at 340 nm due to oxidation of NADPH (measured, for example, on a Shimadzu UV-2101PC UV-VIS spectrophotometer) is proportional to the ammonia concentration in the sample. Conveniently, a positive ammonia control (e.g. an inorganic ammonium salt) may be used to indicate the validity of the assay performance. By ‘the anion protects against deamidation of the albumin’, we include the meaning that any anion that, by virtue of its presence, reduces (partially or substantially all of) the deamidation compared to the level of deamidation when the anion is not present. Preferably, the anion having multiple negative charges housed on a flexible backbone protects against deamidation such that in a sample comprising 10 mg/mL albumin incubated at 25° C., the ammonia concentration remains at less than 100 M (micromolar) over 36 months.

In yet another embodiment, the anion having multiple negative charges housed on a flexible backbone protects against N-terminal degradation of the albumin. By ‘the anion protects against N-terminal degradation of the albumin’, we include the meaning that any anion that, by virtue of its presence, reduces (partially or substantially all of) the N-terminal degradation of the albumin compared to the level of N-terminal degradation when the anion is not present. N-terminal degradation has been identified and characterised as an auto-degradation occurring upon storage at temperatures higher than 30° C. (Chan et al, 1995). The degradation is specific to human derived albumin and is due to the particular N-terminal sequence. N-terminal degradation can be detected by ESI-MS from the presence of a less abundant species which corresponded to the albumin intact mass (i.e. the total molecular weight determined by mass spectrometry without prior digestion or fragmentation) minus the first two amino acids (Δ=−186 Da; Des-Asp′-Ala′-HSA (HSA lacking the first two N-terminal amino acid residues)). Preferably, N-terminal degradation of albumin is measured by Mass Analysis of Albumin by Electrospray Mass Spectrometry (ESI-MS) intact mass after desalting or any suitable technique as is known in the art and as described further in the Examples. Typically, samples to be analysed are at concentrations of 0.3 mg/ml albumin or less. When measured by ESI-MS, the level of N-terminal degradation is typically expressed as a percentage relative to the highest abundant peak (attributable to no N-terminal degradation) taken as 100%. Preferably, in the presence of the anion, N-terminal degradation of the albumin does not exceed 12.5%, for example when the albumin is incubated at 25° C., more preferably no more than 12, 11.5, 11, 10.5, 10, 9.5, 9, 8.5, 8, 7.5, 7, 6.5, 6, 5.5, 5, 4.5, 4, 3.5, 3, 2.5, 2, 1.75, 1.5, 1.25, 1, 0.75, 0.50, 0.25 or 0%, for example from 0, 0.25, 0.5, 0.75, 1, 1.1, 1.2, 1.3, 1.4, 1.5, 1.6, 1.7, 1.8, 1.9, 1.95, 1.96, 1.97, 1.98, 1.99, 2, 2.5, 3, 3.5, 4, 4.5, 5, 5.5, 6, 6.5, 7, or 7.5% to 0.25, 0.5, 0.75, 1, 1.1, 1.2, 1.3, 1.4, 1.5, 1.6, 1.7, 1.8, 1.9, 1.95, 1.96, 1.97, 1.98, 1.99, 2, 2.5, 3, 3.5, 4, 4.5, 5, 5.5, 6, 6.5, 7, 7.5, 8, 8.5, 9, 9.5, 10, 10.5, 11, 11.5, 12, or 12.5%. For example, when the albumin is incubated at 25° C. for the desired duration, in the presence of the anion, and ESI-MS is used to measure N-terminal degradation, the peak with the highest abundance (attributable to non-N-terminally degraded albumin) is set to 100% and all peaks attributable to N-terminal degraded albumins should be less than 12.5% of the “100% peak”. The measurement is an intensity height measurement and “12.5%” corresponds to “12.5% of the intensity of the highest peak”.

A preferred formulation according to the use of the first aspect of the invention comprises from 2 to 500 mM of anion having multiple negative charges housed on a flexible backbone, such as 10 to 500 mM of anion having multiple negative charges housed on a flexible backbone. The anion concentration may be from about 2, 5, 10, 15, 20, 25, 30, 35, 40, 45 or 50 to about 50, 55, 60, 65, 70, 75, 100, 200, 300, 400 or 500 mM. An anion concentration of from about 2 to about 100 mM, about 5 mM to about 100 mM, for example about 10 mM to about 100 mM or about 25 mM to about 100 mM, such as from about 2 mM to about 60 mM, about 10 mM to about 60 mM, about 40 mM to about 60 mM is preferred, particularly about 2 mM, about 10 mM or about 50 mM. In one embodiment of this preferred formulation, the anion having multiple negative charges housed on a flexible backbone is not one of, or both of, glutamic acid and ATP.

The formulation according to the use of the first aspect of the invention may or may not comprise a buffer in addition to the unaggregated albumin and the anion having multiple negative charges housed on a flexible backbone. It is preferred that the buffer is one that maintains or increases and/or does not reduce the stability of the albumin in the presence of the anion. By ‘does not reduce the stability of the albumin in the presence of the anion’, we include the meaning that in the presence of the buffer, the stability of the albumin in the presence of the anion is not substantially less than the stability of the albumin in the presence of the anion in the absence of the buffer. By ‘maintains or increases the stability of albumin in the presence of the anion’ we include the meaning that, in the presence of the buffer, the stability of the albumin in the presence of the anion is either the same or more than the stability of the albumin in the presence of the anion in the absence of the buffer. The stability of albumin can be measured by any suitable technique known in the art, including those described above, such as techniques to assess aggregation and thermal stability. Typically, in the presence of the buffer, the stability of the albumin in the formulation is no less than 95% of the stability of the albumin in the formulation in the absence of the buffer, and is preferably the same or more than the stability of the albumin in the formulation in the absence of the buffer.

The buffer may or may not be charged. The term ‘charged’ as described herein includes the meaning of the standard chemical definition of the total charge of the molecule, namely the sum of charges of all charged groups, e.g. carboxyl anion (−1), protonated amine (+1), when measured at pH 7.4. The charge of each potentially charged group can be calculated at a given pH by reference to the pKa for each potentially charged group, according to methods well known in the art. The pKa for each potentially charged group may also be determined according to methods well known in the art.

In an embodiment, the buffer is positively charged. In an alternative embodiment, the buffer is negatively charged. In yet another embodiment, the buffer is uncharged.

It will be appreciated that the final pH of the formulation will be influenced by the presence of all of the constituents of the formulation, including the albumin, the anion having multiple negative charges housed on a flexible backbone, the buffer, and one or more other excipients mentioned herein.

Given that the anion having multiple negative charges housed on a flexible backbone (e.g. citrate) is already negatively charged, it may be desirable to have a buffer that is positively charged such that the final pH of the formulation is a pH of 6 to 8, for example a pH of around 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, or 7.9 to around 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0.

As described in the Examples, formulations according to the invention were found to have the stabilising effect on virus across at least a pH range of 4-8. Hence, in an embodiment, the buffer is selected such that the final pH of the formulation is a pH of 4 to 8, for example a pH of around 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 to around 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0.

In one embodiment, the buffer has a pKa higher than the pH of the formulation, for example such that the buffer is positively charged.

Particularly preferred forms of buffer include Tris, Bis-Tris and/or Bis-Tris propane. Other buffers may or may not include phosphate buffered saline (PBS) and/or PBS+0.001% poloxamer (Vigene Biosciences/Charles River Laboratories). Other buffers may or may not include bis-trisphosphate (BTP) and/or 2-[4-(2-hydroxyethyl) piperazin-1-yl]ethane-sulfonic acid (HEPES). HEPES and BTP are preferred buffers that may be used. In one embodiment when such a buffer is present, preferably the anion having multiple negative charges housed on a flexible backbone is not one of, or both of, glutamic acid and ATP.

When a buffer is present, a preferred formulation comprises from 0.1 mM to 250 mM, such as 1 to 250 mM of buffer. The buffer concentration may be from about 1, 5, 10, 15, 20, 25, 30, 35, 40, 45, 50, 55, 60, 65, 70, 75, 80, 85, 90, 95, or 100 mM to about 40, 45, 50, 55, 60, 65, 70, 75, 80, 85, 90, 95, 100, 150, 200 or 250 mM. A buffer concentration of from about 10 mM to about 200 mM is preferred, particularly about 150 mM, particularly wherein the buffer is Tris. When the buffer is Tris, the buffer may be present at a concentration of from about 0.1 to about 50 mM, such as about 30 to about 50 mM, preferably 50 mM. When the buffer is BTP or HEPES, the buffer may be present at a concentration of from about 0.1 to about 50 mM, such as about 30 to about 50 mM, and preferably about 30 mM. In one embodiment when such a buffer is present, the anion having multiple negative charges housed on a flexible backbone is not one of, or both of, glutamic acid and ATP.

In an embodiment, the formulation in the use of the first aspect of the invention comprises from 0 to 50 mM of magnesium chloride. For example, the formulation may comprise from about 0, 1, 2, 3, 4, 5, 10, 15, 20, 25, 30, 35 or 40 mM to about 20, 25, 35, 40, 45, or 50 mM magnesium chloride. The formulation may comprise from about 0 mM to about 10 mM such as about 1 mM to about 10 mM magnesium chloride, or from about 2 mM to about 10 mM magnesium chloride, such as from about 5 mM to about 10 mM. A magnesium chloride concentration of from about 5 to about 10 mM or from about 15 mM to about 35 mM is preferred, and a magnesium chloride concentration of about 5 mM, about 10 mM or about 25 mM is particularly preferred. In one embodiment when magnesium chloride is present, the anion having multiple negative charges housed on a flexible backbone is not one of, or both of, glutamic acid and ATP.

In an embodiment, the formulation in the use of the first aspect of the invention comprises from 0 to 500 mM of sulfate salts, such as 0 to 200 mM, such as sulfate salts selected from sodium sulfate, sodium bisulfate, sodium thiosulfate, ammonium sulfate, ammonium bisulfate, magnesium sulfate, magnesium bisulfate, potassium sulfate, potassium bisulfate. For example, the formulation may comprise from about 0, 10, 20, 30, 40, 50, 60, 70, 80, 90, 100, 125, 150, 175, 200, 225, 250, 275, 300, 325, 350, 375, 400, 425, 475 to about 70, 80, 90, 100, 125, 150, 175, 200, 225, 250, 275, 300, 325, 350, 375, 400, 425, 475, 500 mM of sulfate salts. A sulfate salt concentration of about 50 mM is preferred or of about 200 mM is preferred. Particularly preferred concentrations of sulfate salts include about 25-500 mM, such as about 25-250, such as about 50-200 mM (e.g. about 200 mM or about 50 mM) sodium sulfate, about 25-500 mM, such as about 25-250, such as about 50-200 (e.g. about 200 mM or about 50 mM) ammonium sulfate, about 25-500 mM, such as about 25-250, such as about 50-200 mM (e.g. about 200 mM or about 50 mM) sodium thiosulfate, about 25-500 mM, such as about 25-250, such as about 50-200 mM (e.g. about 200 mM or about 50 mM) sodium bisulfate, and about 25-500 mM, such as about 25-250, such as about 50-200 mM (e.g. about 200 mM or about 50 mM) magnesium sulfate. In one embodiment when sulfate salts are present, the anion having multiple negative charges housed on a flexible backbone is not one of, or both of, glutamic acid and ATP.

In an embodiment, the formulation in the use of the first aspect of the invention comprises from 0 to 500 mM of disodium phosphate (Na₂HPO₄), such as 0 to 200 mM. For example, the formulation may comprise from about 0, 1, 2.5, 5, 7.5, 10, 12.5, 15, 17.5, 20, 30, 40, 50, 60, 70, 80, 90, 100, 125, 150, 175, 200, 225, 250, 275, 300, 325, 350, 375, 400, 425, 450, 475 to about 70, 80, 90, 100, 125, 150, 175, 200, 225, 250, 275, 300, 325, 350, 375, 400, 425, 500 mM of disodium phosphate. Particularly preferred concentrations of disodium phosphate include about 10-500 mM, such as about 10-250 mM, such as about 10-200 mM (e.g. about 10 mM or about 200 mM). A disodium phosphate concentration of about 10 mM or about 200 mM is preferred. In one embodiment when disodium phosphate is present, the anion having multiple negative charges housed on a flexible backbone is not one of, or both of, glutamic acid and ATP.

As discussed further in the Examples, it was found that albumin confers stability to the formulation even in the presence of EDTA. Thus, in an embodiment, the formulation in the use of the first aspect of the invention comprises from 0 to 50 mM of EDTA. For example, the formulation may comprise from about 0, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, or 10 to about 2, 3, 4, 5, 6, 7, 8, 9, 10, 20, 30, 40, or 50 mM of EDTA. An EDTA concentration of about 1 mM is preferred. In one embodiment when EDTA is present, the anion having multiple negative charges housed on a flexible backbone is not one of, or both of, glutamic acid and ATP.

In an embodiment, the formulation in the use of the first aspect of the invention comprises from 0 to 200 mM of glutamic acid. For example, the formulation may comprise from about 0, 10, 20, 30, 40, 50, 60, 70, 80, 90 or 100 to about 30, 40, 50, 60, 70, 80, 90, 100, 125, 150, 175, or 200 mM of glutamic acid. A glutamic acid concentration of about 50 mM is preferred. When glutamic acid is present in the formulation, it is preferred if the formulation comprises one or more additional components in addition to the albumin, anion having multiple negative charges housed on a flexible backbone, and optionally the buffer. In one embodiment when glutamic acid is present, the anion having multiple negative charges housed on a flexible backbone is not ATP.

In an embodiment, the formulation in the use of the first aspect of the invention comprises from 0 to 500 mM of mannitol. For example, the formulation may comprise from about 0, 25, 50, 75, 100, 125, 150, 175, 200, 225, 250, 275, 300, 325, or 350 to about 175, 200, 225, 250, 275, 300, 325, 350, 375, 400, 425, 450, 475, or 500 mM of mannitol. A mannitol concentration of about 300 mM is preferred. When mannitol is present in the formulation, it is preferred if the formulation comprises one or more additional components in addition to the albumin, anion having multiple negative charges housed on a flexible backbone, and optionally the buffer. In one embodiment when mannitol is present, the anion having multiple negative charges housed on a flexible backbone is not one of, or both of, glutamic acid and ATP.

In an embodiment, the formulation in the use of the first aspect of the invention comprises from 0 to 500 mM of sucrose. For example, the formulation may comprise from about 0, 25, 50, 75, 100, 125, 150, 175, 200, 225, 250, 275, 300, 325, or 350 to about 175, 200, 225, 250, 275, 300, 325, 350, 375, 400, 425, 450, 475, or 500 mM of sucrose. A sucrose concentration of about 300 mM is preferred. When sucrose is present in the formulation, it is preferred if the formulation comprises one or more additional components in addition to the albumin, anion having multiple negative charges housed on a flexible backbone, and optionally the buffer. In one embodiment when sucrose is present, the anion having multiple negative charges housed on a flexible backbone is not one of, or both of, glutamic acid and ATP.

In an embodiment, the formulation in the use of the first aspect of the invention comprises from 0 to 500 mM of arginine such as 0 to 200 mM. For example, the formulation may comprise from about 0, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475 to about 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 425, 450, 475, 500 mM of arginine. An arginine concentration of about 25 mM or about 50 mM or about 200 mM is preferred. In one embodiment when arginine is present, the anion having multiple negative charges housed on a flexible backbone is not one of, or both of, glutamic acid and ATP.

In an embodiment, the formulation in the use of the first aspect of the invention comprises from 0 to 500 mM of chloride salts, such as 0 to 200 mM. For example, the formulation may comprise from about 0, 10, 20, 30, 40, 50, 60, 70, 80, 90, 100, 125, 150, 175, 200, 225, 250, 275, 300, 325, 350, 375, 400, 425, 450, 475 to about 30, 40, 50, 60, 70, 80, 90, 100, 125, 150, 175, 200, 225, 250, 275, 300, 325, 350, 375, 400, 425, 450, 475, 500 mM of chloride salts. Preferred concentrations of chloride salts include about 25-500, such as about 25-250, such as about 50-200 mM. A chloride salt concentration of about 50 mM or about 200 mM is particularly preferred. Thus, in an embodiment, the formulation in the use of the first aspect of the invention comprises from 0 to 500 mM of sodium chloride, such as 0 to 200 mM. For example, the formulation may comprise from about 0, 1, 2.5, 5, 7.5, 10, 12.5, 15, 17.5, 20, 30, 40, 50, 60, 70, 80, 90, 100, 125, 150, 175, 200, 225, 250, 275, 300, 325, 350, 375, 400, 425, 450, 475 to about 70, 80, 90, 100, 125, 150, 175, 200, 225, 250, 275, 300, 325, 350, 375, 400, 425, 450, 475, 500 mM of sodium chloride. Preferred concentrations of sodium chloride include about 25-500, such as about 25-250, such as about 50-200 mM. A sodium chloride concentration of about 50-200 mM is particularly preferred, even more preferably about 200 mM. In one embodiment when chloride salts are present, the anion having multiple negative charges housed on a flexible backbone is not one of, or both of, glutamic acid and ATP.

In an embodiment, the formulation in the use of the first aspect of the invention comprises from 0 to 200 mM of histidine. For example, the formulation may comprise from about 0, 5, 10, 15, 20, 25, 30, 35, 40, 45, 50, 55, 60, 65, 70, 75, 80, 85, 90, 95 or 100 to about 35, 40, 45, 50, 55, 65, 70, 75, 80, 85, 90, 95, 100, 105, 110, 115, 120, 125, 130, 135, 140, 145, 150, 155, 160, 165, 170, 175, 180, 185, 190, 195, 200 mM of histidine. When present, a histidine concentration of about 50 mM is preferred. However, in another preferred embodiment, the formulation does not include histidine. In one embodiment when histidine is present, the anion having multiple negative charges housed on a flexible backbone is not one of, or both of, glutamic acid and ATP.

In an embodiment, the formulation in the use of the first aspect of the invention comprises from 0 to 200 mM of glycine. For example, the formulation may comprise from about 0, 10, 20, 30, 40, 50, 60, 70, 80, 90 or 100 to about 30, 40, 50, 60, 70, 80, 90, 100, 125, 150, 175, 200 mM of glycine. When present, a glycine concentration of about 50 mM or about 200 mM is preferred. However, in another preferred embodiment, the formulation does not include glycine. In one embodiment when glycine is present, the anion having multiple negative charges housed on a flexible backbone is not one of, or both of, glutamic acid and ATP.

In an embodiment, the formulation in the use of the first aspect of the invention comprises from 0 to 200 mM of glutamate. For example, the formulation may comprise from about 0, 10, 20, 30, 40, 50, 60, 70, 80, 90 or 100 to about 30, 40, 50, 60, 70, 80, 90, 100, 125, 150, 175, 200 mM of glutamate. When present, a glutamate concentration of about 50 mM is preferred. However, in another preferred embodiment, the formulation does not include glutamate. In one embodiment when glutamate is present, the anion having multiple negative charges housed on a flexible backbone is not ATP.

In an embodiment, the formulation in the use of the first aspect of the invention comprises from 0 to 200 mM of aspartate. For example, the formulation may comprise from about 0, 10, 20, 30, 40, 50, 60, 70, 80, 90 or 100 to about 30, 40, 50, 60, 70, 80, 90, 100, 125, 150, 175, 200 mM of aspartate. An aspartate concentration of about 50 mM is preferred. In one embodiment when aspartate is present, the anion having multiple negative charges housed on a flexible backbone is not one of, or both of, glutamic acid and ATP.

In an embodiment, the formulation in the use of the first aspect of the invention comprises from 0 to 0.01% (w/v) of non-ionic surfactant, such as a non-ionic surfactant selected from poloxamers such Pluronic F-68 as (Poloxamer 188, P188, MST-188) (https://www.selleckchem.com/products/pluronic-f-68.html; Guler et al, 2017) or polysorbate. Preferably, the polysorbate is polysorbate 20 or polysorbate 80. For example, the formulation may comprise from about 0, 0.00025, 0.0005, 0.00075, 0.001, 0.002, 0.003, 0.004, 0.005, 0.006 or 0.007 to about 0.002, 0.003, 0.004, 0.005, 0.006, 0.007, 0.008, 0.009, or 0.01% (w/v) of non-ionic surfactant. A non-ionic surfactant concentration of about 0.001% or about 0.01% (w/v) is preferred. In one embodiment when non-ionic surfactant is present, the anion having multiple negative charges housed on a flexible backbone is not one of, or both of, glutamic acid and ATP.

In an embodiment, the formulation in the use of the first aspect of the invention has a pH of from about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5 to about 5.5, 5.6, 5.7, 5.8, 5.9, 6.0, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8.0. A pH of from about 6.0 to about 8.0 is preferred, preferably about 5.0 to about 7.5, such as about 5.5 to about 7.0, such as about 5.5 to about 6.5, such as about 6.0 to about 6.5, or preferably about 6.0 or about 6.5. In another preferred embodiment, the pH of the formulation is 6.8. In an alternative preferred embodiment, a pH of from about 4.0 to about 5.0 is preferred. It will be appreciated that one advantage of adding or including albumin is that it enables stability at a more commercially acceptable or physiologically relevant pH range. In an embodiment when the formulation has such a pH, the anion having multiple negative charges housed on a flexible backbone is not one of, or both of, glutamic acid and ATP.

In a preferred embodiment, the formulation for use in the first aspect of the invention comprises a “good” albumin (e.g. 0.4 to 300 mg/mL, such as 0.5 to 300 mg/mL, preferably 0.5 to 50 mg/mL, more preferably 1 to 20 mg/mL, and even more preferably about 1 mg/ml, about 2.5 mg/ml, about 5 mg/mL);
Tris (e.g. 10 to 200 mM, preferably about 150 mM);
citrate (e.g. 2 to 100 mM or 25 to 100 mM, preferably 40 to 60 mM, more preferably about 50 mM); and
magnesium chloride (e.g. 1 to 10 mM or 15 to 35 mM, preferably 25 mM);
and has a pH of from 5 to 7.5 such as 5.5 to 7, preferably about 6 to 6.5.

In another preferred embodiment, the formulation for use in the first aspect of the invention comprises:
a “good” albumin (e.g. 0.4 to 300 mg/mL, such as 0.5 to 300 mg/mL, preferably 0.5 to 50 mg/mL, more preferably 1 to 20 mg/mL, and even more preferably about 1 mg/ml, about 2.5 mg/mL, about 5 mg/mL);
Tris (e.g. 10 to 200 mM, preferably about 150 mM);
citrate (e.g. 2 to 100 mM or 25 to 100 mM, preferably 40 to 60 mM, more preferably about 50 mM);
magnesium chloride (e.g. 1 to 10 mM or 15 to 35 mM, preferably 25 mM);
sodium sulfate (e.g. 25 to 75 mM or 25 to 74 mM, preferably about 50 mM);
and has a pH of from 5 to 7.5 such as 5.5 to 7, preferably about 6 to 6.5.

In another preferred embodiment, the formulation for use in the first aspect of the invention comprises:
a “good” albumin (e.g. 0.4 to 300 mg/mL, such as 0.5 to 300 mg/mL, preferably 0.5 to 50 mg/mL, more preferably 1 to 20 mg/mL, and even more preferably about 1 mg/ml, about 2.5 mg/mL or about 5 mg/ml);
citrate (e.g. 2 to 100 mM or 25 to 100 mM, preferably 40 to 60 mM, more preferably about 50 mM);
magnesium chloride (e.g. 1 to 10 mM or 15 to 35 mM, preferably 25 mM);
and has a pH from 5 to 7.5 such as 5.5 to 7, preferably 6 to 6.5.

In another preferred embodiment, the formulation for use in the first aspect of the invention comprises
a “good” albumin (e.g. 0.4 to 300 mg/mL, such as 0.5 to 300 mg/mL, preferably 0.5 to 50 mg/mL, more preferably 1 to 20 mg/mL, and even more preferably about 1 mg/mL, about 2.5 mg/mL, about 5 mg/ml);
Tris (e.g. 10 to 200 mM, preferably about 30 to 50 mM, more preferably about 50 mM);
citrate (e.g. 2 to 100 mM, preferably about 10 mM); and
magnesium chloride (e.g. 1 to 10 mM, such as 2-10 mM, preferably 5 mM);
and has a pH of from 4 to 8, such as from 5 to 7.5, preferably about 6.5 to 7, more preferably about 6.8).

In another preferred embodiment, the formulation for use in the first aspect of the invention comprises
a “good” albumin (e.g. 0.4 to 300 mg/mL, such as 0.5 to 300 mg/mL, preferably 0.5 to 50 mg/mL, more preferably 1 to 20 mg/mL, and even more preferably about 1 mg/mL, about 2.5 mg/mL, about 5 mg/mL);
Tris (e.g. 10 to 200 mM, preferably about 30 to 50 mM, more preferably 30 mM);
oxaloacetate (e.g. 2 to 100 mM, preferably about 10 mM); and
magnesium chloride (e.g. 1 to 10 mM, such as 2-10 mM, preferably 5 mM);
and has a pH of from 4 to 8, such as from 5 to 7.5, preferably about 6.5 to 7, more preferably about 6.8).

In another preferred embodiment, the formulation for use in the first aspect of the invention comprises
a “good” albumin (e.g. 0.4 to 300 mg/mL, such as 0.5 to 300 mg/mL, preferably 0.5 to 50 mg/mL, more preferably 1 to 20 mg/mL, and even more preferably about 1 mg/mL, about 2.5 mg/mL, about 5 mg/mL);
Tris (e.g. 10 to 200 mM, preferably about 30 to 50 mM, more preferably 30 mM);
fumarate (e.g. 2 to 100 mM, preferably about 10 mM); and
magnesium chloride (e.g. 1 to 10 mM, such as 2-10 mM, preferably 5 mM);
and has a pH of from 4 to 8, such as from 5 to 7.5, preferably about 6.5 to 7, more preferably about 6.8).

In another preferred embodiment, the formulation for use in the first aspect of the invention comprises
a “good” albumin (e.g. 0.4 to 300 mg/mL, such as 0.5 to 300 mg/mL, preferably 0.5 to 50 mg/mL, more preferably 1 to 20 mg/mL, and even more preferably about 1 mg/mL, about 2.5 mg/mL, about 5 mg/mL);
Tris (e.g. 10 to 200 mM, preferably about 30 to 50 mM, more preferably 30 mM);
succinate (e.g. 2 to 100 mM, preferably about 10 mM); and
magnesium chloride (e.g. 1 to 10 mM, such as 2-10 mM, preferably 5 mM);
and has a pH of from 4 to 8, such as from 5 to 7.5, preferably about 6.5 to 7, more preferably about 6.8).

In another preferred embodiment, the formulation for use in the first aspect of the invention comprises
a “good” albumin (e.g. 0.4 to 300 mg/mL, such as 0.5 to 300 mg/mL, preferably 0.5 to 50 mg/mL, more preferably 1 to 20 mg/mL, and even more preferably about 1 mg/mL, about 2.5 mg/mL, about 5 mg/ml);
Tris (e.g. 10 to 200 mM, preferably about 30 to 50 mM, more preferably 30 mM);
maleic acid (e.g. 2 to 100 mM, preferably about 10 mM); and
magnesium chloride (e.g. 1 to 10 mM, such as 2-10 mM, preferably 5 mM);
and has a pH of from 4 to 8, such as from 5 to 7.5, preferably about 6.5 to 7, more preferably about 6.8).

In another preferred embodiment, the formulation for use in the first aspect of the invention comprises
a “good” albumin (e.g. 0.4 to 300 mg/mL, such as 0.5 to 300 mg/mL, preferably 0.5 to 50 mg/mL, more preferably 1 to 20 mg/mL, and even more preferably about 1 mg/mL, about 2.5 mg/mL, about 5 mg/mL);
BTP (e.g. 10 to 200 mM, preferably about 30 to 50 mM, more preferably 30 mM);
citrate (e.g. 2 to 100 mM, preferably about 10 mM); and
magnesium chloride (e.g. 1 to 10 mM, such as 2-10 mM, preferably 5 mM);
and has a pH of from 4 to 8, such as from 5 to 7.5, preferably about 6.5 to 7, more preferably about 6.8).

In another preferred embodiment, the formulation for use in the first aspect of the invention comprises
a “good” albumin (e.g. 0.4 to 300 mg/mL, such as 0.5 to 300 mg/mL, preferably 0.5 to 50 mg/mL, more preferably 1 to 20 mg/mL, and even more preferably about 1 mg/mL, about 2.5 mg/mL, about 5 mg/mL);
HEPES (e.g. 10 to 200 mM, preferably about 30 to 50 mM, more preferably 30 mM);
citrate (e.g. 2 to 100 mM, preferably about 10 mM); and
magnesium chloride (e.g. 1 to 10 mM, such as 2-10 mM, preferably 5 mM);
and has a pH of from 4 to 8, such as from 5 to 7.5, preferably about 6.5 to 7, more preferably about 6.8).

By “to stabilise a virus”, we include the meaning that when a virus is contacted with the formulation as described herein, the stability of the virus is increased compared to the stability of the virus in the absence of the formulation (e.g. an otherwise identical or substantially identical formulation but which contains no albumin and/or no anion having multiple negative charges housed on a flexible backbone). In other words, when the stability of a virus is assessed under particular conditions, then contacting the virus with the formulation as described herein should increase the stability of the virus when assessed under the same particular conditions. The conditions selected may be any of the “stress conditions” described in the Examples, for example any of freeze-thaw cycles, temperature incubation (e.g. at a set temperature (e.g. 40° C. or 60° C.) for a set period of time (e.g. 2 weeks, 1 month or 2 months) and 2 months storage or incubation at a temperature of from 2 to 8° C., such as from 2, 3, 4, 5, 6 or 7 to 3, 4, 5, 6, 7 or 8° C., for example about 4° C. Freeze-thaw cycles may involve cycles of storing at a temperature of around −20° C. for a set period of time (e.g. at least 6, 7, 8, 9, 10, 11, 12, 13, 14, 15, 16, 17, 18, 19, 20, 21, 22, 23 or 24 hours) followed by thawing (e.g. at room temperature (15 to 25° C., 20° C. is preferred) for a set period of time such as at least 3, 4, 5, 6, 7, or 8 hours). The freeze-thaw stress applied to the virus may involve at least 2, 3, 4, 5, 6, 7, 8, 9 or 10 freeze-thaw cycles.

Typically, the increase in stability of the virus is manifest by decreased aggregation of the virus over a given period of time. Thus, when the virus is contacted with the formulation described herein, the virus will exhibit decreased aggregation compared to the level of aggregation of the virus in the absence of the formulation but otherwise under the same conditions. Assessing the level of aggregation of viruses may be carried out using any suitable technique in the art, and as described below and in the Examples.

In one embodiment, the increased stability of the virus is manifest by assessing the hydrodynamic radius of the formulation containing the virus (e.g. by DLS), for example when the formulation is incubated at 25° C. or 40° C., preferably 40° C., for a duration of at least 20 hours, or when the formulation has undergone multiple freeze-thaw cycles (e.g. at least 2, 3, 4, 5, 6, 7, 8, 9 or 10 freeze-thaw cycles). Although the particular hydrodynamic radius will be dependent on the virus that is present, the more stable the virus, the less variation there will be in the hydrodynamic radius (e.g. as measured by DLS) over the duration of the study. Thus, in a further embodiment, the increased stability of the virus is manifest by the hydrodynamic radius of the formulation containing the virus being no more than 6× (preferably no more than 5.5×, 5×, 4.5×, 4×, 3.5×, 3×, 2.5×, 2×, 1.5×) the hydrodynamic radius of the virus in its monomeric form over the duration of the study, for example following freeze-thaw stress (e.g. subjecting the formulation to 1, 2, 3, 4, 5, 6, 7, 8, 9 or 10 cycles of storing at a temperature of around −20° C. for a set period of time (e.g. at least 6, 7, 8, 9, 10, 11, 12, 13, 14, 15, 16, 17, 18, 19, 20, 21, 22, 23 or 24 hours) followed by thawing (e.g. at room temperature (15 to 25° C., 20° C. is preferred) for at least 3, 4, 5, 6, 7, or 8 hours)). A preferred free-thaw cycle comprises storage at about-20° C. for at least 6 hours, followed by thawing at room temperature for about 3 hours. By the virus in its monomeric form we include the meaning of a solution of the virus (e.g. a solution of the virus in the absence of the albumin and/or in the absence of the anion having multiple negative charges housed on a flexible backbone), that has not been subjected to any of the “stress conditions” described herein, for example any of freeze-thaw cycles, temperature incubation (e.g. at a set temperature (e.g. 40° C. or 60° C.) for a set period of time (e.g. 2 weeks, 1 month or 2 months) and 2 months storage or incubation at a temperature of from 2 to 8° C., such as from 2, 3, 4, 5, 6 or 7 to 3, 4, 5, 6, 7 or 8° C., for example about 4° C. The virus in its monomeric form may be prepared by providing a stock solution of the virus, which may be diluted to assess the hydrodynamic radius. It will be appreciated that the virus in monomeric form will represent a mono-disperse system.

The variation in the hydrodynamic radius can be assessed by determining the coefficient of variance. For example, the virus may be stabilised such that the coefficient of variance in the hydrodynamic radius of the formulation containing the virus being not more than 5% when the formulation is incubated at 40° C. for a duration of at least 20 hours. It will be appreciated that for formulations containing multiple components, additional analyses may be required to calculate the hydrodynamic radius, such as the method described in Example 14, which can be used to provide a mass percentage recovery for the virus under the conditions of the stability test. Thus, in one embodiment, the increased stability is manifest by a mass percentage recovery for the virus of at least 50%, such as at least 55%, 60%, 65%, 70%, 75%, 80%, 85% 90%, 95%, 98% or 99% over the duration of the study, for example following freeze-thaw stress (e.g. subjecting the formulation to 2, 3, 4, 5, 6, 7, 8, 9 or 10 cycles of storing at a temperature of around −20° C. for a set period of time (e.g. at least 6, 7, 8, 9, 10, 11, 12, 13, 14, 15, 16, 17, 18, 19, 20, 21, 22, 23 or 24 hours) followed by thawing (e.g. at room temperature (15 to 25° C., 20° C. is preferred) for at least 3, 4, 5, 6, 7, or 8 hours)). Preferably, the increased stability is manifest by a mass percentage recovery for the virus of at least 50%, such as at least 55%, 60%, 65%, 70%, 75%, 80%, 85% 90%, 95%, 98% or 99% following ten freeze-thaw cycles (e.g. where the formulation is frozen at around −20° C. for a set period, e.g. 6 hours, and thawed at room temperature for a set period, e.g. at least 3 hours, before being frozen again at around −20° C. and repeating the cycle).

Similarly, in another embodiment, the increased stability is manifest by the mass percentage recovery for the virus being higher than the mass percentage recovery for the virus in the absence of the formulation described herein (i.e. in the absence of the formulation that is used to increase the stability of the virus accordingly to the first aspect of the invention) over the duration of the study, for example following freeze-thaw stress (e.g. subjecting the formulation to 1, 2, 3, 4, 5, 6, 7, 8, 9 or 10 cycles of storing at a temperature of around −20° C. for a set period of time (e.g. at least 6, 7, 8, 9, 10, 11, 12, 13, 14, 15, 16, 17, 18, 19, 20, 21, 22, 23 or 24 hours) followed by thawing (e.g. at room temperature (15 to 25° C., 20° C. is preferred) for at least 3, 4, 5, 6, 7, or 8 hours)). For example, the mass percentage recovery for the virus may be increased or improved by at least 1, 2, 3, 4, 5, 10, 15, 20, 25, 30, 35, 40, 45, or 50 percentage points relative to the mass percentage recovery of the virus in the absence of the formulation over the duration of the study, e.g. following at least 1, 2, 3, 4, 5, 6, 7, 8, 9, 10 freeze-thaw cycles, where 1 freeze thaw cycle comprises storage at −20° C. for at least 6 hours followed by thawing at room temperature for about or at least 3 hours. It will be appreciated that the mass percentage recovery for the virus in the absence of the formulation described herein is typically the mass percentage recovery for the virus in an otherwise identical or substantially identical formulation but which contains no albumin and/or no anion having multiple negative charges housed on a flexible backbone. Mass percentage recovery may be measured by DLS as described herein.

Another way of assessing aggregation of viruses is by particle coverage. Thus, in another embodiment, the increased stability is manifest by the % particles coverage being less than 15%, more preferably less than 15, 14, 13, 12, 11, 10, 9, 8, 7, 6, 5, 4, 3, 2, 1 or 0.5% particles coverage, and preferably about 5% particles coverage or less. Preferably, the less than 15, 14, 13, 12, 11, 10, 9, 8, 7, 6, 5, 4, 3, 2, 1 or 0.5% particles coverage is observed at a storage or incubation at a temperature of from 2 to 8° C., such as from 2, 3, 4, 5, 6 or 7 to 3, 4, 5, 6, 7 or 8° C., for example about 4° C., for a duration of at least 1 month, preferably at least 2 months, 3 months, 4 months, 5 months, 6 months, 7 months, 8 months, 9 months, 10 months, 11 months, 12 months or 1 year. Alternatively, the less than 15, 14, 13, 12, 11, 10, 9, 8, 7, 6, 5, 4, 3, 2, 1 or 0.5% particles coverage is observed at a storage temperature of up to 25° C. for a duration of at least 2 weeks to 2 months, preferably 2 weeks, 3 weeks, 4 weeks, 1 month, 1.5 months or 2 months. Alternatively, the less than 15, 14, 13, 12, 11, 10, 9, 8, 7, 6, 5, 4, 3, 2, 1 or 0.5% particles coverage is observed when the formulation has undergone multiple freeze-thaw cycles (e.g. at least 2, 3, 4, 5, 6, 7, 8, 9 or 10 freeze-thaw cycles). Particles coverage may be measured by backgrounded membrane imaging (BMI) using brightfield microscopy and imaging 50 μL (microlitres) of a formulation comprising 5 mg/mL and ~ E+13 viral particle (vp)/mL. One technique that may be used is the Aura system from Halo Labs as described in Examples 1 and 15. By ‘particles coverage’, we include the meaning of the % of coverage of the membrane in a BMI measurement and is related to the number of aggregates.

It will be appreciated that it may be desirable to express the ‘particle count’ data as a fold-improvement or value relative to the ‘particle count’ of a control or stock solution of the virus (e.g. a solution of the virus in the absence of the albumin and/or in the absence of the anion having multiple negative charges housed on a flexible backbone), for example using the calculation ‘stock virus particle count’/‘sample particle count’. Thus, in one embodiment, the increased stability is manifest by the result of the calculation ‘stock virus particle count’/‘sample particle count’ being greater than 1, such as at least 2, 3, 4, 5, 6, 7, 8, 9 or 10, at least 15, 20, 25, 30, 35, 40, 45, 50, 55, 60, 65, 70, 75, 80, 85, 90, 95 or 100, or at least 150, 200, 250, or 300. In other words, the increased stability may be manifest by the particle count of the formulation containing the virus being less than the particle count of a control solution of the virus (e.g. a solution of the virus in the absence of the albumin and/or in the absence of the anion having multiple negative charges housed on a flexible backbone) after freeze thaw stress (e.g. following 1, 2, 3, 4, 5, 6, 7, 8, 9, 10 cycles of storing at a temperature of around −20° C. for at least 6 hours, followed by thawing at room temperature for about 3 hours.

In yet another embodiment, the increased stability is manifest by the particle count of less than 60 000 particles, preferably less than 60 000, 55 000, 50 000, 45 000, 40 000, 35 000, 30 000 or 25 000 particles for from at least 1 month to at least 12 months storage or incubation at a temperature of from 2 to 8° C., such as from 2, 3, 4, 5, 6, or 7 to 3, 4, 5, 6, 7, or 8° C., for example about 4° C., wherein the estimated circular diameter (ECD) of the particle size is equal or less than 2 μm (microns). Preferably, a storage time for from at least 1.5, 2, 3, 4 or 5 to at least 6, 6.5, 7, 8, 9, 10, 11 or 12 months. For example, around 1, 2, 3, 4, 5, 6, 7, 8, 9, 10, 11 or 12 months. By ‘particle count’, we include the meaning of the number of particles per mL. The number of particles may be calculated by counting the number of particles with an ECD ≥2 μm as measured by backgrounded membrane imaging (BMI) using brightfield microscopy.

Alternatively, the particle count is less than 200 000 particles, preferably less than 200 000, 150 000, 100 000 or 50 000 particles after four freeze-thaw cycles, wherein the estimated circular diameter (ECD) of the particle size is equal or less than 2 μm. By ‘freeze-thaw cycles’, we include the meaning of cycles where the formulation is frozen at around −20° C. for a set period (e.g. at least 6, 7, 8, 9, 10, 11, 12, 13, 14, 15, 16, 17 or 18 hours, or at least 19 hours or at least 20, 21, 23 or 24 hours) and thawed at room temperature for a set period (e.g. at least 3 or 4 hours or at least 5 hours) before being frozen again at around −20° C. and repeating the cycle. The number of particles may be calculated by counting the number of particles with an ECD ≥2 μm as measured in brightfield and subtracting the ones in the same size range as measured by Side Illumination Membrane Intensity (SIMI) i.e. Brightfield-SIMI.

In another embodiment, the increased stability is manifest by the particle count of less than 15 000 particles, preferably less than 15 000, 12 500, 10 000, 7 500, 5 000, 2 500 or 1 000 particles for from at least 1 month to at least 12 months storage or incubation at a temperature of from 2 to 8° C., such as from 2, 3, 4, 5, 6, or 7 to 3, 4, 5, 6, 7, or 8° C., for example about 4° C., wherein the estimated circular diameter (ECD) of the particle size is equal or less than 2 μm and the particles are Thioflavin T-positive. Preferably, a storage time for from at least 1.5, 2, 3, 4 or 5 to at least 6, 6.5, 7, 8, 9, 10, 11 or 12 months. For example, around 1, 2, 3, 4, 5, 6, 7, 8, 9, 10, 11 or 12 months. The number of particles may be calculated by counting the number of particles with an ECD ≥2 μm i.e. which fluoresce when dyed with the protein stain Thioflavin T (ThT) as measured by backgrounded membrane imaging (BMI) using brightfield and fluorescent microscopy.

As discussed further in the Examples, and in particular in Example 16, it will be appreciated that (i) the mass percentage recovery for the virus, e.g. as measured by DLS, and (ii) the particle count, e.g. as measured by Halo analysis to the particle count of a stock solution of the virus, may be combined to provide an overall rank of the stabilising effect of any given formulation on a virus. A formulation with a high mass percentage recovery and a high normalised particle count may be considered to be a very stable formulation, whereas a formulation with a low mass % recovery and a low normalised particle count may be considered to be a not very stable formulation. A formulation with a high mass % recovery and a low normalised particle count may still be considered to provide stabilising effects for virus. A formulation with low mass % recovery and a high normalised particle count requires judgement to be exercised by considering the overall results of hydrodynamic radius, mass percentage recovered and aggregation behaviour.

In another embodiment, the increased stability is manifest by the retention of virus integrity. In another embodiment, the increased stability is manifest by the virus retaining its ability to bind to a specific binding partner (e.g. monoclonal antibody) in the presence of the formulation. Binding can be assessed using enzyme-linked immunosorbent assay (ELISA). By ‘the virus retaining its ability to bind to a specific binding partner in the presence of the formulation’, we include the meaning that in the presence of the formulation, the virus is more likely to retain its binding ability to the specific binding partner, compared to its binding ability in the absence of the formulation. Where the virus comprises capsid proteins, it will be appreciated that the increased stability may be manifest by the capsid protein retaining its ability to bind to a specific binding partner, such as an antibody that selectively binds to an epitope on the capsid. Commercially available ELISA kits can be used to assess such retention in binding ability. For example, Progen manufactures a commercial ELISA kit that can be used to assess antibody binding to AAV capsids (https://www.progen.com/AAV/AAV-ELISA/), whereby a monoclonal antibody specific for a conformational epitope on assembled AAV capsids is coated onto microtiter strips and is used to capture AAV particles from the specimen.

In another embodiment, the increased stability is manifest by the virus retaining its ability to infect a cell in the presence of the formulation. Cell infection can be assessed using infectivity testing (TU assays) or any other techniques used in the art. By ‘the virus retaining its ability to infect a cell in the presence of the formulation’, we include the meaning that in the presence of the formulation, the virus is more likely to retain its infection ability, compared to its infection ability in the absence of the formulation.

The term ‘virus’ as described herein includes a virus, a viral particle, a viral fragment, a virus-like particle (VLP), a viral vector or a vector virus. As used herein, the terms ‘viral particle’ and ‘virus-like particle’ refer to a non-replicating, viral shell, derived from any of several viruses discussed further below. Viral particles and VLPs are generally composed of one or more viral proteins, such as, but not limited to those proteins referred to as capsid, coat, shell, surface and/or envelope proteins, or particle-forming polypeptides derived from these proteins. In addition, they may or may not contain additional genetic material. The term ‘virus particle’ refers to the whole virus being infectious unless attenuated or inactivated. VLPs are non-infectious particles. As used herein, the term ‘viral fragment’ refers to a part or portion of the virus or viral particle, that is preferably non-replicating. As used herein, the terms ‘viral vector’ and ‘vector virus’ refer to carriers (e.g. modified viruses) used to deliver genetic material into cells. Viral vectors use the mechanisms that viruses have evolved to efficiently transport their genomes into cells they infect. Viral vectors and vector viruses are commonly used in basic research, gene therapy and the development of vaccines.

In one embodiment, the virus can be a live virus, an attenuated virus, a live-attenuated virus, an inactivated virus.

In another embodiment, the virus can be a vector virus or a virus-like particle.

In another embodiment, the virus is in the form of a vaccine, preferably wherein the vaccine is a viral-based vaccine.

In another embodiment, the virus is a hybrid virus or chimeric virus. As used herein, the terms ‘chimeric virus’ or ‘hybrid virus’ refers to a virus in which functional nucleic acid or amino acid sequences, preferably amino acid sequences, from two or more distinct viruses are combined to form a viable organism.

In a preferred embodiment, the virus, preferentially a simian virus may be a parvovirus (e.g. an adeno-associated virus (AAV) or a recombinant adeno-associated virus (rAAV)), an adenoviridae (e.g. Adenovirus), a herpesviridae (e.g. Herpes Simplex Virus), a rhabdoviridae (e.g. vesicular stomatitis virus or Maraba Virus), a poxviridae (e.g. Vaccinia virus), a paramyxoviridae (e.g. Measles virus or Newcastle Disease virus), a reoviridae (e.g. Rotavirus or Reovirus), a picornavirus (e.g. Type I Poliovirus, Coxsackievirus A₂₁ or Seneca Valley virus), a flavivirus (e.g. Dengue virus, Yellow fever virus, West Nile virus or zika virus), a togaviridae (e.g. Alphavirus) or a coronaviridae (e.g. Coronavirus). By ‘simian’, we include monkeys, apes and humans.

In one embodiment, the AAV or the rAAV may be AAV serotype 1 (AAV1), AAV2, AAV3, AAV4, AAV5, AAV6, AAV7, AAV8, AAV9, AAV10, AAV11, AAV12, AAV-DJ, AAV-DJ/8, AAV-Rh10, AAV-retro, AAV-PHP.B, AAV8-PHP.eB, AAV-PHP.S, preferably AAV2, AAV9, AAV8 or AAV5.

In a preferred embodiment, the virus may be found at a concentration of 1×10⁶ to 1×10²⁴ vg/ml. The concentration of the virus may be from about 1×10⁶, 1×10⁷, 1×10⁸, 1×10⁹, 1×10¹⁰, 1×10¹¹ or 1×10¹² to about 1×10¹⁸, 1×10¹⁹, 1×10²⁰, 1×10²¹, 1×10²², 1×10²³, 1×10²⁴. A concentration of the virus of about 1×10¹¹ to about 1×10¹³ is preferred. It will be appreciated that it may be desirable to vary the concentration depending, for example, on the purpose of the formulation, its route of delivery to a subject, and the nature of the virus.

A second aspect of the invention provides a formulation comprising albumin, wherein the albumin is in a substantially unaggregated form and an anion having multiple negative charges housed on a flexible backbone.

In an embodiment, the formulation is a liquid formulation.

Preferences for the albumin and anion include those described above in relation to the first aspect of the invention. Preferences for the pH of the formulation include those described above in relation to the first aspect of the invention.

For example, in an embodiment, the formulation according to the second aspect of the comprises from 0.4 to 300 mg/mL, such as 0.5 to 300 mg/ml of albumin. For example, the formulation may comprise from about 25, 50, 75, 100, 125, 150, 175, 200, 225, 250, or 275 mg/ml to about 75, 100, 125, 150, 175, 200, 225, 250, 275 or 300 mg/ml albumin. In a particular example, the formulation may comprise from about 50 to 300 mg/mL, such as from about 100 to 200 mg/ml albumin. It will be appreciated that when the formulation of the second aspect of the invention has a concentration of albumin around 50 to 300 mg/mL, it may constitute a stock solution. It may be desirable to dilute the stock solution so that it has a concentration of albumin around 0.4 to 50 mg/mL, such as 0.5 to 50 mg/ml prior to use of the formulation to stabilise the virus according to the first aspect of the invention.

In another embodiment, the formulation of the second aspect of the invention comprises from 0.4 to 50 mg/mL, such as 0.5 to 50 mg/ml of albumin. For example, the formulation may comprise from about 0.4, 0.5, 1, 2, 5, 5, 10, 15, 20, 25, 30, 35 or 40 to about 25, 30, 35, 40, 45, or 50 mg/ml of albumin. An albumin concentration of from about 1 to 20 mg/mL is preferred, and albumin concentrations of about 1 mg/ml, about 2.5 mg/ml or about 5 mg/ml are particularly preferred.

The formulation may be provided in a container such as a rigid or flexible vial, bottle or bag e.g. a bioprocess container (BPC). Suitable container volumes include from about 50 mL to about 10 000 mL, e.g. from about 50, 100, 250, 500, 750, 1000, 2500, 5000 mL to about 100, 250, 500, 750, 1000, 2500, 5000, 10 000 mL, e.g. 50 mL, 1000 mL, 5000 mL and 10 000 mL. It is preferred that the container comprises one or more (e.g. several) inlets or outlets to allow filling of the container and/or dispensing from the container. The formulation may be sterilised, e.g. prior to or after being filled in the container. The formulation may or may not be provided in unit dosage form.

The formulation according to the second aspect of the invention may or may not comprise a buffer in addition to the unaggregated albumin and the anion having multiple negative charges housed on a flexible backbone. Suitable buffers and concentrations thereof include those described above in relation to the first aspect of the invention. Particularly preferred forms of buffer include Tris, Bis-Tris and/or Bis-Tris propane. Other preferred buffers include BTP and/or HEPES.

Preferably, the buffer has a pKa higher than the pH of the formulation, for example such that the buffer is positively charged, such as net positively charged. In this way, it is believed that the binding between the anion (e.g. citrate) and the albumin is not weakened.

It will be understood that the formulation of the second aspect of the invention may further comprise any one or more of the following constituents as described in relation to the first aspect of the invention: sulfate salts, sodium chloride, disodium phosphate, EDTA, glutamic acid, mannitol, sucrose, arginine, chloride salts, histidine, glycine, glutamate, aspartate, non-ionic surfactant. Suitable examples of these constituents and appropriate concentrations include those described above.

Without being bound by theory, constituents may act as osmolytes for aqueous preparations of viral vectors and are expected to provide stability by inducing protein folding and reducing denaturation. Also, the use of stabilising salts may increase surface tension at water-protein interface and strengthen hydrophobic interactions by keeping hydrophobic groups away from water molecules, inducing preferential hydration of proteins.

In one embodiment, two or more constituents are provided in combination. Preferable combinations include: sodium sulfate and arginine; sodium sulfate and histidine; sodium sulfate and EDTA; sodium sulfate and Pluronic acid; disodium phosphate and glutamic acid; disodium phosphate and mannitol; sodium chloride and arginine; sodium chloride and histidine; sodium chloride and EDTA; sodium sulfate, arginine and Pluronic acid.

The formulation according to the second aspect of the invention may or may not comprise a virus in addition to the unaggregated albumin and the anion having multiple negative charges housed on a flexible backbone. Suitable viruses include any of those described above in relation to the first aspect of the invention. When the formulation comprises a virus (e.g. one to be stabilised), it is preferred if the albumin is present at a concentration of 0.4 to 50 mg/ml, such as 0.5 to 50 mg/mL, such as 1 to 20 mg/mL, and more preferably about 1 mg/mL, about 2.5 mg/mL, about 5 mg/mL.

A third aspect of the invention provides a kit of parts comprising:
(i) albumin, wherein the albumin is in a substantially unaggregated form, and
(ii) an anion having multiple negative charges housed on a flexible backbone.

Preferences for the albumin and anion include those described above in relation to the first aspect of the invention.

It will be appreciated that the parts of the kit (i) and (ii) may be provided in separate containers, for example to allow further processing of one or both of the parts before the parts are mixed and used in whatever application (e.g. to stabilise a virus). For example, it may be desirable for one or both parts to be provided in a concentrated form (e.g. as a stock solution), which can be diluted prior to being, or when, added to the other part, prior to use in whatever application. Additionally or alternatively, it may be desirable for one or more other components to be added to one or both parts prior to the parts being mixed ready for use.

Preferably, the albumin part (i) of the kit may be provided in a concentrated form (e.g. as a stock solution), which can be diluted prior to being, or when, added to the anion having multiple negative charges housed on a flexible backbone. It will be appreciated that any of the albumin compositions (a)-(d) described above may constitute such a stock solution. In one example, the stock solution of the albumin part (i) of the kit may be from a 20× to 40× stock solution. Typically, the stock solution comprises from 50 to 300 mg/ml of albumin. For example, the stock solution may comprise from about 50, 75, 100, 125, 150, 175, 200, 225, 250, or 275 mg/ml to about 75, 100, 125, 150, 175, 200, 225, 250, 275 or 300 mg/mL albumin. In a particular example, the stock solution formulation may comprise from about 100 to 200 mg/mL albumin. It will be appreciated that the stock solution may then be diluted, for example to have a concentration of albumin around 0.4 to 50 mg/mL, such as 0.5 to 50 mg/mL, and mixed with the anion part (ii) of the kit, prior to use in whatever application (e.g. to stabilise a virus).

Preferably, the anion part (ii) of the kit may be provided in a concentrated form (e.g. as a stock solution), which can be diluted prior to being, or when, added to the albumin. In one example, the stock solution of the anion part (ii) of the kit may be from about 2× to 10× stock solution, such as about a 5× stock solution.

By a 2× stock solution we include the meaning of a solution that is two times more concentrated than the working concentration for whatever application (e.g. to stabilise a virus), by a 10× stock solution we include the meaning of a solution that is ten times more concentrated than the working concentration for whatever application (e.g. to stabilise a virus), and so on for 20× and 40×. Where a solution comprises more than one ingredient, it is preferred that, for example a 2× stock solution, is two times more concentrated than the working concentration in respect of each of those ingredients, and so on for a 10×, 20× and 40× stock solution. By “working concentration”, we include the meaning of a concentration that is suitable for whatever application the solution is being used for. For example, when the application is to stabilise a virus, the working concentration of various components (e.g. albumin in a substantially unaggregated form, anion having multiple negative charges housed on a flexible backbone, buffer, from sulfate salts, sodium chloride, disodium phosphate, EDTA, glutamic acid, mannitol, sucrose, arginine, chloride salts, histidine, glycine, glutamate, aspartate, and non-ionic surfactant) include those described above in relation to the first aspect of the invention.

It will be appreciated that the parts of the kit of the third aspect of the invention may be mixed together to provide the formulation that is to be used in accordance with the first aspect of the invention. Preferably, when the parts of the kit are mixed, the resultant formulation has a pH of from about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5 to about 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0. A pH of from about 6.0 to about 8.0 such as above 5.0 to about 7.5 is preferred, such as about 5.5 to about 7.0, such as about 5.5 to about 6.5, such as about 6.0 to about 6.5, or preferably about 6.0 or about 6.5. In an alternative embodiment, a pH of about 4.0 to about 5.0 is preferred.

It will be understood that the kit of the third aspect of the invention may further comprise a buffer in addition to the albumin and the anion having multiple negative charges housed on a flexible backbone. Suitable buffers and concentrations thereof include any of those described above in relation to the first aspect of the invention. Particularly preferred forms of buffer include Tris, Bis-Tris and/or Bis-Tris propane. Other buffers may or may not include phosphate buffered saline (PBS) and/or PBS+0.001% poloxamer (Vigene biosciences). Other buffers may or may not include BTP and/or HEPES. Preferred buffers include BTP and HEPES.

Preferably, the buffer has a pKa higher than the pH of the albumin part (i) and/or anion part (ii) of the kit. It will be appreciated that the buffer may be selected so as to ensure that when parts (i) and (ii) of the kit are mixed, the resultant formulation has a pH as described above, i.e. of from about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5 to about 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0.

Conveniently, the buffer may be used to dilute the albumin part (i) and/or the anion part (ii) of the kit (when the part(s) of the kit are provided as stock solution(s)) prior to use in whatever application (e.g. to stabilise a virus).

The buffer may be provided in a separate container to the respective containers housing parts (i) and (ii) of the kit, or it may be in the same container as one or both of the parts.

In a preferred embodiment, the anion part (ii) of the kit also contains the buffer. For example, the kit may comprise:
(i) albumin, wherein the albumin is in a substantially unaggregated form, preferably in the form of a stock solution (e.g. one from about 20× to 40× stock solution), for example one that comprises from 50 to 300 mg/mL, more preferably, 100 to 200 mg/mL), and
(ii) an anion having multiple negative charges housed on a flexible backbone and a buffer (e.g. any one of more of Tris, Bis-Tris Bis-Tris propane, BTP and/or HEPES), preferably in the form of a stock solution (e.g. one from about 2× to 10× stock solution, such as about a 5× stock solution).

It will further be understood that the kit of the third aspect of the invention may further comprise any one or more of the following constituents as described in relation to the formulation of the first and second aspects of the invention: sulfate salts, sodium chloride, disodium phosphate, EDTA, glutamic acid, mannitol, sucrose, arginine, chloride salts, histidine, glycine, glutamate, aspartate, non-ionic surfactant. Suitable examples of these constituents and appropriate concentrations include those described above.

It will be appreciated that such one or more constituents may be provided in separate one or more containers to the respective containers housing parts (i) and (ii) of the kit, or they may be in the same container that houses one of those parts (i) and (ii) of the kit.

When the kit further comprises a buffer, and the buffer is provided in a separate container to parts (i) and (ii) of the kit, it will be appreciated that such one or more constituents may be provided in separate one or more containers to the respective containers housing parts (i) and (ii) of the kit and to the container housing the buffer, or they may be in the same container as that housing one of parts (i) and (ii) of the kit or the same container as that housing the buffer.

When the kit further comprises a buffer, and the buffer is provided in the same container as one or both of parts (i) and (ii) of the kit, it will be appreciated that such one or more constituents may be provided in one or more separate containers to the respective containers housing parts (i) and (ii) of the kit, or may be in the same container as that housing one or both of parts (i) and (ii) of the kit.

Thus, in the preferred embodiment when the anion part (ii) of the kit also contains the buffer, the kit may comprise:
(i) albumin, wherein the albumin is in a substantially unaggregated form, preferably in the form of a stock solution (e.g. one from about 20× to 40× stock solution), for example one that comprises from 50 to 300 mg/mL, more preferably, 100 to 200 mg/mL), and
(ii) an anion having multiple negative charges housed on a flexible backbone, a buffer (e.g. any one or more of Tris, Bis-Tris Bis-Tris propane, BTP and/or HEPES), and one or more constituents selected from sulfate salts, glutamic acid, mannitol, sucrose, arginine, chloride salts (e.g. sodium chloride), histidine, glycine, glutamate, aspartate, and non-ionic surfactant, glutamic acid, mannitol, sucrose, preferably in the form of a stock solution (e.g. one from about 2× to 10× stock solution, such as about a 5× stock solution). It will be appreciated that the 2× stock solution of part (ii) means that the solution is twice as concentrated for the anion, buffer and one or more constituents than the working concentration for the anion, buffer and one or more constituents for whatever application (e.g. to stabilise a virus), and so on for 5× and 10×.

A fourth aspect of the invention provides a pharmaceutical composition comprising a formulation as defined in the first or second aspects of the invention, and a pharmaceutically excipient, carrier or diluent thereof.

Preferences for the formulation, buffer and virus include those described above in relation to the first and second aspects of the invention.

A fifth aspect of the invention provides a formulation for use in medicine wherein the formulation is as defined in the first or second aspect of the invention. Also included in this aspect is a kit of parts according to the third aspect of the invention for use in medicine.

Preferences for the formulation and virus include those described above in relation to the first or second aspect of the invention, and preferences for the kit include those described above in relation to the third aspect of the invention.

A sixth aspect of the invention provides a formulation for use in gene therapy wherein the formulation is as defined in the first or second aspect of the invention and further comprises a virus. Also included in this aspect is a kit of parts according to the third aspect of the invention for use in gene therapy.

By ‘gene therapy’, we include the meaning of a technique that modifies a subject's genes to treat, cure or prevent a disease or disorder. Gene therapies can work by several mechanisms: e.g. replacing a disease-causing gene with a healthy copy of the gene, inactivating a disease-causing gene that is not functioning properly or introducing a new or modified gene into the body to help treat a disease. The transferred genetic material can change how a single protein or group of proteins is produced by the cell.

A seventh aspect of the invention provides a formulation for use in cell therapy wherein the formulation is as defined in the first or second aspect of the invention and further comprises a virus. Also included in this aspect is a kit of parts according to the third aspect of the invention for use in cell therapy.

By ‘cell therapy’, we include the meaning of a technique where intact, live cells are transferred into a patient to treat, cure or prevent a disease or disorder. Such cells are typically modified using a viral vector before being administered to the patient. The cells may originate from the patient (autologous cells) or a donor (allogeneic cells). The cells used in cell therapy can be classified by their potential to transform into different cell types.

Preferences for the formulation and virus include those described above in relation to the first or second aspect of the invention, and preferences for the kit include those described above in relation to the third aspect of the invention.

In one embodiment, gene therapy and cell therapy can be combined to treat a genetic disease.

Stem cells are altered by gene therapy in culture (ex vivo) to express the relevant functional protein. The improved stem cells are then administered or returned to the patient.

In another embodiment, cells are modified before transplant or shipments using a viral vector. The stabilisation of these vectors will improve the loss of activity following, for example, thawing. Moreover, when these vectors are produced, there may be technical issues related to their stability during transit that would also be addressed by a stabilising formulation.

An eighth aspect of the invention provides a formulation for use in vaccination and/or immunisation wherein the formulation is as defined in the first or second aspect of the invention and further comprises a virus. Also included in this aspect is a kit of parts according to the third aspect of the invention for use in vaccination and/or immunisation.

By vaccination, we include the meaning of administration of a vaccine wherein the vaccine is used to stimulate the immune response to recognise a pathogen (a disease-causing organism) or part of a pathogen. Once the immune system has been trained to recognise this, if the body is later exposed to the pathogen, it will be removed from the body, destroyed or otherwise rendered inactive or non-problematic. Specifically, the immune system recognises foreign ‘antigens’, parts of the pathogen on the surface or inside the pathogen, that are not normally found in the body. Different types of vaccines may include: whole pathogen vaccines or viral vector vaccines.

Preferences for the formulation and virus include those described above in relation to the first or second aspect of the invention, and preferences for the kit include those described above in relation to the third aspect of the invention.

A ninth aspect of the invention provides a formulation for use in immunotherapy wherein the formulation is as defined in the first or second aspect of the invention and further comprises a virus, preferably wherein the immunotherapy is oncolytic virotherapy. Also included in this aspect is a kit of parts according to the third aspect of the invention for use in immunotherapy.

By immunotherapy, we include the meaning of the treatment or prevention of a disorder or disease that involves the activation, enhancement, reduction, suppression, or desensitisation of the immune system. Preferably, the disorder or disease is an autoimmune disorder, an allergy, or a cancer.

By oncolytic virotherapy (OV), we include the meaning of a form of immunotherapy that uses competent replicating viruses to infect and destroy cancer cells. Preferably, the competent viruses specifically attack tumour cells but not healthy cells.

Current OV platforms require large doses of viral product, well in excess of vaccine doses, in order to see effective delivery to tumour sites and therapeutic effects. Higher virus concentrations often come with the accumulation of impurities like residual cellular host DNA.

To eliminate impurities, more extensive purification procedures are required. Therefore, the stabilisation of higher dose of gene therapy products allows for their systemic use. This is important in gene therapy but in particular in oncolytic virotherapy, where a local administration would have the limitation of not affecting metastatic sites.

Preferences for the formulation and virus include those described above in relation to the first or second aspect of the invention, and preferences for the kit include those described above in relation to the third aspect of the invention.

A tenth aspect of the invention provides a method of stabilising a virus comprising combining the virus with a formulation as defined in the first and second aspects of the invention, or a pharmaceutical composition as defined in the fourth aspect of the invention.

Preferences for the formulation include those described above in relation to the first and second aspects of the invention. In a preferred embodiment, the formulation is one that comprises from 0.4 to 50 mg/mL, such as 0.5 to 50 mg/ml of albumin. For example, the formulation may comprise from about 0.4, 0.5, 1, 2.5, 5, 10, 15, 20, 25, 30, 35 or 40 to about 25, 30, 35, 40, 45, or 50 mg/ml of albumin. An albumin concentration of from about 1 to 20 mg/mL is particularly preferred, and albumin concentrations of about 1 mg/mL, about 2.5 mg/mL, about 5 mg/ml are more particularly preferred.

It will be appreciated that the kit of the third aspect of the invention may be useful in such a method. For example, the method may comprise (a) providing albumin wherein the albumin is in a substantially unaggregated form, (b) providing an anion having multiple negative charges housed on a flexible backbone, and (c) contacting the virus with the albumin of (a) and anion of (b) so as to stabilise the virus. The provision of albumin and the anion in steps (a) and (b), may correspond to respective parts (i) and (ii) of the kit of the third aspect of the invention, which may or may not be pre-mixed prior to contacting the albumin and anion, with the virus. Conveniently, for example, the “albumin” and “anion” are provided in the form of stock solutions that can be diluted to provide working concentrations of the “albumin” and “anion” suitable for stabilising the virus. The method may comprise the use of a buffer and/or one or more other constituents such as sulfate salts and/or chloride salts (e.g. sodium chloride), as described above in relation to the first and second aspects of the invention.

Preferences for the formulation, buffer, virus and techniques to assess stability include any of those described herein.

An eleventh aspect of the invention provides a method of selecting a suitable albumin for use in stabilising a virus, the method comprising determining whether the albumin is in a substantially unaggregated form.

It will be appreciated that if an albumin is considered to be suitable for use in stabilising a virus, the albumin will be suitable for use in formulating a virus or in a formulation comprising a virus (e.g. a formulation comprising a viral vector). Thus, the method of the eleventh aspect of the invention may be considered to be a method of selecting a suitable albumin for use in formulating a virus or in a formulation comprising a virus, the method comprising determining whether the albumin is in a substantially unaggregated form.

Preferences for the albumin, anion, virus and techniques to assess stability include any of those described herein.

In one embodiment, determining whether the albumin is in a substantially unaggregated form comprises (i) assessing whether there is less than 0.025% (w/w) of albumin in a polymeric form, optionally wherein the assessment is performed by high-performance liquid chromatography, wherein a value of less than 0.025% (w/w) of albumin in a polymeric form is indicative of the albumin being in a substantially unaggregated form; and/or (ii) assessing whether the hydrodynamic radius of albumin does not change more than 4.5% when incubated at 40° C. for a duration of 3 days, optionally wherein the assessment is performed by dynamic light scattering, wherein a hydrodynamic radius that does not change more than 4.5% when incubated at 40° C. for a duration of 3 days is indicative of the albumin being in a substantially unaggregated form; and/or (iii) assessing whether the polydispersity index of albumin is equal or less than 0.05.

It will be appreciated that if the method identifies an albumin that satisfies the criteria in one or more of (i)-(iii) above, preferably two or more or all three of (i)-(iii) above, then the albumin is identified as being in a substantially unaggregated form, and therefore of value in stabilising viruses and/or in formulating viruses or for use in viral formulations (e.g. viral vector formulations).

It will be appreciated that any of the desirable traits of albumin described herein as being beneficial for stabilising viruses may be assessed as part of the method of the eleventh aspect of the invention.

In yet another embodiment, the method according to the eleventh aspect of the invention further comprises formulating the albumin with an anion having multiple negative charges housed on a flexible backbone, and optionally assessing whether the formulation comprising the albumin and the anion stabilises a virus.

The invention will now be described by reference to the following embodiments, figures and examples.

### PART B—PREFERRED EMBODIMENTS OF THE INVENTION

1. Use of a formulation comprising:
(i) albumin, wherein the albumin is in a substantially unaggregated form; and
(ii) an anion having multiple negative charges housed on a flexible backbone, to stabilise a virus.
2. The use according to Embodiment 1, wherein:
(i) less than 0.025% (w/w) of albumin is in a polymeric form; and/or
(ii) the hydrodynamic radius of albumin does not change more than 4.5% when incubated at 40° C. for a duration of 3 days; and/or
(iii) the polydispersity index of albumin is equal or less than 0.05; and/or
(iv) the albumin is in a substantially an unaggregated form as determined by a technique selected from the group consisting of high-performance liquid chromatography (HPLC), differential light scattering (DLS); and/or
(v) less than 75% of albumin molecules are post-translationally modified, by means other than oxidation; and/or
(vi) the total amount of lipids or fatty acids (excluding octanoate) in the formulation is equal or less than 50 μg (microgram) lipids or fatty acids/mL of 100 mg/mL of albumin; and/or (vii) the albumin is a recombinant albumin; and/or
(viii) the albumin is (a) a yeast-derived albumin, optionally wherein the yeast is Pichia such as Pichia pastoris, Saccharomyces such as Saccharomyces cerevisiae, Candida, or Kluyveromyces such as Kluyveromyces lactis or Kluyveromyces marxianus, Hansenula polymorpha, Schizosaccharomyces pombe, Yarrowia lipolytica, Arxula adeninivorans, Candida such as Candida utilis) or Zygosaccharomyces bailii; or
(b) a plant-derived albumin such as a rice-derived albumin; and/or
(ix) the formulation is a liquid formulation; and/or
(x) the formulation comprises at least 0.5 moles free thiol per mole albumin; and/or
(xi) the albumin is provided by an albumin composition selected from the group consisting of any of:
a. a composition comprising 130 to 160 mM, preferably 145 mM, of sodium, 29 to 35 mM, preferably 32 mM, of octanoate, 10 to 20 mg/L, preferably 10 to 15 mg/L, of Polysorbate 80, 190 to 210 mg/mL, preferably 200 mg/mL, of albumin (w/v), wherein the pH is 6.7 to 7.3, preferably 7 and wherein the percentage of albumin (w/w) in a polymeric form is 0 to 1%, more preferably less than 0.025%;
b. a composition comprising 120 to 160 mM, preferably 145 mM, of sodium, 4 to 12 mM, preferably 8 mM, of octanoate, 0 to 50 mg/L, preferably 25 to 45 mg/L, of Polysorbate 80, 95 to 105 mg/mL, preferably 100 mg/mL, of albumin (w/v), wherein the pH is 6.4 to 7.4, preferably 7, and wherein the percentage of albumin (w/w) in a polymeric form is 0 to 1%, more preferably less than 0.025%;
c. a composition comprising 120 to 160 mM, preferably 145 mM, of sodium, 8 to 24 mM, preferably 16 mM, of octanoate, 0 to 100 mg/L, preferably 50 to 70 mg/L, of Polysorbate 80, 190 to 210 mg/mL, preferably 200 mg/mL, of albumin (w/v), wherein the pH is 6.4 to 7.4, preferably 7, and wherein the percentage of albumin (w/w) in a polymeric form is 0 to 1%, more preferably less than 0.025%;
d. a composition comprising 200 to 300 mM, preferably 230 to 260 mM of sodium, 0 to 3 mM, preferably 0 to 1 mM of octanoate, 95 to 105 mg/mL, preferably 100 mg/mL, of albumin (w/v), wherein the pH is 6 to 7, preferably 6.5, and wherein the percentage of albumin (w/w) in a polymeric form is 0 to 1%, preferably less than 0.025%; and/or
(xii) the formulation comprises from 0.4 to 300 mg/ml albumin, such as 0.4-50 mg/mL of albumin, optionally wherein the formulation comprises 1-20 mg/ml of albumin; and/or
(xiii) the anion having multiple negative charges housed on a flexible backbone is one that thermally stabilises the albumin; and/or
(xiv) the anion having multiple negative charges housed on a flexible backbone protects against deamidation of the albumin; and/or
(xv) the anion having multiple negative charges housed on a flexible backbone protects against N-terminal degradation of the albumin; and/or
(xvi) the anion having multiple negative charges housed on a flexible backbone is a multivalent carboxylic acid such as citrate, fumarate, tartaric acid, α-ketoglutarate, malate, maleic acid, succinate, succinic acid, aconitate, isocitrate, oxaloacetate, adenosine triphosphate or sodium tripolyphosphate or any intermediate anion of the citric acid cycle; and/or
(xvii) the anion having multiple negative charges housed on a flexible backbone is present at a concentration of 2-500 mM or 10-500 mM, optionally wherein the anion is present at a concentration of 2-100 mM or 25-100 mM, optionally wherein the anion is present at a concentration of 2-100 mM, such as around 2-60 mM, 10-60 mM, 40-60 mM, such as around 2 mM, around 10 mM or around 50 mM; and/or
(xviii) the formulation further comprises a buffer, optionally wherein:
(a) the buffer is one that maintains or increases the stability of the albumin in the presence of the anion having multiple negative charges housed on a flexible backbone; and/or
(b) the buffer is one that does not reduce the stability of the albumin in the presence of the anion having multiple negative charges housed on a flexible backbone; and/or
(c) the buffer is positively charged; and/or
(d) the buffer has a pKa higher than the pH of the formulation; and/or
(e) the buffer is any one or more of Tris, Bis-Tris, Bis-Tris propane, BTP and/or HEPES; and/or
(f) the formulation comprises 0-250 mM or 1-250 mM of buffer, optionally wherein the formulation comprises 10-200 mM of buffer, optionally wherein the formulation comprises (ﬁ) about 150 mM of buffer, optionally wherein the buffer is Tris, or (fii) about 0-50 mM or about 30-50 mM of buffer, optionally where the buffer is any of Tris, BTP and/or HEPES; and/or
(xix) wherein the formulation comprises:
(a) 0-50 mM of magnesium chloride, optionally wherein the formulation comprises about 25 mM of magnesium chloride or about 0 to about 10 mM, such as a about 1 to about 10 mM magnesium chloride, such as about 5 mM or about 10 mM magnesium chloride; and/or
(b) 0-500 mM, such as about 0-200 mM of sulfate salts, optionally wherein the formulation comprises about 25 to 250 or about 50-200 mM sulfate salts such as about 50 mM or about 200 mM of sulfate salts, and further optionally wherein the sulfate salt is sodium sulfate, sodium bisulfate, sodium thiosulfate, ammonium sulfate, ammonium bisulfate, magnesium sulfate, magnesium bisulfate, potassium sulfate, potassium bisulfate; and/or
(c) 0-500 mM, such as about 0-200 mM of disodium phosphate, optionally wherein the formulation comprises about 10 mM of disodium phosphate or about 200 mM of disodium phosphate; and/or
(d) 0-50 mM of EDTA, optionally wherein the formulation comprises about 1 mM of EDTA; and/or
(e) 0-200 mM of glutamic acid, optionally wherein the formulation comprises about 50 mM of glutamic acid; and/or
(f) 0-500 mM of mannitol, optionally wherein the formulation comprises about 300 mM of mannitol; and/or
(g) 0-500 mM of sucrose, optionally wherein the formulation comprises about 300 mM of sucrose; and/or
(h) 0-500 mM of arginine such as about 0-200 mM, optionally wherein the formulation comprises about 25 mM to about 50 mM of arginine or wherein the formulation comprises about 200 mM of arginine; and/or
(i) 0-500 mM of chloride salts, such as about 0-200, optionally wherein the formulation comprises about 25 to 250 or about 50-200 mM, such as about 200 or about 50 mM of chloride salts
(j) 0-200 mM of histidine, optionally wherein the formulation comprises about 50 mM of histidine
(k) 0-200 mM of glycine, optionally wherein the formulation comprises about 50 mM of glycine or about 200 mM of glycine; and/or
(I) 0-200 mM of glutamate, optionally wherein the formulation comprises about 50 mM of glutamate; and/or
(m) 0-200 mM of aspartate, optionally wherein the formulation comprises about 50 mM of aspartate; and/or
(n) 0-0.01% (w/v) of non-ionic surfactant, optionally wherein the formulation comprises about 0.001% or about 0.01% (w/v) of non-ionic surfactant, further optionally wherein the non-ionic surfactant is a poloxamer such as Pluronic F-68 or a polysorbate; and/or
(o) 0-500 mM of sodium chloride, optionally wherein the formulation comprises about 25 to 250 or about 50-200 mM of sodium chloride, such as about 200 mM or about 50 mM sodium chloride.
3. The use according to any one of the preceding embodiments, wherein the pH of the formulation is from about 4 to about 8, optionally wherein the pH of the formulation is from about 5 to about 7.5, preferably wherein the pH of the formulation is about 6 or about 6.5.
4. The use according to any one of the preceding embodiments, wherein:
(I) when the virus is contacted with the formulation, the stability of the virus is increased compared to the stability of the virus in the absence of the formulation, optionally wherein the increased stability:
   (a) is manifest by decreased aggregation of the virus over a given period of time; and/or
   (b) is manifest by the coefficient of variance of the hydrodynamic radius of the formulation containing the virus being not more than 5% when the formulation is incubated at 40° C. for a duration of at least 20 hours; and/or
   (c) is manifest by the hydrodynamic radius of the formulation containing the virus being no more than 6× (preferably no more than 5.5×, 5×, 4.5×, 4×, 3.5×, 3×, 2.5×, 2×, 1.5×) the hydrodynamic radius of the virus in its monomeric form, after freeze-thaw stress (e.g. following 1, 2, 3, 4, 5, 6, 7, 8, 9 or 10 cycles of storing at a temperature of around −20° C. for at least 6 hours, followed by thawing at room temperature for about 3 hours); and/or
   (d) is manifest by a mass percentage recovery for the virus of at least 50%, such as at least 55%, 60%, 65%, 70%, 75%, 80%, 85% 90%, 95%, 98% or 99%, after freeze-thaw stress (e.g. following 1, 2, 3, 4, 5, 6, 7, 8, 9 or 10 cycles of storing at a temperature of around −20° C. for at least 6 hours, followed by thawing at room temperature for about 3 hours); and/or
   (e) is manifest by the mass percentage recovery for the virus being increased or improved by at least 1, 2, 3, 4, 5, 10, 15, 20, 25, 30, 35, 40, 45, or 50 percentage points relative to the mass percentage recovery of the virus in the absence of the formulation (e.g. the virus in an otherwise identical or substantially identical formulation but which contains no albumin and/or no anion having multiple negative charges housed on a flexible backbone), after freeze thaw stress (e.g. following 1, 2, 3, 4, 5, 6, 7, 8, 9, 10 cycles of storing at a temperature of around −20° C. for at least 6 hours, followed by thawing at room temperature for about 3 hours; and/or
   (f) is manifest by the particle count of the formulation containing the virus being less than the particle count of a control solution of the virus (e.g. a solution of the virus in the absence of the albumin and/or in the absence of the anion having multiple negative charges housed on a flexible backbone) after freeze thaw stress (e.g. following 1, 2, 3, 4, 5, 6, 7, 8, 9, 10 cycles of storing at a temperature of around −20° C. for at least 6 hours, followed by thawing at room temperature for about 3 hours; and/or
   (g) is manifest by a particles coverage measured by backgrounded membrane imaging (BMI) of less than 15% after storage or incubation at a temperature of from 2 to 8° C., such as from 2, 3, 4, 5, 6 or 7 to 3, 4, 5, 6, 7 or 8° C., for example at about 4° C., for a duration of at least 1 month, for example, at least 2, 3, 4, 5 or 6 months; and/or
   (h) is manifest by a particle count of less than 60 000 particles measured by backgrounded membrane imaging (BMI) at after storage or incubation at a temperature of from 2 to 8° C., such as from 2, 3, 4, 5, 6 or 7 to 3, 4, 5, 6, 7 or 8° C., for example at about 4° C., for a duration of at least 1 month, for example, at least 2, 3, 4, 5 or 6 months, wherein the estimated circular diameter (ECD) of the particle is equal or less than 2 μm (microns); and/or
   (i) is manifest by a particle count of less than 15 000 particles measured by backgrounded membrane imaging (BMI) after storage or incubation at a temperature of from 2 to 8° C. such as from 2, 3, 4, 5, 6 or 7 to 3, 4, 5, 6, 7 or 8° C., for example at about 4° C., for a duration of at least 1 month, for example, at least 2, 3, 4, 5 or 6 months, wherein the estimated circular diameter (ECD) of the particle is equal or less than 2 μm (microns) and the particles are Thioflavin T-positive; and/or
   (j) is manifest by a particle count of less than 200 000 particles in Brightfield-Side illumination membrane intensity (SIMI) measured by backgrounded membrane imaging after at least four rounds of freeze-thaw cycles, wherein the estimated circular diameter (ECD) of the particle is equal or less than 2 μm (microns); and/or
   (k) is manifest by retention of virus integrity; and/or
   (l) is manifest by retention of the ability of the virus to bind to a specific binding partner of the virus; and/or
   (m) is manifest by retention of the ability of the virus to infect a cell; and/or
(II) the virus is
   (a) any of a viral particle, a viral fragment, a virus-like particle, a viral vector or a vector virus; and/or
   (b) a simian virus, optionally wherein the simian virus is an adeno-associated virus (AAV), a recombinant AAV, Adenovirus, Herpes Simplex Virus, vesicular stomatitis virus, Maraba virus, Vaccinia virus, Measles virus, Newcastle Disease Virus, Rotavirus, Reovirus, Type I Poliovirus, Coxsackievirus A₂₁, Seneca Valley virus, Dengue virus, Yellow fever virus, West Nile virus, zika virus, Alphavirus or Coronavirus, optionally wherein the AAV is AAV2, AAV9, AAV8 or AAV5; and/or
(III) the virus is a live virus, an attenuated virus, a live-attenuated virus or an inactivated virus; and/or
(IV) the virus is in the form of a vaccine; and/or
(V) the virus is a hybrid or chimeric virus; and/or
(VI) the virus that is stabilised is at a concentration of 1×10⁶-1×10²⁴ vg/mL.
5. A formulation or a kit of parts comprising:
(i) albumin, wherein the albumin is in a substantially unaggregated form; and
(ii) an anion having multiple negative charges housed on a flexible backbone.
6. The formulation or the kit according to Embodiment 5, wherein the formulation or kit further comprises:
(iii) a buffer, wherein the buffer is one that maintains or increases the stability of the albumin in the presence of the anion having multiple negative charges housed on a flexible backbone; or
(iv) a buffer, wherein the buffer is one that does not reduce the stability of the albumin in the presence of the anion having multiple negative charges housed on a flexible backbone.
7. The formulation or kit according to Embodiment 5 or 6, wherein:
(i) less than 0.025% (w/w) of albumin is in a polymeric form; and/or
(ii) the hydrodynamic radius of albumin does not change more than 4.5% when incubated at 40° C. for a duration of 3 days; and/or
(iii) the polydispersity index of albumin is equal or less than 0.05; and/or
(iv) the albumin is in a substantially unaggregated form as determined by a technique selected from the group consisting of high-performance liquid chromatography (HPLC), differential light scattering (DLS); and/or
(v) less than 75% of albumin molecules are post-translationally modified, by means other than oxidation; and/or
(vi) the total amount of lipids (excluding octanoate) is equal or less than 50 μg (microgram) lipids/mL in a 100 mg/mL solution of albumin; and/or
(vii) the albumin is a recombinant albumin; and/or
(viii) the albumin is
   (a) a yeast-derived albumin, optionally wherein the yeast is Pichia such as Pichia pastoris, Saccharomyces such as Saccharomyces cerevisiae, Candida, or Kluyveromyces such as Kluyveromyces lactis or Kluyveromyces marxianus, Hansenula polymorpha, Schizosaccharomyces pombe, Yarrowia lipolytica, Arxula adeninivorans, Candida such as Candida utilis or Zygosaccharomyces bailii or
   (b) a plant-derived albumin such as a rice-derived albumin; and/or
(ix) the formulation or kit comprises at least 0.5 moles free thiol per mole albumin; and/or
(x) the albumin is provided by an albumin composition selected from the group consisting of any of:
   a. a composition comprising 130 to 160 mM, preferably 145 mM, of sodium, 29 to 35 mM, preferably 32 mM, of octanoate, 10 to 20 mg/L, preferably 10 to 15 mg/L, of Polysorbate 80, 190 to 210 mg/mL, preferably 200 mg/mL, of albumin (w/v), wherein the pH is 6.7 to 7.3, preferably 7 and wherein the percentage of albumin (w/w) in a polymeric form is 0 to 1%, more preferably less than 0.025%;
   b. a composition comprising 120 to 160 mM, preferably 145 mM, of sodium, 4 to 12 mM, preferably 8 mM, of octanoate, 0 to 50 mg/L, preferably 25 to 45 mg/L, of Polysorbate 80, 95 to 105 mg/mL, preferably 100 mg/mL, of albumin (w/v), wherein the pH is 6.4 to 7.4, preferably 7, and wherein the percentage of albumin (w/w) in a polymeric form is 0 to 1%, more preferably less than 0.025%;
   c. a composition comprising 120 to 160 mM, preferably 145 mM, of sodium, 8 to 24 mM, preferably 16 mM, of octanoate, 0 to 100 mg/L, preferably 50 to 70 mg/L, of Polysorbate 80, 190 to 210 mg/mL, preferably 200 mg/mL, of albumin (w/v), wherein the pH is 6.4 to 7.4, preferably 7, and wherein the percentage of albumin (w/w) in a polymeric form is 0 to 1%, more preferably less than 0.025%;
   d. a composition comprising 200 to 300 mM preferably 230 to 260 mM of sodium, 0 to 3 mM, preferably 0 to 1 mM of octanoate, 95 to 105 mg/mL, preferably 100 mg/mL, of albumin (w/v), wherein the pH is 6 to 7, preferably 6.5, and wherein the percentage of albumin (w/w) in a polymeric form is 0 to 1%, preferably less than 0.025%; and/or
(xi) the formulation or kit comprises from 0.4 to 300 mg/mL albumin, such as from about 50 to 300 mg/mL or from about 100 to 200 mg/mL albumin, or from about 0.4 to 50 mg/ml of albumin, optionally wherein the formulation comprises 1-20 mg/ml of albumin; and/or
(xii) the anion having multiple negative charges housed on a flexible backbone is one that thermally stabilises the albumin; and/or
(xiii) the anion having multiple negative charges housed on a flexible backbone protects against deamidation of the albumin; and/or
(xiv) the anion having multiple negative charges housed on a flexible backbone protects against N-terminal degradation of the albumin; and/or
(xv) the anion having multiple negative charges housed on a flexible backbone is a multivalent carboxylic acid such as citrate, fumarate, tartaric acid, α-ketoglutarate, malate, maleic acid, succinate, succinic acid, aconitate, isocitrate, oxaloacetate, adenosine triphosphate or sodium tripolyphosphate or any intermediate anion of the citric acid cycle; and/or
(xvi) the anion having multiple negative charges housed on a flexible backbone is present at a concentration of 2-500 mM or 10-500 mM, optionally wherein the anion is present at a concentration of 2-100 mM or 25-100 mM; and/or
(xvii) the anion is present at a concentration of 2-100 mM, such as around 2-60 mM, 10-60 mM, 40-60 mM, such as around 2 mM, around 10 mM or around 50 mM.
8. The formulation or kit according to Embodiment 6 or 7, wherein:
(I) the buffer is positively charged; and/or
(II) the buffer has a pKa higher than (a) the pH of the formulation or (b) the pH of parts (i) and/or (ii) of the kit; and/or
(III) the buffer is any one or more of Tris, Bis-Tris, Bis-Tris propane, BTP and/or HEPES; and/or (IV) the formulation or kit comprises:
   (a) 0-250 mM of buffer such as 1-250 mM of buffer, optionally wherein the formulation or kit comprises 10-200 mM of buffer; and/or
   (b) about 150 mM of buffer, optionally wherein the buffer is Tris or about 0-50 mM such as about 30-50 mM of buffer, optionally wherein the buffer is any of Tris, BTP and/or HEPES; and/or
   (c) 0-50 mM of magnesium chloride, optionally wherein the formulation or kit comprises about 25 mM of magnesium chloride or about 0 to about 10 mM, such as about 1 to about 10 mM of magnesium chloride, such as about 5 mM or about 10 mM magnesium chloride; and/or
   (d) 0-500 mM such as 0-200 mM of sulfate salts, optionally wherein the formulation or kit comprises about 50-500 mM sulfate salts such as about 25-250 mM, such as about 50 mM or about 200 mM sulfate salts, and further optionally wherein the sulfate salt is sodium sulfate, sodium bisulfate, sodium thiosulfate, ammonium sulfate, ammonium bisulfate, magnesium sulfate, magnesium bisulfate, potassium sulfate, potassium bisulfate; and/or
   (e) 0-500 mM of disodium phosphate, such as about 10 to 250 mM, optionally wherein the formulation or kit comprises about 10 mM of disodium phosphate or about 200 mM of disodium phosphate; and/or
   (f) 0-50 mM of EDTA, optionally wherein the formulation or kit comprises about 1 mM of EDTA; and/or
   (g) 0-200 mM of glutamic acid, optionally wherein the formulation comprises about 50 mM of glutamic acid; and/or
   (h) 0-500 mM of mannitol, optionally wherein the formulation comprises about 300 mM of mannitol; and/or
   (i) 0-500 mM of sucrose, optionally wherein the formulation comprises about 300 mM of sucrose; and/or
   (j) 0-500 mM of arginine such as about 25-250 mM, optionally wherein the formulation or kit comprises about 25 mM to about 50 mM of arginine or wherein the formulation or kit comprises about 200 mM of arginine; and/or
   (k) 0-500 mM of chloride salts, about 25-250 mM, optionally wherein the formulation or kit comprises about 50 mM or about 200 mM of chloride salts; and/or
   (l) 0-200 mM of histidine, optionally wherein the formulation or kit comprises about 50 mM of histidine; and/or
   (m) 0-200 mM of glycine, optionally wherein the formulation or kit comprises about 50 mM of glycine or about 200 mM glycine; and/or
   (n) 0-200 mM of glutamate, optionally wherein the formulation or kit comprises about 50 mM of glutamate.
   (o) 0-200 mM of aspartate, optionally wherein the formulation or kit comprises about 50 mM of aspartate; and/or
   (p) 0-0.01% (w/v) of non-ionic surfactant, optionally wherein the formulation or kit comprises about 0.001% or about 0.01 (w/v) of non-ionic surfactant, further optionally wherein the non-ionic surfactant is a poloxamer such as Pluronic F-68 or a polysorbate; and/or
   (q) 0-500 mM of sodium chloride, such as about 25 to 250 mM, optionally wherein the formulation comprises about 50-200 mM of sodium chloride, such as about 200 mM or about 50 mM sodium chloride.
9. The formulation or the kit according to any one of Embodiments 6-8, wherein the pH of the formulation or the kit is:
(i) from about 4 to about 8, optionally wherein the pH of the formulation is from about 5 to about 7.5; or
(ii) about 6 or about 6.5; or
(iii) about 4 or about 5.
10. The formulation or kit according to any one of Embodiments 5-9, wherein the formulation or kit further comprises a virus, optionally wherein the virus is any of a viral particle, a viral fragment, a virus-like particle, a viral vector or a vector virus, optionally wherein the virus is:
(i) a simian virus, optionally wherein the simian virus is an adeno-associated virus (AAV), a recombinant AAV, Adenovirus, Herpes Simplex Virus, vesicular stomatitis virus, Maraba virus, Vaccinia virus, Measles virus, Newcastle Disease Virus, Rotavirus, Reovirus, Type I Poliovirus, Coxsackievirus A21, Seneca Valley virus, Dengue virus, Yellow fever virus, West Nile virus, zika virus, Alphavirus or Coronavirus, and further optionally wherein the AAV is AAV2, AAV9, AAV8 or AAV5; and/or
(ii) a live virus, an attenuated virus, a live-attenuated virus, or an inactivated virus; and/or
(iii) in the form of a vaccine; and/or
(iv) a hybrid or chimeric virus; and/or
(v) present at a concentration of 1×10⁶-1×10²⁴ vg/ml.
11. A pharmaceutical composition comprising a formulation as defined in any one of Embodiments 5-10, and a pharmaceutically excipient, carrier or diluent thereof.
12. A formulation or kit according to any one of Embodiments 5-10 for use in medicine.
13. A formulation or kit according to Embodiment 10 for use in:
(i) gene therapy; and/or
(ii) cell therapy; and/or
(iii) vaccination and/or immunisation; and/or
(iv) immunotherapy, optionally wherein the immunotherapy is oncolytic virotherapy.
14. A method of stabilising a virus comprising combining the virus with a formulation as defined in any one of Embodiments 5-10 or with a pharmaceutical composition as defined in Embodiment 11.
15. A method of selecting a suitable albumin for use in stabilising a virus, the method comprising determining whether the albumin is in a substantially unaggregated form, optionally wherein:
(i) determining whether the albumin is in a substantially unaggregated form comprises:
   (a) assessing whether there is less than 0.025% (w/w) of albumin in a polymeric form, optionally wherein the assessment is performed by high-performance liquid chromatography, wherein a value of less than 0.025% (w/w) of albumin in a polymeric form is indicative of the albumin being in a substantially unaggregated form; and/or
   (b) assessing whether the hydrodynamic radius of albumin does not change more than 4.5% when incubated at 40° C. for a duration of 3 days, optionally wherein the assessment is performed by dynamic light scattering, wherein a hydrodynamic radius that does not change more than 4.5% when incubated at 40° C. for a duration of 3 days is indicative of the albumin being in a substantially unaggregated form; and/or
   (c) assessing whether the polydispersity index of albumin is equal or less than 0.05; and/or
(ii) the method further comprises formulating the albumin with an anion having multiple negative charges housed on a flexible backbone, and assessing whether the formulation comprising the albumin and the anion stabilises a virus, optionally wherein the anion having multiple negative charges housed on a flexible backbone is as defined in any one of Embodiments 2(xiii)-2(xvii) and 7(xii)-7(xvii).

The present invention is further described by the following figures and examples that should not be construed as limiting the scope of the invention.

### PART B—BRIEF DESCRIPTION OF THE FIGURES

FIG. 1 (A) Time course of the hydrodynamic radii (cumulant analysis) upon incubation at 25° C. followed by incubation at 40° C.—solutions in Tris-Citrate+Na₂SO₄ at high AAV2 concentration; Hydrodynamic radius at the beginning and end of the incubation at 25° C. and 40° C. of the AAV2 at high concentration in, respectively (B) Tris-Citrate buffer and (C) Tris-Citrate buffer+Na₂SO₄. The radius was not reported for those samples showing precipitate.

FIG. 2 Hydrodynamic radius at the beginning and end of the incubation at 25° C. and 40° C. Larger errors bars are due to the sample aggregating but have been plotted for comparison. AAV2 at low concentration and Albumins at 5 mg/mL. (A) Tris-Citrate buffer and (B) Tris-Citrate buffer+Na₂SO₄.

FIG. 3 Backgrounded Membrane Imaging-Images of the wells of the (A) Tris-Citrate and (B) Tris-Citrate+Na₂SO₄. Solutions stored at 4° C. for two months. Scale bar is 1000 μm (microns).

FIG. 4 (A) Number of particles with an ECD ≥2 μm and (B) ThT fluorescent particles with an ECD ≥2 μm in both formulations tested and in the presence of albumins from different sources. Solutions stored at 4° C. for two months.

FIG. 5 (Brightfield-SIMI) number of particles with an ECD ≥2 μm of the AAV2 solutions after 4 cycles of Freeze-Thaw in (A) Tris-Citrate and (B) Tris-Citrate+Na₂SO₄.

FIG. 6 Number of ThT positive particles with an ECD ≥2 μm of the AAV2 solutions after 4 cycles of Freeze in (A) Tris-Citrate and (B) Tris-Citrate+Na₂SO₄.

FIG. 7 Correlations between the (A) % Polymer, as measured by SEC-HPLC, (B) Polydispersity index and (C) hydrodynamic radius (as measured by DLS), (D) Amount of total lipids (albumins at 100 mg/mL) and the Temperature incubation scoring for the T=25° and 40° C. incubation experiments.

FIG. 8 Correlations between the (A) % Polymer, as measured by SEC-HPLC, (B) Polydispersity index and (C) hydrodynamic radius (as measured by DLS) and the Temperature incubation scoring for the T=40° C. incubation experiments.

FIG. 9 Correlations between the (A) the amount of total lipids and (B) the Free Cysteine and the Temperature incubation scoring for the T=40° C. incubation experiments.

FIG. 10 Correlations between the % Particles coverage, as measured by BMI and respectively: (A) % Polymer (SEC-HPLC), (B) Total Lipids, (C) Free cysteine and (D) Normalised Free Cysteine-Norm NTD (Ellman's assay and ESI-MS). 4° C. storage.

FIG. 11 Correlations between the number of ThT positive particles and respectively: (A) Free cysteine, (B) Total Lipids, (C) Ratio between the two peaks in the fluorescence spectrum and (D) Zinc amount. 4° C. storage.

FIG. 12 Correlations between the number (BMI-SIMI) particles and respectively: (A) the amount of modification (except Oxidation) as measured by ESI-MS, (B) Free Gibbs energy and (C) m-value as calculated by chemical denaturation by using Guanidinium Chloride (GdnHCI) as denaturant. Freeze-Thaw stress.

FIG. 13 Correlations between the number (BMI-SIMI) particles and different groups of metals. Freeze-Thaw stress.

FIG. 14 Correlations between the number of ThT positive particles and respectively (A) ANS K_{d}, (B) and (C) various metals. Freeze-Thaw stress.

FIG. 15 Aggregation: (A) Area % of the polymer peak as determined by SEC-HPLC, (B) hydrodynamic radius and (C) Polydispersity index as determined by DLS. To note that the serum derived albumin was polydisperse so no value of cumulant derived radius or polydispersity is reported. The dashed lines in the graphs are the proposed thresholds for a “good” albumin.

FIG. 16 Ellman's assay result expressed as mole Cys per mole protein. The dotted line is the threshold for a “good” albumin.

FIG. 17 ESI-MS PTMs: Percentage of PTM products as measured by ESI-MS. 100% is the peak with highest abundance. The dashed line is the proposed sum of % of PTM threshold to define a “good” albumin.

FIG. 18 ESI-MS PTMs: Percentage of N-terminal degradation products as measured by ESI-MS. 100% is the peak with highest abundance. The dashed lines are the proposed thresholds to define a “good” albumin.

FIG. 19 Time course of the changes in the hydrodynamic radius of the albumins incubated at 40° C. All the albumins were diluted in DPBS at 5 mg/mL. (A) “Good” albumin only. (B) Comparison of “bad” albumins with some “good” albumins.

FIG. 20 (A) Time course of the changes in the hydrodynamic radius of Sacch.2.1 in DPBS (black squares) and Rice 1 in, respectively: DPBS (empty circles), Sacch.2 formulation buffer (high salt-crossed hexagons) and Sacch. 1 formulation buffer (octanoate containing buffer-black stars) following incubation at 40° C. (B) reports the same data but uses a different scale on the Y axes to highlight the changes that occur even in octanoate containing buffer. These changes were not visible in (A) because the high instability in one of the buffers tested (high salt).

FIG. 21 Stern-Volmer plots. Only one batch is shown for Sacch. 1 and 2 and no plot for Sacch.3: all are equivalent.

FIG. 22 Stern-Vomer constants calculated from the first part of the Stern-Volmer plots. The dashed line is the proposed threshold of KSV for a “good” albumin.

FIG. 23 Total amount of lipids (excluded Octanoate) present in various albumin stocks.

FIG. 24 ANS K_{d} values.

FIG. 25 Differential scanning calorimetry traces of Sacch.2 at 1 mg/ml in citrate and phosphate buffers. Effect of added NaCl.

FIG. 26 Differential scanning calorimetry traces of Sacch.2 at 1 mg/mL in various buffers.

FIG. 27 Hydrodynamic radius measured after two hours of incubation at 60° C. for a non-octanoate containing albumin.

FIG. 28 Stability study with Sacch. 1 in different buffers. Total protein at (A) 6 months and (B) 36 months as measured by SEC-HPLC.

FIG. 29 Monomer concentration (normalised to 0 to 1 scale to take into consideration the small variations between the solutions) measured by SEC-HPLC at the 36 months' time point. (A) 25° C. storage and (B) 5° C. storage.

FIG. 30 Comparison of the residues in contact with citrate (1TF0) with those involved in Octanoate binding (Kawai et al, 2017).

FIG. 31 Compounds with the same structural features as citrate.

### PART B—EXAMPLES

In the Examples, albumins from serum and recombinant sources were used. When different albumin sources from the same recombinant species were used, they are designated “1, “2”, etc. When multiple batches were used from the same source, they are designated “1.1”, “1.2”, etc (i.e. source 1, batch 1; source 1, batch 2). The majority of the albumins tested were mature sequence wt human albumin (SEQ ID NO: 2).

### Example 1: Determining the Ability of Albumin to Stabilise AAV Against Temperature Stress

The following experiments were aimed at testing albumins from different sources for their ability to protect the AAV2 against different stresses.

The stresses used were:
Incubation at 25° C. and 40° C.
Freeze-Thaw, 4 cycles
Storage at 4° C. for 2 months

We selected five albumins, all stated by the manufacturer to be suitable for use as an excipient for final product formulation: serum-derived, one recombinant albumin from Saccharomyces cerevisiae (Saccharomyces 1.2), one recombinant albumin from Pichia (Pichia 1), and two recombinant albumins from rice (Rice 3 and Rice 4). All of these albumins were mature sequence wt human albumin (SEQ ID NO: 2).

After some rounds of formulation scouting, we selected the following conditions:
Tris-Citrate or TC=150 mM Tris, 50 mM Citrate, 25 mM MgCl₂, PH=6
Tris-Citrate+Na₂SO₄ or TCN=150 mM Tris, 50 mM Citrate, 25 mM MgCl₂, PH=6, 50 mM Na₂SO₄

In all the experiments the albumin concentration was 5 mg/mL.

The incubation at 25° C. and 40° C. was carried out both at low (vp/mL~E+11) and high (vp/mL~E+13) AAV2 concentrations. In either case, the solutions were prepared by diluting the AAV2 stock (Vigene, vg/mL=1.06E+13, vp/mL~5.52E+13, phosphate buffer saline with 0.001% Poloxamer 188) in the target buffer.

Dynamic Light Scattering (DLS-Dyna Pro Plate reader II, Wyatt, serial #: 221-WPR2) was then used to follow the aggregation over time. To perform the experiment, 25 μL of each sample were transferred to a 384 Aurora well plate (Wyatt). Each formulation was analysed in triplicate. The wells were sealed with 20 μL of paraffin oil to prevent evaporation during the experiment. Before measurement, the plate was spun at 2000×g for 2 minutes to remove air bubbles. The changes in hydrodynamic radii (R_{H}—cumulant analysis, see also Example 8) following incubation at 25° C., followed by the incubation at 40° C., were monitored over time.

The samples for freeze-thaw (FT) and 4° C. storage experiments were prepared as described above, but only at high AAV2 concentration (vp/mL~E+13). Then the solutions were transferred into 0.5 mL Eppendorf® LoBind microcentrifuge tubes and these were, respectively, placed in an incubator (4° C. storage) or frozen at −20° C. These last solutions were then subsequently subjected to 4 cycles of freeze-thaw before measurement. The aggregation was measured by using Backgrounded Membrane Imaging (Aura system, Halo Labs, Burlingame, CA, USA). 50 μL of the samples, after storage for 2 months at 4° C. or after 4 freeze-thaw cycles, were spotted, without dilution, onto the membrane of Black Halo Labs plates (Halo Labs) suitable for fluorescence imaging. After imaging in Brightfield and Side Illumination Membrane Intensity (SIMI) the plates were removed from the instrument, a solution of the protein stain Thioflavin T (ThT) was added and incubated for 10 minutes. After the excess dye was removed, the fluorescence was acquired on the fluorescence channel (Ex 440/40 Em 500/40). The ThT stock was prepared by dissolving ~3 mg of ThT dry powder in 1 mL water. The solution was then filtered through a 0.22 μm syringe filter followed by measurement of the concentration by diluting the stock solution in ethanol and using an extinction coefficient of 26,620 M-1 cm-1 at 416 nm (Khurana et al, 2005). The number of particles with an Estimated Circular Diameter (ECD) ≥2 μm were reported as a measure of aggregation. We have also used the number of ThT fluorescent particles with an ECD ≥2 μm as a measure of amyloid-like protein aggregates. In some cases, we reported the number of particles with an ECD ≥2 μm as measured in brightfield subtracted to the ones in the same size range with a SIMI intensity higher than 10. This is named in the plots as: (Brightfield-SIMI). The rationale behind this treatment of the experimental data is that in this way, only protein aggregates are counted, eliminating non-protein particles from the scoring (e.g. due to environmental contamination). Indeed, non-protein aggregates are not flat on the membrane and are characterised by a high SIMI value. On the contrary, protein aggregates are flat on the membrane, with SIMI values around ~0±10. Therefore, by subtracting the SIMI particles from the ones counted in Brightfield, only the protein aggregates are considered.

FIG. 1 (A) reports an example of the raw data obtained in the temperature incubation experiments and shows the changes of the hydrodynamic radius over time upon incubation at 25° C. and, subsequently, at 40° C. of AAV2 at 9.2E+12 vp/mL in Tris-Citrate+Na₂SO₄ formulated with three of the five albumins tested (for clarity in this figure we have restricted the number of samples shown): serum-derived, rice-derived (“Rice 3”) and Saccharomyces 1.2. While the Saccharomyces 1.2 radius did not change during the experiments, the serum-derived albumin and Rice 3 aggregated, as indicated by the increase in R_{H} with time. FIGS. 1 (B) and (C) show the hydrodynamic radii at the beginning (TO) and after incubation at 25° and 40° C. in, respectively, Tris-Citrate and Tris-Citrate+Na₂SO₄ buffers for all five of the albumins tested (serum-derived, Saccharomyces 1.2, Rice 3, Rice 4 and Pichia 1) and for the no-albumin controls. Similar results were obtained at low AAV2 concentration (FIG. 2). Overall Saccharomyces 1.2 and the Pichia 1 performed better across both temperatures and in both buffers when compared to the rice-derived and the serum-derived albumin.

Similar results were obtained for the solutions stored at 4° C. for 2 months where we observed that the protection against aggregation was, again, dependent on the albumin source (FIG. 3). In particular, Saccharomyces 1.2 and Pichia 1 performed the best at stabilising the AAV2 during storage at 4° C., not only by reducing the total number of particles formed (FIG. 4 (A)), but also by minimising the amyloid-like protein aggregates (ThT positive particles-FIG. 4 (B)).

The same differential stabilisation of AAV2 by the albumins was observed after 4 freeze-thaw cycles in both buffers tested (FIG. 5). The improved performance of two of the albumins tested (Saccharomyces 1.2 and Pichia 1) is more evident when considering the number of amyloid-like (ThT positive) protein particles with an ECD≥than 2 μm (FIG. 6).

The results described in this example show that the AAV2 stability against aggregation was not only dependent on the presence of albumin but also on the properties of the albumin used, therefore this merited further investigation.

Without wishing to be bound by theory, the consistency of the performance of the albumins across different conditions might be related to structural integrity, and therefore might explain the differences in the albumins' abilities to stabilise viruses such as AAVs.

### Example 2: Identifying the Key Quality Parameters of an Albumin

To try to understand the observed difference in performance, we correlated the albumin stock solutions' properties with their ability to stabilise viral vectors. The scoring was done by allocating “Quality Parameters” to the albumins and “Performance Indicator Parameters” to the AAV2 formulated with albumins, and then correlating each Quality Parameter with a Performance Indicator Parameter. Therefore, a Performance Indicator Parameter is an albumin attribute which is a predictor of albumin performance.

First, a range of albumins from different sources (Saccharomyces derived, serum derived, rice-derived and Pichia-derived) were analysed for identity, post-translational modifications, purity, and structure with an array of methods as schematically reported in Table 1. These assays were done on albumin solutions only, after a suitable dilution in Dulbecco's Phosphate Buffered Saline (DPBS) whenever was necessary to bring the solution into the dynamic range of the relevant technique. The output of each assay was summarised by a number and this value, in turn, used to score the albumins for a total of 72 “Quality Parameters”.

TABLE 1
List of methods used to analyse albumins from different sources

| Method | Aspect |
|---|---|
| Mass Analysis of Recombinant | Mass and Homogeneity |
| Albumin by Electrospray Mass | |
| Spectrometry (ESI-MS) | |
| UV-Vis spectra | Concentration, other chromophores, |
| | presence of coloured impurities |
| Intrinsic Fluorescence | Integrity of the Tertiary Folding |
| ANS binding | Integrity of the Tertiary Folding |
| Ligand Binding | Secondary Structure and Tertiary |
| | Folding |
| Nal and succinimide quenching | Accessibility of Trp214 |
| Chemical Stability using | Integrity of the Tertiary Folding |
| Chemical denaturation | |
| DLS-Temperature ramp | Integrity of the Tertiary Folding and |
| (Tagg), incubation at T = 60° C. | aggregation |
| and at T = 40° C. | |
| Comparison of rHA and I by | Oligomeric and Degradative species |
| Isoelectric Focusing (rHA = | (as indicated by primary structure) |
| recombinant human albumin) | |
| Charge Variants assay | Oligomeric and Degradative species |
| (IEX-HPLC) | (as indicated by primary structure) |
| SEC-HPLC-UV (Monomer | Oligomeric Species |
| and Polymer) | |
| SEC-HPLC-MALS | Oligomeric Species |
| DLS at 25° C. | Hydrodynamic radius of the protein |
| | in solution and high molecular |
| | weight aggregates |
| DLS k_{D} measurement (derived | self-interaction, such as weak |
| first-order diffusion interaction | intermolecular self-interaction |
| parameter) | |
| Determination of the Free | Free SH |
| Thiol Content | |
| SEC-HPLC AGE | Amount of glycation |
| Visual Inspection | Differences in colour |
| Fluorescence of AGE | Advanced Glycation end products |
| Lipid Analysis | Lipid contents |
| Trace metals | metals |
| Osmolality | Overall number of solutes presents |

The performance of each albumin in stabilising the AAV2 against different stresses (freeze-thaw, temperature incubation and 2 months storage at 4° C.), as described in Example 1, was scored, for each stress test, and summarised with a number. These scores (six in total), or “Performance Indicator Parameters”, were then correlated to the albumin quality parameters. In the temperature incubation experiments, where the changes in hydrodynamic radius were monitored over time at two different temperatures, the (0-1) normalised standard deviation of the averaged albumin hydrodynamic radius (in the formulation with an AAV) across the 25° and 40° C. incubation was taken as the performance indicator parameter because, for an aggregating system, the standard deviation of the average radius will be high. The % of particles coverage and the number of ThT positive particles (amyloid-like protein aggregates) were taken as performance indicator parameters in the 4° C. storage experiments. The scoring in the two buffers were combined to have a reduced number of parameters for the correlation analysis. A similar data simplification was done in the freeze-thaw (FT) experiments. As a performance indicator parameter in FT, we used the number of particles in Brightfield minus the number of particles in SIMI as explained in Example 1. In parallel we also correlated the total number of ThT positive particles, as done for the 4° C. storage. Correlation was assessed using the value of the Pearson Coefficient and parameters were considered to correlate if the Pearson Coefficient was equal or higher than |0.90| (“I” indicates the modulus). From this correlation analysis, some quality parameters of the albumin stock solutions were identified to be suitable for use as performance indicator parameters. It must be noted that aggregation is a complex phenomenon and different kinds of aggregates can arise from different stress tests via different mechanisms. Our aim was to identify the minimum set of quality parameters which is predictive of albumin performance across various conditions.

The first performance indicator parameter, calculated for the temperature incubation experiment, was the overall hydrodynamic radius standard deviation across the incubation at both 25° and 40° C. We found that this parameter correlates with the % polymer, the albumin hydrodynamic radius and the polydispersity index (FIG. 7). If this is the case, then Polymer % (FIG. 7 (A)) would be considered to not correlate. The correlation with quality parameters related to albumin aggregation, is stronger when the same correlation is carried out by considering only the incubation at 40° C. (FIG. 8).

One additional inverse correlation is also evident, relating to the total lipids (FIG. 9 (A)). FIG. 10 shows the correlations found when analysing aggregation following 4° C. storage: the percentage of polymer (SEC-HPLC) and the amount of total lipids (GC-MS) correlates well with the stability of the formulations, confirming the aggregation as a quality parameter. Free metals and the metal binding capacity of albumin correlations were also observed, indicating that free metals in solution can be detrimental for performance. Indeed, the albumin metal binding capacity can also be related to both free cysteine and N-terminal degradation (NTD). When the same kind of correlation is done by using the total number of ThT positive particles (amyloid-like protein aggregates), the correlation observed indicates that, in the stress at 4° C., the most important parameters are the free cysteine, the amount of total lipids and the overall structural integrity of the protein. Finally, the strong correlation with zinc points to a role of metals in the quality of the albumins (FIG. 11). The total number of particles, formed during the FT stress, was found to be strongly correlated with the total percentage of modifications (excluded oxidation) indicating that these modifications (cysteinylation, addition of extra mass, glycation) can impair albumin structure and therefore performance (FIG. 12).

Also interesting is the observation that some metals can cause a decrease in albumin performance (FIG. 13) i.e. the ability to limit aggregation in a virus and albumin formulation.

The correlation of the ThT positive particles, instead, is with both metals but also with the K_{d} of the binding of 8-anilino naphthalene sulfonic acid (ANS) (FIG. 14). ANS is known to bind specifically to albumin but, it is also a dye that binds to exposed hydrophobic patches. Therefore, it is likely that the observed increase in ANS-binding, for some of the albumins, is correlated to a non-specific binding to partially unfolded regions of these proteins.

As a result of these correlation studies, some quality parameters of the albumin stock solutions, such as aggregation, free-sulfhydryl, presence of post-translational modifications and N-terminal degradation, can be considered as suitable performance indicator parameters. Details on how key quality parameters vary between different albumin sources were investigated in more detail.

### Example 3: Albumin Quality Parameters—Aggregation

Higher order aggregates (higher than dimer and trimer) can nucleate the formation of larger aggregates when a protein is subjected to stress (thermal or interfacial). Therefore, we wanted to score the albumin preparations according to the aggregation level. Because the % polymer, as measured by SEC-HPLC, is quantitative, we took this as a measure of the albumin quality. To corroborate this choice, we also described other parameters related to aggregation.

We analysed several albumins (between these also those used in Example 2):
Various batches of albumin produced in S. cerevisiae:
   Saccharomyces 1.1, Saccharomyces 1.2, Saccharomyces 1.3
   Saccharomyces 2.1, Saccharomyces 2.2, Saccharomyces 2.3
   Saccharomyces 3.1, Saccharomyces 3.2 (expired batches)·
Recombinant albumins produced in Pichia from two suppliers: Pichia 1 and Pichia 2
Four recombinant albumins produced in rice: Rice 1, Rice 2, Rice 3 and Rice 4
A serum-derived albumin.

All albumins tested were mature sequence wt human albumin (SEQ ID NO: 2) except for Pichia 2 which is believed to have an approximately 4-amino acid N-terminal extension relative to SEQ ID NO: 2.

By “expired batches”, we mean batches which had exceeded 60 months since being bottled following their production and purification.

Albumin samples were prepared, as mentioned in Example 2, by diluting the albumin stocks in DPBS (liquid formulations) or, in the case of the lyophilised formulations by reconstituting the powder in DPBS (Sigma-Aldrich, Cat. No. D8537, Dulbecco's Phosphate Buffered Saline, Modified, without calcium chloride and magnesium chloride, liquid, sterile-filtered, suitable for cell culture) at ~100 mg/mL.

All concentrations were checked spectrophotometrically by diluting the albumins in DPBS to ~1 mg/mL. The spectrum was acquired in the near-UV/Visible region, at 1 mg/mL, I=1 cm (range: 240-600 nm). For the concentration determination the absorbance was first corrected for the scattering (A₃₅₀), then the corrected values of the absorbances at 280 nm were used for the determination of the stock solution concentration. The molar extinction coefficient used is: ε₂₈₀=0.53 for 1 mg/mL solution.

The aggregation level was quantified by SEC-HPLC as “percentage polymer” and qualitatively evaluated by Dynamic Light Scattering (DLS) via changes in the values of the hydrodynamic radius (R_{H}) and polydispersity index (PD index). The polymer amount was measured by loading 20 to 40 mg/ml albumin on a TSK G3000SWXL GPC column (7.8 mm id×300 mm) equilibrated in 20 mM sodium phosphate (Fisher Chemicals), 100 mM sodium sulfate (Fisher Chemicals), 0.05% (w/v) sodium azide (Sigma Chemicals), pH 7.0. The flow rate was 1 mL/min, and the albumin were diluted from the stock into double distilled water. The percentage of albumin polymer in the sample was calculated using:
wherein:

$$Result = \frac{r u}{r T} \times 100$$

rᵤ=peak response of albumin polymer; and
r_{T}=sum of all albumin related peak responses.

The DLS measurements were done on a DynaPro™ Plate reader II (Wyatt, serial #: 221-WPR2−λ=826.150 nm). The albumin samples were diluted to 5 mg/ml in DPBS and then plated in triplicate in a 96-well Corning 3880 Half Area non-treated plate. Before measurement the plate was spun at 2000×g for 2 minutes to remove bubbles.

In Example 2 we described the observed correlation between the performance of the albumin in stabilising the viral vectors (AAV2) and some of the albumin quality parameters. One of these correlations was with the extent of aggregation like the polydispersity index, as measured in Dynamic Light Scattering, and the percentage of polymer, as measured by SEC-HPLC. In FIG. 15 the albumins are scored according to (A) % Polymer, (B) hydrodynamic radius and (C) PD index. The Rice 3 albumin, which performed poorly in the AAV2 stabilisation, is also characterised by high levels of aggregates and by a large R_{H} compared to that expected for an albumin. The serum-derived albumin, which also performed poorly, presented a high amount of polymer. The serum-derived albumin is not reported in the DLS results because, due to its polydispersity, it was not possible to perform a cumulant analysis.

Consequently, we propose that a “good” albumin has one or more, and ideally all three, of the following properties when analysed during their manufacturer-designated shelf-life (reported in FIG. 15 as a dashed line):
% Polymer <0.025% by SEC-HPLC analysis (quantitative)
Hydrodynamic Radius (nm) of the stock solution at 5 mg/ml=3.55±0.15 by DLS.
Polydispersity index of the stock solution at 5 mg/mL-PD index≤0.05 by DLS

### Example 4: Albumin Quality Parameters-Free Thiol Level

One of the many physiological roles of albumin is to prevent oxidation. Indeed, it represents the major antioxidant component in plasma. This antioxidant activity is performed both via ligand-binding (e.g. binding to metals, fatty acids and drugs) and by direct antioxidant activity. Albumin contains oxidation-prone amino acids, which can work as scavengers, for example the free thiol (Cys34) and some methionine residues (Met87, 123 in DI (i.e. Domain I); Met298, 329 in Domain II; Met446, 548 in Domain III) (Roche et al, 2008). Oxidation was found to alter albumin thermal stability, aggregation kinetics, type of intermediates, aggregates morphology and also to impair albumin function (Sancataldo et al, 2014; Kawakami et al, 2006) and the wide variations of the oxidation state between the available sources of albumin (serum derived and recombinant) can affect albumin batch to batch performance consistency. It has also been reported that oxidative modification can alter albumin binding capacity and the extent of the changes is dependent on the ligand and the type of modification (Oettl et al, 2007). Therefore, the oxidative changes in albumin samples could potentially have an impact on all its applications. Cys34 is highly conserved within mammalian serum albumins. This residue is surface exposed and surrounded by Asp38, His39, and Tyr84 which affect the ionization state and modulate the reactivity of Cys34 (Fanali et al, 2012). Because of the importance of the free Cys in albumin preparations it has been even suggested to consider the levels of free Cys34-HSA as a predictive marker for the functional impairment of albumin preparations (Watanabe and Maruyama, 2016; Miyamura et al, 2016).

The amount of free cysteine was determined by using the Ellman's reagent, DTNB ((5,5′-Dithiobis(2-nitrobenzoic acid-Sigma, Cat Number: D8130). A stock solution of DTNB was prepared at 4 mg/mL in assay buffer (0.1 M phosphate buffer, pH 8.0). The reagent was prepared on the day of the measurement and was protected from light.

For the standard curve, a 1.5 mM stock solution of L-Cysteine (Sigma #30089; MW=121.165 g/mol) was prepared in water on the same day of the assay.

From this stock, the Cysteine standards solutions were prepared by dilution in assay buffer at the following concentrations: 1.5, 1.25, 1.0, 0.75, 0.5, 0.25, 0 mM.

Assay solutions were prepared as follows:
500 μL assay buffer
50 μL standard or sample
10 μL Ellman's reagent

And then were incubated in the dark for 10 minutes before measuring the absorbance at 412 nm.

The molar ratio [Cys]/[Albumin] was calculated for each sample. Values of the relative ratio (relative to the highest amount of free cysteine sample) are also reported.

In Example 2 we found a correlation between the albumin performance and free cysteine, with those albumins with poor performance characterised by a low value of free cysteine. In FIG. 16 we show the ranking of different albumin preparations against this value with Rice 3 and Serum-derived appearing low in the ranking Saccharomyces 1.1, 1.2 and 1.3 and Pichia 1 appearing higher in the ranking. Therefore, we put forward a threshold of the free cysteine content, to qualify as a “good” albumin, of ≥0.5 mol/mol protein.

### Example 5: Further Albumin Quality Parameters-Post-Translational Modifications

Albumin is the most abundant protein in plasma and it is involved in a number of diverse physiological functions (Fanali et al, 2012). Because of the inherent flexibility of the albumin structure, its conformation, and therefore performance, is affected also by post-translational modifications (PTMs). Glycation is a non-enzymatic reaction between a monosaccharide and a protein. Albumin, due to its long circulating half-life, is prone to glycation and the extent to which this occurs is also linked to disease states. Glycation is also concurrent with other modifications and alters ligand binding and has been demonstrated to be immunogenic when injected into animals (Fanali et al, 2012; Neelofar and Ahmad, 2017; Maciążek-Jurczyk et al, 2018). But glycation is not only present in serum derived products. In a study from Health Canada (Frahm et al, 2014), it was found that albumins produced in different expression systems had different levels of modifications and these were linked to the expression system used. In this work they compared a set of recombinant albumins and they found tertiary structure alterations correlated with a high degree of glycation. An overview of the changes in structure and function following glycoxidative process is given in References (Maciążek-Jurczyk et al, 2018; Awasthi et al, 2016). In this example we scored the albumins for the extent of not only glycation but, in general, the sum of those modifications potentially important for albumin quality: extra mass present, cysteinylation and various glycated forms.

The ESMS (electrospray mass spectroscopy) method used comprised first the desalting of the albumin samples and the current reference standard (USP) on a Reverse Phase-Ultra Performance Liquid Chromatography (RP-UPLC) column. Following this step, the samples were analysed by ESMS for intact mass by sample injection (direct infusion) into an electrospray Mass Spectrometer, having a mass accuracy of 0.01% for proteins up to 100,000 Daltons. Data were acquired in positive mode and MS (mass spectroscopy) scans performed over m/z 625-3000. Following the run, the data were processed (smoothed and background subtracted) then deconvoluted to analyse the intact mass of the protein present in the reference standard and sample. The mass assigned to the main component in the test samples must be 66438+20 Da. The amounts of protein required for injection and the settings under which they are run are system (high performance liquid chromatography HPLC/UPLC, mass spectrometer MS) specific.

Method using Waters Acquity UPLC and Bruker micrOTOF II MS (using Hystar 3.2 control software):
1. Sample/STD (USP) Injection 5 μl at 0.3 mg/ml
2. LC solutions: Solvent A1: 100% Laboratory grade water (LGW) with 0.1% Formic Acid (FA); Solvent B1: 100% Acetonitrile (ACN) with 0.1% FA
3. LC Column: Waters Acquity UPLC BEH300 C4 1.7 μm 2.1×50 mm analytical column, flow rate 0.4 mL/min and column oven at 50° C.
4. Gradient:

| Time (min) | Flow (mL/min) | % A | % B |
|---|---|---|---|
| Initial | 0.400 | 95.0 | 5.0 |
| 0.4 | 0.400 | 95.0 | 5.0 |
| 0.5 | 0.400 | 50.0 | 50.0 |
| 2.5 | 0.400 | 30.0 | 70.0 |
| 2.6 | 0.400 | 10.0 | 90.0 |
| 3.2 | 0.400 | 10.0 | 90.0 |
| 3.3 | 0.400 | 95.0 | 5.0 |
| 5.0 | 0.400 | 95.0 | 5.0 |

In FIG. 17 the albumins are scored accordingly to the sum of the % of all these post-translational modifications, with 100% taken as a normalised valued based on the peak with the highest abundance. This, for one of the albumins, was the one with an extra mass added therefore, when summed to the other modifications, it resulted in a value higher than 100%. Similarly, Rice 1 albumin highest abundance peak is the cysteinylated product, leading in this case as well, to a sum higher than 100%.

We therefore put forward a threshold of these summed PTMs as <75% as measured by ESI-MS.

### Example 6: Further Albumin Quality Parameters-N-Terminal Degradation

It is well known that metals bind to serum albumin at different sites, one of which is the N-Terminal sequence Asp-Ala-His (ACTUN motif=Amino-Terminal Copper and Nickel) where metals like Cu(II), Co(II) and Ni(II) binds with a square-planar geometry. There are other metal binding sites accommodating metals, like Zn(II), with different valency and coordination geometry (Bal et al, 2013). It has also been demonstrated that binding of metals and fatty acid can be mutually affected (Barnett et al, 2013; Amirtharaj et al, 2008) or cause structural changes to the albumin (Chen et al, 2014).

Due to the correlations between the percentage of N-terminal degradation and the amount of some metals (see Example 2), we considered whether the extent of N-Terminal degradation (NTD) was a relevant quality parameter.

From FIG. 18, it is evident that the albumins that were less capable of stabilising AAV2 were those characterised by having the highest levels of total NTD and as such we put forward a threshold of N-terminal degradation for a good performing albumin at a value below 12.5% as determined by ESI-MS.

### Example 7: Further Albumin Quality Parameters-Metal Content

Approximately 0.2 g of sample from each sample was accurately weighed into the micro-insert vessels of the Milestone Ethos UP microwave system (Analytix, UK). Each sample was prepared in triplicate. The highest purity grade chemicals were used throughout (Ultra High Purity Acids & Reagents, Romil, UK). Sample digestion was performed using 1 mL nitric acid (HNO₃), and 0.2 mL of hydro peroxide. Vessels were closed after the addition of the chemicals and digested using the following program:
Temperature ramped to 180° C. over 10 minutes,
Hold at 180° C. for 30 minutes.
Cool to room temperature

The digest solutions were transferred into 50 mL polyethylene tubes, and made up to 20 g with ultra-pure water 18 MQcm (megaohm-cm) (Elga Water, UK). This resulted in a final acid concentration in the samples of 5% HNO₃ and with an overall dilution factor of 100 for the samples. Two microwave batches were run each contained 3 reagent blanks and 1 replicate of a quality control (QC) material. Additionally, two samples per batch were selected at random and spiked with a multi-element standard.

Semi-quantitative analysis of the digested samples was performed using an Agilent 7700 ICP-MS (Agilent Technologies, UK). The samples were introduced into the plasma using a glass nebuliser (Micromist) and a Scott double pass spray chamber cooled at 2° C. Analysis was performed in pumping mode using the instrument in collision mode with helium as cell gas to reduce potential interferences. General instrumental parameters for semi-quantitative analysis are shown in Table 2. The instrument was optimised and tuned for maximum sensitivity. Semi-quantitative analysis is a screening method which uses relative response factors and a multi-element standard solution containing 0.05 μg·g⁻¹ of Li, Al, Co, Y, Cd, Ce, Tl and U and with Si, Ca and P at 0.3 μg·g⁻¹ (Romil, UK) which was prepared in 5% HNO₃ to match the sample acid concentration.

TABLE 2
General parameters for semi-quantitative
analysis using Agilent 7700 ICP-MS
Instrumental parameters:

| | | |
|---|---|---|
| RF Power | 1550 | W |
| Plasma Gas | 15 | L min⁻¹ |
| Auxiliary Gas | 0.9 | L min⁻¹ |
| Carrier Gas Flow | 1.15 | L min⁻¹ |
| Nebuliser pump | 0.1 | rps |
| He Flow | 5.0 | mL min⁻¹ |

Acquisition parameters:

| | |
|---|---|
| Masses Monitored | m/z 6 to 238 |
| Sweeps per Replicate | 50 |
| Replicates | 3 |
| Dwell Time | 0.12 s (all |
| | isotopes) |

The Limit of Quantification (LOQ) was evaluated using at least 6 measurements of the reagent blank in the analytical run. The LOQ was calculated as the mean blank concentration plus 10 times the standard deviation of the blank measurements. All sample results were corrected for the individual dilution factors and the procedural blank. The concentrations for the samples are reported in μg·g⁻¹ for the sample as received for all elements except Na, K and P which are reported in % (m/m). The results relate only to the items tested.

Two preparations of the certified reference material LGC6027 (Soft drinking water) were subjected to the digestion procedure. Also, four selected samples were spiked with a multi element standard (LGC Standards, UK) containing 48 elements prior to digestion.

Furthermore, LGC6027 (without digestion) and Aquacheck 569 Groups 4 and 5 were analysed by the semi-quantitative method and all relevant results of the quality control materials agreed with the expected values within +/−50% with the exception of Vanadium (V) and Antimony (Sb). A marginal positive bias was obtained for these elements for LGC₆₀₂₇ (without digestion) and Aquacheck 569 Group 5, which could potentially affect the reported values for the samples. However, it was anticipated that these elements could be subsequently analysed using external calibration but the customer requested to suspend the second phase of the project. A check standard was analysed every 6-8 samples to ensure there was no drift.

The result for copper levels in the different albumin samples can be found below:

TABLE 3
Copper levels of different albumin samples

| | 65 Cu (μg/g in | |
|---|---|---|
| | sample as received) | Std dev |
| Saccharomyces 3.2 | 0 | 0 |
| Saccharomyces 1.2 | 0 | 0 |
| Saccharomyces 1.3 | 0 | 0 |
| Saccharomyces 2.1 | 0 | 0 |
| Saccharomyces 3.1 | 0 | 0 |
| Saccharomyces 1.1 | 0.03459 | 0 |
| Saccharomyces 2.2 | 0.04063 | 0.0027 |
| Saccharomyces 2.3 | 0.05297 | 0.00381 |
| Pichia 1 | 0.12814 | 0.00138 |
| Serum Derived | 0.20443 | 0.00227 |
| Rice 1 | 0.26608 | 0.00829 |
| Pichia 2 | 0.34723 | 0.01995 |
| Rice 3 | 0.57288 | 0.00542 |
| Rice 2 | 0.5763 | 0.02436 |

### Example 8: Further Albumin Quality Parameters-Stability at 40° C.

As a quick method to determine an inherent albumin instability we suggest monitoring the hydrodynamic radii of an albumin solution (5 mg/mL) diluted in a common buffer (e.g. DPBS) over time. 90 μL of each sample was plated (triplicate) into a 96-well Corning 3880 Half Area non-treated plate and before measurement the plate was spun at 2000×g for 2 minutes to remove bubbles. The wells were sealed with 50 μL paraffin oil to prevent evaporation during the experiment. The changes in hydrodynamic radii (cumulant analysis) following incubation at 40° C. were then monitored over time.

We found that those albumins that performed poorly in the AAV2 stabilisation, tended to aggregate following incubation at this temperature (FIG. 19). This instability is likely to be due to some structural damage of the albumin molecule during or following production, processing and storage, because it persists even when these samples are reformulated in two buffers which have previously been able to maintain the stability of albumin (for example buffers containing octanoate) (FIG. 20).

Therefore, the incubation of the albumin stock, diluted at 5 mg/mL in DPBS, could be used as a test for determining the quality of an albumin and therefore its suitability for stabilising viruses such as AAVs.

### Example 9: Further Albumin Quality Parameters

The structural impairment, as a possible explanation of the poor performance of some of the albumins, was also supported by other results, such as the tryptophan quenching studies done by using sodium iodide (NaI) as a quencher. Sodium iodide was chosen because of its negative charge and its ability to probe the Trp environment of albumin (Chadborn et al, 1999). Human serum albumin contains a single Tryptophan residue in position 214 which is close to the drug site 1 and surrounded by positively charged amino acids. Because of this peculiarity, iodide quenching of the only Trp in human serum albumin is more complex and efficient. The positively charged amino acids attract the iodide ion increasing the quenching efficiency in the first part of the titration curve. Once this has reached saturation the quenching proceeds via pure collisional mechanism leading to a decrease in the steepness in the quenching curve (Chadborn et al, 1999). Any disruption of the charged Trp environment will affect the lodide quenching efficiency. Therefore, differences in the shape of the quenching curves can indicate a structural perturbation or the presence of a ligand, with negative charges, which are likely to be bound to the drug site 1.

Experimentally, a 2 M NaI stock solution was prepared in a volumetric flask in buffer (DPBS). The stock was further diluted to 0.1 M, always in a volumetric flask, for the titrations. The albumins were diluted at 0.2 mg/mL for the assay. The measurements were done in triplicate over the following NaI concentration range: 0 to 0.06 M. Tryptophan fluorescence was recorded for each solution in the 315-500 nm range (excitation wavelength=295 nm). Hunk (Unchained Labs, Woburn, MA, USA) was used for both sample preparation and fluorescence measurement (Gain 100/Slit 10). Data was plotted as:

$$[ NaI ] v s ( {F}_{351 nm} ( 0 ) / {F}_{351 n m} ( x ) )$$

Where F₃₅₁ nm (0) is the Tryptophan fluorescence in the absence of quencher and F_{351 nm} (×) is the fluorescence at [NaI]=x(M).

The Kₛᵥ (Stern-Vomer constant) was calculated using the Stern-Volmer equation:

$${F}_{351 n m} ( 0 ) / {F}_{351 n m} ( x ) = 1 + {{K}_{s v}}^{*} [ Q ] .$$

In the case of the albumin, only the first part of the plot was fitted to a line because of the non-linear relationship when using NaI as quencher.

All the plots, except for Rice 1, Rice 2 and Rice 3, present a downward curvature (FIG. 21). This trend has been observed in other studies (Insert Reference 18). This suggests that the charged environment of the Trp is similar for all tested albumins apart from for the three Rice-derived products. The Stern Volmer constants (Kₛᵥ) were calculated by fitting to a linear function the first part (10-15 points) of the quenching curves. The values obtained confirmed the increased quenching efficiency of iodide for all but the three Rice-derived albumins.

While lipid content is not necessarily disclosed by manufacturers, we think it should be considered as an additional quality parameter because of its likely impact on albumin performance and reproducibility. Albumin has at least seven fatty acids binding sites characterised by different affinities (Curry et al, 1999; Fasano et al, 2005). Five of the seven sites (FA1 to FA5) bind via both hydrophobic and ionic interactions with the polar head of the lipid. The last two binding sites seems not to involve polar interactions and are likely to be characterised by a weaker binding. Binding of fatty acids to albumin causes, in general, a conformational change which could affect indirectly also the accessibility of the Trp residue (Chadborn et al, 1999) involving rotation of domains (Curry et al, 1999; Fasano et al, 2005), but this was reported not to occur with the shorter fatty acids like octanoate (Kawai et al, 2017). Therefore, caution should be taken when analysing the effect of fatty acids and we aim to separately assess shorter chain FAs, usually added as an excipient, from longer chain FAs that may have more dramatic effects on the albumin structure.

The presence of fatty acids, both added (e.g. octanoate) and as impurities may influence the albumin characteristics. For example, it is known that fatty acids facilitate the Cys 34 oxidation (Gryzunov et al, 2003; Takabayashi et al, 1983) or affect ligand binding (Soltys et al, 1977). Therefore, the total amount of fatty acids (except for octanoate which may be present as an excipient and therefore disclosed by manufacturers) may be an important parameter to define the quality of an albumin.

Gas chromatography-mass spectrometry (GC-MS) was used to determine the amount of lipids in the albumin preparations. The method is based on the derivatisation of the Fatty Acids (FAS) with Fatty Acid Methyl Ester (FAME) prior to GC-MS analysis according to the protocol from Ichihara and Fukubayashi (2010). Following derivatisation, by using a specific temperature ramp, the MS was switched off after the last of the FAMEs of interest (C22) eluted. A 9-points calibration curve was used to quantify the FAs. Duplicate measurements were done for each sample. All the albumins were prepared at 100 mg/mL: the liquid albumins formulated at 200 mg/mL were diluted 1:1 in DPBS, while those formulated at 100 mg/ml were analysed without any dilution while the lyophilised samples were weighed, diluted in DPBS, and sterile filtered under a laminar flow hood through a 0.2 μm filter. The precise concentration of all the stocks was determined spectrophotometrically.

In FIG. 23, the total amount of fatty acids (except for octanoate) present in several albumins was reported. Notably, both the serum derived albumin and the Rice 3 albumin have a higher lipid content than Sacch. 1.1 and Pichia 1, both of which are derived from yeasts and showed higher performance.

In one of the correlation studies, we found the degree of formation of ThT positive aggregates correlated with the binding of the hydrophobic probe 8-anilino naphthalene sulfonic acid (ANS). This probe was historically used to detect the existence of kinetic or thermodynamic “molten globule” states (Ptitsyn et al, 1995) and is believed to interact mainly with exposed hydrophobic patches (but many studies have confirmed that the electrostatic component to the binding should not be ignored (Matulis et al, 1998). ANS in water has a maximum absorbance at 374 nm with an emission maximum around 520 nm characterized by a very low quantum yield. When ANS is bound to a protein there is both a shift of the fluorescence maximum and an increase of the fluorescence intensity. Generally, proteins in the native state do not bind ANS, but this is not the case for albumin. Albumin possesses two ANS binding sites with different affinities: a high affinity site which some references locate in subdomain III A (Kₐ=0.87×10⁶ M⁻¹ in one reference, Kₐ=6.3×10⁶ M⁻¹ in another reference) and a lower affinity site in domain II (Kₐ=0.079×10⁶ M⁻¹, in one reference and Kₐ=7.1×10⁵ M⁻¹ in another reference) leading to a biphasic titration curve (Yadav et al, 2013).

ANS binding to various albumins was investigated to see if differences were visible between sources. In the titration conditions used, we expected to see both high affinity and low affinity binding. Moreover, we were not in the approximation conditions where we could consider the Ligand_{free}~Ligand_{added} (conditions are Ligand concentration ~K_{d} and Protein concentration<<K_{d}). As such the K_{d} values obtained, if treated in the canonical way, represent a relative scale.

No differences in ANS binding were observed between Sacch. 1 batches (1.1, 1.2 and 1.3) and Sacch.2 batches (2.1, 2.2 and 2.3) but small differences were observed between Sacch. 1 and Sacch.2, which were expected because Sacch. 1 is formulated with octanoate, while Sacch.2 is not. Comparison of the K_{d} values showed that Rice 3 and Rice 2 have the lowest K_{d} (and therefore tighter binding), followed by Pichia 2 and Sacch.2.2. The other two Sacch.2 batches were in a third group with Rice 1, Pichia 1 and the serum-derived albumin. It is interesting to note that Pichia 1, even though it contains similar levels of octanoate to Sacch. 1, has a lower K_{d} (FIG. 24). So, while the samples can be grouped between octanoate and non-octanoate containing albumins, some of the albumins fall outside this broad division indicating that non-specific ANS binding occurs (for example in Rice 2 and Rice 3). This might be explained by potential exposure of hydrophobic patches (which are a well-known site for non-specific ANS binding to proteins), and hence structural perturbation in these two rice albumins.

In summary, in this example we have given substantial evidence to support the hypothesis of a structural perturbation of some of the albumins leads to a decrease in their ability to stabilise AAV2.

### Example 10: Stabilisation of Albumin by Citrate

In a series of differential scanning calorimetry (DSC) experiments, the stability of albumin in various buffers was measured. It was found that citrate buffer stabilises albumin against thermal denaturation. The negative dependence of this stabilisation on the ionic strength suggests binding of citrate to albumin as a possible explanation for the stabilisation of albumin observed in citrate buffer. Indeed, if the binding is driven by electrostatic interactions, an increase of ionic strength will lead to a decrease of the strength of the interaction (FIG. 25). The highest stabilisation observed was with pure citrate buffer (FIG. 26) and both the onset of the transition as well as the transition temperature (Tₘ) were shifted towards higher temperatures. Moreover, the area underneath the curve was higher indicating a higher proportion of folded protein in these conditions.

The presence of octanoate in a formulation stabilises against thermal denaturation. To further demonstrate the ability of citrate to stabilise against thermal denaturation, an albumin not containing octanoate in the original formulation buffer was diluted in DPBS or Tris-citrate buffer (albumin concentration at 5 mg/mL). After two hours of incubation at 60° C., the albumin in DPBS showed an increased hydrodynamic radius (R_{H}) indicative of aggregation, whereas the albumin in Tris-citrate maintained a radius of 3.7 nm (close to the theoretical value for an albumin molecule) (FIG. 27).

To investigate the effect of citrate on albumin against other “non-thermal” denaturation, a longer stability study was carried out to measure the stability of Sacch. 1 formulated at 10 mg/ml by measuring various parameters. Citrate buffer at pH 6 lead to a significantly higher protein recovery following 40° C. storage in the accelerated stability study compared to phosphate buffer at pH 6 and pH 7 with a P-value of 0.0072 and 0.0001 respectively (FIG. 28). Citrate buffer at pH 6 also lead to a higher monomer recovery following 36 months storage at 5° and 25° C. compared to phosphate buffer at pH 6 and 7 (FIG. 29). The formulation in citrate was protected against deamidation (Table 4), N-terminal degradation (Table 5) and fragmentation (Table 6).

TABLE 4
Ammonia concentration measured at 36 months for long
term storage and 12 months for accelerated conditions

| | Ammonia | | Ammonia |
|---|---|---|---|
| | concentration (μM) | | concentration (μM) at |
| | at 36 months | | 12 months |
| | T = 5° C. | T = 25° C. | T = 40° C. |
| Citrate pH 6 | 62.3 | 50.2 | 84.4 |
| Phosphate pH 6 | 50.6 | 120.6 | 90.4 |
| Phosphate pH 7 | 44.9 | 65.6 | 111.2 |

TABLE 5
N-terminal degradation measured at 36 months for long term storage and 12 months
for accelerated conditions

| | % N-Terminal | | % N-Terminal | | % N-Terminal |
|---|---|---|---|---|---|
| | degradation | | degradation | | degradation |
| | (12 months) | | (36 months) | | (36 months) |
| Citrate | 6.2 | Citrate | <1% | Citrate | 10.5 |
| pH 6, 40° C. | | pH 6, 5° C. | | pH 6, 25° C. | |
| Phosphate | 34.5 | Phosphate | <1% | Phosphate | 14.0 |
| pH 6, 40° C. | | pH 6, 5° C. | | pH 6, 25° C. | |
| Phosphate | 68.6 | Phosphate | 2.3 | Phosphate | 39.7 |
| pH 7, 40° C. | | pH 7, 5° C. | | pH 7, 25° C. | |

TABLE 6
Fragmentation measured at 12 months for
accelerated conditions with Native PAGE
(≤0.02% (w/w) rHA limit)

| | % Fragments (w/w) |
|---|---|
| Citrate pH 6, 40° C. | 0.01 |
| Phosphate pH 6, 40° C. | 0.04 |
| Phosphate pH 7, 40° C. | 0.11 |

We propose that albumin binds citrate, and that this binding is the basis of the stabilisation. Citrate imparts a new property to albumin by providing an improved conformational stability leading to a widening of the range of virus stabilisation. Therefore, to optimise an albumin formulation, the components of the formulation should be chosen in such a way they do not adversely affect this binding. For example, in the formulation used in our experiments, Tris, at pH 6-7, is positively charged and, therefore, does not interfere with citrate binding (Tris pKa=8.1 at 25° C.). Citrate is a chelating agent and contains negative charges on a flexible backbone (single bonds). Note that citrate at pH 7 is outside the buffering range but it was tested for the AAV2 at this pH because, according to our hypothesis, in this range it is present mainly a triple negative ion.

From the crystallographic structure of citrate bound to albumin (PDB: 1TF0) we have highlighted those residues at 4 Å (angstrom) from Citrate. All residues selected are on helical structures and these are: Lys195, 199, Trp214, Arg218, 222, Ala291 Asp451. In an alpha helical structure, residues located at i and i+4 are on the same side of a helix and usually are engaged in h-bond (NH with CO). Therefore, the Lys 195 and 199 are two positively charged residues facing the same side of the helix as well as Arg218 and 222, creating in this way a suitable site for citrate binding.

By comparing citrate and octanoate binding sites (this last one is in Subdomain IIA-Sudlow Site I) we noticed that many of the albumin residues in close proximity to citrate (1TF0) are also engaged in octanoate binding (Kawai et al, 2017). The residues are reported in FIG. 30. The unfolding of HSA that occurs at elevated temperatures involves multiple steps: N←→E→I→U
N is the native form of the protein
E is the expanded form
I is an intermediate where Domain II is unfolded while Domain I is intact
U is the unfolded protein

Increasing the temperature to about 50° C. results in the reversible separation of Domains I and II (Soltys et al, 1977). Our hypothesis is that citrate, and structurally similar anions (anions with multiple negative charges on a flexible backbone), “staple” the helices leading to thermal stabilisation. This would explain the reduction in stability of the citrate formulations measured by DSC in the presence of salt: the increase in ionic strength decreases the strength of binding of anionic drug to the site.

The subdomain IIA cavity is an anion receptor with a localised positive charge to aid anion binding (Watanabe and Maruyama, 2016). This positive charge is irrespective of the overall charge of the albumin molecule at the given pH (albumin at pH 6-7).

To summarise:
Albumin binds to citrate and this binding appears to be a basis for stabilisation.
To optimise an albumin formulation, the components of the formulation should be chosen in such a way they do not adversely affect such binding.
Citrate imparts a new property to albumin widening the range of stabilisation and giving both conformational and chemical stability.

We believe that the distribution of negative charges on a flexible backbone is a pre-requisite for the observed improvement in stabilisation and therefore that the observed stabilisation will extend to other compounds with similar structural features to citrate, such as tartaric acid, α-ketoglutarate, succinate, aconitate, isocitrate, oxaloacetate, maleic acid, succinic acid, adenosine triphosphate or sodium triphosphate (FIG. 31).

Examples 11 to 16 describe preparation of components and techniques used to further investigate the influence of various factors on AAV stability:

Factors and components explored included the following, generally in the presence and absence of albumin:
pH (Example 17)
Tris/citrate/MgCl₂ concentration (Example 18)·
Different anions, i.e. citrate alternatives (Example 19)·
Different buffers, i.e. Tris alternatives (Example 20)
   Different combinations of the following excipients (Example 21):
   Sugars and sugar alcohols (Example 21a)
   Pluronic acid and EDTA (Example 21b)
   Amino acids (Example 21c)
   Phosphates, sulphates and chlorides (Example 21d)

The albumin used in Examples 11 to 21 was a recombinant mature sequence wt human albumin (SEQ ID NO: 2) produced in S. cerevisiae and is designated “Saccharomyces 1.4” (Sacch. 1.4) since it was a batch of the product designated Sacch. 1.1, 1.2 and 1.3 in earlier Examples. Throughout Examples 11 to 21, Sacch. 1.4 is referred to as “albumin”.

### Example 11: Preparations of Buffers for Example 14 and 15

### Example 11a—Base Buffer Preparation

A 5-times strength stock of base buffer was prepared by using 150 mM Tris, 50 mM Citrate, 25 mM MgCl₂ in laboratory grade water and pH adjusted to 6.8 using 1M NaOH or 1M HCl followed by filtration through a 0.45 μm membrane (Milli-Q). When needed, this buffer was diluted with albumin, other excipients, viral vector and laboratory grade water to make a final concentration of the base buffer of 30 mM Tris, 10 mM Citrate, 5 mM MgCl₂.

### Example 11b—Change of pH of Base Buffer

To investigate the effect of change of pH, 5 times strength base buffer was prepared as per Example 11a and then the pH adjusted between 4 to 8 using 1M NaOH or 1M HCl such that the final buffer pH was pH 4.0, 5.0, 6.0, 6.8, 7.5 and 8.0

### Example 11c—Concentration Variation of Tris, Citrate and MgCl₂ from Base Buffer

5-times strength stock buffers were made to investigate different concentrations of the main components as follows:
Buffer was prepared at pH 6.8, such that the buffer had no Tris. i.e, 50 mM citrate and 25 mM MgCl₂ were present in the buffer.
Buffer was prepared at pH 6.8, such that the buffer had high concentration of Tris i.e, 250 mM Tris, 50 mM citrate and 25 mM MgCl₂.
Buffer was prepared at pH 6.8, such that the buffer had low citrate. i.e, 150 mM Tris, 10 mM citrate and 25 mM MgCl₂.
Buffer was prepared at pH 6.8, such that the buffer had high citrate. i.e, 150 mM Tris, 500 mM citrate and 25 mM MgCl₂
Buffer was prepared at pH 6.8, such that the buffer had high MgCl₂ i.e, 150 mM Tris, 50 mM citrate and 50 mM MgCl₂
Buffer was prepared at pH 6.8, such that the buffer had no MgCl₂. i.e, 150 mM Tris, 50 mM citrate and no MgCl₂

### Example 11d—Citrate Alternatives

5-times strength stock buffers were made to investigate alternatives to citrate as follows:
a. Buffer was prepared at pH 6.8, such that the buffer had 150 mM Tris, 50 mM of citrate alternative and 25 mM of MgCl₂. Citrate alternatives were structural variants of citrate with negatively charged backbones such as succinate, fumarate, maleic acid, and oxaloacetic acid.
b. Buffer was prepared at pH 6.8, such that the buffer had 150 mM Tris, 50 mM of citrate alternative and 25 mM of MgCl₂. Citrate alternatives were negatively charged additives that were not derivatives of citric variants from the tricarboxylic acid (TCA) cycle such as adenosine triphosphate (ATP), glutamic acid (GA) and ethylenediaminetetraacetic acid (EDTA).

### Example 11e—Tris Alternatives

5-times strength stock buffers were made to investigate alternatives to the buffering component (Tris) in the base as follows:
a. Buffer was prepared at pH 6.8, such that the buffer had 150 mM of Tris alternative, 50 mM citrate and 50 mM MgCl₂. Tris alternatives were bis-trisphosphate (BTP) and 2-[4-(2-hydroxyethyl) piperazin-1-yl]ethane-sulfonic acid (HEPES).

### Example 11f—Excipients Stock Solutions

To investigate the addition of other excipients to the base buffer, high concentration stock solutions of the various excipients were made as follows:
a. 1M Stock solutions of excipients were prepared in filtered laboratory grade water and pH adjusted to 6.8. Excipients used were sodium sulphate, magnesium sulphate, sodium bisulphate, sodium chloride, mannitol, glycine, ammonium sulphate and sodium thiosulphate.
b. Stock solutions of 0.5M sodium phosphate dibasic, 0.9 M sucrose, 0.26M sodium-ethylenediaminetetraacetic acid (EDTA) were prepared in filtered laboratory grade water and pH adjusted to 6.8.
c. Stock solutions of 0.6M arginine, 0.6M aspartic acid, 0.1M glutamic acid, 0.1M histidine were prepared in filtered laboratory grade water and heating the solutions as per recommendation in Certificate of Analysis of each component. pH was adjusted to 6.8 using 1M NaOH or 1M HCl.
d. 0.01% Pluronic acid solution was prepared by diluting a 10% stock solution using laboratory grade filtered water.

These excipients were added to the samples at an appropriate amount such that when all additions were made, and the samples diluted, the excipients were at the desired concentration.

### Example 12: Preparation of Formulations for Examples 14 and 15

The following formulations were prepared prior to samples of them undergoing freeze/thaw cycles to assess the stabilising effect of each formulation (Example 13) through analysis as per Examples 14 (DLS based analysis) and 15 (Halo based analysis).

### Example 12a—Formulations Containing AAV2

Formulations containing AAV2 were prepared by diluting 1.9 e13 Vg/ml of AAV2 stock (Charles River, formerly Vigene) to 1.0e12 Vg/ml using base buffer and laboratory grade filtered (0.45 μm, Millipore) water such that base buffer was diluted to 1-times strength.

### Example 12b—Formulations Containing AAV2 and Albumin

Formulations containing AAV2 and albumin were prepared by diluting 1.9 e13 Vg/ml of AAV2 stock to 1.0e12 Vg/ml and albumin from 200 mg/ml to 1 mg/ml using base buffer and laboratory grade filtered (0.45 μm, Millipore) water such that base buffer was diluted to 1-times strength.

### Example 12c—pH Variants of Base Buffer

a. Formulations containing AAV2 and albumin were prepared by diluting 1.9 e13 Vg/ml of AAV2 stock to 1.0e12 Vg/ml and albumin from 200 mg/ml to 1 mg/ml using pH variants of the base buffer (see Example 11b) and laboratory grade filtered (0.45 μm, Millipore) water such that base buffer was diluted to 1-times strength. pH variations were pH 4.0, 5.0, 6.0, 6.8, 7.5, 8.0.
b. Formulations containing AAV2 without albumin were prepared by diluting 1.9 e13 Vg/ml of AAV2 stock to 1.0e12 Vg/ml using pH variants of base buffer (see Example 11b) and laboratory grade filtered (0.45 μm, Millipore) water such that base buffer was diluted to 1-times strength. pH variations were pH 4.0, 5.0, 6.0, 6.8, 7.5, 8.0.

### Example 12d—Tris, Citrate or MgCl₂ Concentration Variation

Formulations containing AAV2 and albumin were prepared in buffers containing Tris, citrate or MgCl₂ concentration variants (see Example 11c) by diluting buffer from 5-times strength to 1-times strength and AAV2 from 1.9 e13 Vg/ml to 1.0e12 Vg/ml and albumin from 200 mg/ml to 1 mg/ml using laboratory grade filtered (0.45 μm, Millipore) water. Buffers were prepared such that the final concentrations of buffer components were:
a. Buffer containing 10 mM citrate, 5 mM MgCl₂ but no Tris
b. Buffer containing 50 mM Tris, 10 mM citrate, 5 mM MgCl₂
c. Buffer containing 30 mM Tris, 2 mM citrate, 5 mM MgCl₂
d. Buffer containing 30 mM Tris, 100 mM citrate, 5 mM MgCl₂
e. Buffer containing 30 mM Tris, 10 mM citrate, 10 mM MgCl₂
f. Buffer containing 30 mM Tris, 10 mM citrate, 0 mM MgCl₂

### Example 12e—Citrate Alternatives

a. Formulations containing AAV2 and albumin were prepared in buffers containing citrate alternatives (see Example 11d) such that the 5-times strength buffer contained 150 mM Tris, 25 mM MgCl₂ and 50 mM of citrate alternative where the alternatives to citrate were succinate, fumarate, maleic acid, adenosine triphosphate, glutamic acid or oxaloacetic acid. Formulations were prepared by diluting buffer from 5-times strength to 1-times strength (30 mM Tris, 10 mM citrate alternative and 5 mM MgCl₂) and AAV2 from 1.9 e13 Vg/ml to 1.0e12 Vg/ml and albumin from 200 mg/ml to 1 mg/ml using laboratory grade filtered (0.45 μm, Millipore) water.
b. Formulations containing AAV2 were prepared without albumin in buffers containing citrate alternatives (see Example 11d) such that the 5-times strength buffer contained 150 mM Tris, 25 mM MgCl₂ and 50 mM of citrate alternative where the alternatives to citrate were succinate, fumarate, maleic acid, adenosine triphosphate, glutamic acid or oxaloacetic acid. Formulations were prepared by diluting buffer from 5-times strength to 1-times strength (30 mM Tris, 10 mM citrate alternative and 5 mM MgCl₂) and AAV2 from 1.9 e13 Vg/ml to 1.0e12 Vg/ml using laboratory grade filtered (0.45 μm, Millipore) water.

### Example 12f—Tris Alternatives

a. Formulations containing AAV2 and albumin were prepared in buffers containing Tris alternatives (see Example 11e) such that the 5-times strength buffer contained 50 mM citrate, 25 mM MgCl₂ and 150 mM of Tris alternative where the alternatives to Tris were 2-[4-(2-hydroxyethyl) piperazin-1-yl]ethane-sulfonic acid (HEPES) and bis-tris-phosphate (BTP). Formulations were prepared by diluting buffer from 5-times strength to 1-times strength (30 mM Tris alternative, 10 mM citrate and 5 mM MgCl₂) and AAV2 from 1.9 e13 Vg/ml to 1.0e12 Vg/ml and albumin from 200 mg/ml to 1 mg/ml using laboratory grade filtered (0.45 μm, Millipore) water.
b. Formulations containing AAV2 were prepared without albumin in buffers containing Tris alternatives (see Example 11e) such that the 5-times strength buffer contained 50 mM citrate, 25 mM MgCl₂ and 150 mM of Tris alternative where the alternatives to Tris were 2-[4-(2-hydroxyethyl) piperazin-1-yl]ethane-sulfonic acid (HEPES) and bis-tris-phosphate (BTP). Formulations were prepared by diluting buffer from 5-times strength to 1-times strength (30 mM Tris alternative, 10 mM citrate and 5 mM MgCl₂) and AAV2 from 1.9 e13 Vg/ml to 1.0e12 Vg/ml using laboratory grade filtered (0.45 μm, Millipore) water.

### Example 12g—Formulations with Excipients

a. Formulations containing AAV2, albumin, 5-times strength base buffer and excipients (see Example 11f) were prepared by diluting 1.9 e13 Vg/ml of AAV2 stock to 1.0e12 Vg/ml and albumin from 200 mg/ml to 1 mg/ml with base buffer and excipients. The formulations were diluted to the required molarities using laboratory grade filtered (0.45 μm, Millipore) water such that base buffer was diluted to 1-times strength (30 mM Tris, 10 mM Citrate and 5 mM MgCl₂).
   i. Stock solutions of sodium sulphate, sodium bisulphate, magnesium sulphate sodium chloride, mannitol, sucrose, aspartate, arginine, histidine, glycine and glutamic acid, ammonium sulphate and sodium thiosulphate were diluted with albumin and AAV2 to obtain 50 mM and 200 mM of excipients concentration within the formulation.
   ii. Stock solutions of sodium phosphate dibasic was diluted with albumin and AAV2 to obtain 10 mM and 200 mM of excipient concentration within the formulation.
   iii. Stock solution of EDTA was diluted to obtain 1 and 50 mM of excipient concentration in the formulation.
b. Formulations containing AAV2, albumin, were prepared in Pluronic acid solution so that AAV2 stock was diluted to 1.0e12 Vg/ml from 1.9 e13 Vg/ml and albumin from 200 mg/ml to 1 mg/ml and Pluronic acid to 0.01% from 10% in lab grade filtered (0.45 μm, Millipore) water.

### Example 13: Freeze Thaw Stress

In order to stress the samples and therefore assess which formulations conferred greater stability on the viral vector, the samples were subjected to freeze-thaw cycles. All formulations were transferred to Corning 384 well plates. Controls (AAV2 undiluted 1.03e13 Vg/ml, albumin 1 mg/ml), buffers (respective buffers) and tests (samples tested) were set in duplicate with 30 μl of sample in each well. The plate was covered with microplate sealing tape to protect against evaporation and cross contamination. Immediately after preparation of the formulations, a first measurement was done using dynamic light scattering (see Example 14). Subsequently, all formulations were subjected to freeze-thaw stress by incubation at −20° C. for 6 hours and thawing at room temperature for 3 hours, and DLS measurements were taken after each freeze-thaw cycle up to 10 freeze-thaw cycles. After the 10th freeze-thaw cycle, the samples were assessed using DLS as well as Halo Labs Aura GT System to measure subvisible particles (see Example 15).

### Example 14: Aggregation Analysis Using Dynamic Light Scattering (DLS)

DLS was used to identify aggregation behaviour of components present in the formulation by assessing their hydrodynamic radius (R_{H}). Hydrodynamic radius calculation is based on diffusivity of suspended particles in the solution and time dependent fluctuation of light scattered from suspended particles. DLS was used to identify differences in the R_{H} values of individual components in the formulation and whether the freeze-thaw stress had induced aggregation (higher R_{H}), or the presence of albumin had kept it under control. Any deviation from the base formulation's particle size measurement was used to assess the extent of stability of a particular formulation.

For this study, a Wyatt DLS plate reader (800-WPR3) was used with Dynamics version 8.20 software for data collection and analysis. Instrument parameters were set to collect data at a fixed temperature of 25° C., to get 3 scans per well along with 10 DLS acquisitions with an acquisition time of 10 sec.

For data analysis, both cumulant and regularisation methods were applied. The cumulant method is recommended for mono-disperse system such as stock solution of AAV2. The cumulant method provides mean values of the diffusion coefficient hence the hydrodynamic radius but not the distribution of diffusion coefficients.

With an increase in polydispersity over time for the base formulation and also formulations prepared with multiple components, additional analysis was required to calculate radius of hydration, R_{H}. Therefore, for non-monomodal distribution, number weighted regularization formula was used in addition to cumulant method. Individual peak range was set so that peaks represent certain particle sizes. Therefore, peaks 1, 2 and 3 represented particle sizes between 0 to 10 nm, 10-50 nm and 50-1000 nm, respectively. Particle sizes greater than 1000 nm were excluded from calculation as they were beyond the physical limits of DLS.

Analysis of mass percentage of AAV2 and aggregates in formulations containing albumin was carried out by subtracting mass of albumin from the overall mass calculation. The following formula was applied for these calculations.

$${M}^{⋆} = ( Mn / ΣM 2 \% , M 3 \% ) \times 100$$

Where Mn is the mass percentage of either AAV2 species or aggregates calculated by the dynamic software, M2 is mass percentage of AAV2 species and M3 is the mass percentage of aggregates.

Based on the mass % recovery for AAV2 species in formulations, a ranking system was created where the highest mass % was given rank 1 and so on.

### Example 15: Aggregation Analysis Using Halo Labs Aura GT System

Halo Labs Aura GT System (Aura GT, Halo Labs, Burlingame, CA, USA; S/N: 02-10073) was used to identify aggregates greater than >1 μm in the formulation by using Backgrounded Membrane Imaging (BMI) and Side Illumination Membrane Imaging (SIMI).

Formulations from the completed DLS Freeze-Thaw (FT) experiments were transferred from the DLS Aurora 384 Well Plates for measurement on the Halo Labs Aura GT System (Aura GT, Halo Labs, Burlingame, CA, USA; S/N: 02-10073). 30 μl of the samples, after 10 Freeze-Thaw cycles, were spotted, without dilution, onto the membrane of White Halo Labs Plates (Halo Labs). Background images of all plates were taken to subtract noise from the actual image.

Controls included all potential components such as air, Milli-Q Water S.F. 0.1 μm, all buffers, all excipients (see Example 11), and undiluted albumin (200 mg/ml). (S.F.=sterile filtered).

BMI and SIMI were used to analyse end point formulation samples, post DLS analysis. Particle Counts and Particle Coverage were taken from these readings. All samples were analysed in duplicate, and all controls were analysed in triplicate. Experiments were divided into two rounds with two plates analysed in each round.

Formulations containing albumin were made comprising 1 mg/ml of albumin and ~1E+12 of viral particles Vg/ml.

All samples and controls were aliquoted in 30 μl volumes from the DLS Aurora 384 Well Plates onto the wells of the White Plate membrane, the White Plate was then vacuumed using a vacuum manifold every nine samples, however viscous samples were vacuumed every two or three samples dependant on how many replicates were in that sample group, these were all then dried with the wicking paper. This practice was repeated until all samples were dispensed, and then the White Plate was vacuumed again and then dried by vacuuming on the wicking paper for at least 5 minutes or until every well was dry. Samples were then scanned using the Halo Labs Aura GT System, via the Particle Vue Software (Version 4.2) with the below parameters:

| Plate Details | Chosen Parameters |
|---|---|
| Dilution Factor | 1 |
| Volume (ul) | 30 |
| Reference | None |

After the initial analysis of the White Plate, the samples and controls were washed using Milli-Q Water S.F. 0.1 μm. Milli-Q Water S.F. 0.1 μm was Milli-Q Ultrapure water sterilised using Sartorius Minisart High Flow Hydrophilic PES 0.1 μm filters, by syringe-filtering into sterile 15 ml or 50 ml centrifuge tubes. 100 μl of Milli-Q Water S.F. 0.1 μm was used, in two 50 μl washing steps, this was done to prevent the overflow of the wells as the maximum capacity for a sample well is 50 μl. The White Plate was then vacuumed between wash steps, and then it was dried by vacuuming on the wicking paper for at least 5 minutes or until every well was dry. Samples were then scanned again using the Halo Labs Aura GT System, via the Particle Vue Software (Version 4.2) with the same parameters as previously stated.

Data was extracted from the Vue Software and presented in particles per millilitre (p/ml), all data analysed was from the post-wash step scan of the White Plate.

All data was processed by excluding any samples which were present in the Exclusion Table with a % Particle Coverage at 10% or above saturation, so all samples at Particle Coverage 9.9% and below were accepted into the data set.

BMI and SIMI data was extracted from the Vue Software via the Experiment Report and then this was processed by removing any samples which had a % Particle Coverage at 10% or above saturation. Then an average of the remaining BMI replicates, and an average of the remaining SIMI replicates were taken, and then these averages were subtracted from each other to generate BMI-SIMI data.

This BMI-SIMI data was then processed by a Fold Improvement calculation, all formulations were compared against a single standard sample, in this case the stock AAV2 sample was used as the standard sample, the calculation: stock AAV2 particle count/sample particle count was used to calculation the aggregation improvement between each sample.

The stock AAV2 sample was tested in both experimental rounds, as such the subsequent BMI-SIMI data from both rounds was averaged into one comparative value. This was used for the subsequent Fold Improvement calculation.

Samples with a Fold Improvement of greater than one (>1) were deemed to be better than the stock AAV2 samples, and samples with a Fold Improvement of less than or equal to one (≤1) were deemed to be the same or worse than the stock AAV2 samples.

These Fold Improvement values were then ranked based on positive improvement, with rank 1 being the most improved formulation. In these Examples, the “Fold Improvement value” is typically referred to as “normalised Halo data” “normalised aggregation score” or “Halo score”.

### Example 16: Selection Criteria

DLS data and Halo particle analysis results were ranked separately and then combined to generate an overall rank for the most stable formulation. Preference was given to the highest mass % recovery in any formulation, followed by normalised aggregation score from Halo analysis. For example,
A sample with a high mass percentage and high Halo score from normalisation (meaning lower aggregation) was given a high rank (where number 1 is the highest) and was considered as a very stable formulation.
If the formulation had lower mass percentage and low Halo score from normalisation (presence of large aggregates), it was considered as not a very stable formulation.
If the formulation had lower mass percentage and high Halo score from normalisation (presence of lesser number of large aggregates), it was considered as a mid-range stable formulation. In such mid-range formulations, judgment was made by looking at the overall results including R_{H}, mass percentage and aggregation behaviour.

If the formulation had higher mass percentage and low Halo score from normalisation (presence of large aggregates), it was considered as a not a stable formulation but still conferred protective effects for AAV2 species.

### Example 17: Variation in pH of Base Buffer

Formulations were prepared as described in Example 12c and analysed as explained in Examples 15 and 16. These formulations were tested for stable and non-stable interactions among the components by evaluation of aggregation behaviour by variation of formulation buffer composition. Data is provided in Table 7.

For example, AAV2 formulation prepared in base buffer (30 mM Tris, 10 mM Citrate 5 mM MgCl₂) was tested for its stability and aggregation behaviour at a range of pH values in the presence of albumin (as in Example 12b) as well as in the absence of albumin (as in Example 12a). The base buffer had a pH of 6.8 (“Control”), 51% mass % recovery for AAV2 species and aggregation score from Halo was 3.22. The rest of the formulations were compared against the values obtained for formulation prepared in base buffer (Table 7).
a. AAV2 formulation prepared in base buffer at pH5 in the presence of albumin-had R_{H} ~16 nm and higher mass % recovery of AAV2 species (68%) compared to AAV2 formulation prepared in base buffer at pH5 in the absence of albumin where only 5.47% AAV2 mass was recovered. Halo scores did not indicate presence of larger sized aggregates in the presence or absence (69.5 vs 100) of albumin. Although mass % recovery for AAV2 was lower for this formulation compared to others in the same group, it was considered as best in the group (rank 1) because it had a balance between AAV2 mass % recovery and aggregation control.
b. AAV2 formulation prepared in base buffer at pH4 in the presence of albumin-had R_{H} ~16 nm and higher mass % recovery of AAV2 species (72%) while AAV2 formulation prepared at pH 4 in the absence of albumin had only 37% of AAV2 species recovered from the freeze-thaw stress. Larger size aggregates were recorded by Halo in albumin-containing formulation (8 vs 64). AAV2 formulation prepared in base buffer with albumin at pH 4 was given a rank of 2 because it had high mass % recovery for AAV2 but aggregation was higher than the same formulation prepared at pH5.
c. AAV2 formulation with albumin in base buffer at pH7.5- had R_{H} ~16 nm and higher mass % recovery of AAV2 species (82%). Mass percentage for AAV2 was higher in the formulation with albumin at pH 7.5 (82%), than in the formulation without albumin at the same pH (14.6%). However, irrespective of the presence of albumin, both formulations had aggregates of larger size (>1 μm) with a Halo score of 1.44 and 1.28 in the presence and absence of albumin. Besides the presence of larger aggregates, higher mass % recovery made this formulation at pH 7.5 reasonably stable with a rank of 3.
d. AAV2 formulation with albumin at pH8-had 56% mass % recovery of AAV2 species while same formulation without albumin, only had 0.61% of AAV2 species present at the end of the ten freeze-thaw cycles. Halo detected larger aggregates formation in both formulations (AAV2 with albumin vs AAV2 without albumin; 30.0 vs 24.5). AAV2 formulation with albumin at pH 8 was given a rank of 4 because it had lowest mass % recovery for AAV2 but aggregation was lower than most of the formulations in the group. Nevertheless, AAV2 with albumin at pH8 would be considered as a reasonably stable formulation with a potential to improve with some adjustments, such as by testing the concentration variation of base buffer components (see Example 18) and/or by addition of one or more excipients (see Examples 21c and d).
e. AAV2 with albumin at pH6 had R_{H} ~19 nm and higher mass % recovery of AAV2 species (59%). AAV2 formulation without albumin, pH6.0, had a lower mass % recovery for AAV2 species (8.74%). The Halo score, however, shows the presence of larger aggregates in both formulations, irrespective of the presence or absence of albumin (2.57 and 5.41) in formulations, respectively. AAV2 with albumin at pH6 would be considered as a reasonably stable formulation with a rank of 5.

Overall, formulations of AAV2 prepared in base buffer at all pH ranges appeared to have potential to become a stable formulation in the presence of albumin as albumin-containing formulations had the highest mass % recovery. This supports a protective effect of albumin towards AAV2 species.

TABLE 7
Mass percentages for individual species for the formulation of AAV2 (1.0e12 Vg/ml)
at pH variation of base buffer with albumin (1 mg/ml) and without albumin in formulation.

| | | M % | Normalised | Overall | |
|---|---|---|---|---|---|
| | pH | AAV2 | Halo data | rank | Comments |
| With | 7.5 | 82 | 1.44 | 3 | Lowest Halo score, but highest |
| albumin | | | | | mass % recovery |
| | 4.0 | 72 | 7.79 | 2 | Reasonably high Halo score, |
| | | | | | reasonably high mass% recovery |
| | 5.0 | 68 | 69.47 | 1 | Highest Halo score, reasonably |
| | | | | | high mass% recovery |
| | 6.0 | 59 | 2.57 | 5 | Low Halo and mass% score |
| | 8.0 | 56 | 30.03 | 4 | High Halo score but mass% |
| | | | | | recovery is lowest in the group |
| Control | 6.8 | 51 | 3.22 | | |
| Without | 6.8 | 56 | 3.22 | | |
| albumin | 4.0 | 37 | 63.75 | | |
| | 7.5 | 14.6 | 1.28 | | |
| | 6.0 | 8.74 | 5.41 | | |
| | 5.0 | 5.47 | 100.09 | | |
| | 8.0 | 0.61 | 24.49 | | |

M %: mass percentage recovery of specific species;
SD, Standard deviation.
Control: AAV2 (1.0e12 Vg/ml), albumin (1 mg/ml) in 30 mM Tris, 10 mM Citrate and 5 mM MgCl₂, at pH 6.8

### Example 18: Tris/Citrate/MgCl₂ Concentration Variations

To identify the impact of concentration change of the base buffer component on the stability of AAV2, formulations were prepared as in Example 12d and analysed as in Examples 15 and 16. Data is provided in Table 8.
a. Among the citrate variants, the formulation containing 2 mM citrate, 30 mM Tris, 5 mM MgCl₂ at pH 6.8 appeared to be better than formulations containing either 10 or 100 mM citrate. This formulation (2 mM citrate) had a good R_{H} of ~13 nm and the highest mass % recovery (75%,) of AAV2 species at all FT time points compared to 10 mM (mass % recovery of 51%, R_{H}~19 nm) and 100 mM (mass % recovery of −58%, R_{H}~31 nm). However, aggregation was significantly lower for the 100 mM citrate-containing formulation, and therefore, a higher rank is given to the formulation with higher concentration of citrate than with lower concentration (Table 8).
b. Within Tris concentration variation, 50 mM Tris is the better with R_{H} of AAV2 14 nm and mass % recovery of 68% compared to the formulation containing no-Tris at all with R_{H} of AAV2 of 20 nm and 52% mass % recovery. However, the result for the formulation with no Tris was still high and comparable to that for the control, proving there is benefit even when no buffer is included within the base formulation.
C. Formulations containing no MgCl₂ (R_{H}~15 nm, M % 74%) or 10 mM MgCl₂ (R_{H}~13 nm, M %-84%) both appeared to be stable formulations with a normalised Halo data score of 74 and 40, respectively. It seems that other components of the buffer or albumin has an impact on control of aggregation behaviour of the formulation when there was no MgCl₂ present in the formulation.

TABLE 8
Mass percentage recoveries for individual species for the formulation of 1.0e12 Vg/ml AAV2
with 1 mg/ml albumin at pH 6.8 with variation in concentration of base buffer components

| Formulation variation | Tris | Citrate | MgCl₂ | M % | Normalised | Overall |
|---|---|---|---|---|---|---|
| (summary of variant) | (mM) | (mM) | (mM) | AAV2 | Halo data | Rank |
| AAV2-albumin-TC | 30 | 10 | 10 | 84 | 40.47 | 3 |
| (10 mM MgCl₂) | | | | | | |
| AAV2-albumin-TC | 30 | 2 | 5 | 75 | 3.57 | 5 |
| (2 mM Citrate) | | | | | | |
| AAV2-albumin-TC | 30 | 10 | — | 74 | 74.48 | 4 |
| (0 mM MgCl₂) | | | | | | |
| AAV2-albumin-TC | 50 | 10 | 5 | 68 | 300.35 | 1 |
| (50 mM TRIS) | | | | | | |
| AAV2-albumin-TC | 30 | 100 | 5 | 58 | 238.67 | 2 |
| (100 mM Citrate) | | | | | | |
| AAV2-albumin-TC | 0 | 10 | 5 | 52 | 51.14 | 6 |
| (0 mM Tris) | | | | | | |
| Control | 30 | 10 | 5 | 51 | 3.22 | |

*;
M %: mass percentage of specific species;
SD, Standard deviation.
Control: AAV2 (1.0e12 Vg/ml), albumin (1 mg/ml) in 30 mM Tris, 10 mM citrate and 5 mM MgCl₂, at pH 6.8

All formulations, prepared with variable concentrations of base buffer components remained stable after the freeze-thaw stress. Therefore, it can be assumed that at least the citrate concentration range (2-100 mM), Tris concentration range (0-50 mM) and MgCl₂ concentration range (0-10 mM), would stabilise the formulation.

### Example 19: Citrate Alternatives

Formulations were prepared as described in Example 12e to assess the effect of different anions on forming either stable or non-stable interactions among the components and hence stabilising the formulation. Analysis was performed as explained in Examples 15 and 16. Data is provided in Table 9.
a. Oxaloacetate (OAA) was the best amongst the citrate alternatives, in the presence and absence of albumin in the formulation with R_{H}~22, M %-74% and R_{H}~15 nm, M %-65% for AAV2 species, respectively. Halo score of 4.38 vs 121 was recorded for formulations with albumin and without albumin.
b. This was followed by EDTA (R_{H}~23 nm, M % 73), fumarate (R_{H}~20 nm, M % 73), succinate (R_{H}~21, M % 66), and maleic acid (R_{H}~24 nm, M % 61%)), all in the presence of albumin.
c. ATP and glutamic acid resulted in the lowest AAV2 mass % recovery which was less than the control (mass % 51 and R_{H}~19 nm) with size and mass % of R_{H}~40 nm, M % 26 R_{H}~21 nm, M % 46, respectively, both in the presence of albumin. The latter excipient had higher Halo score, hence less aggregation than the rest of the group, however, mass % recovery for AAV2 species was less than the control and therefore, it was assigned a lower rank.

Like citrate, the citrate alternatives such as OAA, fumarate, succinate and maleic acid are also derivatives of TCA cycle, having negative charged backbones with carbonyl groups present at both ends of the chain.

EDTA is commonly used to enhance the stability of different formulations by chelating metal ions that may catalyse degradation reactions. In the current situation, EDTA was only effective in the presence of albumin.

Overall, addition of albumin increased the mass % recovery for AAV2 in formulations containing oxaloacetate, fumarate, succinate, and maleic acid. We also observed that formulations containing ATP and EDTA had a better mass % recovery in the presence of albumin which supports albumin's contributions towards AAV2 species structural integrity. In the case where glutamic acid was present as an excipient, addition of albumin resulted in less mass % recovery of AAV2 species however, it also helped to reduce aggregation in the formulation.

TABLE 9
Mass percentage recoveries for individual species for the formulation of AAV2 (1.0e12 Vg/ml)
with albumin (1 mg/ml) and without albumin at pH 6.8 using citrate-alternatives in base buffer,
compared to control which contained 10 mM citrate in base buffer.

| | | Mass % | | |
|---|---|---|---|---|
| | Citrate-alterative | recovery | Normalised | Overall |
| | (10 mM) | of AAV2 | Halo data | Rank |
| With albumin | OAA | 74 | 4.38 | 1 |
| | Fumarate | 73 | 0.45 | 2 |
| | EDTA | 73 | 0.78 | 3 |
| | Succinate | 66 | 0.36 | 4 |
| | Maleic acid | 61 | 0 | 7 |
| | GA | 46 | 25.86 | 5 |
| | ATP | 26 | 6.43 | 6 |
| Control | — | 51 | 3.22 | |
| Without | Succinate | 67.29 | 0.81 | |
| albumin | OAA | 65 | 120.87 | |
| | Fumarate | 43.21 | 143.18 | |
| | GA | 69 | 2.38 | |
| | Maleic Acid | 26.07 | 0.61 | |
| | EDTA | 23.12 | 11.78 | |
| | ATP | 5.16 | 2.32 | |

M %: mass percentage recovery of specific species;
SD, Standard deviation.
Control: AAV2 (1.0e12 Vg/ml), albumin (1 mg/ml) in 30 mM Tris, 10 mM citrate and 5 mM MgCl₂, at pH 6.8

### Example 20: Tris Alternatives

Formulations were prepared as described in Example 12f and analysed as explained in Examples 15 and 16. Formulations were prepared in buffer with 10 mM citrate, 5 mM MgCl₂ and, instead of 30 mM Tris, they contained 30 mM of one of the Tris alternatives. These formulations were tested in the absence or presence of albumin. Data is provided in Table 10.
a. Formulations prepared in buffer using Tris alternative such as 30 mM of BTP or HEPES had overall good mass % recovery of viral vector species compared to formulations containing Tris in the same conditions.
b. In the presence of albumin, formulation of AAV2 with HEPES-Citrate-MgCl₂ had a similar mass % recovery 73% and better R_{H}, ~14 nm, compared to formulation with BTP-Citrate-MgCl₂ (M %-72%, R_{H}~21 nm).
c. Without albumin, AAV2 in HEPES based formulation had M % of 55% and R_{H}~ 14 nm and in BTP-Citrate-MgCl₂ had R_{H} of 17~nm, and M %-65%.

It was observed that:
albumin-containing formulations had better mass % recovery for AAV2 species, supporting a protective effect of albumin.
Nonetheless, aggregation was lower in non-albumin BTP formulation than others. HEPES and BTP were used as Tris alternatives because of their buffering capacity and pH range. The tested buffers had a net positive charge with a pKa higher than the pH of the formulation. HEPES pH range is between 6.8-8.2, pKa 7.48 at 25° C. and BTP 6.3-9.5, pKa 6.8, 9.0 at 25° C., respectively. Tris has pH range of 7.0-9.0 and pKa 8.06 at 25° C. As both alternative buffers, as well as Tris, also worked within the formulation, it can be assumed that any buffer with a pKa higher than the buffer pH would work.

TABLE 10
Mass percentage recoveries for individual species for the formulation of AAV2 (1.0e12 Vg/ml)
with albumin (1 mg/ml) at pH 6.8 using Tris alternative in base buffer.

| | | M % | Normalised | | Overall |
|---|---|---|---|---|---|
| | Tris-alternative | AAV2 | data | Halo | rank |
| With albumin | 30 mM BTP | 72 | 3.09 | | 1 |
| | 30 mM HEPES | 73 | 3.01 | | 1 |
| Control | | 51 | 3.22 | | |
| Without albumin | 30 mM BTP | 65 | 372.02 | | 1 |
| | 30 mM HEPES | 55 | 4.45 | | 2 |

M %: mass percentage recovery of specific species;
SD, Standard deviation.
Control: AAV2 (1.0e12 Vg/ml), albumin (1 mg/ml) in 30 mM Tris, 10 mM citrate and 5 mM MgCl₂, at pH 6.8
Note that in Table 10, for 30 mM BTP without albumin, the value for the normalised Halo data is surprisingly high. It is unclear whether this reflects an extremely low level of aggregates, or whether there is an anomaly with this data point.

### Example 21: Formulations with Excipients Using Albumin and Base Buffer at pH 6.8

Formulations were prepared as described in Example 12g and analysed as in Examples 15 and 16.

### Example 21a—Sugar, Sugar Alcohol

Both DLS and HALO data seemed to agree on aggregation behaviour of formulation of AAV2 prepared with albumin in Tris Citrate MgCl₂ at pH 6.8 in the presence of sugars and sugar alcohols. Data is provided in Table 11.
a. Sucrose at 50 and 200 mM showed 14 and 46 mass % of AAV2, respectively.
b. Mannitol showed a lower mass % than sucrose which was 12 and 21% at 50 and 200 mM, respectively. This means most of the mass would be present in the form of aggregates as indicated by normalised Halo score.

Results showed that the formulations containing excipients such as sugars and sugar alcohols did not control aggregation or stabilise AAV2 species. Also, the formulation prepared in base buffer and albumin and containing none of the excipients, performed better in terms of AAV2 stability and aggregation control.

TABLE 11
Mass percentage recoveries for individual species for the formulation of AAV2
(1.0e12 Vg/ml) with albumin (1 mg/ml) and sugar-based excipients at pH 6.8 in base buffer

| | M % | Normalised | |
|---|---|---|---|
| Excipient | AAV2 | Halo data | Overall rank |
| Control | 51 | 3.22 | |
| 200 mM Sucrose | 46 | 0.62 | 1 |
| 200 mM Mannitol | 21 | 15.28 | 2 |
| 50 mM Sucrose | 14 | 1.18 | 3 |
| 50 mM Mannitol | 12 | 2.47 | 4 |

M %: mass percentage recovery of specific species;
SD, Standard deviation.
Control: AAV2 (1.0e12 Vg/ml), albumin (1 mg/ml) in 30 mM Tris, 10 mM citrate and 5 mM MgCl₂, at pH 6.8

### Example 21b—Pluronic Acid and EDTA

Formulations were prepared as described in Example 12g and analysed as in Examples 15 and 16.
a. Formulation with 0.01% Pluronic acid performed better than formulation with EDTA, formulation with Pluronic acid showed R_{H}~12 nm and M %-74% of AAV2 species. Aggregation score from Halo indicated that there is less aggregation even after 10 freeze-thaw cycles at 20° C. (Table 12).
b. EDTA mass % recovery was similar to the sugar and sugar alcohol (see Example 21a, Table 11) where 1 and 50 mM of EDTA-containing formulation had the overall mass % recoveries of 32 and 7%, respectively (Table 12).

Formulations prepared in Pluronic acid had stable AAV2 species with reduced aggregation. The formulation containing EDTA as an excipient was not found to be effective to prevent aggregation of AAV2 species at both tested concentrations.

TABLE 12
Mass percentage recoveries for individual species for the formulation of AAV2 (1.0e12Vg/ml)
with albumin (1 mg/ml), Pluronic acid or EDTA based excipients at pH 6.8 in base buffer.

| | M % | Normalised | |
|---|---|---|---|
| Excipient | AAV2 | Halo data | Overall rank |
| 0.01% Pluronic acid | 74 | 38.47 | 1 |
| Control | 51 | 3.22 | |
| 1 mM EDTA | 32 | 5.59 | 2 |
| 50 mM EDTA | 7 | 8.07 | 3 |

M %: mass percentage recoveries of specific species;
SD, Standard deviation.
Control: AAV2 (1.0e12 Vg/ml), albumin (1 mg/ml) in 30 mM Tris, 10 mM citrate and 5 mM MgCl₂, at pH 6.8

### Example 21c: Amino Acids

Formulations were prepared as described in Example 12g and analysed as in Examples 15 and 16. Data is provided in Table 13.
a. Among the amino acids, 50 mM aspartate, was the best with an R_{H} ~18 nm for AAV2 species and M % of 79%. This formulation had the highest Halo score of 10.41 amongst the amino acid excipient group which shows that this formulation had the least aggregation and is the most stable in this group (Table 13)
b. Formulation with either 50 or 200 mM arginine seems to have the potential to maintain stability of the formulation where AAV2 species R_{H} were 22 nm and 14 nm and M % were 47 and 62, respectively. Lower Halo scores of 5.37 and 1.38 indicated that aggregation increased as the concentration of arginine increased in the formulation. Nevertheless, arginine is believed to be a useful stabilising excipient.
c. Similarly, a formulation containing 50 mM histidine had a low mass % recovery for AAV2 (34%), and aggregates seemed to be larger in size and mass. However, in this formulation very large aggregates were not detected by Halo which could mean that although the histidine containing formulation was aggregating, the aggregate sizes were smaller than 1 μm.
d. Formulation containing 50 mM glutamic acid had R_{H} ~26 nm and M %-74 for AAV2 species, respectively. However, this formulation indicated higher aggregate sizes in Halo and therefore, was excluded from Halo calculations. There is a possibility that aggregates greater than 1 μm were formed and hence were not physically detectable by DLS but were identified by Halo.
e. This was followed by 200 mM histidine (M % for AAV2 of 16), 200 mM glutamic acid (M % for AAV2 of 26) and 200 mM aspartate (M % for AAV2 of 19) where although R_{H} of AAV2 was apparently in the acceptable range (25, 27 and 21 nm, respectively), the mass % was lower than control and a higher aggregation (with >10% particle coverage) was detected by Halo.
f. Formulations containing 50 and 200 mM of glycine showed 5 and 10% of mass % recovery of AAV2, respectively, with both methods agreeing on higher aggregation and hence lower stability.

Formulations prepared with amino acids as excipients resulted in stable formulations depending on the identity of the amino acid and concentration. These included formulations prepared with 50 mM of aspartate, 50 mM glutamic acid and 200 mM arginine. Higher/lower concentrations of the same excipients did not confer the same stability for AAV2.

TABLE 13
Mass percentage recoveries (M %) for individual species for the formulation of AAV2
(1.0e12 Vg/ml) with albumin (1 mg/ml) and amino acid-based excipients at pH 6.8 in base buffer.

| | M % | Normalised | |
|---|---|---|---|
| Excipient | AAV2 | Halo data | Overall rank |
| 50 mM Aspartate | 79 | 10.41 | 1 |
| 50 mM Glutamic acid | 74 | 0 | 3 |
| 200 mM Arginine | 62 | 1.38 | 2 |
| Control | 51 | 3.22 | |
| 50 mM Arginine | 47 | 5.37 | 4 |
| 50 mM Histidine | 34 | 9.20 | 5 |
| 200 mM Glutamic acid | 26 | 0 | 6 |
| 200 mM Aspartate | 19 | 0 | 7 |
| 200 mM Histidine | 16 | 0 | 8 |
| 200 mM Glycine | 10 | 1 | 9 |
| 50 mM Glycine | 5 | 3.41 | 10 |

M %: mass percentage recoveries of specific species;
M % rank: mass% recovery within thegroup;
SD, Standard deviation
Control: AAV2 (1.0e12 Vg/ml), albumin (1 mg/ml) in 30 mM Tris, 10 mM citrate and 5 mM MgCl₂,at pH 6.8

### Example 21d: Phosphates, Sulphates and Chlorides

Formulations were prepared as described in Example 12g and analysed as in Examples 15 and 16. Data is provided in Table 14.
a. Sodium sulphate (200 mM) with R_{H} 13 nm and AAV2 mass % recovery of 98% was found to be the best within the group as well as amongst all formulations tested during this experiment. This was followed by 200 mM sodium chloride with mass % recovery for AAV2 species of 97%, R_{H} ~12 nm which is the second highest among all formulations tested. For these two formulations, minimum aggregation was detected by Halo. Therefore, sodium sulphate and sodium chloride were considered as the best excipients to stabilise AAV2 with albumin in Tris Citrate MgCl₂ at pH 6.8.
b. Ammonium sulphate (200 mM), R_{H}~18 nm, M % 86% and Halo score of 31.02 for AAV2 species, was also considered to be a stable formulation as agreed by the results obtained from both instruments.
c. 200 mM of sodium bisulphate, 50 mM sodium sulphate, 200 and 50 mM of magnesium sulphate had the mass % recovery for AAV2 species of 82, 84, 65, 65% and R_{H}~ 17 nm, 13 nm, 17 nm and 20 nm respectively. Nevertheless, these formulations showed larger aggregates in Halo. These would be considered as mid-range stable formulations where aggregation was detected but mass % recovery of AAV2 species was high and Halo score was not too low compared to control formulation.
d. For formulations containing 200 mM sodium thiosulphate, the DLS and Halo outcomes did not align with each other. This could be due to formation of aggregates in the presence of 200 mM sodium thiosulphate being greater than 1 μm i.e. that were too large to be detected by DLS. However, mass % recovery for the AAV2 species is 86%. There is a possibility that this formulation was protective of the viral vector species against the freeze-thaw stress but could not control formation of large size aggregates. Therefore, it was given a rank of 7 in the group instead of 4 as the ranking is based on best of both methods with major focus on AAV2 species i.e. mass % recovery followed by lower aggregation (normalised Halo data).
e. Additionally, with 10 mM sodium phosphate dibasic, Halo did not detect very large particle sizes but due to lower AAV2 mass % recovery (45%) it was not considered as one of the most stable formulations.

This is the best group of formulations where, most of the formulations had high mass % recovery of AAV2 species (Table 14). Most salts were effective to stabilise the formulation at the concentration range of 50-200 mM of sodium sulphate, ammonium sulphate, sodium thiosulphate, magnesium sulphate. A few salts were only effective at higher concentrations, e.g. 200 mM sodium chloride, 200 mM sodium bisulphate, 200 mM sodium phosphate dibasic.

TABLE 14
Mass percentage recoveries for individual species for the formulation of AAV2 (1.0e12 Vg/ml)
with albumin (1 mg/ml) and salt-based excipients at pH 6.8 in base buffer

| | M % | Normalised | |
|---|---|---|---|
| Excipient | AAV2 | Halo data | Overall rank |
| 200 mM Sodium sulphate | 98 | 108.22 | 1 |
| 200 mM Sodium chloride | 97 | 127.77 | 2 |
| 200 mM Ammonium sulphate | 86 | 31.02 | 3 |
| 200 mM Sodium thiosulphate | 86 | 2.57 | 7 |
| 50 mM Sodium sulphate | 84 | 5.96 | 4 |
| 200 mM Sodium bis sulphate | 82 | 4.80 | 5 |
| 200 mM Magnesium sulphate | 65 | 8.38 | 8 |
| 50 mM Magnesium sulphate | 65 | 6.73 | 9 |
| 200 mM Sodium phosphate dibasic | 65 | 65.55 | 6 |
| 50 mM Sodium thiosulphate | 59 | 4.66 | 10 |
| 50 mM Ammonium sulphate | 56 | 0 | 11 |
| Control | 51 | 3.22 | |
| 10 mM Sodium phosphate dibasic | 45 | 8.89 | 12 |
| 50 mM Sodium chloride | 38 | 1.29 | 13 |
| 50 mM Sodium bisulphate | 37 | 0 | 0 |

M %: mass percentage recovery of specific species;
M % rank: mass % recovery within the group;
SD, Standard deviation
Control: AAV2 (1.0e12 Vg/ml), albumin (1 mg/ml) in 30 mM Tris, 10 mM citrate and 5 mM MgCl₂, at pH 6.8

### PART B—REFERENCES

Abe K, Ikeda M, Ariumi Y, Dansako H, Kato N. Serum-free cell culture system supplemented with lipid-rich albumin for hepatitis C virus (strain O of genotype 1b) replication. Virus Res. 2007; 125(2):162-168;
Amirtharaj G J, Natarajan S K, Mukhopadhya A, Zachariah U G, Hegde S K, Kurian G, Balasubramanian K A, Ramachandran A. Fatty acids influence binding of cobalt to serum albumin in patients with fatty liver. Biochim Biophys Acta. 2008 May; 1782(5):349-54;
Awasthi, Saurabh & Nambiappan Thangavel, Saraswathi. (2016). Non-enzymatic glycation mediated structure function changes in proteins: Case of serum albumin. RSC Adv. 6. 10.1039/C₆RA08283A;
Bal W, Sokolowska M, Kurowska E, Faller P. Binding of transition metal ions to albumin: sites, affinities and rates. Biochim Biophys Acta. 2013 December; 1830(12):5444-55;
Barnett, Blindauer, Kassaar, Khazaipoul, Martin, Sadler, Stewart. Allosteric modulation of zinc speciation by fatty acids, Biochimica et Biophysica Acta (BBA)—General Subjects, Volume 1830, Issue 12, 2013, Pages 5456-5464;
Bedu-Addo et al, 2004 F. K. Understanding lyophilization formulation development. Pharm. Technol., 28, pp. 10-18;
Bovarnick M. R., Miller J. C., Snvder J. C., 1950. The influence of certain salts, amino acids, sugars, and proteins on the stability of rickettsiae. J. Bacteriol., 59, pp. 509-522;
Brandau D T, Jones L S, Wiethoff C M, Rexroad J, Middaugh C R. Thermal stability of vaccines. J Pharm Sci 2003; 92:218-231;
Celik E, Calik, P. Production of recombinant proteins by yeast cells. Biotechnology Advances. 2012; 30(5):1108-1118;
Chadborn N, Bryant J, Bain A J, O'Shea P. Ligand-dependent conformational equilibria of serum albumin revealed by tryptophan fluorescence quenching. Biophys J. 1999; 76(4):2198-2207;
Chan B, Dodsworth N, Woodrow J, Tucker A, Harris R. Site-specific N-terminal auto-degradation of human serum albumin. Eur J Biochem. 1995 Jan. 15; 227(1-2):524-8;
Chen M, Guo H, Liu Y, Zhang Q. Structural changes of human serum albumin induced by cadmium acetate. J Biochem Mol Toxicol. 2014 June; 28(6):281-7;
Clarkson B R, Schön A, Freire E. Conformational stability and self-association equilibrium in biologics. Drug Discov Today. 2016; 21(2):342-347;
Curry S, Brick P, Franks N P. Fatty acid binding to human serum albumin: new insights from crystallographic studies. Biochim Biophys Acta. 1999 Nov. 23; 1441(2-3):131-40;
Domingo E., Current Topics on Microbiology and Immunology Series, 2006, vol. Vol. 289 BerlinSpringer Verlag;
Fanali G, di MA, Trezza V, Marino M, Fasano M, et al. (2012) Human serum albumin: from bench to bedside. Mol Aspects Med 33:209-290;
Fasano M, Curry S, Terreno E, Galliano M, Fanali G, Narciso P, Notari S, Ascenzi P. The extraordinary ligand binding properties of human serum albumin. IUBMB Life. 2005 December; 57(12):787-96;
Frahm G E, Smith D G S, Kane A, Lorbetskie B, Cyr T D, et al. (2014) Determination of Supplier-to-Supplier and Lot-to-Lot Variability in Glycation of Recombinant Human Serum Albumin Expressed in Oryza sativa. PLOS ONE 9(10):e109893;
Gunduz Ergun B, Huccetogullari D, Ozturk S, Celik E, Calik P (2019). Established and Upcoming Yeast Expression Systems. Part of the book series: Methods in Molecular Biology ((MIMB, volume 1923))
Gryzunov Y A, Arroyo A, Vigne J L, Zhao Q, Tyurin V A, Hubel C A, Gandley R E, Vladimirov Y A, Taylor R N, Kagan V E. Binding of fatty acids facilitates oxidation of cysteine-34 and converts copper-albumin complexes from antioxidants to prooxidants. Arch Biochem Biophys. 2003 May 1; 413 (1):53-66;
Guler N, Abro S, Emanuele M, Iqbal O, Hoppensteadt D, Fareed J (2017) Clin Appl Thromb Hemost. 23(8):987-991;
Gum E T, Swanson R A, Alano C, Liu J, Hong S, Weinstein P R, Panter S S. Human serum albumin and its N-terminal tetrapeptide (DAHK) block oxidant-induced neuronal death. Stroke. 2004 February; 35(2):590-5;
Harrington, Michielin, Malvehy, Pezzani Grüter, Grove, Frauchiger, Dummer (2017) Onco Targets Ther. 2; 10:3867-3880;
Hosokawa, Seiji Hama, Koichi Mandai, Kazuaki Okuda, Shigemitsu Takashima, Hisao Tajiri, Kenji Eguchi, Yuji Heike (2002) Journal of Virological Methods Volume 103, Issue 2, Pages 191-199;
Ichihara K, Fukubayashi Y. Preparation of fatty acid methyl esters for gas-liquid chromatography. J Lipid Res. 2010 March; 51(3):635-40; Kawai A, Chuang V T G, Kouno Y, Yamasaki K, Miyamoto S, Anraku M, Otagiri M. Crystallographic analysis of the ternary complex of octanoate and N-acetyl-I-methionine with human serum albumin reveals the mode of their stabilizing interactions. Biochim Biophys Acta Proteins Proteom. 2017 August; 1865(8):979-984;
Kawakami A, Kubota K, Yamada N, Tagami U, Takehana K, Sonaka I, Suzuki E, Hirayama K. Identification and characterization of oxidized human serum albumin. A slight structural change impairs its ligand-binding and antioxidant functions. FEBS J. 2006 July; 273(14):3346-57;
Khurana R, et al. Mechanism of thioflavin T binding to amyloid fibrils. J Struct Biol. 2005 September; 151(3):229-38;
Kissmann J, Ausar S F, Foubert T F, Detzi E J, Vedvick T S, Middaugh C R (2008) Physical Stabilization of Norwalk Virus-Like Particles. 97(10), 4208-4218;
Kragh-Hansen et al, 2002, Biol. Pharm. Bull. 25, 695;
Lovalenti, P. M. et al. Stabilization of live attenuated influenza vaccines by freeze drying, spray drying, and foam drying. Pharm. Res. 33, 1144-1160 (2016);
Maciążek-Jurczyk M, Szkudlarek S, Chudzik M, Pożycka J, Sułkowska A, Alteration of human serum albumin binding properties induced by modifications: A review, Spectrochimica Acta Part A: Molecular and Biomolecular Spectroscopy, Volume 188, 2018, Pages 675-683;
Matulis D, Lovrien R. 1-Anilino-8-naphthalene sulfonate anion-protein binding depends primarily on ion pair formation. Biophys J. 1998; 74(1):422-429;
Miyamura S, Imafuku T, Anraku M, Taguchi K, Yamasaki K, Tominaga Y, Maeda H, Ishima Y, Watanabe H, Otagiri M, Maruyama T (2016) Comparison of post-translational modification and the functional impairment of human serum albumin in commercial preparations. J Pharm Sci 105:1043-1049;
Needleman & Wunsch. A general method applicable to the search for similarities in the amino acid sequence of two proteins. J Mol Biol 48(3):443-453 (1970);
Neelofar, K. & Ahmad, J. An overview of in vitro and in vivo glycation of albumin: a potential disease marker in diabetes mellitus Glycoconj J (2017) 34:575;
Nielsen, Engelbrecht, Brunak, von Heijne. Identification of prokaryotic and eukaryotic signal peptides and prediction of their cleavage sites. Protein Engineering. 1997 January; 10(1):1-6;
Oettl, K. and Stauber, R. E. (2007), Physiological and pathological changes in the redox state of human serum albumin critically influence its binding properties. British Journal of Pharmacology, 151:580-590;
Ohtake, S. et al. Room temperature stabilization of oral, live attenuated Salmonella enterica serovar Typhi-vectored vaccines. Vaccine 29, 2761-71 (2011);
Petersen, Brunak, von Heijne, Nielsen. SignalP 4.0: discriminating signal peptides from transmembrane regions. Nat Methods 8(10):785-786 (2011);
Plotkin S A, Orenstein W A. Vaccines. Philadelphia: Saunders 2004;
Price P J, Evege E K. Serum-free medium without animal components for virus production. Focus. 1997; 19(3):67-69;
Ptitsyn O B. Molten globule and protein folding. Adv Protein Chem. 1995; 47:83-229; Rice, Longden, Bleasby. EMBOSS: the European Molecular Biology Open Software Suite. 2000, Trends Genet 16(6):276-277;
Roche M, Rondeau P, Singh N R, Tarnus E, Bourdon E. The antioxidant properties of serum albumin. FEBS Lett. 2008 Jun. 11; 582(13):1783-7;
Sancataldo G, Vetri V, Foderà V, Di Cara G, Militello V, et al. (2014) Oxidation Enhances Human Serum Albumin Thermal Stability and Changes the Routes of Amyloid Fibril Formation. PLOS ONE 9(1):e84552;
Shibata et al, 2009 Thermostabilization of the Neurotensin Receptor NTS1. J Mol Biol; Volume 390, Issue 2, 10 Jul. 2009, Pages 262-277;
Soltys B J, Hsia J C. Fatty acid enhancement of human serum albumin binding properties. A spin label study. J Biol Chem. 1977 Jun. 25; 252(12):4043-8;
Takabayashi K, Imada T, Saito Y, Inada Y. Coupling between fatty acid binding and sulfhydryl oxidation in bovine serum albumin. Eur J Biochem. 1983 Nov. 2; 136(2):291-5;
Thomas. The determination of log normal particle size distributions by dynamic light scattering. J Colloid and Interface Science. 117(1):187-192 (1987);
Watanabe, H.; Maruyama, T. Albumin as a biomarker. In Albumin in Medicine: Pathological and Clinical Applications; Otagiri, M., Chuang, V. T. G., Eds.; Springer: Singapore, 2016; pp. 51-69. ISBN 978-981-10-2116-9;
Wiedmann R T, Reisinger K S, Hartzel J et al. M-M-R(®) II manufactured using recombinant human albumin (rHA) and M-M-R (®) II manufactured using human serum albumin (HSA) exhibit similar safety and immunogenicity profiles when administered as a 2-dose regimen to healthy children. Vaccine. 2015; 33(18):2132-2140;
Yadav, Rajeev & Sen, Pratik. (2013). Mechanistic investigation of domain specific unfolding of human serum albumin and the effect of sucrose. Protein science: a publication of the Protein Society. 22. 10.1002/pro.2357;

1.-167. (canceled)

168. Use of a liquid formulation comprising:
(i) albumin; and
(ii) an anion having multiple negative charges housed on a flexible backbone, to stabilise a virus, wherein the albumin in the formulation is one that meets the following parameters:
the polydispersity index of albumin as assessed by dynamic light scattering is equal to or less than 0.05; and
less than 0.025% (w/w) of albumin in the formulation is in a polymeric form of higher order aggregates or multimers excluding dimers and trimers; and
wherein the stabilising of the virus is manifest by decreased aggregation of the virus when subjected to stress conditions selected from freeze-thaw cycles or incubating at a set temperature for a set period of time, compared to the level of aggregation of the virus in the absence of the formulation but otherwise under the same conditions.

169. The use according to claim 168, wherein the hydrodynamic radius of albumin as assessed by dynamic light scattering does not change more than 4.5% when incubated at 40° C. for a duration of 3 days.

170. The use according to claim 168 or 169, wherein the polymeric form of higher order aggregates or multimers excluding dimers and trimers elutes in the void volume of a gel permeation HPLC column which separates molecules in the molecular weight range 10,000 to 500,000 Da.

171. The use according to any one of the preceding claims, wherein the amount of albumin in a polymeric form is assessed by high-performance liquid size exclusion chromatography (SEC-HPLC).

172. The use according to any one of the preceding claims, wherein the albumin polymer level and polydispersity index parameters are observed within 6 months of formulation.

173. The use according to any one of claims 168-172, wherein the stabilising of the virus is manifest by decreased aggregation of the virus when subjected to freeze-thaw cycles wherein each cycle comprises storage at a temperature of around −20° C. for at least 6 hours followed by thawing at room temperature for at least 3 hours.

174. The use according to any one of claims 168-172, wherein the stabilising of the virus is manifest by decreased aggregation of the virus when subjected to a temperature of 40° C. for at least 20 hours.

175. The use according to claim 173 or 174, wherein the decreased aggregation is assessed by assessing the hydrodynamic radius of the formulation containing the virus by dynamic light scattering.

176. The use according to any one of the preceding claims, wherein the anion having multiple negative charges housed on a flexible backbone is not one of or both of glutamate and EDTA.

177. The use according to any one of the preceding claims, wherein the anion having multiple negative charges housed on a flexible backbone is not one of or both of glutamate and ATP.

178. The use according to any one of the preceding claims, wherein the anion having multiple negative charges housed on a flexible backbone is a multivalent carboxylic acid such as citrate, fumarate, tartaric acid, α-ketoglutarate, malate, maleic acid, succinate, succinic acid, aconitate, isocitrate, oxaloacetate, adenosine triphosphate or sodium tripolyphosphate or any intermediate anion of the citric acid cycle.

179. The use according to any one of the preceding claims, wherein the formulation is substantially free of a sugar and/or a sugar alcohol.

180. The use according to any one of the preceding claims, wherein the amount of albumin in a polymeric form is as assessed by high-performance liquid size exclusion chromatography (SEC-HPLC) using an injection sample of albumin provided at or diluted to less than 100 mg/mL.

181. The use according to any one of claims 168-180, wherein less than 75% of albumin molecules are post-translationally modified, by means other than oxidation.

182. The use according to any one of claims 168-181, wherein the total amount of lipids or fatty acids (excluding octanoate) in the formulation is equal to or less than 50 μg (microgram) lipids or fatty acids/mL of 100 mg/mL of albumin.

183. The use according to any one of the preceding claims, wherein the albumin is a recombinant albumin.

184. The use according to any one of claims 168-183, wherein the albumin is a yeast-derived albumin, optionally wherein the yeast is Pichia such as Pichia pastoris, Saccharomyces such as Saccharomyces cerevisiae, Candida, or Kluyveromyces such as Kluyveromyces lactis or Kluyveromyces marxianus, Hansenula polymorpha, Schizosaccharomyces pombe, Yarrowia lipolytica, Arxula adeninivorans, Candida such as Candida utilis) or Zygosaccharomyces bailii.

185. The use according to any one of claims 168-183, wherein the albumin is a plant-derived albumin such as a rice-derived albumin.

186. The use according to any one of the preceding claims, wherein the formulation comprises at least 0.5 moles free thiol per mole albumin.

187. The use according to any one of the preceding claims, wherein the albumin is provided by an albumin composition selected from the group consisting of any of:
a. a composition comprising 130 to 160 mM, preferably 145 mM, of sodium, 29 to 35 mM, preferably 32 mM, of octanoate, 10 to 20 mg/L, preferably 10 to 15 mg/L, of Polysorbate 80, 190 to 210 mg/mL, preferably 200 mg/mL, of albumin (w/v), wherein the pH is 6.7 to 7.3, preferably 7 and wherein the percentage of albumin (w/w) in a polymeric form is 0 to 1%, more preferably less than 0.025%;
b. a composition comprising 120 to 160 mM, preferably 145 mM, of sodium, 4 to 12 mM, preferably 8 mM, of octanoate, 0 to 50 mg/L, preferably 25 to 45 mg/L, of Polysorbate 80, 95 to 105 mg/mL, preferably 100 mg/mL, of albumin (w/v), wherein the pH is 6.4 to 7.4, preferably 7, and wherein the percentage of albumin (w/w) in a polymeric form is 0 to 1%, more preferably less than 0.025%;
c. a composition comprising 120 to 160 mM, preferably 145 mM, of sodium, 8 to 24 mM, preferably 16 mM, of octanoate, 0 to 100 mg/L, preferably 50 to 70 mg/L, of Polysorbate 80, 190 to 210 mg/mL, preferably 200 mg/mL, of albumin (w/v), wherein the pH is 6.4 to 7.4, preferably 7, and wherein the percentage of albumin (w/w) in a polymeric form is 0 to 1%, more preferably less than 0.025%;
d. a composition comprising 200 to 300 mM, preferably 230 to 260 mM of sodium, 0 to 3 mM, preferably 0 to 1 mM of octanoate, 95 to 105 mg/mL, preferably 100 mg/mL, of albumin (w/v), wherein the pH is 6 to 7, preferably 6.5, and wherein the percentage of albumin (w/w) in a polymeric form is 0 to 1%, preferably less than 0.025%.

188. The use according to any one of the preceding claims, wherein the formulation comprises from 0.4 to 300 mg/mL albumin, such as 0.4-50 mg/mL of albumin, optionally wherein the formulation comprises 1-20 mg/mL of albumin.

189. The use according to any one of the preceding claims, wherein the anion having multiple negative charges housed on a flexible backbone is one that thermally stabilises the albumin.

190. The use according to any one of the preceding claims, wherein the anion having multiple negative charges housed on a flexible backbone protects against deamidation of the albumin.

191. The use according to any one of the preceding claims, wherein the anion having multiple negative charges housed on a flexible backbone protects against N-terminal degradation of the albumin.

192. The use according to any one of the preceding claims, wherein the anion having multiple negative charges housed on a flexible backbone is present at a concentration of 2-500 mM or 10-500 mM, optionally wherein the anion is present at a concentration of 2-100 mM or 25-100 mM.

193. The use according to claim 192, wherein the anion is present at a concentration of 10-100 mM or 2-60 mM, 10-60 mM, 40-60 mM, such as around 2 mM, around 10 mM or around 50 mM.

194. The use according to any one of the preceding claims, wherein the formulation further comprises a buffer.

195. The use according to claim 194, wherein the buffer is one that maintains or increases the stability of the albumin in the presence of the anion having multiple negative charges housed on a flexible backbone.

196. The use according to claim 194 or 195, wherein the buffer is one that does not reduce the stability of the albumin in the presence of the anion having multiple negative charges housed on a flexible backbone.

197. The use according to any one of claims 194-196, wherein the buffer is positively charged.

198. The use according to any one of claims 194-197, wherein the buffer has a pKa higher than the pH of the formulation.

199. The use according to any one of claims 194-198, wherein the buffer is any one or more of Tris, Bis-Tris, Bis-Tris propane, BTP and/or HEPES.

200. The use according to any one of claims 194-199, wherein the formulation comprises 0-250 mM of buffer, such as 1-250 mM of buffer, optionally wherein the formulation comprises 10-200 mM of buffer.

201. The use according to claim 200, wherein the formulation comprises about 150 mM of buffer, optionally wherein the buffer is Tris, or wherein the formulation comprises about 0-50 mM such as about 30-50 mM of buffer, optionally wherein the buffer is any of Tris, BTP or HEPES.

202. The use according to any one of the preceding claims, wherein the formulation comprises 0-50 mM of magnesium chloride, optionally wherein the formulation comprises about 25 mM of magnesium chloride or about 5 to about 10 mM magnesium chloride, such as about 5 mM or about 10 mM magnesium chloride.

203. The use according to any one of the preceding claims, wherein:
(i) the formulation comprises 0-500 mM, such as 0-200 mM of sulfate salts, optionally wherein the formulation comprises about 50-500 mM of sulfate salts, such as about 50-200 mM, such as about 50 mM of sulfate salts or about 200 mM of sulfate salts, and further optionally wherein the sulfate salt is sodium sulfate, sodium bisulfate, sodium thiosulfate, ammonium sulfate, ammonium bisulfate, magnesium sulfate, magnesium bisulfate, potassium sulfate, potassium bisulfate; and/or
(ii) the formulation comprises 0-500 mM of disodium phosphate, such as 0-200 mM optionally wherein the formulation comprises about 10 mM of disodium phosphate or about 200 mM of disodium phosphate; and/or
(iii) the formulation comprises 0-50 mM of EDTA, optionally wherein the formulation comprises about 1 mM of EDTA; and/or
(iv) the formulation comprises 0-200 mM of glutamic acid, optionally wherein the formulation comprises about 50 mM of glutamic acid; and/or
(v) the formulation comprises 0-500 mM of mannitol, optionally wherein the formulation comprises about 300 mM of mannitol; and/or
(vi) the formulation comprises 0-500 mM of sucrose, optionally wherein the formulation comprises about 300 mM of sucrose; and/or
(vii) the formulation comprises 0-500 mM of sodium chloride, such as about 0-200 mM, optionally wherein the formulation comprises about 50-200 mM of sodium chloride, such as about 200 mM or about 50 mM sodium chloride.

204. The use according to any one of the preceding claims, wherein the formulation comprises 0-500 mM of arginine, such as about 0-200 mM, optionally wherein the formulation comprises about 25 mM to about 50 mM of arginine.

205. The use according to any one of the preceding claims, wherein the formulation comprises 0-500 mM such as 0-200 mM of chloride salts, optionally wherein the formulation comprises about 50 mM of chloride salts.

206. The use according to any one of the preceding claims, wherein the formulation comprises 0-200 mM of histidine, optionally wherein the formulation comprises about 50 mM of histidine.

207. The use according to any one of the preceding claims, wherein the formulation comprises 0-200 mM of glycine, optionally wherein the formulation comprises about 50 mM of glycine or about 200 mM of glycine.

208. The use according to any one of the preceding claims, wherein the formulation comprises 0-200 mM of glutamate, optionally wherein the formulation comprises about 50 mM of glutamate.

209. The use according to any one of the preceding claims, wherein the formulation comprises 0-200 mM of aspartate, optionally wherein the formulation comprises about 50 mM of aspartate.

210. The use according to any one of the preceding claims, wherein the formulation comprises 0-0.01% (w/v) of non-ionic surfactant, optionally wherein the formulation comprises about 0.001% or about 0.01% (w/v) of non-ionic surfactant, further optionally wherein the non-ionic surfactant is a poloxamer such as Pluronic F-68 or a polysorbate.

211. The use according to any one of the preceding claims, wherein the pH of the formulation is from about 4 to about 8, optionally wherein the pH of the formulation is from about 5 to about 7.5, preferably wherein the pH of the formulation is about 6 or about 6.5.

212. The use according to any one of the preceding claims, wherein the increased stability is manifest by the coefficient of variance of the hydrodynamic radius of the formulation containing the virus being not more than 5% when the formulation is incubated at 40° C. for a duration of at least 20 hours.

213. The use according to claim 212, wherein the increased stability is manifest by a particles coverage measured by backgrounded membrane imaging (BMI) of less than 15% after storage or incubation at a temperature of from 2 to 8° C., such as from 2, 3, 4, 5, 6 or 7 to 3, 4, 5, 6, 7 or 8° C. for a duration of at least 1 month, for example, at least 2, 3, 4, 5 or 6 months.

214. The use according to claim 213 wherein the increased stability by a particles coverage measured by backgrounded membrane imaging (BMI) of less than 15% after storage or incubation at a temperature of about 4° C. for a duration of at least 1 month, for example, at least 2, 3, 4, 5 or 6 months.

215. The use according to any one of the preceding claims, wherein the increased stability is manifest by a particle count of less than 60 000 particles measured by backgrounded membrane imaging (BMI) at after storage or incubation at a temperature of from 2 to 8° C., such as from 2, 3, 4, 5, 6 or 7 to 3, 4, 5, 6, 7 or 8° C., for a duration of at least 1 month, for example, at least 2, 3, 4, 5 or 6 months, wherein the estimated circular diameter (ECD) of the particle is equal to or less than 2 μm (microns).

216. The use according to claim 215, wherein the increased stability is manifest by a particle count of less than 60 000 particles measured by backgrounded membrane imaging (BMI) at after storage or incubation at a temperature of about 4° C. for a duration of at least 1 month, for example, at least 2, 3, 4, 5 or 6 months, wherein the estimated circular diameter (ECD) of the particle is equal to or less than 2 μm (microns).

217. The use according to any one of the preceding claims, wherein the increased stability is manifest by a particle count of less than 15 000 particles measured by backgrounded membrane imaging (BMI) after storage or incubation at a temperature of from 2 to 8° C. such as from 2, 3, 4, 5, 6 or 7 to 3, 4, 5, 6, 7 or 8° C., for example at about 4° C., for a duration of at least 1 month, for example, at least 2, 3, 4, 5 or 6 months, wherein the estimated circular diameter (ECD) of the particle is equal to or less than 2 μm (microns) and the particles are Thioflavin T-positive.

218. The use according to any one of the preceding claims, wherein the increased stability is manifest by a particle count of less than 200 000 particles in Brightfield-Side illumination membrane intensity (SIMI) measured by backgrounded membrane imaging after at least four rounds of freeze-thaw cycles, wherein the estimated circular diameter (ECD) of the particle is equal to or less than 2 μm (microns).

219. The use according to any one of the preceding claims, wherein the increased stability is manifest by the hydrodynamic radius of the formulation containing the virus being no more than 6× (preferably no more than 5.5×, 5×, 4.5×, 4×, 3.5×, 3×, 2.5×, 2×, 1.5×) the hydrodynamic radius of the virus in its monomeric form, after freeze-thaw stress (e.g. following 1, 2, 3, 4, 5, 6, 7, 8, 9 or 10 cycles of storing at a temperature of around −20° C. for at least 6 hours, followed by thawing at room temperature for about 3 hours).

220. The use according to any one of the preceding claims, wherein the increased stability is manifest by a mass percentage recovery for the virus of at least 50%, such as at least 55%, 60%, 65%, 70%, 75%, 80%, 85% 90%, 95%, 98% or 99%, after freeze-thaw stress (e.g. following 1, 2, 3, 4, 5, 6, 7, 8, 9 or 10 cycles of storing at a temperature of around −20° C. for at least 6 hours, followed by thawing at room temperature for about 3 hours).

221. The use according to any one of the preceding claims, wherein the increased stability is manifest by the mass percentage recovery for the virus being increased or improved by at least 1, 2, 3, 4, 5, 10, 15, 20, 25, 30, 35, 40, 45, or 50 percentage points relative to the mass percentage recovery of the virus in the absence of the formulation (e.g. the virus in an otherwise identical or substantially identical formulation but which contains no albumin and/or no anion having multiple negative charges housed on a flexible backbone), after freeze thaw stress (e.g. following 1, 2, 3, 4, 5, 6, 7, 8, 9, 10 cycles of storing at a temperature of around −20° C. for at least 6 hours, followed by thawing at room temperature for about 3 hours.

222. The use according to any one of the preceding claims, wherein the increased stability is manifest by the particle count of the formulation containing the virus being less than the particle count of a control solution of the virus (e.g. a solution of the virus in the absence of the albumin and/or in the absence of the anion having multiple negative charges housed on a flexible backbone) after freeze thaw stress (e.g. following 1, 2, 3, 4, 5, 6, 7, 8, 9, 10 cycles of storing at a temperature of around −20° C. for at least 6 hours, followed by thawing at room temperature for about 3 hours.

223. The use according to any one of the preceding claims, wherein the increased stability is manifest by retention of virus integrity.

224. The use according to any one of the preceding claims, wherein the increased stability is manifest by retention of the ability of the virus to bind to a specific binding partner of the virus.

225. The use according to any one of the preceding claims, wherein the increased stability is manifest by retention of the ability of the virus to infect a cell.

226. The use according to any one of the preceding claims, wherein the virus is (i) any of a viral particle, a viral fragment, a virus-like particle, a viral vector or a vector virus and/or (ii) a simian virus, optionally wherein the simian virus is an adeno-associated virus (AAV), a recombinant AAV, Adenovirus, Herpes Simplex Virus, vesicular stomatitis virus, Maraba virus, Vaccinia virus, Measles virus, Newcastle Disease Virus, Rotavirus, Reovirus, Type I Poliovirus, Coxsackievirus A21, Seneca Valley virus, Dengue virus, Yellow fever virus, West Nile virus, zika virus, Alphavirus or Coronavirus.

227. The use according to claim 226, wherein the AAV is AAV2, AAV9, AAV8 or AAV5.

228. The use according to any one of the preceding claims, wherein the virus is a live virus, an attenuated virus, a live-attenuated virus or an inactivated virus.

229. The use according to any one of the preceding claims, wherein the virus is in the form of a vaccine.

230. The use according to any one of the preceding claims, wherein the virus is a hybrid or chimeric virus.

231. The use according to any one of the preceding claims, wherein the virus that is stabilised is at a concentration of 1×10⁶-1×10²⁴ vg/mL.

232. A formulation comprising:
(i) albumin; and
(ii) an anion having multiple negative charges housed on a flexible backbone, wherein the albumin in the formulation is one that meets the following parameters:
the polydispersity index of albumin as assessed by dynamic light scattering is equal to or less than 0.05; and
less than 0.025% (w/w) of albumin is in a polymeric form of higher order aggregates or multimers excluding dimers and trimers.

233. A kit of parts comprising:
(i) albumin; and
(ii) an anion having multiple negative charges housed on a flexible backbone, wherein the albumin in the kit is one that meets the following parameters:
the polydispersity index of albumin as assessed by dynamic light scattering is equal to or less than 0.05; and
less than 0.025% (w/w) of albumin is in a polymeric form of higher order aggregates or multimers excluding dimers or trimers.

234. The formulation according to claim 232 or the kit according to claim 233, wherein the hydrodynamic radius of albumin does not change more than 4.5% when incubated at 40° C. for a duration of 3 days.

235. The formulation according to claim 232 or 234 or the kit according to claim 233 or 234, wherein the polymeric form of higher order aggregates or multimers excluding dimers and trimers elutes in the void volume of a gel permeation HPLC column which separates molecules in the molecular weight range 10,000 to 500,000 Da.

236. The formulation according to any one of claim 232, 234, or 235 or the kit according to any one of claims 233-235, wherein the amount of albumin in a polymeric form is assessed by high-performance liquid size exclusion chromatography (SEC-HPLC).

237. The formulation according to any one of claims 232 and 234-236 or the kit according to any one of claims 233-236, wherein the polymer level and polydispersity index parameters are observed within 6 months of formulation.

238. The formulation according to any one of claims 232 and 234-237 or the kit according to any one of claims 233-237, wherein the anion having multiple negative charges housed on a flexible backbone is not one of or both of glutamate and EDTA.

239. The formulation according to any one of claims 232 and 234-238 or the kit according to any one of claims 233-238, wherein the anion having multiple negative charges housed on a flexible backbone is not one of or both of glutamate and ATP.

240. The formulation according to any one of claims 232 and 234-239 or the kit according to any one of claims 233-239, wherein the anion having multiple negative charges housed on a flexible backbone is a multivalent carboxylic acid.

241. The formulation or kit according to claim 240, wherein the multivalent carboxylic acid is any of citrate, fumarate, tartaric acid, α-ketoglutarate, malate, maleic acid, succinate, succinic acid, aconitate, isocitrate, oxaloacetate, adenosine triphosphate or sodium tripolyphosphate or any intermediate anion of the citric acid cycle.

242. The formulation or kit according to claim 239 or 240, wherein the multivalent carboxylic acid is an intermediate anion of citric acid cycle.

243. The formulation of kit according to any one of claims 240-242, wherein the multivalent carboxylic acid is citrate.

244. The formulation according to any one of claims 232 and 234-243, or the kit according to any one of claims 233-243, wherein the kit or formulation is substantially free of a sugar and/or a sugar alcohol.

245. The formulation according to any one of claims 232 and 234-244, or the kit according to any one of claims 233-244, wherein the formulation or kit further comprises:
(iii) a buffer, wherein the buffer is one that maintains or increases the stability of the albumin in the presence of the anion having multiple negative charges housed on a flexible backbone.

246. The formulation according to any one of claims 232 and 234-244, or the kit according to any one of claims 233-244, wherein the formulation further comprises:
(iii) a buffer, wherein the buffer is one that does not reduce the stability of the albumin in the presence of the anion having multiple negative charges housed on a flexible backbone.

247. The formulation according to any one of claims 232 and 234-246, or the kit according to any one of claims 233-246, wherein the amount of albumin in a polymeric form is as assessed by high-performance liquid size exclusion chromatography (SEC-HPLC) using an injection sample of albumin provided at or diluted to less than 100 mg/mL.

248. The formulation according to any one of claims 232 and 234-247, or the kit according to any one of claims 233-27, wherein less than 75% of albumin molecules are post-translationally modified, by means other than oxidation.

249. The formulation according to any one of claims 232 and 234-248, or the kit according to any one of claims 233-248, wherein the total amount of lipids (excluding octanoate) is equal to or less than 50 μg (microgram) lipids/mL in a 100 mg/mL solution of albumin.

250. The formulation according to any one of claims 232 and 234-249, or the kit according to any one of claims 233-249, wherein the albumin is a recombinant albumin.

251. The formulation according to any one of claims 232 and 234-250, or the kit according to any one of claims 233-250, wherein the albumin is a yeast-derived albumin, optionally wherein the yeast is Pichia such as Pichia pastoris, Saccharomyces such as Saccharomyces cerevisiae, Candida, or Kluyveromyces such as Kluyveromyces lactis or Kluyveromyces marxianus, Hansenula polymorpha, Schizosaccharomyces pombe, Yarrowia lipolytica, Arxula adeninivorans, Candida such as Candida utilis or Zygosaccharomyces bailii.

252. The formulation according to any one of claims 232 and 234-251, or the kit according to any one of the claims 233-251, wherein the albumin is a plant-derived albumin such as a rice-derived albumin.

253. The formulation according to any one of claims 232 and 234-252, or the kit according to any one of claims 233-252, wherein the formulation or kit comprises at least 0.5 moles free thiol per mole albumin.

254. The formulation according to any one of claims 232 and 234-253, or the kit according to any one of claims 233-253, wherein the albumin is provided by an albumin composition selected from the group consisting of any of:
a. a composition comprising 130 to 160 mM, preferably 145 mM, of sodium, 29 to 35 mM, preferably 32 mM, of octanoate, 10 to 20 mg/L, preferably 10 to 15 mg/L, of Polysorbate 80, 190 to 210 mg/mL, preferably 200 mg/mL, of albumin (w/v), wherein the pH is 6.7 to 7.3, preferably 7 and wherein the percentage of albumin (w/w) in a polymeric form is 0 to 1%, more preferably less than 0.025%;
b. a composition comprising 120 to 160 mM, preferably 145 mM, of sodium, 4 to 12 mM, preferably 8 mM, of octanoate, 0 to 50 mg/L, preferably 25 to 45 mg/L, of Polysorbate 80, 95 to 105 mg/mL, preferably 100 mg/mL, of albumin (w/v), wherein the pH is 6.4 to 7.4, preferably 7, and wherein the percentage of albumin (w/w) in a polymeric form is 0 to 1%, more preferably less than 0.025%;
c. a composition comprising 120 to 160 mM, preferably 145 mM, of sodium, 8 to 24 mM, preferably 16 mM, of octanoate, 0 to 100 mg/L, preferably 50 to 70 mg/L, of Polysorbate 80, 190 to 210 mg/mL, preferably 200 mg/mL, of albumin (w/v), wherein the pH is 6.4 to 7.4, preferably 7, and wherein the percentage of albumin (w/w) in a polymeric form is 0 to 1%, more preferably less than 0.025%;
d. a composition comprising 200 to 300 mM preferably 230 to 260 mM of sodium, 0 to 3 mM, preferably 0 to 1 mM of octanoate, 95 to 105 mg/mL, preferably 100 mg/mL, of albumin (w/v), wherein the pH is 6 to 7, preferably 6.5, and wherein the percentage of albumin (w/w) in a polymeric form is 0 to 1%, preferably less than 0.025%.

255. The formulation according to any one of claims 232 and 234-254, or the kit according to any one of claims 233-254, wherein the formulation or kit comprises from 0.4 to 300 mg/mL albumin, such as from about 50 to 300 mg/mL or from about 100 to 200 mg/mL albumin, or from about 0.4 to 50 mg/mL of albumin, optionally wherein the formulation comprises 1-20 mg/mL of albumin.

256. The formulation according to any one of claims 232 and 234-255, or the kit according to any one of claims 233-255, wherein the anion having multiple negative charges housed on a flexible backbone is one that thermally stabilises the albumin.

257. The formulation according to any one of claims 232 and 234-256, or the kit according to any one of claims 233-256, wherein the anion having multiple negative charges housed on a flexible backbone protects against deamidation of the albumin.

258. The formulation according to any one of claims 232 and 234-257, or the kit according to any one of claims 233-257, wherein the anion having multiple negative charges housed on a flexible backbone protects against N-terminal degradation of the albumin.

259. The formulation according to any one of claims 232 and 234-258, or kit according to any one of claims 233-258, wherein the anion having multiple negative charges housed on a flexible backbone is present at a concentration of 2-500 mM or 10-500 mM, optionally wherein the anion is present at a concentration of 25-100 mM.

260. The formulation according to any one of claims 232 and 234-259, or kit according to any one of claims 233-259 wherein the anion is present at a concentration of 10-100 mM or 40-60 mM, such as around 50 mM.

261. The formulation or kit according to any one of claims 245-260, wherein the buffer is positively charged.

262. The formulation according to any one of claims 245-261, wherein the buffer has a pKa higher than the pH of the formulation, or the kit according to any one of claims 244-261 wherein the buffer has a pKa higher than the pH of parts (i) and/or (ii) of the kit.

263. The formulation or kit according to any one of claims 245-262, wherein the buffer is any one or more of Tris, Bis-Tris, Bis-Tris propane, BTP and/or HEPES.

264. The formulation or kit according to any one of claims 245-263, wherein the formulation or kit comprises 0-250 mM of buffer such as 1-250 mM of buffer, optionally wherein the formulation or kit comprises 10-200 mM of buffer.

265. The formulation or kit according to any one of claims 245-264, wherein the formulation or kit comprises about 150 mM of buffer, optionally wherein the buffer is Tris, or wherein the formulation or kit comprises about 0-50 mM of buffer such as about 30-50 mM of buffer, optionally wherein the buffer is any of Tris, BTP and/or HEPES.

266. The formulation according to any one of claims 232 and 234-265, or the kit according to any one of claims 233-265, wherein the formulation or kit comprises 0-50 mM of magnesium chloride, optionally wherein the formulation or kit comprises about 25 mM of magnesium chloride or about 1 to about 10 mM of magnesium chloride, such as about 5 mM or about 10 mM of magnesium chloride.

267. The formulation according to any one of claims 232 and 234-266, or the kit according to any one of claims 233-266, wherein:
(i) the formulation or kit comprises 0-500 mM such as 0-200 mM of sulfate salts, optionally wherein the formulation or kit comprises about 50-500 mM of sulfate salts, such as about 50-200 mM, such as about 50 mM sulfate salts or about 200 mM sulfate salts, and further optionally wherein the sulfate salt is sodium sulfate, sodium bisulfate, sodium thiosulfate, ammonium sulfate, ammonium bisulfate, magnesium sulfate, magnesium bisulfate, potassium sulfate, potassium bisulfate;
(ii) the formulation or kit comprises 0-500 mM such as 0-200 mM of disodium phosphate, optionally wherein the formulation or kit comprises about 10 mM of disodium phosphate or about 200 mM or about 500 mM disodium phosphate; and/or
(iii) the formulation or kit comprises 0-50 mM of EDTA, optionally wherein the formulation or kit comprises about 1 mM of EDTA; and/or
(iv) the formulation comprises 0-200 mM of glutamic acid, optionally wherein the formulation comprises about 50 mM of glutamic acid; and/or
(v) the formulation comprises 0-500 mM of mannitol, optionally wherein the formulation comprises about 300 mM of mannitol; and/or
(vi) the formulation comprises 0-500 mM of sucrose, optionally wherein the formulation comprises about 300 mM of sucrose, and/or
(vii) the formulation comprises 0-500 mM of sodium chloride, such as about 0-200 mM, optionally wherein the formulation comprises about 50-200 mM of sodium chloride, such as about 200 mM or about 50 mM sodium chloride.

268. The formulation according to any one of claims 232 and 234-267, or the kit according to any of one claims 233-267, wherein the formulation or kit comprises 0-500 mM of arginine, such as about 0-200 mM, optionally wherein the formulation or kit comprises about 25 mM to about 50 mM of arginine, or wherein the formulation or kit comprises about 200 mM of arginine.

269. The formulation according to any one of claims 232 and 234-268, or kit according to any one of claims 233-268, wherein the formulation or kit comprises 0-500 mM such as 0-200 mM of chloride salts, optionally wherein the formulation or kit comprises about 50 mM of chloride salts.

270. The formulation according to any one of claims 232 and 234-269, or kit according to any one of claims 233-269, wherein the formulation or kit comprises 0-200 mM of histidine, optionally wherein the formulation or kit comprises about 50 mM of histidine.

271. The formulation according to any one of claims 232 and 234-270, according to any one of claims 233-270, wherein the formulation or kit comprises 0-200 mM of glycine, optionally wherein the formulation or kit comprises about 50 mM of glycine or about 200 mM of glycine.

272. The formulation according to any one of claims 232 and 234-271, or kit according to any one of claims 233-271, wherein the formulation or kit comprises 0-200 mM of glutamate, optionally wherein the formulation or kit comprises about 50 mM of glutamate.

273. The formulation according to any one of claims 232 and 234-272, or kit according to any one of claims 233-272, wherein the formulation or kit comprises 0-200 mM of aspartate, optionally wherein the formulation or kit comprises about 50 mM of aspartate.

274. The formulation according to any one of claims 232 and 234-273, or kit according to any one of claims 233-273, wherein the formulation or kit comprises 0-0.01% (w/v) of non-ionic surfactant, optionally wherein the formulation or kit comprises about 0.001% or about 0.01% (w/v) of non-ionic surfactant, further optionally wherein the non-ionic surfactant is a poloxamer such as Pluronic F-68 or a polysorbate.

275. The formulation according to any one of claims 232 and 234-274, wherein the pH of the formulation is from about 4 to about 8, optionally wherein the pH of the formulation is from about 5 to about 7.5, or the kit according to any one of claims 233-274, wherein the pH of part (i) and/or (ii) of the kit is from about 4 to about 8, optionally wherein the pH of the formulation is from about 5 to about 7.5.

276. The formulation according to any one of claims 232 and 234-275, wherein the pH of the formulation is (a) about 6 or about 6.5 or (b) about 4 or about 5, or the kit according to any one of claims 233-275, wherein the pH of part (i) and/or (ii) of the kit is (a) about 6 or about 6.5 or (b) about 4 or about 5.

277. The formulation according to any one of claims 232 and 234-276, or kit according to any one of claims 233-276, wherein the formulation or kit further comprises a virus, optionally wherein the virus is any of a viral particle, a viral fragment, a virus-like particle, a viral vector or a vector virus.

278. The formulation or kit according to claim 277, wherein the virus is a simian virus, optionally wherein the simian virus is an adeno-associated virus (AAV), a recombinant AAV, Adenovirus, Herpes Simplex Virus, vesicular stomatitis virus, Maraba virus, Vaccinia virus, Measles virus, Newcastle Disease Virus, Rotavirus, Reovirus, Type I Poliovirus, Coxsackievirus A21, Seneca Valley virus, Dengue virus, Yellow fever virus, West Nile virus, zika virus, Alphavirus or Coronavirus.

279. The formulation or kit according to claim 278, wherein the AAV is AAV2, AAV9, AAV8 or AAV5.

280. The formulation or kit according to any one of claims 277-279, wherein the virus is a live virus, an attenuated virus, a live-attenuated virus, or an inactivated virus.

281. The formulation or kit according to any one of claims 277-280, wherein the virus is in the form of a vaccine.

282. The formulation or kit according to any one of claims 277-281, wherein the virus is a hybrid or chimeric virus.

283. The formulation or kit according to any one of claims 277-282, wherein the virus is present at a concentration of 1×10⁶-1×10²⁴ vg/ml.

284. A pharmaceutical composition comprising a formulation as defined in any one of claims 232 and 234-283, and a pharmaceutically excipient, carrier or diluent thereof.

285. A formulation according to any one of claims 232 and 234-283 or the kit according to any one of claims 233-283 for use in medicine.

286. A formulation or kit according to any one of claims 277-283 for use in gene therapy.

287. A formulation or kit according to any one of claims 277-283 for use in cell therapy.

288. A formulation or kit according to any one of claims 277-283 for use in vaccination and/or immunisation.

289. A formulation or kit according to any one of claims 277-283 for use in immunotherapy, optionally wherein the immunotherapy is oncolytic virotherapy.

290. A method of stabilising a virus comprising combining the virus with a formulation as defined in any one of claims 232 and 234-283 or with a pharmaceutical composition as defined in claim 284.

291. A method of selecting a suitable albumin for use in stabilising a virus, the method comprising determining whether the albumin meets the following parameters:
the polydispersity index of albumin as assessed by dynamic light scattering is equal to or less than 0.05; and
less than 0.025% (w/w) of albumin is in a polymeric form of higher order aggregates or multimers excluding dimers and trimers; and optionally—the hydrodynamic radius of albumin does not change more than 4.5% when incubated at 40° C. for a duration of 3 days;
wherein an albumin that meets the parameters is one that is useful in stabilising a virus, wherein the stabilising of the virus is manifest by decreased aggregation of the virus when subjected to stress conditions selected from freeze-thaw cycles or incubating at a set temperature for a set period of time.

292. The method of claim 291, wherein the method comprises:
(i) assessing whether there is less than 0.025% (w/w) of albumin in a polymeric form, wherein the assessment is performed by high-performance liquid chromatography; and/or
(ii) assessing whether the hydrodynamic radius of albumin does not change more than 4.5% when incubated at 40° C. for a duration of 3 days, optionally wherein the assessment is performed by dynamic light scattering; and/or
(iii) assessing whether the polydispersity index of albumin is equal to or less than 0.05.

293. The method of claim 291 or 292, wherein the method further comprises formulating the albumin with an anion having multiple negative charges housed on a flexible backbone, and assessing whether the formulation comprising the albumin and the anion stabilises a virus.

294. The method according to claim 293, wherein the anion having multiple negative charges housed on a flexible backbone is as defined in any one of claims 178, 189-193, 243 and 257-261.

295. A liquid formulation of albumin or fragment thereof comprising at least 0.75 moles free thiol per mole albumin or fragment thereof and at most 1% (w/w) albumin polymer after storage or incubation at at least 40° C. for at least 1, 2, 3, 4, 5 or 6 months.

296. A liquid formulation of albumin or fragment thereof comprising at least 0.75 moles free thiol per mole albumin or fragment thereof and at most 1% (w/w) albumin polymer after storage or incubation at at least 25° C. for at least 1, 2, 3, 4, 5, 6, 9 or 12 months.

297. A liquid formulation of albumin or fragment thereof comprising at least 0.75 moles free thiol per mole albumin and at most 1% (w/w) albumin polymer or fragment thereof after storage or incubation at a temperature of from 2 to 8° C., such as from 2, 3, 4, 5, 6, or 7 to 3, 4, 5, 6, 7, or 8° C., for example at about 4° C., for a duration of at least 1, 2, 3, 4, 5 or 6 months or at least 1, 2, 3, 4, 5 or 6 years.

298. The liquid formulation according to any of claims 295-297 comprising at least 0.80, 0.85, 0.90, 0.95, 0.96, 0.97, 0.98 or 0.99 moles free thiol per mole albumin or fragment thereof.

299. The liquid formulation according to claim 298 comprising at least 1 mole free thiol per mole albumin or fragment thereof.

300. The liquid formulation according to any of claims 295-299 comprising at least 75% of the theoretical free thiol level, more preferably at least 80%, 85%, 90%, 95%, 96%, 97%, 98% or 99% of the theoretical free thiol level.

301. The liquid formulation according to claim 300 comprising 100% of the theoretical free thiol level.

302. The liquid formulation according to any of claims 295-301 comprising from about 2.5 to about 7.5 mM fatty acid.

303. The liquid formulation according to claim 302 comprising from about 2.5, 3, 3.5, 4, 4.5, 5, 5.5, 6, 6.5, 7, 7.5 to about 3, 3.5, 4, 4.5, 5, 5.5, 6, 6.5, 7 or 7.5 mM fatty acid.

304. The liquid formulation according to claim 303 comprising from about 4 to about 6 mM fatty acid.

305. The liquid formulation according to claim 304 comprising about 5 mM fatty acid.

306. The liquid formulation according to any of claims 295-305 having a cation concentration of at least about 175 mM.

307. The liquid formulation according to claim 306 having a cation concentration of from, about 200, 225, 250, 275, or 300 mM to about 225, 250, 275, 300, 350, 400, 450, 500, 600, 700, 800, 900 or 1000 mM.

308. The liquid formulation according to claim 307 having a cation concentration of from, about 200 to about 300 mM.

309. The liquid formulation according to claim 308 having a cation concentration of from, about 225 to about 275 mM.

310. The liquid formulation according to any of claims 295-309 having a pH of from about 5.5 to about 6.5.

311. The liquid formulation according to claim 310 having a pH from about 5.5, 5.6, 5.7, 5.8, 5.9, 6.0, 6.1, 6.2, 6.3, 6.4 to about 5.6, 5.7, 5.8, 5.9, 6.0, 6.1, 6.2, 6.3, 6.4, or 6.5.

312. The liquid formulation according to claim 311 having a pH from about 5.7 to about 6.2.

313. The liquid formulation according to claim 312 having a pH about 6.

314. A liquid formulation comprising albumin, fatty acid and cation, in which the formulation lies within a design space defined by both of the following equations:
$$Free thiol = 1.1786 - 0.05167 * pH - 0.0001544 * cation - 0.01133 * FA + ( pH - 6 ) * ( ( cation - 198.3 ) * 0.0002659 ) + ( pH - 6 ) * ( ( FA - 7.125 ) * 0.00316 ) + ( cation - 198.3 ) * ( ( FA - 7.125 ) * 0.000001935 ) + ( pH - 6 ) * ( ( pH - 6 ) * - 0.05 ) + ( cation - 198.3 ) * ( ( cation - 198.3 ) * 0.000002583 ) + ( FA - 7.125 ) * ( ( FA - 7.125 ) * 0.0001413 ) + ( pH - 6 ) * ( ( cation - 198.3 ) * ( ( FA - 7.125 ) * - 0.000009879 ) ) , and \qquad ( a )$$
$$Polymer = 9.1677 - 0.8417 * pH - 0.01044 * cation - 0.269 * FA + ( pH - 6 ) * ( ( cation - 198.3 ) * - 0.004796 ) + ( pH - 6 ) * ( ( FA - 7.125 ) * 0.06523 ) + ( cation - 198.3 ) * ( ( FA - 7.125 ) * 0.001494 ) + ( pH - 6 ) * ( ( pH - 6 ) * 2.25 ) + ( cation - 198.3 ) * ( ( cation - 198.3 ) * 0.00006768 ) + ( FA - 7.125 ) * ( ( FA - 7.125 ) * 0.0249 ) + ( pH - 6 ) * ( ( cation - 198.3 ) * ( ( FA - 7.125 ) * 0.001237 ) ) ; \qquad ( b )$$
where
‘free thiol’ means the level of free thiol,
‘cation’ means the cation concentration in mM,
‘FA’ means the fatty acid concentration in mM.

315. The formulation according to claim 314 comprising at least 0.75 moles free thiol per mole albumin or fragment thereof and at most 1% (w/w) albumin polymer, for example after storage or incubation at at least 40° C. for at least 1, 2, 3, 4, 5 or 6 months.

316. The liquid formulation according to any of claims 295-315, in which the formulation lies within a design space defined by both of the following equations:
$$Free thiol = 1.1786 - 0.05167 * pH - 0.0001544 * cation - 0.01133 * FA + ( pH - 6 ) * ( ( cation - 198.3 ) * 0.0002659 ) + ( pH - 6 ) * ( ( FA - 7.125 ) * 0.00316 ) + ( cation - 198.3 ) * ( ( FA - 7.125 ) * 0.000001935 ) + ( pH - 6 ) * ( ( pH - 6 ) * - 0.05 ) + ( cation - 198.3 ) * ( ( cation - 198.3 ) * 0.000002583 ) + ( FA - 7.125 ) * ( ( FA - 7.125 ) * 0.0001413 ) + ( pH - 6 ) * ( ( cation - 198.3 ) * ( ( FA - 7.125 ) * - 0.000009879 ) ) , and \qquad ( a )$$
$$Polymer = 9.1677 - 0.8417 * pH - 0.01044 * cation - 0.269 * FA + ( pH - 6 ) * ( ( cation - 198.3 ) * - 0.004796 ) + ( pH - 6 ) * ( ( FA - 7.125 ) * 0.06523 ) + ( cation - 198.3 ) * ( ( FA - 7.125 ) * 0.001494 ) + ( pH - 6 ) * ( ( pH - 6 ) * 2.25 ) + ( cation - 198.3 ) * ( ( cation - 198.3 ) * 0.00006768 ) + ( FA - 7.125 ) * ( ( FA - 7.125 ) * 0.0249 ) + ( pH - 6 ) * ( ( cation - 198.3 ) * ( ( FA - 7.125 ) * 0.001237 ) ) ; \qquad ( b )$$
where
‘free thiol’ means the level of free thiol,
‘cation’ means the cation concentration in m,
‘FA’ means the fatty acid concentration in mM.

317. The liquid formulation according to any of claims 302 to 316 in which the fatty acid is selected from C₆ fatty acids or larger, such as C₆, C₈ or C₁₀ fatty acid, preferably C₇ fatty acids or larger, most preferred C₈ fatty acid (octanoate).

318. The liquid formulation according to any of claims 306 to 317 in which the cation is selected from sodium, potassium, calcium, magnesium and ammonium, preferably sodium.

319. The liquid formulation according to any of claims 295 to 318 comprising from about 225 to about 275 mM cation, preferably sodium, and from 4 to 6 mM fatty acid, preferably octanoate, and having a pH from about 5.7 to about 6.2.

320. The liquid formulation according to claim 319 wherein the formulation comprises about 250 mM cation, preferably sodium, about 5 mM fatty acid, preferably octanoate, and about pH 6.0.

321. The liquid formulation according to any of claims 295-320 in which the albumin or fragment thereof is fused to a partner.

322. The liquid formulation according to any of claims 295-321 in which the albumin, fragment or fusion thereof comprises at least 1, 2, 3, 4, 5, 6, 7, 8, 9 or 10 free thiols.

323. The liquid formulation according to any of claims 295-322 in which the free thiol or thiols are provided by a cysteine residue or residues at one or more (e.g. several) positions in albumin or fragment or fusion thereof.

324. The liquid formulation according to claim 323 in which the free thiol is provided by a cysteine at a position corresponding to one or more (e.g. several) of positions selected from 34, 1, 2, 4, 38, 40, 48, 52, 55, 58, 60, 75, 76, 79, 80, 82, 83, 83, 86, 91, 104, 113, 115, 116, 121, 122, 124, 125, 129, 168, 169, 177, 229, 236, 266, 269, 270, 273, 283, 298, 300, 301, 303, 304, 308, 313, 314, 316, 318, 320, 321, 324, 325, 355, 360, 361, 364, 365, 368, 369, 371, 375, 379, 386, 390, 396, 397, 435, 439, 443, 471, 478, 479, 490, 496, 498, 501, 503, 504, 505, 506, 508, 512, 538, 541, 542, 546, 549, 550, 558, 560, 562, 564, 565, 566, 567, 573, 574, 577, 578, 580, 581, 582, 584, and 585 of SEQ ID NO: 2.

325. The liquid formulation according to claim 324 in which the free thiol is provided by a cysteine at a position corresponding to position 34 of SEQ ID NO: 2.

326. The liquid formulation according to any of claims 295-325 in which the albumin or fragment thereof is selected from a mammalian albumin or fragment thereof, preferably from the group consisting of HSA (SEQ ID NO: 2), mouse (SEQ ID NO: 3), rat (SEQ ID NO: 4), macaque (SEQ ID NO: 5), bovine (SEQ ID NO: 6), pig (SEQ ID NO: 7), horse (SEQ ID NO: 8), or rabbit (SEQ ID NO: 9).

327. The liquid formulation according to any of claims 295-326 in which the albumin or fragment or fusion thereof has at least 70% sequence identity to SEQ ID NO: 2.

328. The liquid formulation according to claim 327 in which the albumin or fragment or fusion thereof has at least 75, 80, 85, 90, 91, 92, 93, 94, 95, 96, 97, 98 or 99% sequence identity to SEQ ID NO: 2.

329. The liquid formulation according to claim 327 or 328 in which the albumin or fragment or fusion thereof has from 1, 2, 3, 4, 5, 6, 7, 8 or 9 to 2, 3, 4, 5, 6, 7, 8, 9 or 10 amino acid differences compared with SEQ ID NO: 2.

330. The liquid formulation according to claim 329 in which the albumin or fragment or fusion thereof comprises SEQ ID NO: 2.

331. The liquid formulation according to any of claims 295-330 in which the albumin fragment comprises at least 175 amino acids.

332. The liquid formulation according to any of claims 295-331 in which the albumin fragment comprises at least 200, 225, 250, 275, 300, 325, 350, 375, 400, 425, 450, 475, 500, 525, 500 or 575 amino acids.

333. The liquid formulation according to any of claims 295-332 in which the albumin fragment comprises at least one (e.g. several) of Domain I, Domain II or Domain III of HSA (SEQ ID NO: 2).

334. The liquid formulation according to any of claims 295-333 in which the albumin, fragment or fusion thereof is recombinant.

335. The liquid formulation according to any of claims 295-334 in which the albumin, fragment or fusion thereof is produced in a eukaryotic host or prokaryotic host.

336. The liquid formulation according to claim 335 in which the eukaryotic host is selected from fungus, plant and mammal.

337. The liquid formulation according to claim 336 in which the fungus is selected from Aspergillus, Kluyveromyces, Pichia (e.g. Pichia pastoris) and Saccharomyces (e.g. Saccharomyces cerevisiae).

338. The liquid formulation according to claim 337 in which the Saccharomyces is Saccharomyces cerevisiae.

339. The liquid formulation according to claim 336 in which the plant is selected from potato, tobacco and rice.

340. The liquid formulation according to claim 339 in which the rice is Oryza sativa.

341. The liquid formulation according to claim 336 in which the mammal is a mammalian cell.

342. The liquid formulation according to claim 341 in which the mammalian cell is CHO or HEK.

343. The liquid formulation according to any of claims 295-342 in which the albumin is from human or animal serum.

344. The liquid formulation according to any of claims 295-343 in which the albumin or fragment or fusion thereof is present at from 5 to 30% (w/v), preferably from 10 to 20% (w/v), most preferably about 20%.

345. The liquid formulation according to any of claims 295-344 in which the protein content of the formulation is at least 50% albumin or fragment or fusion thereof, more preferably at least 60%, 70%, 75%, 80%, 85%, 90%, 95%, 96%, 97%, 98%, 99%, 99.5% albumin or fragment or fusion thereof.

346. The liquid formulation according to any of claims 295-345 which is substantially free, or completely free, of protein that is not an albumin, fragment or fusion thereof.

347. The liquid formulation according to any of claims 295-346 in which the formulation is aqueous.

348. The liquid formulation according to any of claims 295-347 presented in a container.
